(19) United States
(12) Patent Application Publication
(10) Pub. No.: US 2010/0160403 A1
(43) Pub. Date: Jun. 24, 2010

(54) HCV NS3 PROTEASE INHIBITORS

(75) Inventors: John O. Link, San Francisco, CA (US); Randall W. Vivian, San Mateo, CA (US)

Correspondence Address: GILEAD SCIENCES INC, 333 LAKESIDE DR, FOSTER CITY, CA, 94404, US

(73) Assignee: Gilead Sciences, Inc., Foster City, CA (US)

(21) Appl. No.: 12/638,253

(22) Filed: Dec. 15, 2009

Related U.S. Application Data
(60) Provisional application No. 61/139,434, filed on Dec. 19, 2008.

(51) Int. Cl. A61K 31/407; C07D 498/08; C07D 498/18

(52) U.S. Cl. 514/410; 540/456

(57) ABSTRACT

The present invention relates to macrocyclic compounds of formula (Ia) that are useful as inhibitors of the hepatitis C virus (HCV) NS3 protease, their synthesis, and their use for treating or preventing HCV infections.

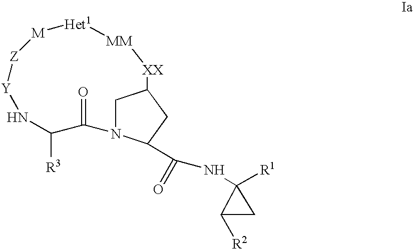

### CROSS-REFERENCE TO RELATED APPLICATIONS

This application claims priority to U.S. Provisional Application Ser. No. 61/139,434 filed Dec. 19, 2008. The contents of these provisional applications are herein incorporated by reference in their entirety for all purposes.

The present invention relates to macrocyclic compounds that are useful as inhibitors of the hepatitis C virus (HCV) NS3 protease, their synthesis, and their use for treating or preventing HCV infection.

### BACKGROUND OF THE INVENTION

Hepatitis C virus (HCV) infection is a major health problem that leads to chronic liver disease, such as cirrhosis and hepatocellular carcinoma, in a substantial number of infected people in the United States alone, according to the U S Center for Disease Control, roughly five times the number of people infected with the infected individuals, estimated to be 2-15% of the world's population. There are an estimated 3.9 million human immunodeficiency virus (HIV). According to the World Health Organization, there are more than 170 million infected individuals worldwide, with at least 3 to 4 million people being infected each year. Once infected, about 20% of people clear the virus, but the rest harbor HCV the rest of their lives. Ten to twenty percent of chronically infected individuals eventually develop liver-destroying cirrhosis or cancer. The viral disease is transmitted parenterally by contaminated blood and blood products, contaminated needles, or sexually and vertically from infected mothers or carrier mothers to their off-spring.

Current treatments for HCV infection, which are restricted to immunotherapy with recombinant interferon-a alone or in combination with the nucleoside analog ribavirin, are of limited clinical benefit. Moreover, there is no established vaccine for HCV. Consequently, there is an urgent need for improved therapeutic agents that effectively combat chronic HCV infection. The current state of the art in the treatment of HCV infection has been discussed in the following references: B. Dymock, et al., “Novel approaches to the treatment of hepatitis C virus infection,” Antiviral Chemistry & Chemotherapy, 11: 79-96 (2000); H. Rosen, et al., “Hepatitis C virus: current understanding and prospects for future therapies,” Molecular Medicine Today, 5: 393-399 (1999); D. Moradpour, et al, “Current and evolving therapies for hepatitis C,” European J. Gastroenterol. Hepatol., 11: 1189-1202 (1999); R Bartenschlager, “Candidate Targets for Hepatitis C Virus-Specific Antiviral Therapy,” Intervirology, 40: 378-393 (1997); G. M. Lauer and B. D Walker, “Hepatitis C Virus Infection,” N. Engl. J. Med., 345: 41-52 (2001); B. W. Dymock, “Emerging therapies for hepatitis C virus infection,” Emerging Drugs, 6: 13-42 (2001); and C. Crabb, “Hard-Won Advances Spark Excitement about Hepatitis C” Science: 506-507 (2001).

Several virally-encoded enzymes are putative targets for therapeutic intervention, including a metalloprotease (NS2-3), a serine protease (NS3), a helicase (NS3), and an RNA-dependent RNA polymerase (NS5B). The NS3 protease is located in the N-terminal domain of the NS3 protein, and is considered a prime drug target since it is responsible for an intramolecular cleavage at the NS3/4A site and for downstream intermolecular processing at the NS4A/4B, NS4B/5A and NS5A/5B junctions. Previous research has identified classes of peptides, such as hexapeptides as well as tripeptides discussed in U.S. patent applications US2005/0020503, US2004/0229818, and US2004/00229776, showing degrees of activity in inhibiting the NS3 protease. The aim of the present invention is to provide further compounds which exhibit activity against the HCV NS3 protease.

### SUMMARY OF THE INVENTION

The present invention relates to novel macrocyclic compounds of formula (I) and/or pharmaceutically acceptable salts or hydrates thereof. These compounds are useful in the inhibition of HCV (hepatitis C virus) NS3 (nonstructural 3) protease, the prevention or treatment of one or more of the symptoms of HCV infection, either as compounds or their pharmaceutically acceptable salts or hydrates (when appropriate), or as pharmaceutical composition ingredients, whether or not in combination with other HCV antivirals, anti-infectives, immunomodulators, antibiotics or vaccines. More particularly, the present invention relates to a compound of formula (Ia), (Ib) or (Ic) and/or a pharmaceutically acceptable salt or hydrate thereof:

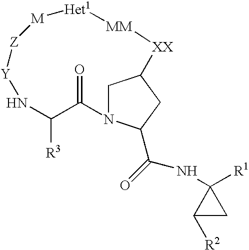

R¹ is:

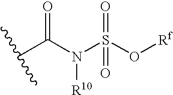

MM is CO or a bond;

XX is O, NH, N(C₁-C₄ alkyl), a bond or CH₂;

Het¹ is a heterocycle and can be substituted with up to ten groups selected independently from WW or R⁵;

R^{f} is A³;

each WW is independently H, halo, OR⁷⁷, C₁-C₆ alkyl, CN, CF₃, NO₂, SR⁷⁷, CO₂R⁷⁷, CON(R⁷⁷)₂, C(O)R⁷⁷, N(R¹⁰⁰)C(O)R⁷⁷, SO₂(C₁-C₆ alkyl), S(O)(C₁-C₆ alkyl), C₃-C₈ cycloalkyl, C₃-C₆ cycloalkoxy, C₁-C₆ haloalkyl, N(R⁷⁷)₂, NH(C1-C₆ alkyl)O(C₁-C₆ alkyl), halo(C₁-C₆ alkoxy), NR¹⁰⁰SO₂R⁷⁷, SO₂N(R⁷⁷)₂, NHCOOR⁷⁷, NHCONHR⁷⁷, aryl, heteroaryl or heterocyclyl; wherein aryl is phenyl or naphthyl, heteroaryl is a 5- or 6-membered aromatic ring having 1, 2 or 3 heteroatoms selected from N, 0 and S, attached through a ring carbon or nitrogen, and heterocyclyl is a 5- to 7-membered saturated or unsaturated nonaromatic ring having 1, 2, 3 or 4 heteroatoms selected from N, 0 and S, attached through a ring carbon or nitrogen; and wherein 2 adjacent WW moieties are optionally taken together with the atoms to which they are attached to form a 5- to 6-membered saturated, unsaturated non-aromatic, or aromatic cyclic ring having 0-2 heteroatoms selected from N, 0 and S;

A³ is independently selected from PRT, H, —OH, —C(O)OH, cyano, alkyl, alkenyl, alkynyl, amino, amido, imido, imino, halogen, CF₃, CH₂CF₃, cycloalkyl, nitro, aryl, aralkyl, alkoxy, aryloxy, heterocycle, —C(A²)₃, —C(A²)₂—C(O)A², C(O)A², —C(O)OA², —O(A²), —N(A²)₂, —S(A²), —CH₂P(Y¹)(A²)(OA²), —CH₂P(Y¹)(A²)(N(A²)₂), —CH₂P(Y¹)(OA²)(OA²), —OCH₂P(Y¹)(OA²)(OA²), —OCH₂P(Y¹)(A²)(OA²), —OCH₂P(Y¹)(A²)(N(A²)₂), —C(O)OCH₂P(Y¹)(OA²)(OA²), —C(O)CH₂P(Y¹)(A²)(OA²), —C(O)OCH₂P(Y¹)(A²)(N(A²)₂), —CH₂P(Y¹)(OA²)(N(A²)₂), —OCH₂P(Y¹)(OA²)(N(A²)₂), —C(O)OCH₂P(Y¹)(OA²)(N(A²)₂), —CH₂P(Y¹)(N(A²)₂)(N(A²)₂), C(O)OCH₂P(Y¹)(N(A²)₂)(N(A²)₂), —OCH₂P(Y¹)(N(A²)₂)(N(A²)₂), —(CH₂)ₘ-heterocycle, —(CH₂)ₘC(O)Oalkyl, —O—(CH₂)ₘ—O—C(O)—Oalkyl, —O—(CH₂)ᵣ—O—C(O)—(CH₂).-alkyl, —(CH₂)ₘO—C(O)—O-alkyl, —(CH₂)ₘ O—C(O)—O-cycloalkyl, —N(H)C(Me)C(O)O-alkyl, SRᵣ, S(O)Rᵣ, S(O)₂Rᵣ, or alkoxy arylsulfonamide, wherein each A³ may be optionally substituted with

1 to 4

—R¹¹¹, —P(Y¹)(OA²)(OA²), —P(Y¹)(OA²)(N(A²)₂), —P(Y¹)(A²)(OA²), —P(Y¹)(A²)(N(A²)₂), or P(Y¹)(N(A²)₂)(N(A²)₂), —C(═O)N(A²)₂), halogen, alkyl, alkenyl, alkynyl, aryl, carbocycle, heterocycle, aralkyl, aryl sulfonamide, aryl alkylsulfonamide, aryloxy sulfonamide, aryloxy alkylsulfonamide, aryloxy arylsulfonamide, alkyl sulfonamide, alkyloxy sulfonamide, alkyloxy alkylsulfonamide, arylthio, —(CH₂)ₘheterocycle, —(CH₂)ₘ—C(O)O-alkyl, —O(CH₂)ₘOC(O)Oalkyl, —O—(CH₂)ₘ—O—C(O)—(CH₂)ₘ-alkyl, —(CH₂)ₘ—O—C(O)-alkyl, —(CH₂)ₘ—O—C(O)—O-cycloalkyl, —N(H)C(CH₃)C(O)O-alkyl, or alkoxy arylsulfonamide, optionally substituted with R¹¹¹;

A² is independently selected from PRT, H, alkyl, alkenyl, alkynyl, amino, amino acid, alkoxy, aryloxy, cyano, haloalkyl, cycloalkyl, aryl, heteroaryl, heterocycle, alkylsulfonamide, or arylsulfonamide, wherein each A² is optionally substituted with A³;

R¹¹¹ is independently selected from H, alkyl, alkenyl, alkynyl, aryl, cycloalkyl, heterocycle, halogen, haloalkyl, alkylsulfonamido, arylsulfonamido, —C(O)NHS(O)₂—, or —S(O)₂—, optionally substituted with one or more A³;

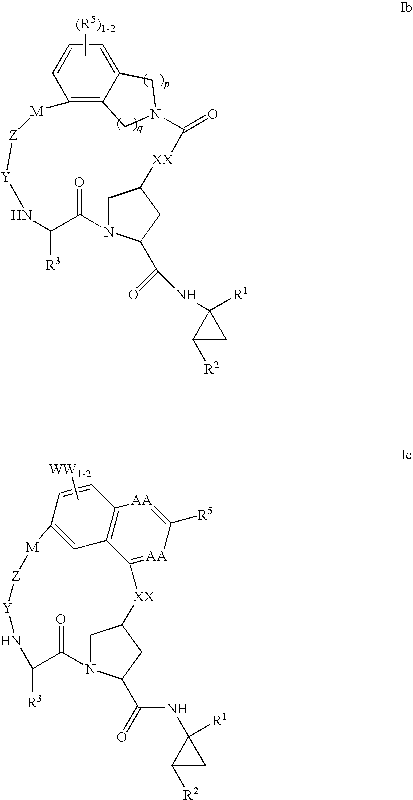

wherein:

R⁵⁵ is H, halo, OH, C₁-C₆ alkoxy, C₁-C₆ alkyl, CN, CF₃, SR¹⁰, SO₂(C₁-C₆ alkyl), C₃-C₆cycloalkyl, C₃-C₆ cycloalkoxy, C₃-C₆ haloalkyl, N(R⁷⁷)₂, aryl, heteroaryl or heterocyclyl; wherein aryl is phenyl or naphthyl, heteroaryl is a 5- or 6-membered aromatic ring having 1, 2 or 3 heteroatoms selected from N, 0 and S, attached through a ring carbon or nitrogen, and heterocyclyl is a 5- to 7-membered saturated or unsaturated non-aromatic ring having 1, 2, 3 or 4 heteroatoms selected from N, 0 and S, attached through a ring carbon or nitrogen; and wherein said aryl, heteroaryl, heterocyclyl, cycloalkyl, cycloalkoxy, alkyl or alkoxy is optionally substituted with 1 to 4 substituents selected from the group consisting of halo, OR¹⁰, SR¹⁰, N(R⁷⁷)₂, N(C₁-C₆ alky1)O(C₁-C₆ alkyl), C₁-C₆ alkyl, C₁-C₆ haloalkyl, halo(C₁-C₆ alkoxy), C₃-C₆ cycloalkyl, C₃-C₆ cycloalkoxy, NO₂, CN, CF₃, SO₂(C₁-C₆ alkyl), NR¹⁰⁰OSO₂R⁶, SO₂N(R⁶)₂, S(O)(C₁-C₆ alkyl), NHCOOR⁶, NHCOR⁶, NHCONHR⁶, CO₂R¹⁰, C(O)R¹⁰, and CON(R¹⁰)₂; wherein the 2 adjacent substituents of said cycloalkyl, cycloalkoxy, aryl, heteroaryl or heterocyclyl are optionally taken together to form a 3-6 membered cyclic ring containing 0-3 heteroatoms selected from N, 0 and S;

R⁶⁶ is C₁-C₆ alkyl, C₃-C₆ cycloalkyl, C₃-C₆ cycloalkyl(C₁-C₅)alkyl, aryl, aryl(C₁-C₄)alkyl, heteroaryl, heteroaryl(C₁-C₄ alkyl), heterocyclyl, or heterocyclyli(C₁-C₆ alkyl), wherein said alkyl, cycloalkyl, aryl, heteroaryl, or heterocyclyl is optionally substituted with 1 to 2 W′ substituents; and wherein each aryl is independently phenyl or naphthyl, each heteroaryl is independently a 5- or 6-membered aromatic ring having 1, 2 or 3 heteroatoms selected from N, 0 and S, attached through a ring carbon or nitrogen, and each heterocyclyl is independently a 5- to 7-membered saturated or unsaturated non-aromatic ring having 1, 2, 3 or 4 heteroatoms selected from N, 0 and S, attached through a ring.carbon or nitrogen;

AA is C(R¹¹⁰) or N;

when R⁵⁵ is other than H, R¹⁰⁰ is H, C₁-C₆ alkyl, halo, OR¹⁰⁰, SR¹⁰⁰, or N(R¹⁰⁰)₂;

when R⁵⁵ is H, R¹⁰⁰ is H, C₁-C₆ alkyl, halo, OH, C₁-C₆ alkoxy, CN, CF₃, SR¹⁰⁰, SO₂(C₁-C₆ alkyl), C₃-C₈ cycloalkyl, C₃-C₈ cycloalkoxy, C₁-C₆ halo alkyl, N(R⁷⁷)₂, aryl, heteroaryl or heterocyclyl; wherein aryl is phenyl or naphthyl, heteroaryl is a 5- or 6-membered aromatic ring having 1, 2 or 3 heteroatoms selected from N, 0 and S, attached through a ring carbon or nitrogen, and heterocyclyl is a 5- to 7-membered saturated or unsaturated non-aromatic ring having 1, 2, 3 or 4 heteroatoms selected from N, 0 and S, attached through a ring carbon or nitrogen; and wherein said aryl, heteroaryl, heterocyclyl, cycloalkyl, cycloalkoxy, alkyl or alkoxy is optionally substituted with 1 to 4 substituents selected from the group consisting of halo, OR¹⁰, SR¹⁰, N(R⁷⁷)₂, N(C₁-C₆ alky1)O(C₁-C₆ alkyl), C₁-C₆ alkyl, C₁-C₆ haloakryl, halo(C₁-C₆ alkoxy), C₃-C₆ cycloalkyl, C₃-C₆ cycloalkoxy, NO₂, CN, CF₃, SO₂(C₁-C₆ alkyl), NR¹⁰⁰SO₂R⁶⁶SO₂N(R⁶⁶)₂, S(O)(C₁-C₆ alkyl), NHCOOR⁶⁶, NHCOR⁶⁶, NHCONHR⁶⁶, CO₂R¹⁰⁰, C(O)R¹⁰⁰, and CON(R¹⁰⁰)₂; wherein the 2 adjacent substituents of said cycloalkyl, cycloalkoxy, aryl, heteroaryl or heterocyclyl are optionally taken together to form a 3-6 membered cyclic ring containing 0-3 heteroatoms selected from N, 0 and S;

or R⁵⁵ and R¹¹⁰ are optionally taken together to form a 5- to 6-membered saturated, unsaturated non-aromatic, or aromatic cyclic ring having 0-2 heteroatoms selected from N, O and S;

each R⁷⁷ is independently H, C₁-C₆ alkyl, C₃-C₆ cycloalkyl, C₃-C₆ cycloalkyl(C₁-C₈)alkyl, aryl, aryl(C₁-C₄)alkyl, heteroaryl, heteroaryl(C₁-C₄ alkyl), heterocyclyl, or heterocyclyl(C₁-C₆ alkyl), wherein said alkyl, cycloalkyl, aryl, heteroaryl, or heterocyclyl is optionally substituted with 1 to 2 W′ substituents; and wherein each aryl is independently phenyl or naphthyl, each heteroaryl is independently a 5- or 6-membered aromatic ring having 1, 2 or 3 heteroatoms selected from N, O and S, attached through a ring carbon or nitrogen, and each heterocyclyl is independently a 5- to 7-membered saturated or unsaturated non-aromatic ring having 1, 2, 3 or 4 heteroatoms selected from N, O and S, attached through a ring carbon or nitrogen;

each W′ is independently halo, OR¹⁰⁰, C₁-C₆ alkyl, CN, CF₃, NO₂, SR¹⁰⁰, CO₂R¹⁰⁰, CON(R¹⁰⁰)₂, C(O)R¹⁰⁰, N(R¹⁰⁰)C(O)R¹⁰⁰, SO₂(C₁-C₆), S(O)(C₁-C₆ alkyl), C₃-C₆ cycloalkyl, C₃-C₆cycloalkoxy, C₁-C₆ haloalkyl, N(R¹⁰⁰)₂, NH(C₁-C₆ alky1)O(C₁-C₆ alkyl), halo(C₁-C₆ alkoxy), NR¹⁰⁰SO₂R¹⁰⁰, SO₂N(R¹⁰⁰)₂, NHCOOR¹⁰⁰, NHCONHR¹⁰⁰, aryl, heteroaryl or heterocyclyl; wherein aryl is phenyl or naphthyl, heteroaryl is a 5- or 6-membered aromatic ring having 1, 2 or 3 heteroatoms selected from N, O and S, attached through a ring carbon or nitrogen, and heterocyclyl is a 5- to 7-membered saturated or unsaturated non-aromatic ring having 1, 2, 3 or 4 heteroatoms selected from N, O and S, attached through a ring carbon or nitrogen; and wherein 2 adjacent W′ moieties are optionally taken together with the atoms to which they are attached to form a 5- to 6-membered saturated, unsaturated non-aromatic, or aromatic cyclic ring having 0-2 heteroatoms selected from N, O and S;

In a specific embodiment of the invention R^{f} is H, alkyl, alkenyl, alkynyl, aryl, heteroaryl, or cycloalkyl, which R^{f} is optionally substituted with one or more R_{g};

each R_{g} is independently H, alkyl, alkenyl, alkynyl, halo, hydroxy, cyano, arylthio, cycloalkyl, aryl, heteroaryl, alkoxy, NRₕRᵢ, —C(═O)NRₕRᵢ, or —C(═O)OR_{d}, wherein each aryl and heteroaryl is optionally substituted with one or more alkyl, halo, hydroxy, cyano, nitro, amino, alkoxy, alkoxycarbonyl, alkanoyloxy, haloalkyl, or haloalkoxy; wherein each alkyl of R_{g} is optionally substituted with one or more halo, alkoxy, or cyano;

each Rₕ and Rᵢ is independently H, alkyl, or haloalkyl;

and

R_{d} and Rₑ are each independently H, (C₁₋₁₀)alkyl, or aryl, which is optionally substituted with one or more halo;

In a specific embodiment of the invention R^{f} is alkyl, aryl, cycloalkyl, which R^{f} is optionally substituted with one or more R_{g} independently selected from alkyl, halo, —C(═O)OR^{d}, or trifluoromethyl, wherein each alkyl of R^{g} is optionally substituted with one or more halo, alkoxy, or cyano.

In a specific embodiment of the invention R^{f} is aryl, heteroaryl, or cycloalkyl, which R^{f} is optionally substituted with one to three A³.

In a specific embodiment of the invention R^{f} is cyclopropyl which R^{f} is optionally substituted by up to four A³.

In a specific embodiment of the invention R^{f} is cyclopropyl which R^{f} is optionally substituted by one A³.

In a specific embodiment of the invention R^{f} is H, alkyl, alkenyl, alkynyl, aryl, heteroaryl, or cycloalkyl, which R^{f} is optionally substituted with one or more R_{g};

each R_{g} is independently H, alkyl, alkenyl, alkynyl, halo, hydroxy, cyano, arylthio, cycloalkyl, aryl, heteroaryl, alkoxy, NRₕRᵢ, —C(═O)NRₕRᵢ, or —C(═O)OR_{d}, wherein each aryl and heteroaryl is optionally substituted with one or more alkyl, halo, hydroxy, cyano, intro, amino, alkoxy, alkoxycarbonyl, alkanoyloxy, haloalkyl, or haloalkoxy; wherein each alkyl of R_{g} is optionally substituted with one or more halo or cyano; and each Rₕ and Rᵢ is independently H, alkyl, or haloalkyl.

In a specific embodiment of the invention R^{f} is H, alkyl, alkenyl, alkynyl, aryl, heteroaryl, or cycloalkyl, which R^{f} is optionally substituted with one or more R_{g};

each R_{g} is independently H, alkyl, alkenyl, alkynyl, halo, hydroxy, cyano, arylthio, cycloalkyl, aryl, heteroaryl, alkoxy, NRₕRᵢ, —C(═O)NRₕRᵢ, wherein each aryl and heteroaryl is optionally substituted with one or more alkyl, halo, hydroxy, cyano, nitro, amino, alkoxy, alkoxycarbonyl, alkanoyloxy, haloalkyl, or haloalkoxy; each Rₕ and Rᵢ is independently H, alkyl, or haloalkyl;

In a specific embodiment of the invention R^{f} is phenyl, cyclopropyl, 2-fluorophenyl, 4-chlorophenyl, 2-chlorophenyl, 2,6-dimethylphenyl, 2-methylphenyl, 2,2-dimethylpropyl, 2,2-difluoroethyl, 2,2,2-trifluoroethyl, or 1-methylcyclopropyl.

In a specific embodiment of the invention R^{f} is cyclopropyl.

In a specific embodiment of the invention R^{f} is 1-methylcyclopropyl.

A³ is independently selected from PRT, H, —OH, —C(O)OH, cyano, alkyl, alkenyl, alkynyl, amino, amido, imido, imino, halogen, CF₃, CH₂CF₃, cycloalkyl, nitro, aryl, aralkyl, alkoxy, aryloxy, heterocycle, —C(A²)₃, —C(A²)₂—C(O)A², —C(O)A², —C(O)OA², —O(A²), —N(A²)₂, —S(A²), —CH₂P(Y¹)(A²)(OA²), —CH₂P(Y¹)(A²)(N(A²)₂), —CH₂P(Y¹)(OA²)(OA²), —OCH₂P(Y¹)(OA²)(OA²), —OCH₂P(Y¹)(A²)(OA²), —OCH₂P(Y¹)(OA²)(N(A²)₂), —C(O)OCH₂P(Y¹)(OA²)(OA²), —C(O)OCH₂P(Y¹)(A²)(OA²), —C(O)OCH₂P(Y¹)(A²)(N(A²)₂), —CH₂P(Y¹)(OA²)(N(A²)₂), —OCH₂P(Y¹)(OA²)(N(A²)₂), C(O)OCH₂P(Y¹)(OA²)(N(A²)₂), —CH₂P(Y₁)(N(A²)₂)(N(A²)₂), —C(O)OCH₂P(Y¹)(N(A²)₂)(N(A²)₂), —OCH₂P(Y¹)(N(A²)₂)(N(A²)₂), —(CH₂)ₘ-heterocycle, —(CH₂)ₘC(O)Oalkyl, —O—(CH₂)ₘ—O—C(O)—Oalkyl, —O—(CH₂)ᵣ-—O—C(O)—(CH₂)ₘ-alkyl, —(CH₂)ₘO—C(O)—O-alkyl, —(CH₂)ₘO—C(O)—O-cycloalkyl, —N(H)C(Me)C(O)O-alkyl, SRᵣ, S(O)Rᵣ, S(O)₂Rᵣ, or alkoxy arylsulfonamide, wherein each A³ may be optionally substituted with

1 to 4

—R¹¹¹, —P(Y¹)(OA²)(OA²), —P(Y¹)(OA²)(N(A²)₂), —P(Y¹)(A²)(OA²), —P(Y¹)(A²)(N(A²)₂), or P(Y¹)(N(A²)₂)(N(A²)₂), —C(═O)N(A²)₂), halogen, alkyl, alkenyl, alkynyl, aryl, carbocycle, heterocycle, aralkyl, aryl sulfonamide, aryl alkylsulfonamide, aryloxy sulfonamide, aryloxy alkylsulfonamide, aryloxy arylsulfonamide, alkyl sulfonamide, alkyloxy sulfonamide, alkyloxy alkylsulfonamide, arylthio, —(CH₂)ₘheterocycle, —(CH₂)ₘ—C(O)O-alkyl, —O(CH₂)ₘOC(O)Oalkyl, —O—(CH₂)ₘ—O—C(O)—(CH₂)ₘ-alkyl, —(CH₂)ₘ—O—C(O)—O-cycloalkyl, —N(H)C(CH₃)C(O)O-alkyl, or alkoxy arylsulfonamide, optionally substituted with R¹¹¹;

A² is independently selected from PRT, H, alkyl, alkenyl, alkynyl, amino, amino acid, alkoxy, aryloxy, cyano, haloalkyl, cycloalkyl, aryl, heteroaryl, heterocycle, alkylsulfonamide, or arylsulfonamide, wherein each A² is optionally substituted with A³;

R¹¹¹ is independently selected from H, alkyl, alkenyl, alkynyl, aryl, cycloalkyl, heterocycle, halogen, haloalkyl, alkylsulfonamido, arylsulfonamido, —C(O)NHS(O)₂—, or —S(O)₂—, optionally substituted with one or more A³;

p and q are independently 1 or 2;

R² is C₁-C₆ alkyl, C₂-C₆ alkenyl or C₃-C₈ cycloalkyl, wherein said alkyl, alkenyl or cycloalkyl is optionally substituted with 1 to 3 halo;

R³ is C₁-C₈ alkyl, C₃-C₈ cycloalkyl, C3 C8 cycloalkyl(C₁-C₈)alkyl, aryl(C₁-C₈)alkyl, or Het, wherein aryl is phenyl or naphthyl and said alkyl, cycloalkyl, or aryl is optionally substituted with 1 to 3 substituents selected from the group consisting of halo, OR¹⁰, SR¹⁰, N(R¹⁰)₂, NH(C₁-C₆ alky1)O(C₁-C₆ alkyl), C₁-C₆ alkyl, C₁-C₆ haloalkyl, halo(C₁-C₆ alkoxy), NO₂, CN, CF₃, SO₂(C₁-C₆ alkyl), S(O)(C₁-C₆ alkyl), NR¹⁰SO₂R⁶, SO₂N(R⁶)₂, NHCOOR⁶, NHCOR⁶, NHCONHR⁶, CO₂R¹⁰, C(O)R¹⁰, and CON(R¹⁰)₂;

Het is a 5-6 membered saturated cyclic ring having 1 or 2 heteroatoms selected from N, O and S, wherein said ring is optionally substituted with 1 to 3 substituents selected from halo, OR¹⁰, SR¹⁰, N(R¹⁰)₂, NH(C₁-C₆ alkyl)O(C₁-C₆ alkyl), C₁-C₆ alkyl, C₁-C₆ haloalkyl, halo(C₁-C₆ alkoxy), NO₂, CN, CF₃, SO₂(C₁-C₆ alkyl), S(O)(C₁-C₆ alkyl), NR¹⁰SO₂R⁶, SO₂N(R⁶₂, NHCOOR⁶, NHCOR⁶, NHCONHR⁶, CO₂R¹⁰, C(O)R¹⁰, and CON(R¹⁰)₂;

R⁴ is H, C₁-C₈ alkyl, C₃-C₈ cycloalkyl(C₁-C₈)alkyl, or aryl(C₁-C₈)alkyl; wherein aryl is phenyl or naphthyl and said alkyl, cycloalkyl, or aryl is optionally substituted with 1 to 3 substituents selected from the group consisting of halo, OR¹⁰, SR¹⁰, N(R¹⁰)₂, NH(C₁-C₆ alky1)O(C₁-C₆ alkyl), C₁-C₆ alkyl, C₁-C₆ haloalkyl, halo(C₁-C₆ alkoxy), NO₂, CN, CF₃, SO₂(C₁-C₆ alkyl), S(O)(C₁-C₆ alkyl), NR¹⁰SO₂R⁶, SO₂N(R⁶)₂, NHCOOR⁶, NHCOR⁶, NHCONHR⁶. CO₂R¹⁰, C(O)R¹⁰, and CON(R¹⁰)₂;

R⁵ is H, halo, OR¹⁰, C₁-C₆ alkyl, CN, CF₃, SR¹⁰, SO₂(C₁-C₆ alkyl), C₃-C₈ cycloalkyl, C₃-C₈ cycloalkoxy, C₁-C₆ haloalkyl, N(R⁷)₂, aryl, heteroaryl or heterocyclyl; wherein aryl is phenyl or naphthyl, heteroaryl is a 5- or 6-membered aromatic ring having 1, 2 or 3 hetematoms selected from N, O and S, attached through a ring carbon or nitrogen, and heterocyclyl is a 5- to 7-membered saturated or unsaturated non-aromatic ring having 1, 2, 3 or 4 heteroatoms selected from N, O and S, attached through a ring carbon or nitrogen; and wherein said aryl, heteroaryl, heterocyclyl, cycloalkyl, cycloalkoxy, alkyl or alkoxy is optionally substituted with 1 to 4 substituents selected from the group consisting of halo, OR¹⁰, SR¹⁰, N(R⁷)₂, N(C₁-C₆ alky1)O(C₁-C₆ alkyl), C₁-C₆ alkyl, C₁-C₆ haloalkyl, halo(C₁-C₆ alkoxy), C₃-C₆ alkyl), C₃-C₆ cycloalkoxy, NO₂, CN, CF₃, SO₂(C₁-C₆ alkyl), NR¹⁰SO₂R⁶, SO₂N(R⁶)₂, S(O)C₁-C₆ NHCOOR⁶, NHCOR⁶, NHCONHR⁶, CO₂R¹⁰, C(O)R¹⁰, and CON(R¹⁰)₂; wherein the 2 adjacent substituents of said cycloalkyl, cycloalkoxy, aryl, heteroaryl or heterocyclyl are optionally taken together to form a 3-6 membered cyclic ring containing 0-3 heteroatoms selected from N, O and S;

R⁶ is C₁-C₆ alkyl, C₃-C₆ cycloalkyl, C₃-C₆ cycloalkyl(C₁-0₅)alkyl, aryl, aryl (C₁-C₄)alkyl, heteroaryl, heteroaryl(C₁-C₄ alkyl), heterocyclyl, or heterocyclyl(C₁-C₈ alkyl), wherein said alkyl, cycloalkyl, aryl, heteroaryl, or heterocyclyl is optionally substituted with 1 to 2 W substituents; and wherein each aryl is independently phenyl or naphthyl, each heteroaryl is independently a 5- or 6-membered aromatic ring having 1, 2 or 3 heteroatoms selected from N, O and S, attached through a ring carbon or nitrogen, and each heterocyclyl is independently a 5- to 7-membered saturated or unsaturated non-aromatic ring having 1, 2, 3 or 4 heteroatoms selected from N, O and S, attached through a ring carbon or nitrogen;

Each Rᵣ is independently H, (C₁-C₁₀) alkyl, (C₂-C₁₀) alkenyl, (C₂-C₁₀) alkynyl, (C₁-C₁₀) alkanoyl, or (C₁-C₁₀) alkoxycarbonyl;

Y¹ is independently O, S, N(A³), N(O)(A³), N(OA³), N(O)(OA³) or N(N(A³)(A³));

r is 0 to 6;

m is 0 to 6;

Y is C(═O), SO₂, or C(═N—CN);

Z is C(R¹⁰)₂, O, or N(R⁴);

M is C₁-C₁₂ alkylene or C₂-C₁₂ alkenylene, wherein said alkylene or alkenylene is optionally substituted with 1 or 2 substituents selected from the group consisting of C₁-C₈ alkyl, C₃-C₈ cycloalkyl(C₁-C₈ alkyl), and aryl(C₁-C₈ alkyl) and further which M can be substituted by up to nine halo; and 2 substituents of M are optionally taken together to form a 3-6 membered cyclic ring containing 0-3 heteroatoms selected from N, 0 and S; and optionally one substituent of M can be taken together with a ring atom within M to form a 3-6 membered ring system containing 0-3 heteroatoms selected from N, 0 and S where the 3-6 membered ring system is fused to the macrocyclic ring system;

each R⁷ is independently H, C₁-C₆ alkyl, C₃-C₆ cycloalkyl, C₃-C₆ cycloalkyl(C₁-C₆)alkyl, aryl, aryl(C₁-C₄)alkyl, heteroaryl, heteroaryl(C₁-C₄ alkyl), heterocyclyl, or heterocyclyl(C₁-C₈ alkyl), wherein said alkyl, cycloalkyl, aryl, heteroaryl, or heterocyclyl is optionally substituted with 1 to 2 W substituents; and wherein each aryl is independently phenyl or naphthyl, each heteroaryl is independently a 5- or 6-membered aromatic ring having 1, 2 or 3 heteroatoms selected from N, 0 and S, attached through a ring carbon or nitrogen, and each heterocyclyl is independently a 5- to 7-membered saturated or unsaturated non-aromatic ring having 1, 2, 3 or 4 heteroatoms selected from N, 0 and S, attached through a ring carbon or nitrogen;

each W is independently halo, OR¹⁰, C₁-C₆ alkyl, CN, CF₃, NO₂, SR¹⁰, CO₂R¹⁰, CON(R¹⁰)₂, C(O)R¹⁰, N(R¹⁰)C(O)R¹⁰, SO₂(C₁-C₆ alkyl), S(O)(C₁-C₆ alkyl), C₃-C₈ cycloalkyl, C₃-C₈ cycloalkoxy, C₁-C₆ haloalkyl, N(R¹⁰)₂, N(C₁-C₆ alky1)O(C₁-C₆ alkyl), halo(C₁-C₆ alkoxy), NR¹⁰SO₂R¹⁰, SO₂N(R¹⁰), NHCOOR¹⁰, NHCONHR¹⁰, aryl, heteroaryl or heterocyclyl; wherein aryl is phenyl or naphthyl, heteroaryl is a 5- or 6-membered aromatic ring having 1, 2 or 3 heteroatoms selected from N, 0 and S, attached through a ring carbon or nitrogen, and heterocyclyl is a 5- to 7-membered saturated or unsaturated non-aromatic ring having 1, 2, 3 or 4 heteroatoms selected from N, 0 and S, attached through a ring carbon or nitrogen;

each R¹⁰⁰ is independently H or C₁-C₆ alkyl.

The present invention also includes pharmaceutical compositions containing a compound of the present invention and methods of preparing such pharmaceutical compositions. The present invention further includes methods of treating or preventing one or more symptoms of HCV infection.

Other embodiments, aspects and features of the present invention are either further described in or will be apparent from the ensuing description, examples and appended claims.

### DETAILED DESCRIPTION OF THE INVENTION

The present invention includes compounds of formula I above, and pharmaceutically acceptable salts and/or hydrates thereof. These compounds and their pharmaceutically acceptable salts and/or hydrates are HCV protease inhibitors (e.g., HCV NS3 protease inhibitors). The present invention also includes compounds of formulae II, II-a, II-b, II-c II-d, III, III-a, III-b, and III-d wherein all variables are as defined for formula I.

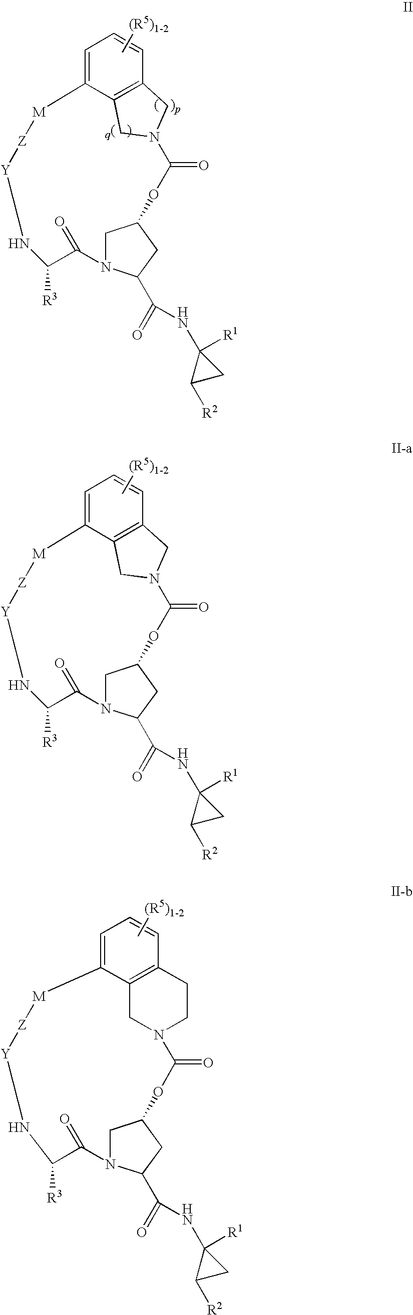

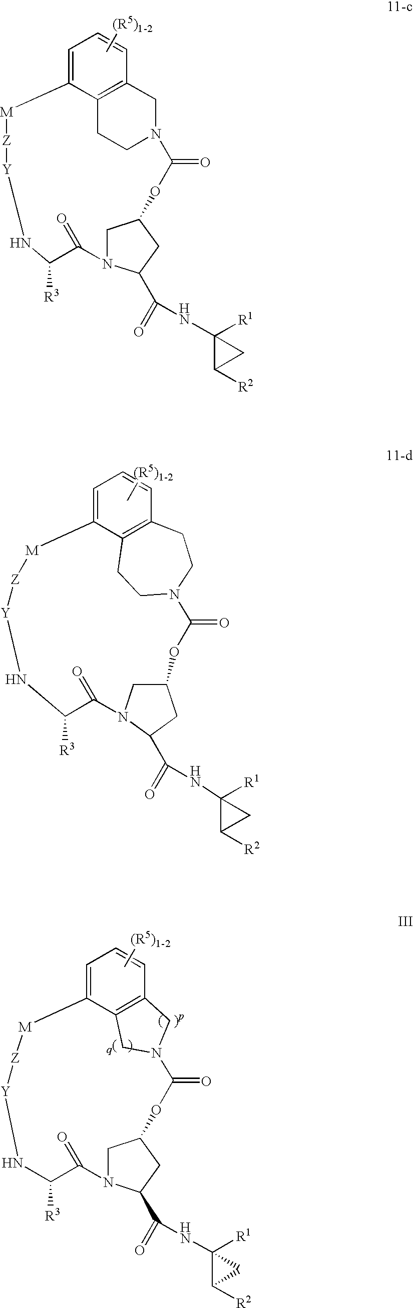

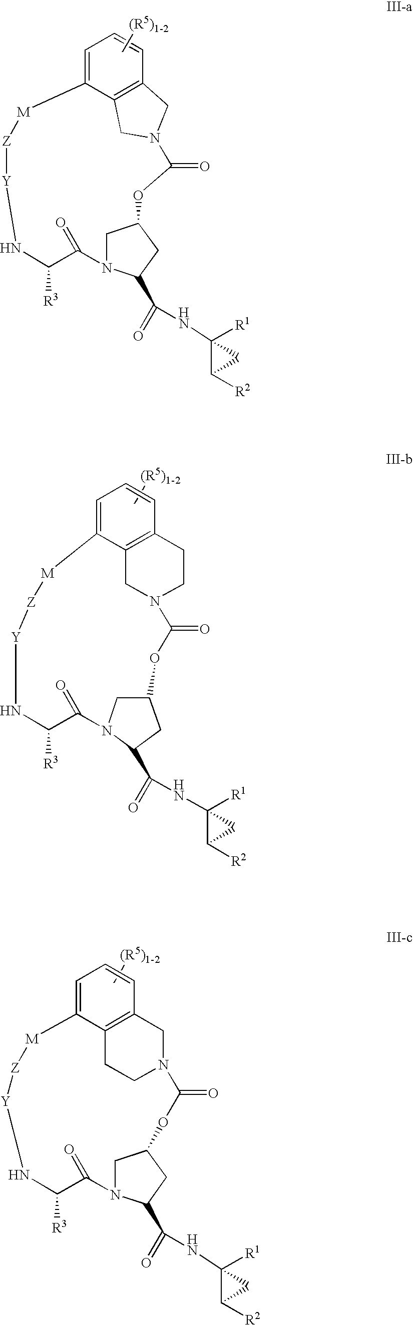

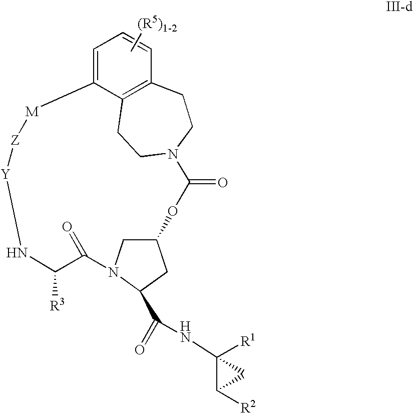

A first embodiment of the present invention is a compound of formula I, II, II-a, II-b, II-c, II-d, III, III-a, or III-d, or a pharmaceutically acceptable salt or hydrate thereof, wherein

R^{f} is A³;

m is 0 to 6.

In a specific embodiment of the invention R^{f} is H, alkyl, alkenyl, alkynyl, aryl, heteroaryl, or cycloalkyl, which R^{f} is optionally substituted with one or more R_{g};

each R_{g} is independently H, alkyl, alkenyl, alkynyl, halo, hydroxy, cyano, arylthio, cycloalkyl, aryl, heteroaryl, alkoxy, NRₕRᵢ; —C(═O)NRₕRᵢ or —C(═O)OR_{d}, wherein each aryl and heteroaryl is optionally substituted with one or more alkyl, halo, hydroxy, cyano, nitro, amino, alkoxy, alkoxycarbonyl, alkanoyloxy, haloalkyl, or haloalkoxy; wherein each alkyl of R_{g} is optionally substituted with one or more halo, alkoxy, or cyano;

each Rₕ, and Rᵢ is independently alkyl, or haloalkyl; and

R_{d} and Rₑ are each independently H, (C₁-C₁₀)alkyl, or aryl, which is optionally substituted with one or more halo;

In a specific embodiment of the invention R^{f} is alkyl, aryl, cycloalkyl, which R^{f} is optionally substituted with one or more R_{g} independently selected from alkyl, halo, —C(═O)OR_{d}, or trifluoromethyl, wherein each alkyl of R_{g} is optionally substituted with one or more halo, alkoxy, or cyano.

In a specific embodiment of the invention R^{f} is aryl, heteroaryl, or cyclopropyl which R^{f} is optionally substituted with one to three A³

In a specific embodiment of the invention R^{f} is cyclopropyl which R^{f} is optionally substituted by up to four A³.

In a specific embodiment of the invention R^{f} is cyclopropyl which R^{f} is optionally substituted by one A³.

In a specific embodiment of the invention R^{f} is H, alkyl, alkenyl, alkynyl, aryl, heteroaryl, or cycloalkyl, which R^{f} is optionally substituted with one or more R_{g}; each R_{g} is independently H, alkyl, alkenyl, alkynyl, halo, hydroxy, cyano, arylthio, cycloalkyl, aryl, heteroaryl, alkoxy, NRₕRᵢ, —C(═O)NRₕRᵢ, or —C(═O)OR_{d}, wherein each aryl and heteroaryl is optionally substituted with one or more alkyl, halo, hydroxy, cyano, nitro, amino, alkoxy, alkoxycarbonyl, alkanoyloxy, haloalkyl, or haloalkoxy; wherein each alkyl of R_{g} is optionally substituted with one or more halo or cyano; and each Rₕ and Rᵢ is independently H, alkyl, or haloalkyl.

In a specific embodiment of the invention R^{f} is H, alkyl, alkenyl, alkynyl, aryl, heteroaryl, or cycloalkyl, which R^{f} is optionally substituted with one or more R_{g}; each R_{g} is independently H, alkyl, alkenyl, alkynyl, halo, hydroxy, cyano, arylthio, cycloalkyl, aryl, heteroaryl, alkoxy, NRₕRᵢ, —C(═O)NRₕRᵢ, wherein each aryl and heteroaryl is optionally substituted with one or more alkyl, halo, hydroxy, cyano, nitro, amino, alkoxy, alkoxycarbonyl, alkanoyloxy, haloalkyl, or haloalkoxy; each Rₕ, and Rᵢ is independently H, alkyl, or haloalkyl;

In a specific embodiment of the invention R^{f} is phenyl, cyclopropyl, 2-fluorophenyl, 4-chlorophenyl, 2-chlorophenyl, 2,6-dimethylphenyl, 2-methylphenyl, 2,2-dimethylpropyl, 2,2-difluoroethyl, 2,2,2-trifluoroethyl, or 1-methylcyclopropyl.

In a specific embodiment of the invention R^{f} is cyclopropyl.

In a specific embodiment of the invention R^{f} is 1-methylcyclopropyl.

A third embodiment of the present invention is a compound of formula I, II, II-a, II-b, II-c, II-d, III, III-a, III-c or III-d, or a pharmaceutically acceptable salt or hydrate thereof, wherein R² is C₁-C₆ alkyl or C₂-C₆ alkenyl; and all other variables are as originally defined or as defined in any one of the preceding embodiments. In a first aspect of the third embodiment, R² is C₁-C₄ alkyl or C₂-C₄ alkenyl; and all other variables are as originally defined or as defined in any one of the preceding embodiments. In a second aspect of the third embodiment, R² is C₂-C₄ alkenyl; and all other variables are as originally defined or as defined in any one of the preceding embodiments. In a feature of the second aspect of the third embodiment, R² is vinyl; and all other variables are as defined in the second embodiment or as defined in any one of the preceding embodiments. In a third aspect of the third embodiment, R² is C₁-C₄ alkyl; and all other variables are as originally defined or as defined in any one of the preceding embodiments. In a feature of the third aspect of the third embodiment, R² is ethyl; and all other variables are as defined in the third embodiment or as defined in any one of the preceding embodiments.

A fourth embodiment of the present invention is a compound of formula I, II, II-a, II-b, II-c, II-d, III, III-a, III-c or III-d, or a pharmaceutically acceptable salt or hydrate thereof, wherein R³ is C₃-C₆ cycloalkyl optionally substituted with C₁-C₆ alkyl; Het; or C₁-C₈ alkyl optionally substituted with 1 to 3 substituents selected from halo and Ole; and all other variables are as originally defined or as defined in any one of the preceding embodiments. In a first aspect of the fourth embodiment, R³ is C₅-C₇ cycloalkyl, piperidinyl, pyrrolidinyl, tetrahydrofuranyl, tetrahydropyranyl, or C₁-C₈ alkyl optionally substituted with 1 to 3 halo substituents; and all other variables are as defined in the fourth embodiment or as defined in any one of the preceding embodiments. In a second aspect of the fourth embodiment, R³ is C₃-C₆ cycloalkyl or C₁-C₈ alkyl optionally substituted with 1 to 3 halo substituents; and all other variables are as defined in the fourth embodiment or as defined in any one of the preceding embodiments. In a third aspect of the fourth embodiment, R³ is propyl or butyl; and all other variables are as defined in the fourth embodiment or as defined in any one of the preceding embodiments. In a feature of the third aspect of the fourth embodiment, R³ is i-propyl, n-butyl or t-butyl; and all other variables are as defined in the fourth embodiment or as defined in any one of the preceding embodiments. In a fourth aspect of the fourth embodiment, R³ is cyclopentyl or cyclohexyl; and all other variables are as defined in the fourth embodiment or as defined in any one of the preceding embodiments. In a fifth aspect of the fourth embodiment, R³ is CH₂CF, or CH₂CHF2; and all other variables are as defined in the fourth embodiment or as defined in any one of the preceding embodiments. In a sixth aspect of the fourth embodiment, R³ is C₃-C₈ cycloalkyl, Het, or C₁-C₈ alkyl optionally substituted with 1 to 3 halo substituents; and all other variables are as originally defined or as defined in any one of the preceding embodiments. In a seventh aspect of the fourth embodiment, R³ is C₃-C₈ cycloalkyl substituted with C₁-C₆ alkyl, or C₁-C₈ alkyl substituted with 1 to 3 Ole substituents; and all other variables are as originally defined or as defined in any one of the preceding embodiments. In an eighth aspect of the fourth embodiment, R³ is cyclohexyl substituted with methyl; and all other variables are as originally defined or as defined in any one of the preceding embodiments. In a ninth aspect of the fourth embodiment, R³ is CH₂O-t-Bu; and all other variables are as originally defined or as defined in any one of the preceding embodiments.

A fifth embodiment of the present invention is a compound of formula I, II, II-a, II-b, II-c, II-d, III, III-a, III-c or III-d, or a pharmaceutically acceptable salt or hydrate thereof, wherein R⁵ is H or halo; and all other variables are as originally defined or as defined in any one of the preceding embodiments. In one aspect of the fifth embodiment, R⁵ is H, F, or Cl; and all other variables are defined in the fifth embodiment or as defined in any one of the preceding embodiments.

A sixth embodiment of the present invention is a compound of formula I, II, II-a, II-b, II-c, III, III-a, III-c or III-d, or a pharmaceutically acceptable salt or hydrate thereof, wherein R⁵ is C₁-C₆ thioalkyl, aryl, heteroaryl, or heterocyclyl; wherein aryl is phenyl or naphthyl, heteroaryl is a 5- or 6-membered aromatic ring having 1, 2 or 3 heteroatoms selected from N, 0 and S, attached through a ring carbon or nitrogen, and heterocyclyl is a 5- to 7-membered saturated or unsaturated non-aromatic ring having 1, 2, 3 or 4 heteroatoms selected from N, 0 and S, attached through a ring carbon or nitrogen; and wherein said aryl, heteroaryl, heterocyclyl, or thioalkyl is optionally substituted with 1 to 4 substituents selected from the group consisting of halo, Ole, SR¹⁰, N(R⁷)₂, NH(C₁-C₆alky1)O(C₁-C₆ alkyl), C₁-C₆ alkyl, C₁-C₆ haloalkyl, halo(C₁-C₆ alkoxy), C₃-C₆ cycloalkyl, cycloalkoxy, NO₂, CN, CF₃, SO₂(C₁-C₆ alkyl), NR¹⁰SO₂R⁶, SO₂N(R⁶)₂, S(O)(C₁-C₆ alkyl), NHCOOR⁶, NHCOR⁶, NHCONHR⁶, CO₂R¹⁰, C(O)R¹⁰, and CON(R¹⁰)₂; and all other variables are as originally defined or as defined in any one of the preceding embodiments.

In one aspect of the sixth embodiment, R⁵ is aryl wherein aryl is optionally substituted with 1 to 4 substituents selected from the group consisting of halo, OR¹⁰, SR¹⁰, N(R⁷)₂, NH(C₁-C₆ alky1)O(C₁-C₆ alkyl, C₁-C₆ alkyl, C₁-C₆ haloalkyl, halo(C₁-C₆ alkoxy), C₃-C₆ cycloalkyl, cycloalkoxy NO₂, CN, CF³, SO₂(C₁-C₆ alkyl), NR¹⁰SO₂R⁶, SO₂N(R⁶)₂, S(O)(C₁-C₆ alkyl), NHCOOR⁶, NHCOR⁶, NHCONHR⁶, CO₂R¹⁰, C(O)R¹⁰, and)CON(R¹⁰)₂; and all other variables are as defined in the sixth embodiment or as defined in any one of the preceding embodiments. In a second aspect of the sixth embodiment, R⁵ is C₁-C₆ thioalkyl,

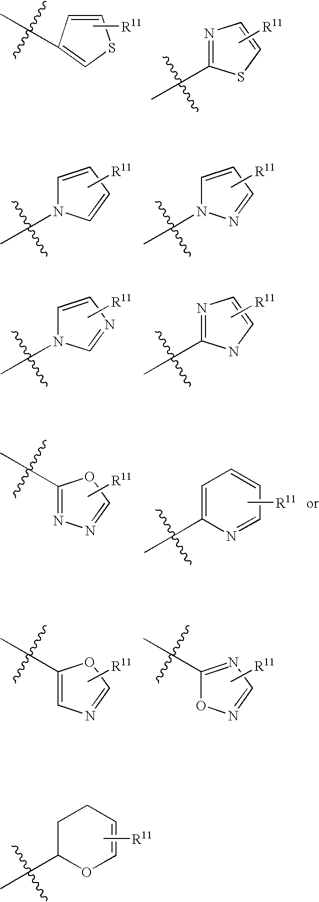

wherein R¹¹ is H, C₁-C₆ alkyl, NHR⁷, NHCOR¹², NHCONHR¹² or NHCOOR¹² and each R¹² is independently C₁-C₆ alkyl or C₃-C₆ cycloalkyl; and all other variables are as defined in the sixth embodiment or as defined in any one of the preceding embodiments In a third aspect of the sixth embodiment, R⁵ is

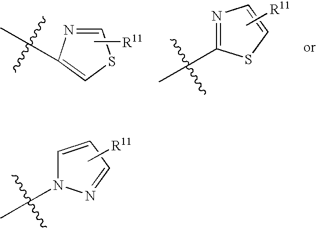

wherein R¹¹ is H, C₁-C₆ alkyl, NHR⁷, NHCOR¹², NHCONHR or NHCOOR¹² and each R¹² is independently C₁-C₆ alkyl or C₃-C₆ cycloalkyl; and all other variables are as defined in the sixth embodiment or as defined in any one of the preceding embodiments.

In a fourth aspect of the sixth embodiment, R^{s} is unsubstituted phenyl; and all other variables are as defined in the sixth embodiment or as defined in any one of the preceding embodiments.

A seventh embodiment of the present invention is a compound of formula I, II, II-a, II-b, II-c, II-d, III, III-a, III-c or III-d, or a pharmaceutically acceptable salt or hydrate thereof, wherein R⁵ is C₁-C₆ alkyl, C₁-C₆ alkoxy, hydroxy, or N(R⁷)₂ wherein feis H or C₁-C₆ alkyl; and all other variables are as originally defined or as defined in any one of the preceding embodiments. In one aspect of the seventh embodiment, R⁵ is C₁-C₆ alkoxy; and all other variables are as defined in the seventh embodiment or as defined in any one of the preceding embodiments. In a second aspect of the seventh embodiment, R⁵ is methoxy; and all other variables are as defined in the seventh embodiment or as defined in any one of the preceding embodiments.

An eighth embodiment of the present invention is a compound of formula I′, II, II¹ or III′, or a pharmaceutically acceptable salt or hydrate thereof, wherein all variables are as originally defined or as defined in any one of the preceding embodiments.

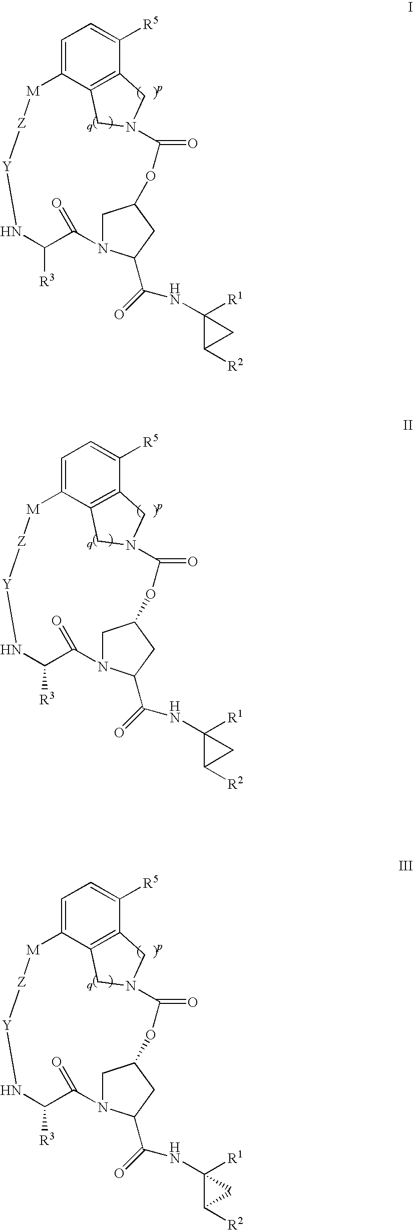

A ninth embodiment of the present invention is a compound of formula I, II, II-a, II-b, II-c, II-d, III, III-a, III-c or III-d, or a pharmaceutically acceptable salt or hydrate thereof, wherein Y is C═O or SO₂; and all other variables are as originally defined or as defined in any one of the preceding embodiments. In one aspect of the ninth embodiment, Y is C═O; and all other variables are as defined in the ninth embodiment or as defined in any one of the preceding embodiments.

A tenth embodiment of the present invention is a compound of formula I, II, II-a, II-b, II-c, II-d, III, III-a, III-c or III-d, or a pharmaceutically acceptable salt or hydrate thereof, wherein Z is O, C(R¹⁰)₂, NH or N(C₁-C₈ alkyl); and all other variables are as originally defined or as defined in any one of the preceding embodiments. In one aspect of the tenth embodiment, Z is O, CH₂, NH, or N(CH₃); and all other variables are as defined in the tenth embodiment or as defined in any one of the preceding embodiments. In another aspect of the tenth embodiment, Z is N(i-Pr) or N(n-Pr); and all other variables are as defined in the tenth embodiment or as defined in any one of the preceding embodiments.

An eleventh embodiment of the present invention is a compound of formula I, II, II-a, II-b, II-c, II-d, III, III-a, III-c or III-d, or a pharmaceutically acceptable salt or hydrate thereof, wherein M is C₁-C₈ alkylene or C₇-C₈ alkenylene, wherein said alkylene or alkenylene is optionally substituted with 1 or 2 substituents selected from C₁-C₈ alkyl, C₃-C₈ cycloalkyl(C₁-C₈ alkyl), or aryl(C₁-C₈ alkyl); and the 2 adjacent substituents of M are optionally taken together to form a 3-6 membered cyclic ring containing 0-2 heteroatoms selected from N, 0 and S; and all other variables are as originally defined or as defined in any one of the preceding embodiments. In a first aspect of the eleventh embodiment, M is C₁-C₈ alkylene or C₂-C₈ alkenylene, wherein said alkylene or alkenylene is optionally substituted with 1 or 2 substituents selected from C₁-C₈ alkyl, C₃-C₈ cycloalkyl(C₁-C₈ alkyl), or aryl(C₁-C₈ alkyl); and all other variables are as originally defined or as defined in any one of the preceding embodiments. In a first feature of the first aspect of the eleventh embodiment, M is unsubstituted C₁-C₈ alkylene or unsubstituted C₂-C₈ alkenylene; and all other variables are as defined in the eleventh embodiment or as defined in any one of the preceding embodiments. In a second feature of the first aspect of the eleventh embodiment, M is unsubstituted C₄ alkylene or unsubstituted C₄ alkenylene; and all other variables are as defined in the eleventh embodiment or as defined in any one of the preceding embodiments. In a third feature of the first aspect of the eleventh embodiment, M is unsubstituted C_{S} alkylene or unsubstituted C_{S} alkenylene; and all other variables are as defined in the eleventh embodiment or as defined in any one of the preceding embodiments. In a fourth feature of the first aspect of the eleventh embodiment, M is unsubstituted C(alkylene or unsubstituted C₆ alkenylene; and all other variables are as defined in the eleventh embodiment or as defined in any one of the preceding embodiments. In a fifth feature of the first aspect of the eleventh embodiment, M is unsubstituted C₈ alkylene or unsubstituted C₈ alkenylene; and all other variables are as defined in the eleventh embodiment or as defined in any one of the preceding embodiments. In a sixth feature of the first aspect of the eleventh embodiment, M is unsubstituted C₈ alkylene or unsubstituted C₈ alkenylene; and all other variables are as defined in the eleventh embodiment or as defined in any one of the preceding embodiments. In a seventh feature of the first aspect of the eleventh embodiment, M is:

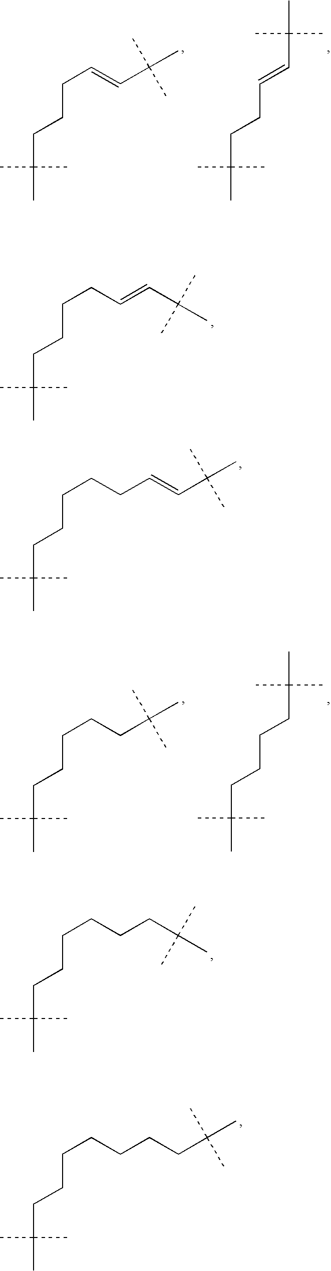

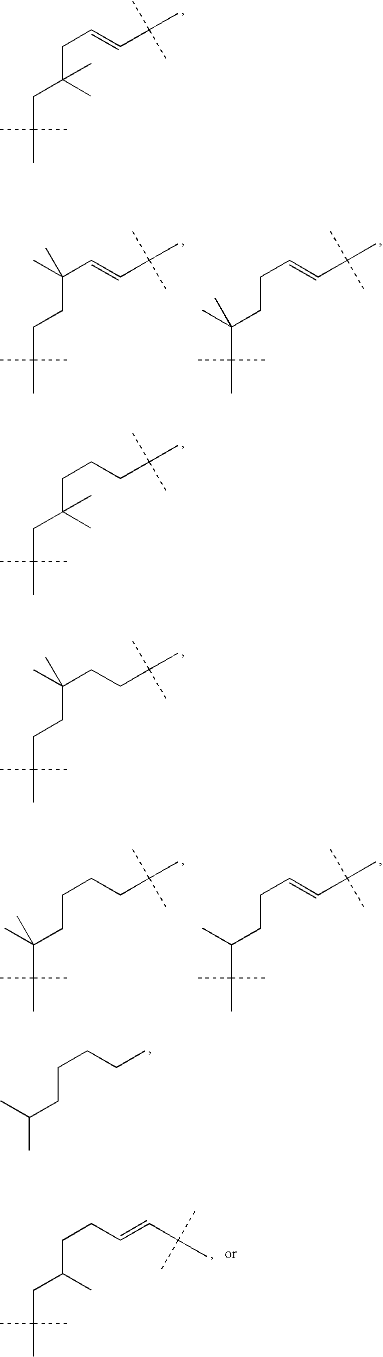

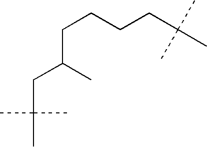

In the eighth feature of the first aspect of the eleventh embodiment, M is

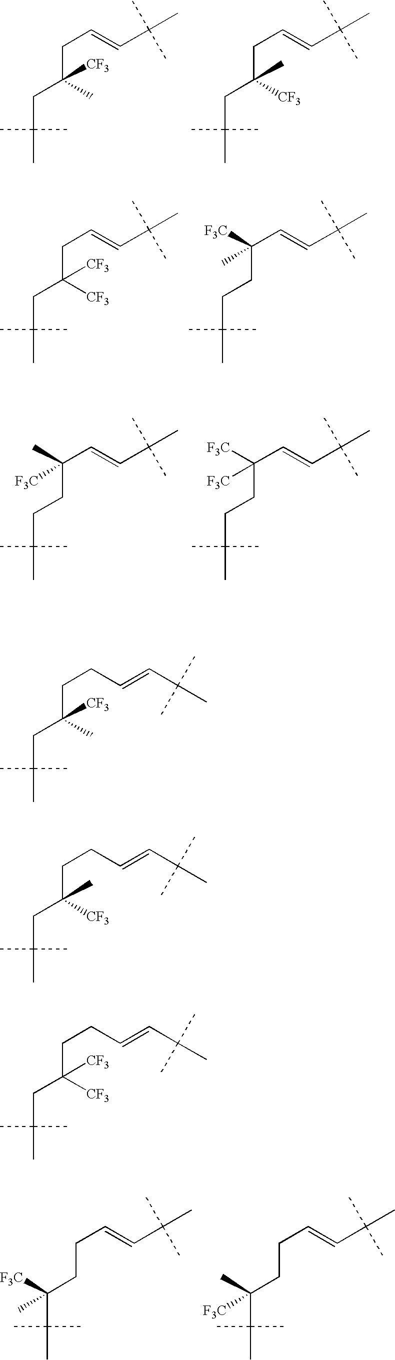

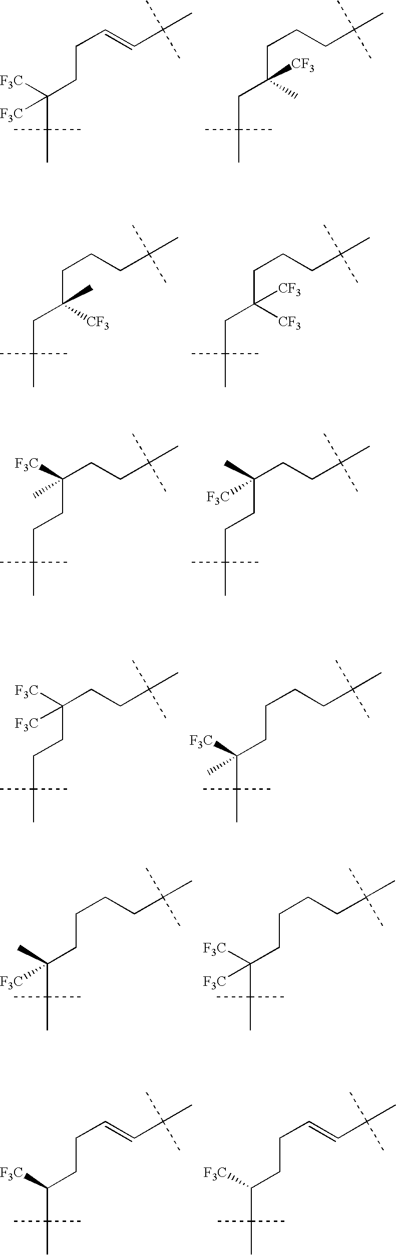

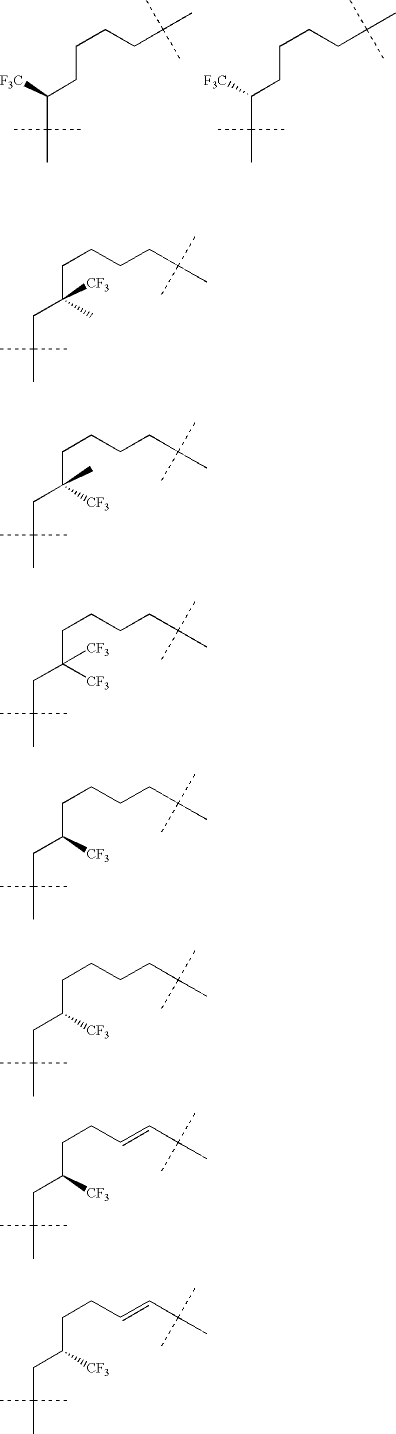

In a second aspect of the eleventh embodiment, M is C₁-C₈ alkylene or C₂-C₈ alkenylene, wherein said alkylene or alkenylene is optionally substituted with 1 or 2 substituents selected from C₁-C₈ alkyl, C₃-C₈ cycloalkyl(C₃-C₈ alkyl), or aryl(C₁-C₈ alkyl); and the 2 adjacent substituents of M are taken together to form a 3-6 membered cyclic ring containing 0 heteroatoms; and all other variables are as originally defined or as defined in any one of the preceding embodiments. In a feature of the second aspect of the eleventh embodiment, M is:

A twelfth embodiment of the present invention is a compound, or a pharmaceutically acceptable salt or hydrate thereof, selected from the group consisting of the compounds III-I to III-252 wherein R⁹⁹ is H, methyl, C₂-C₈ alkyl or C₂-C₈ haloalkyl.

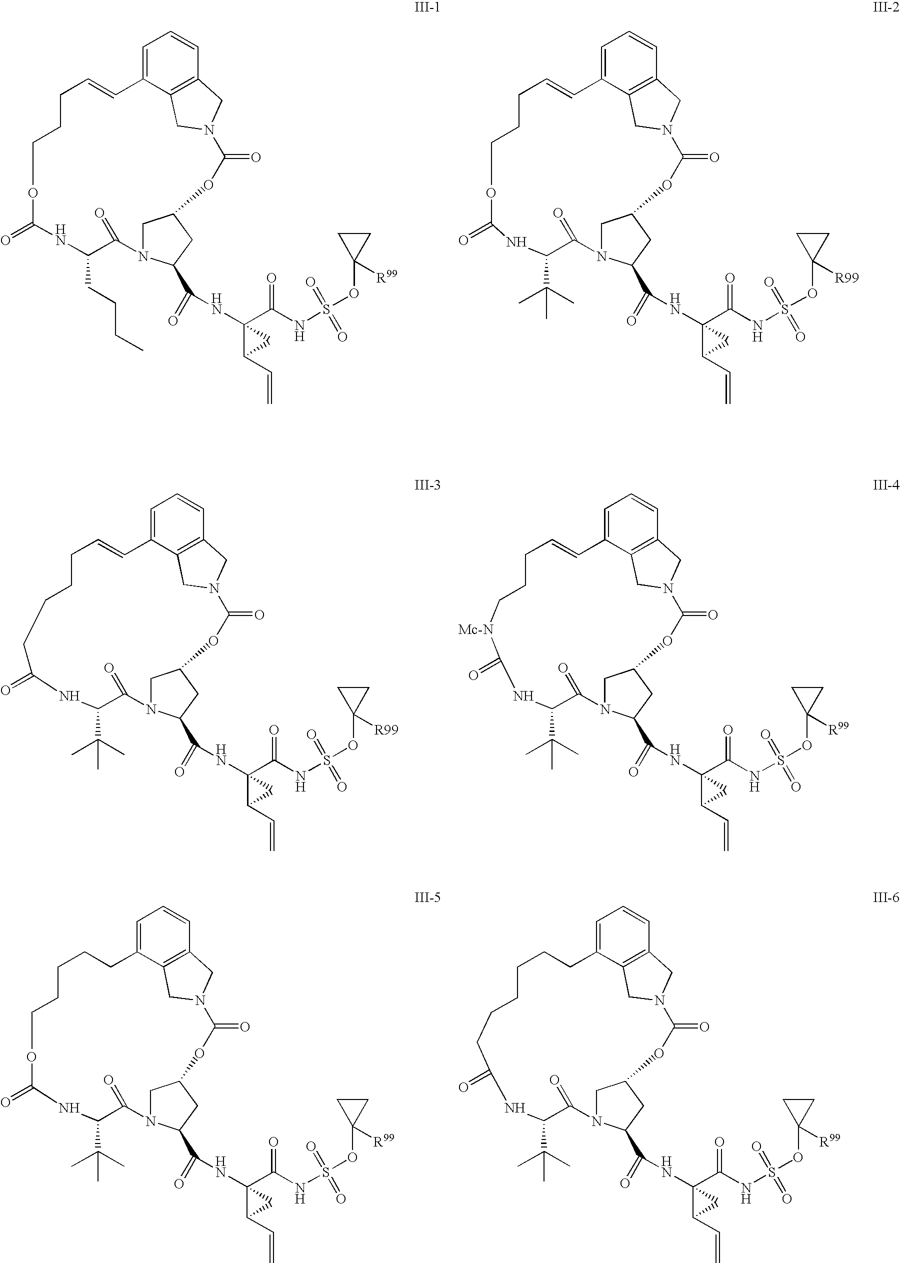

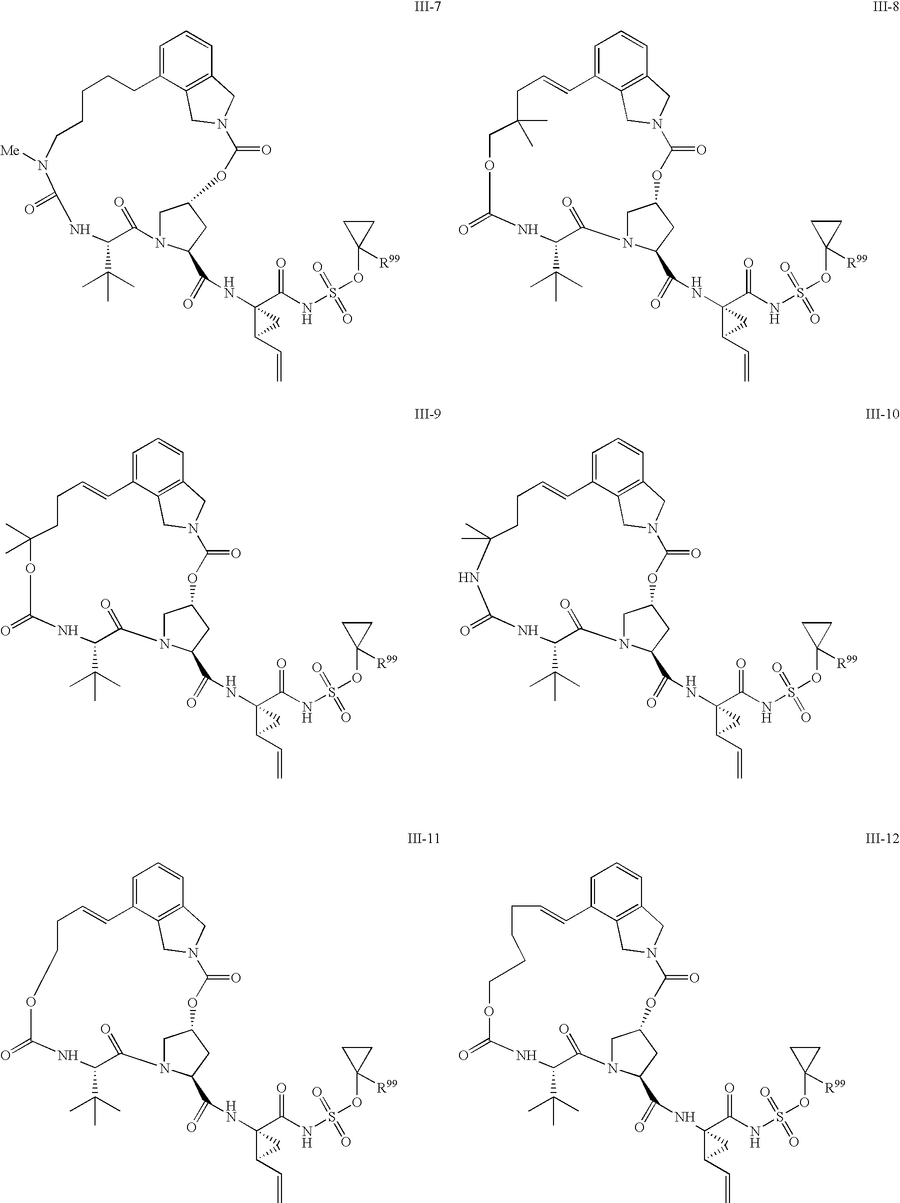

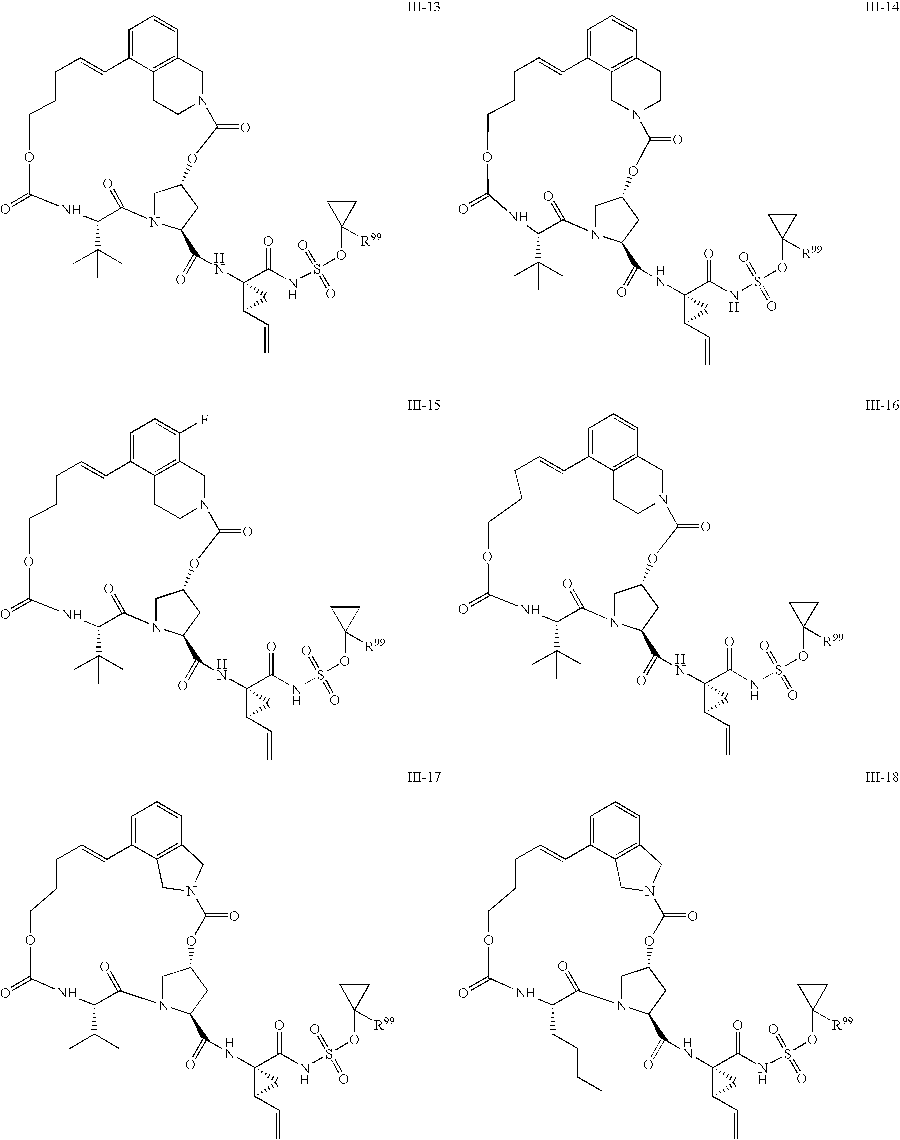

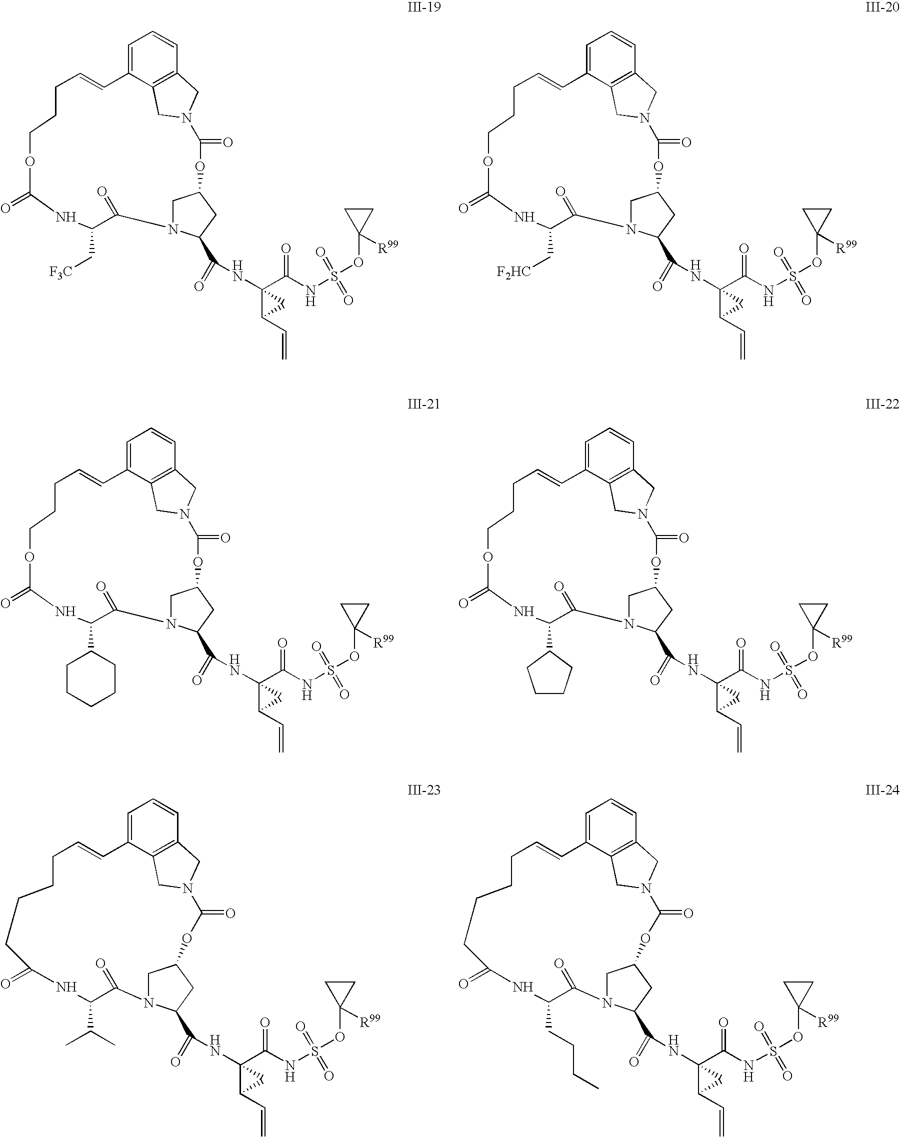

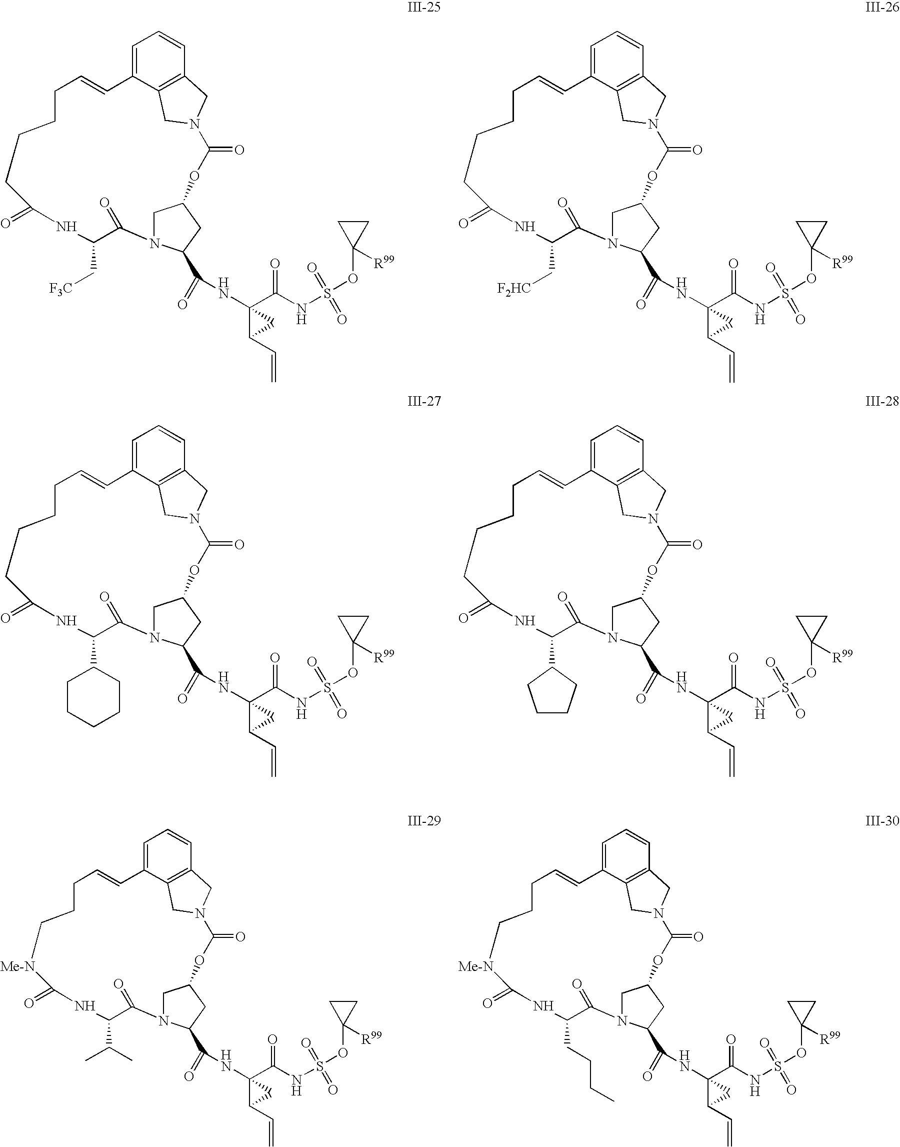

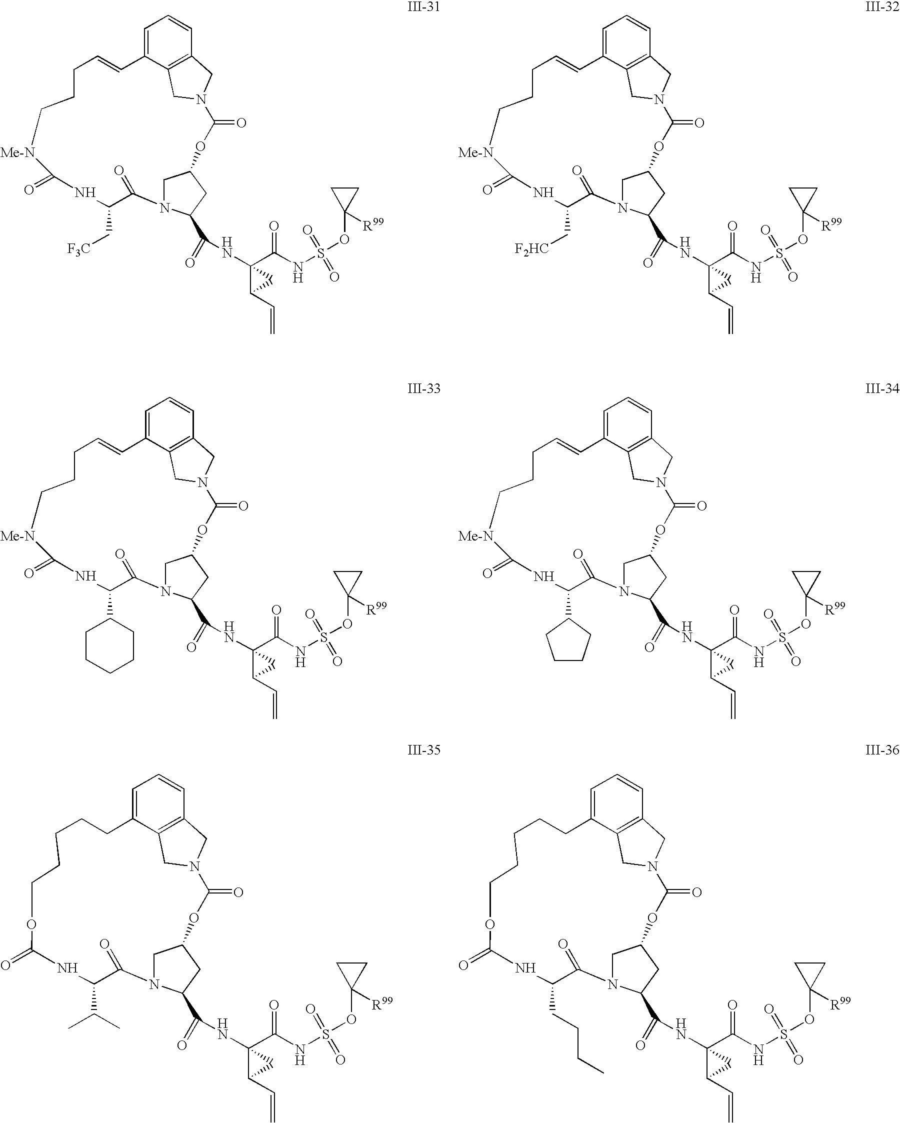

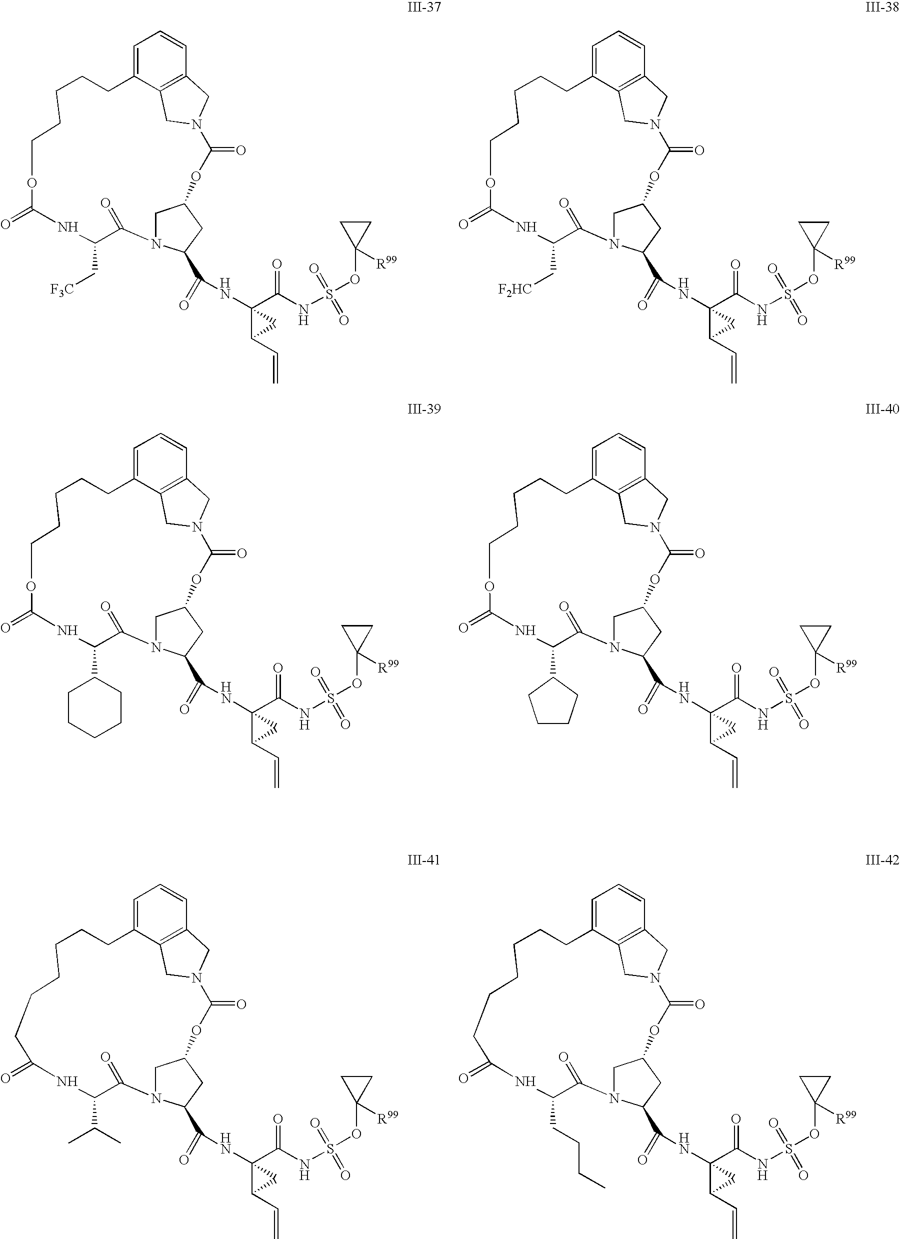

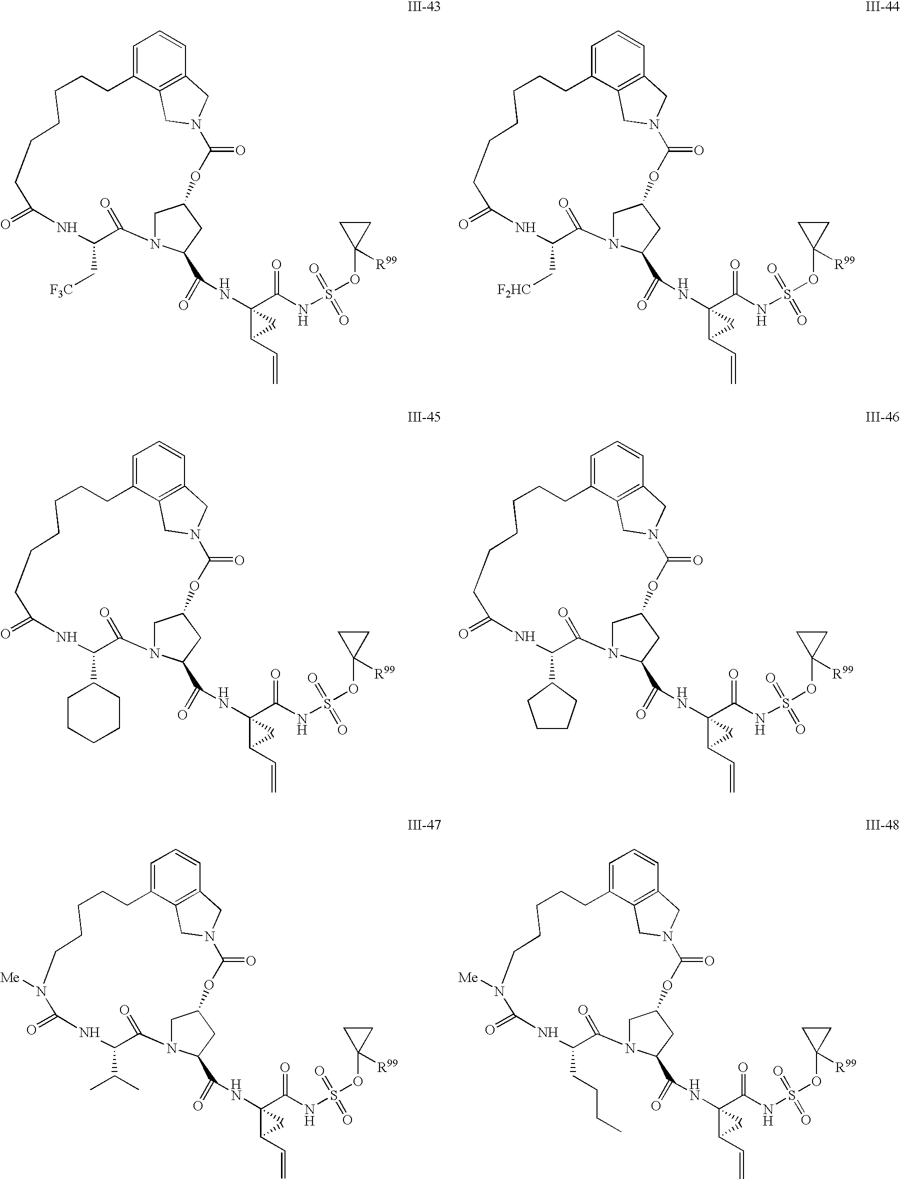

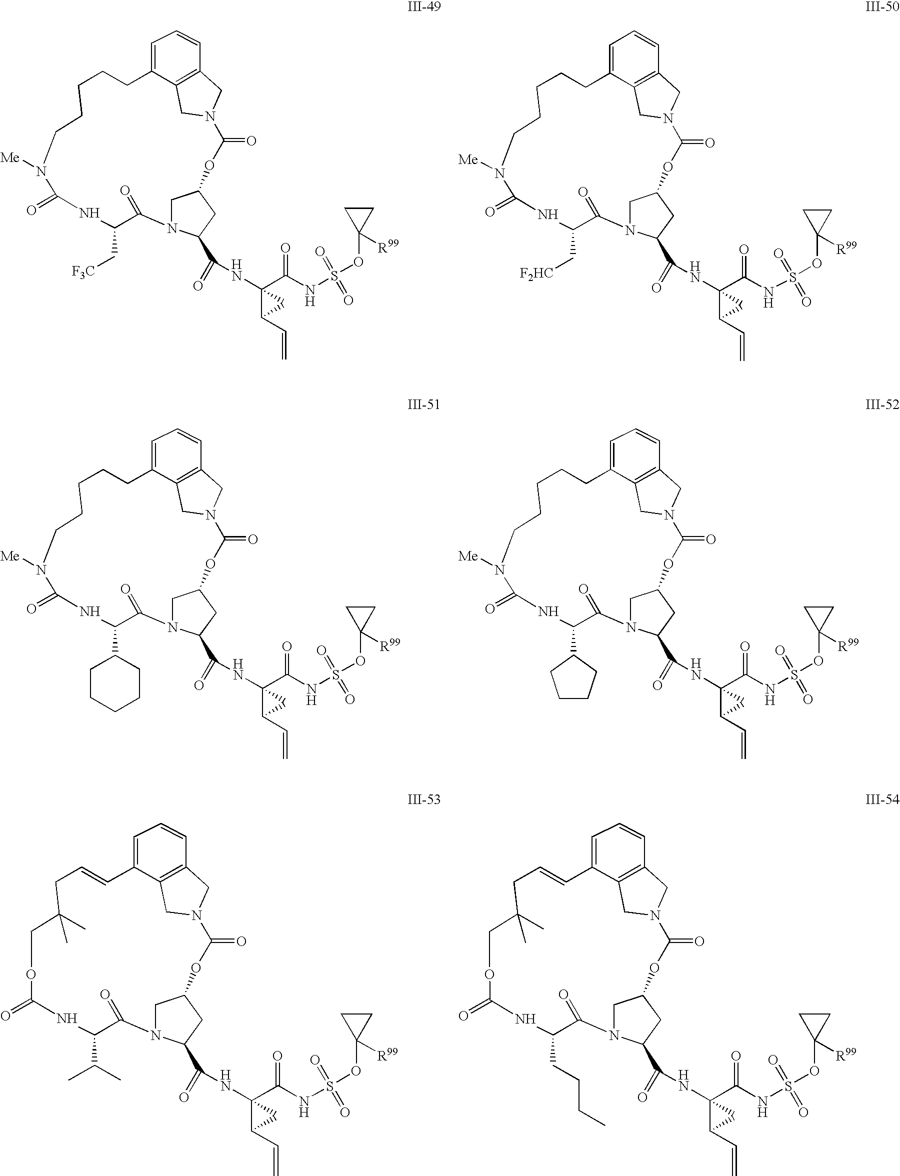

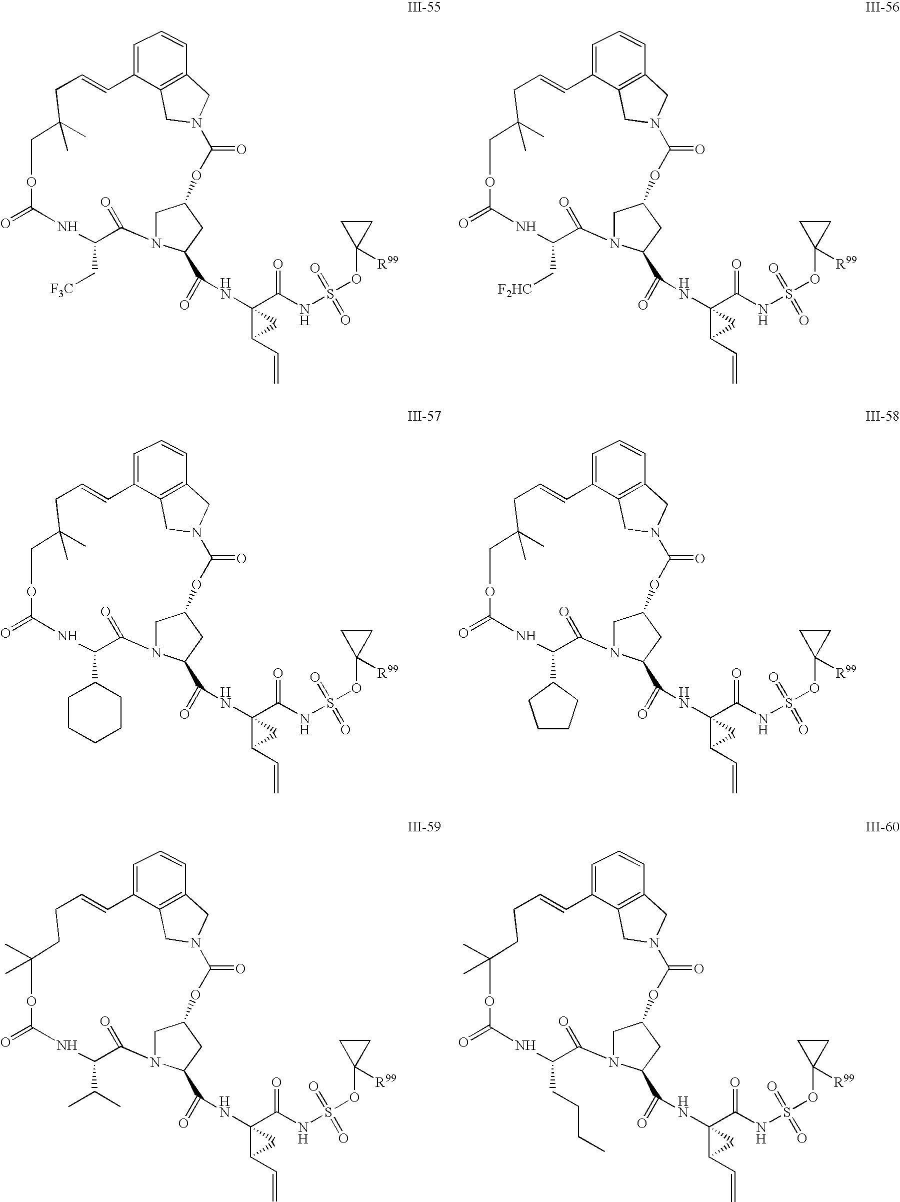

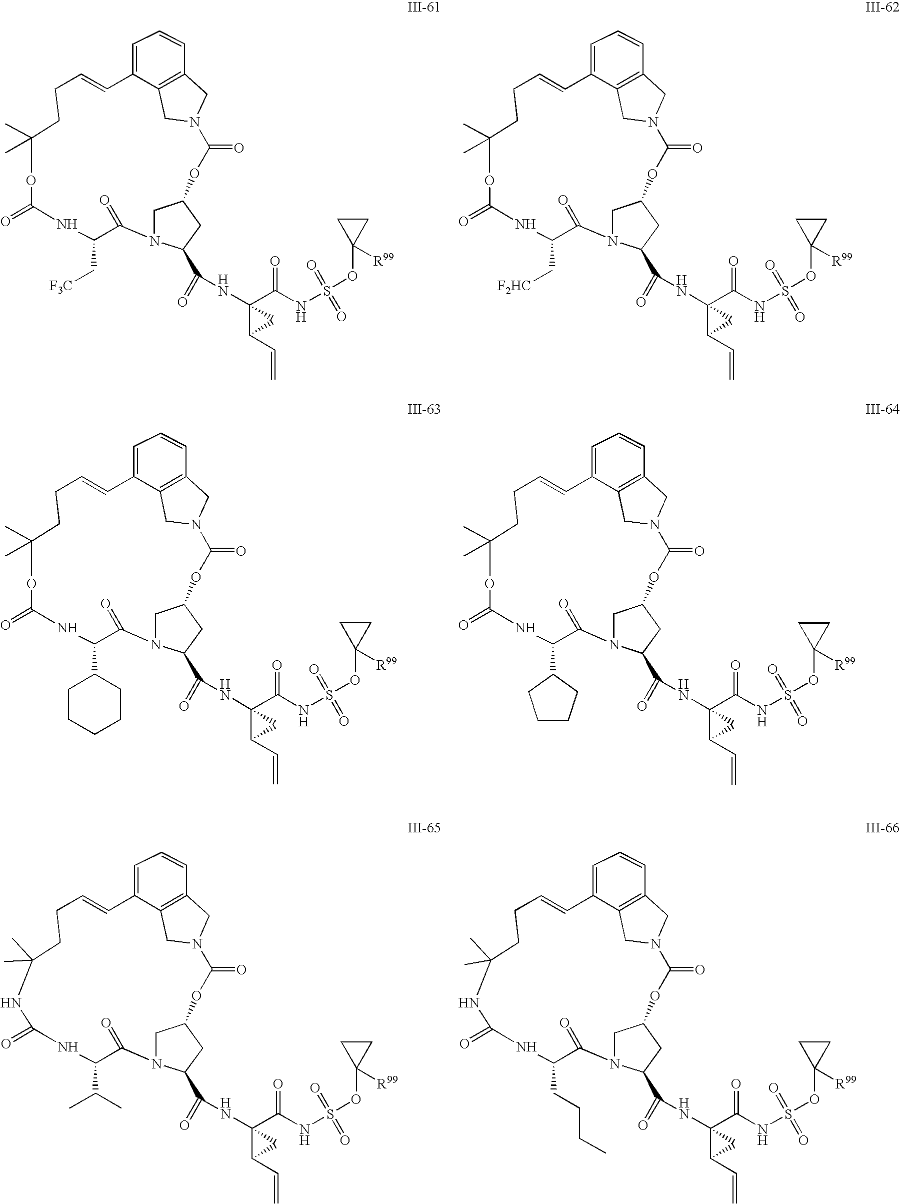

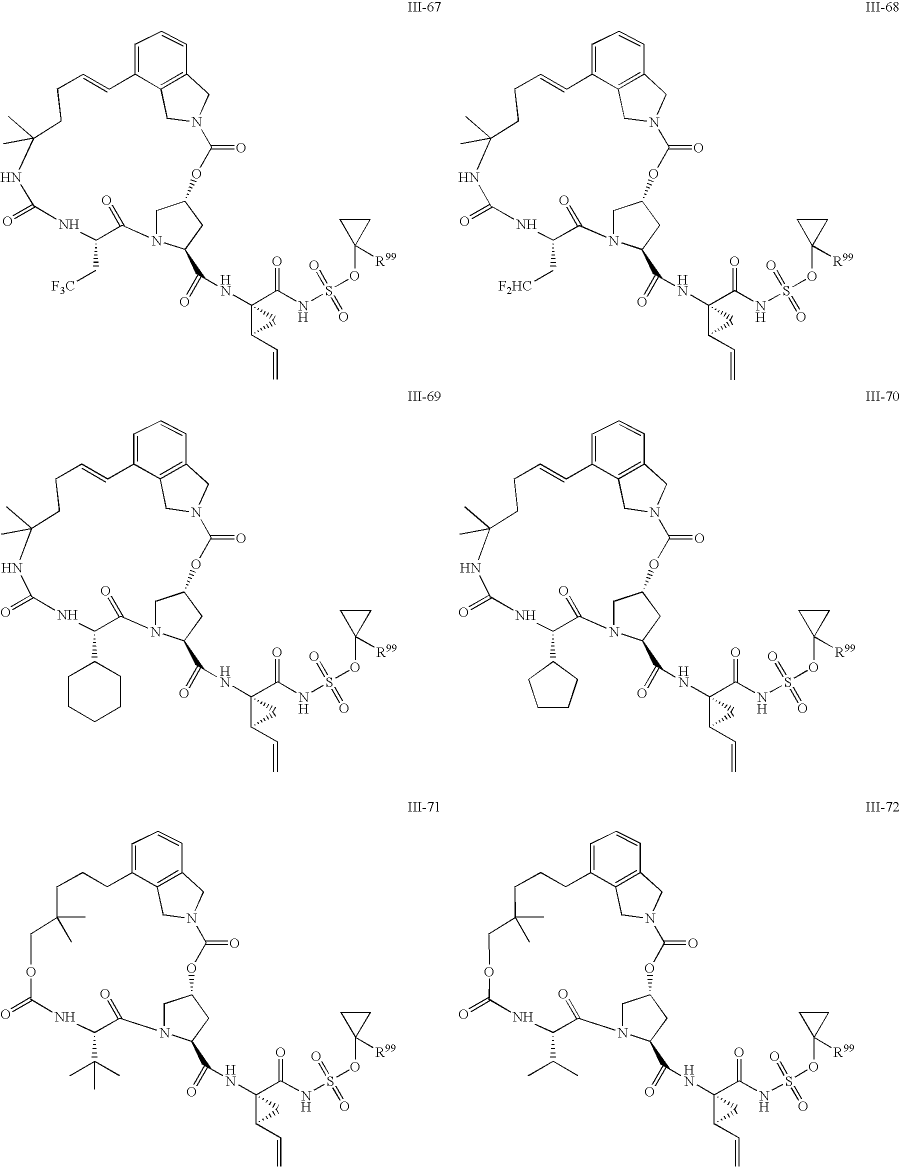

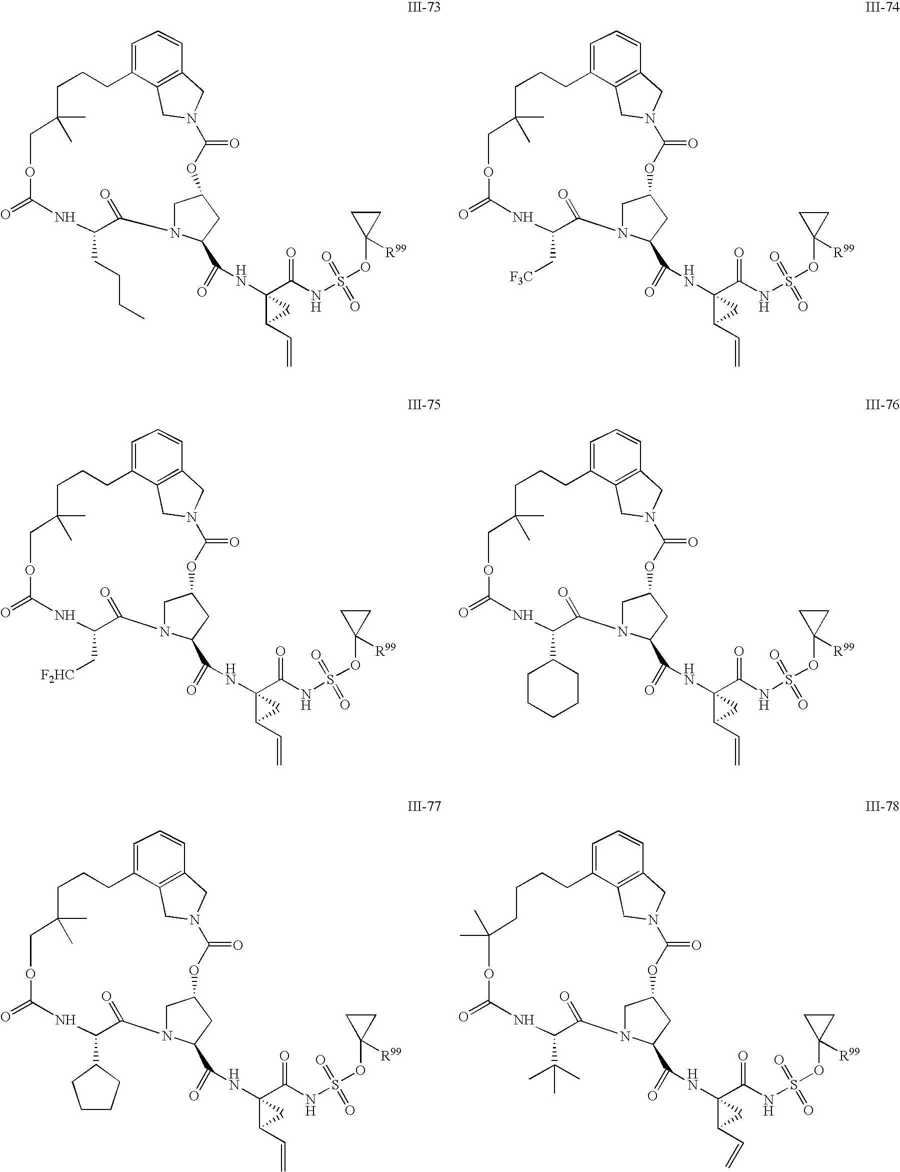

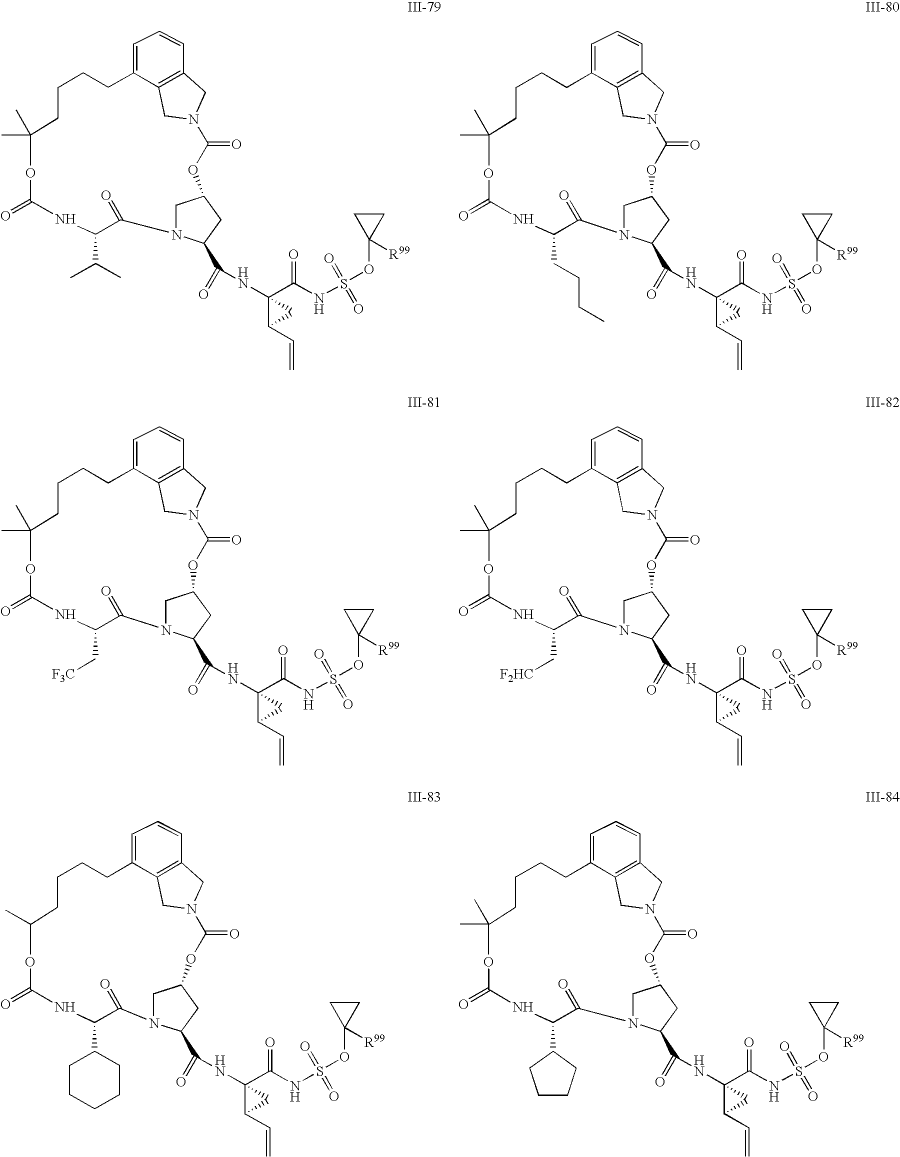

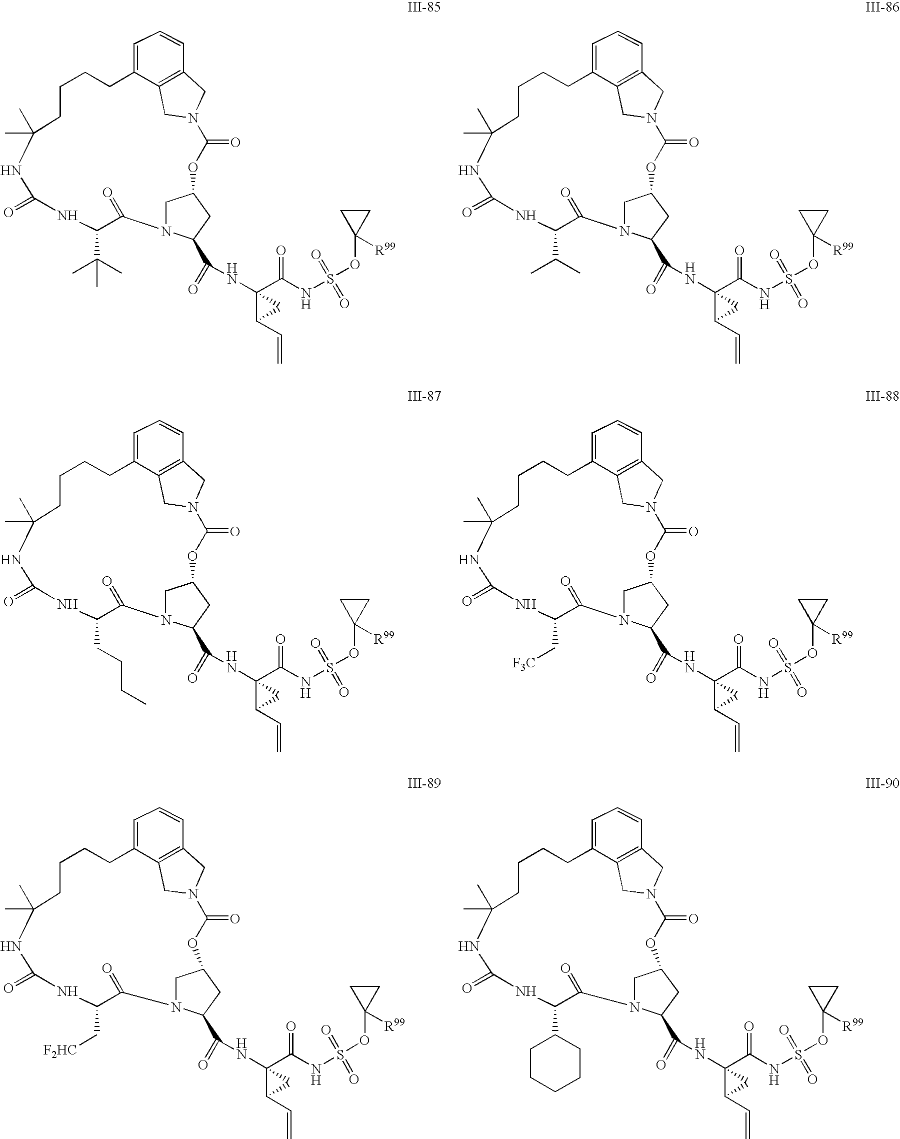

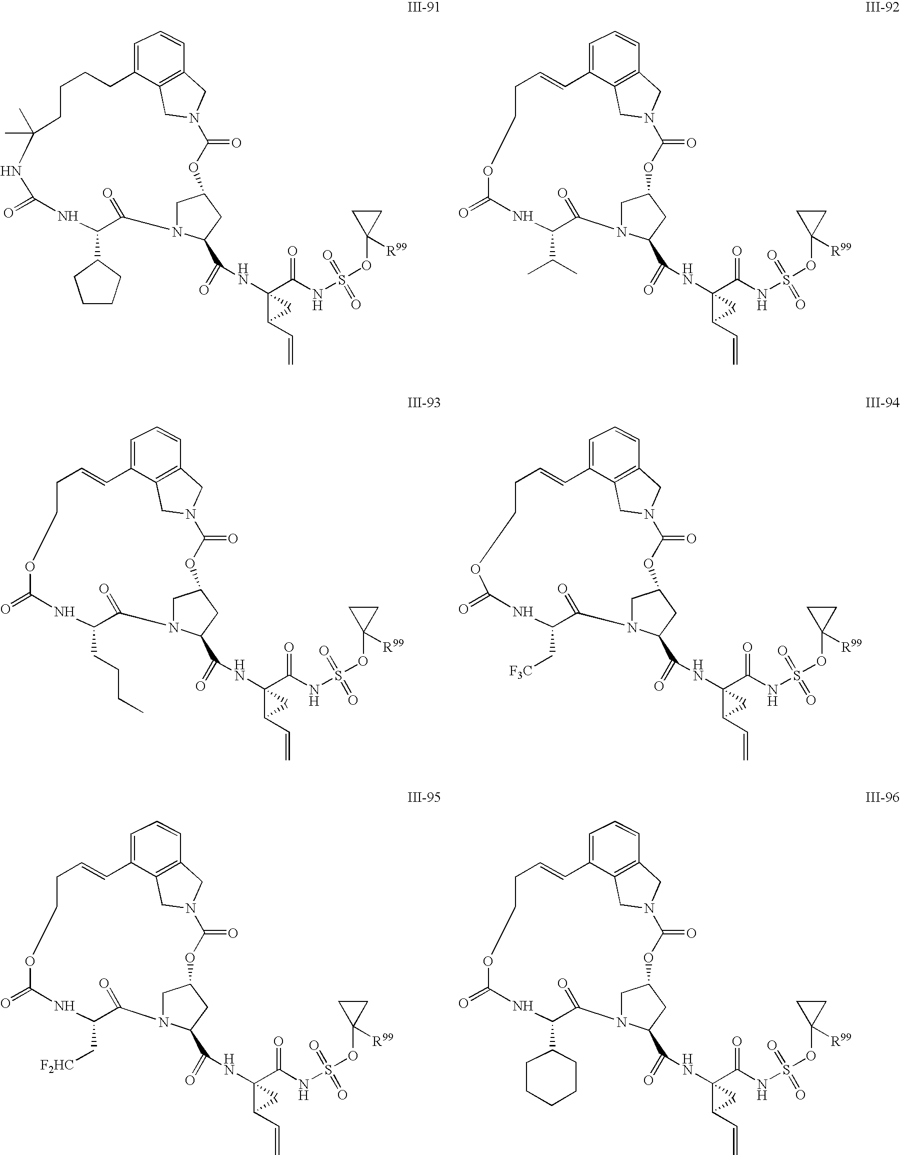

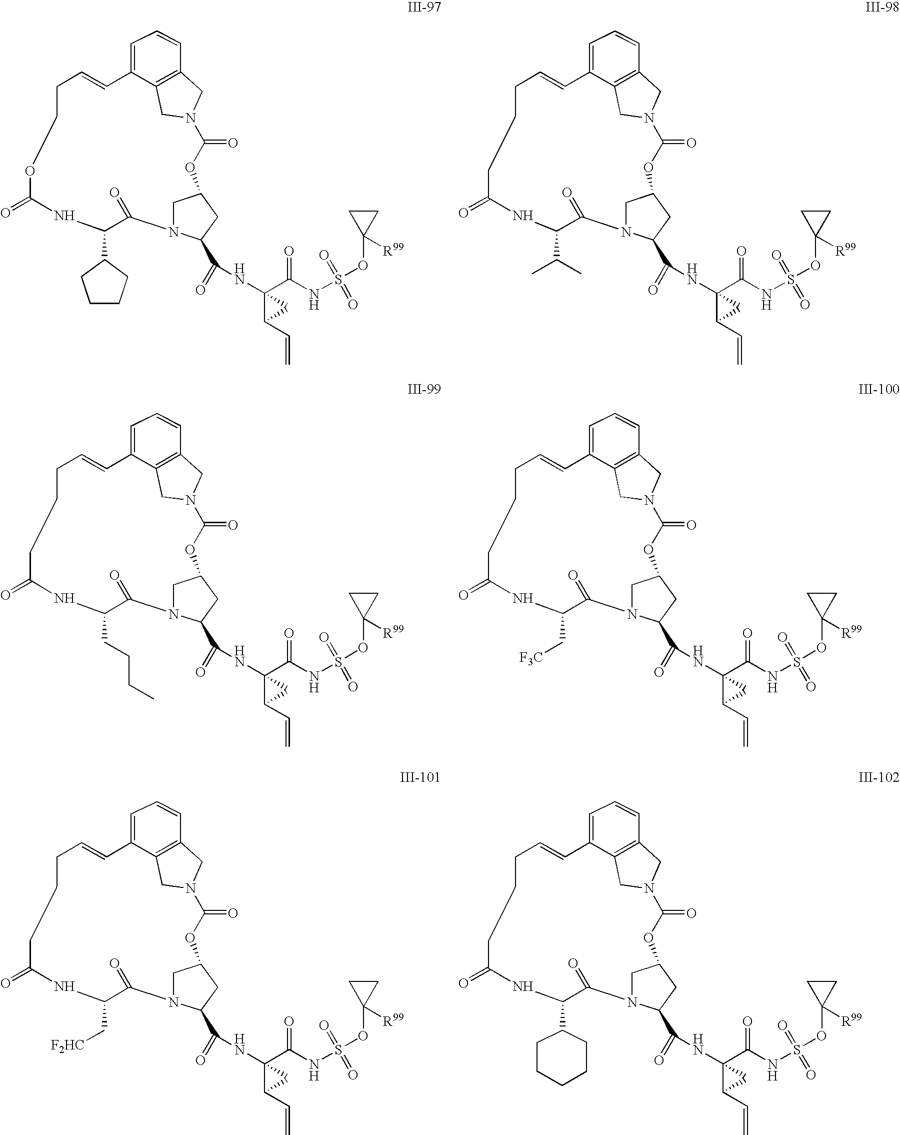

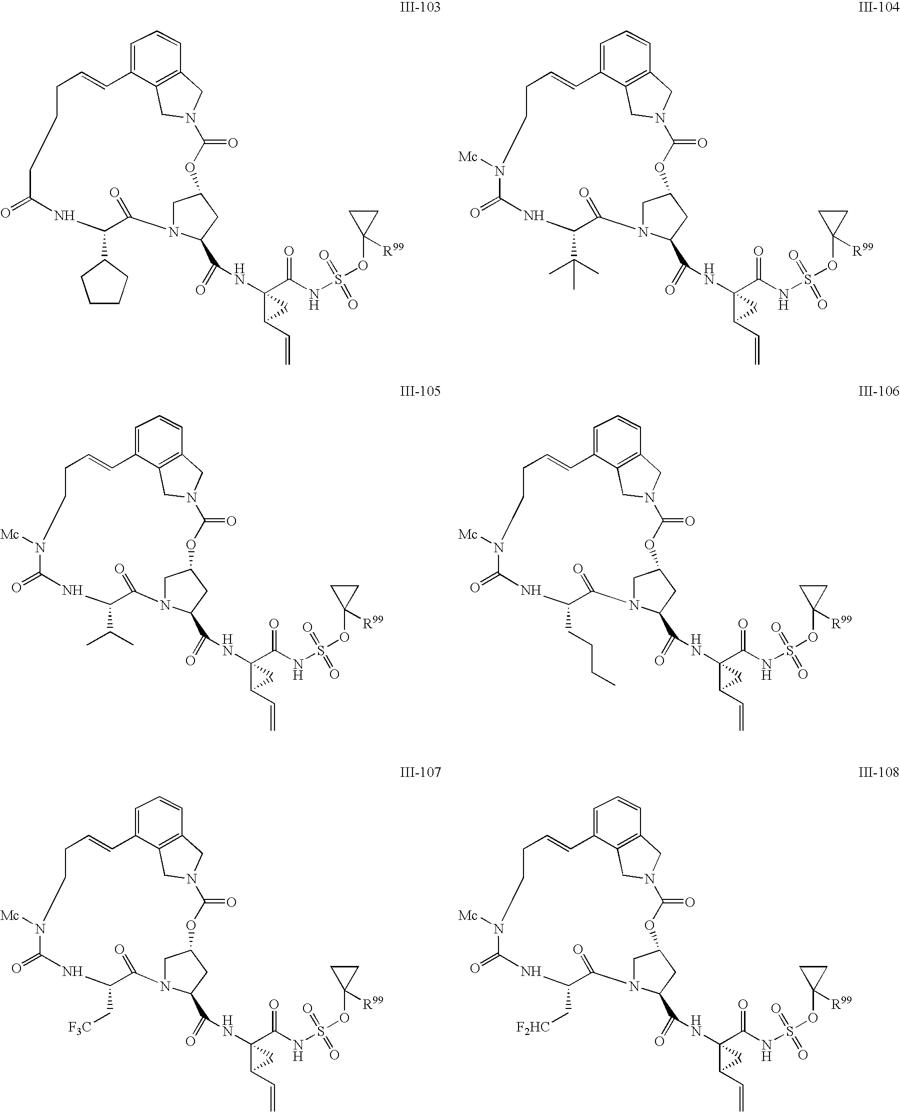

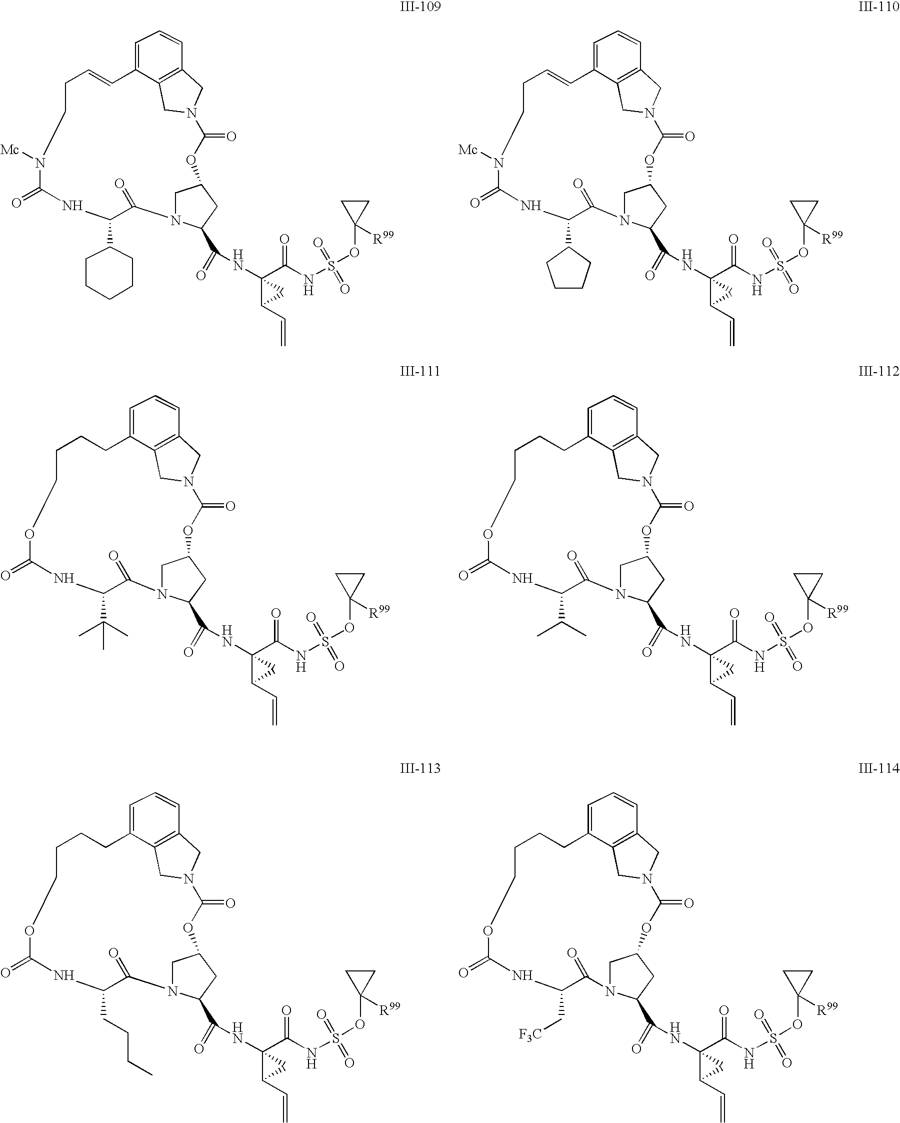

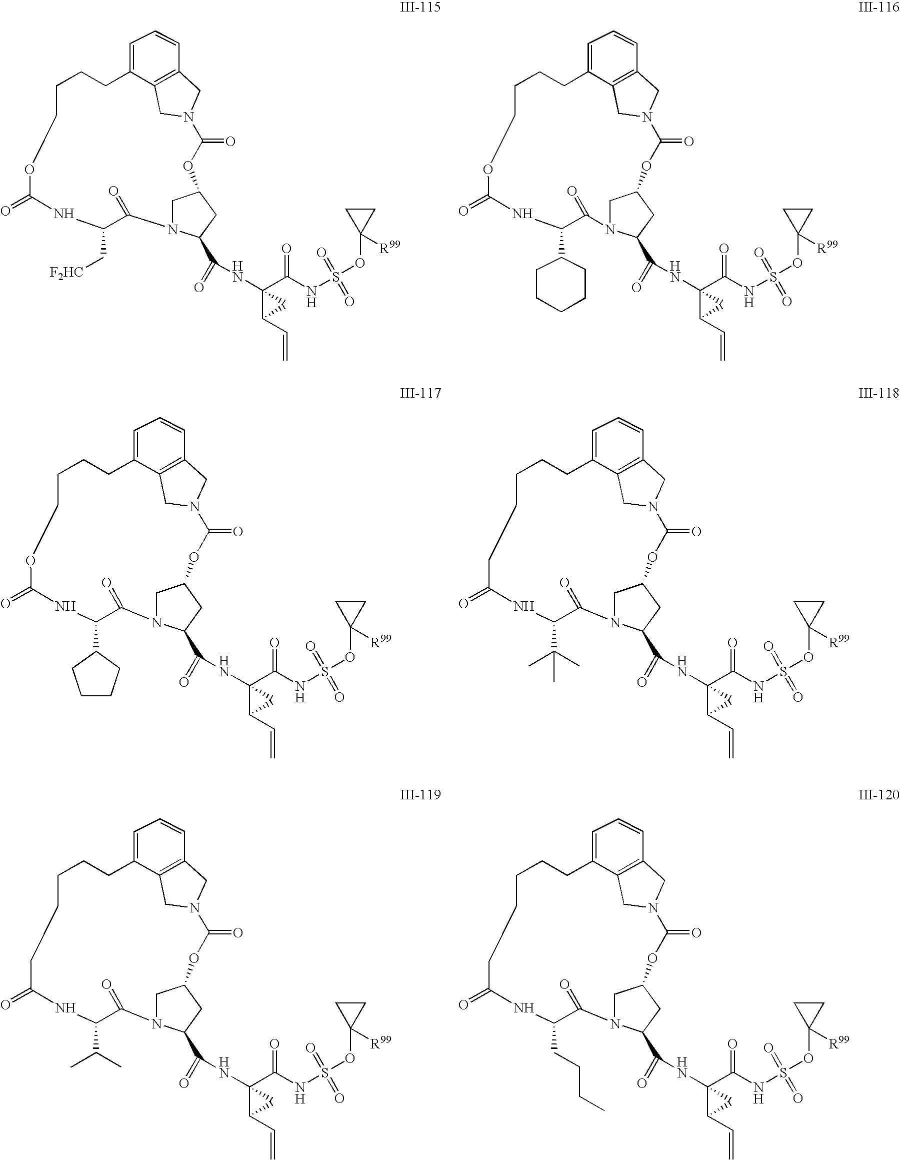

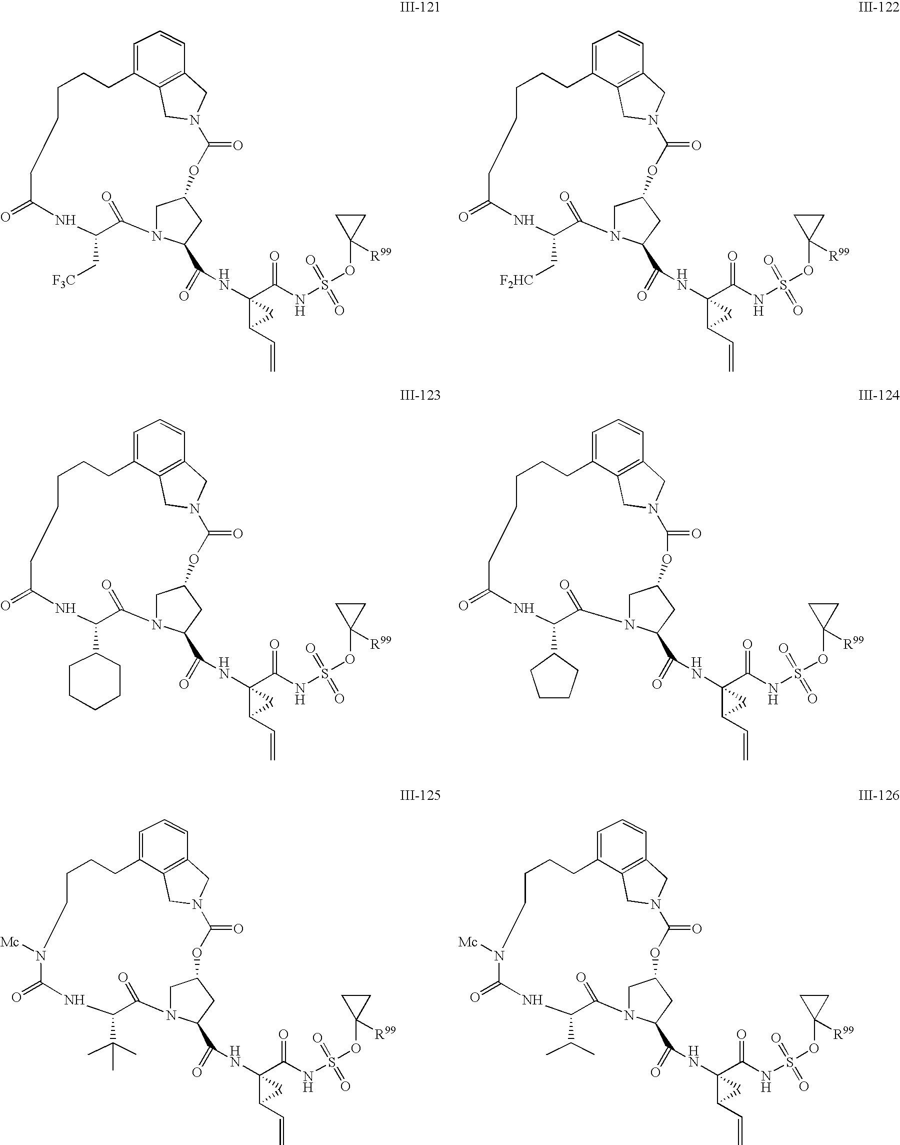

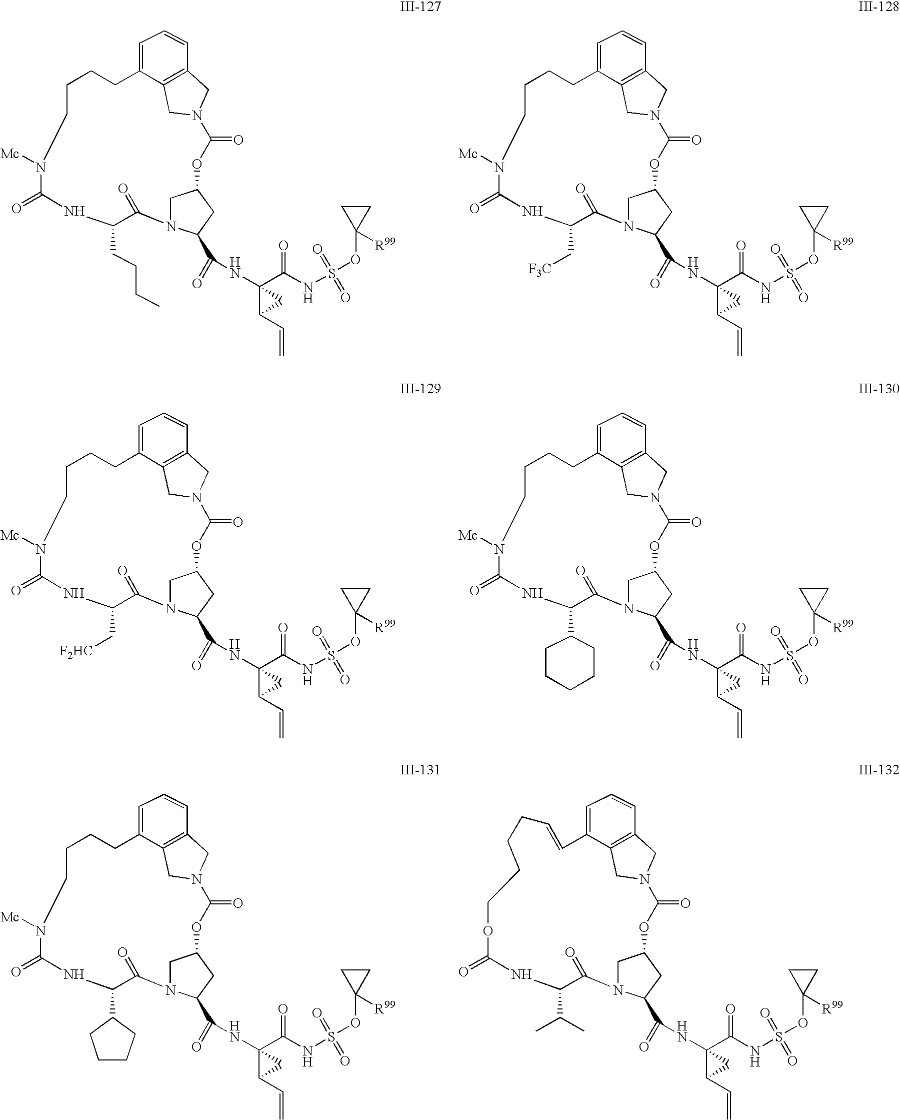

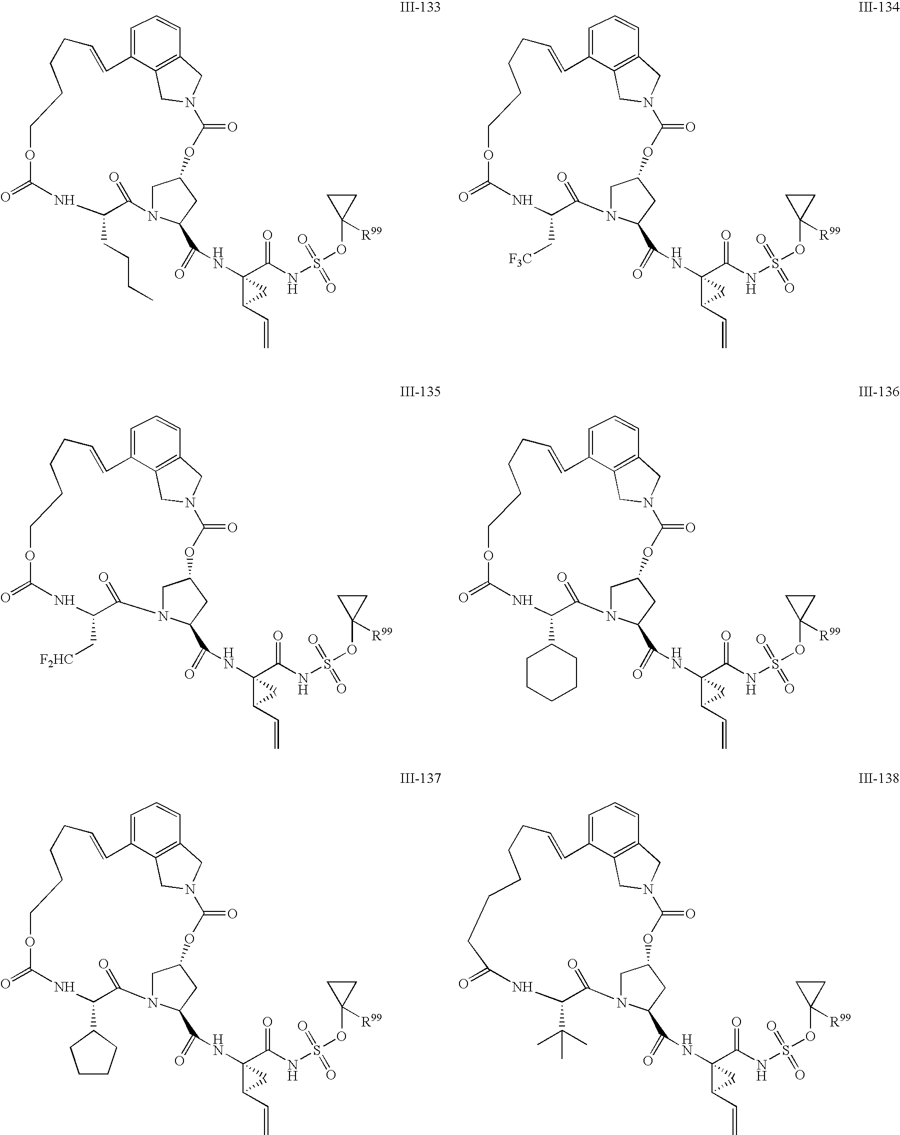

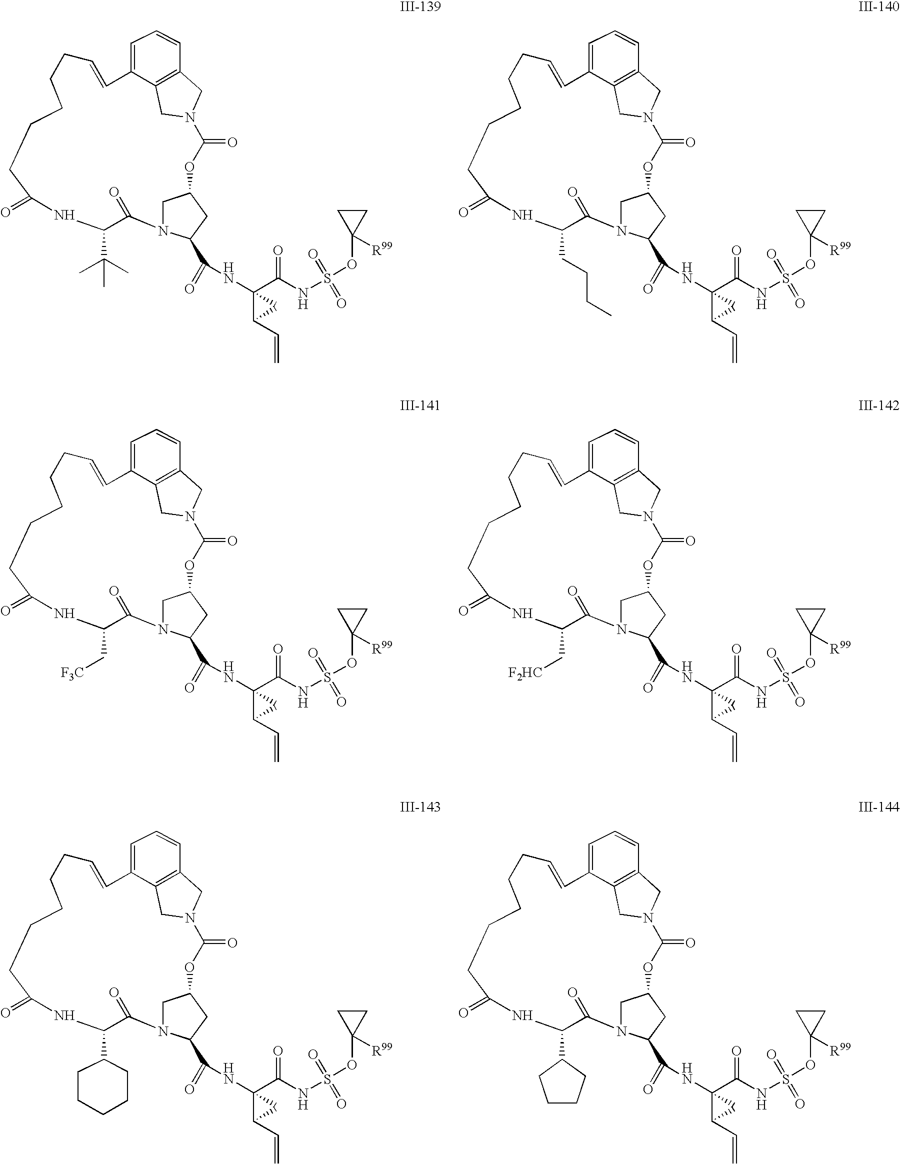

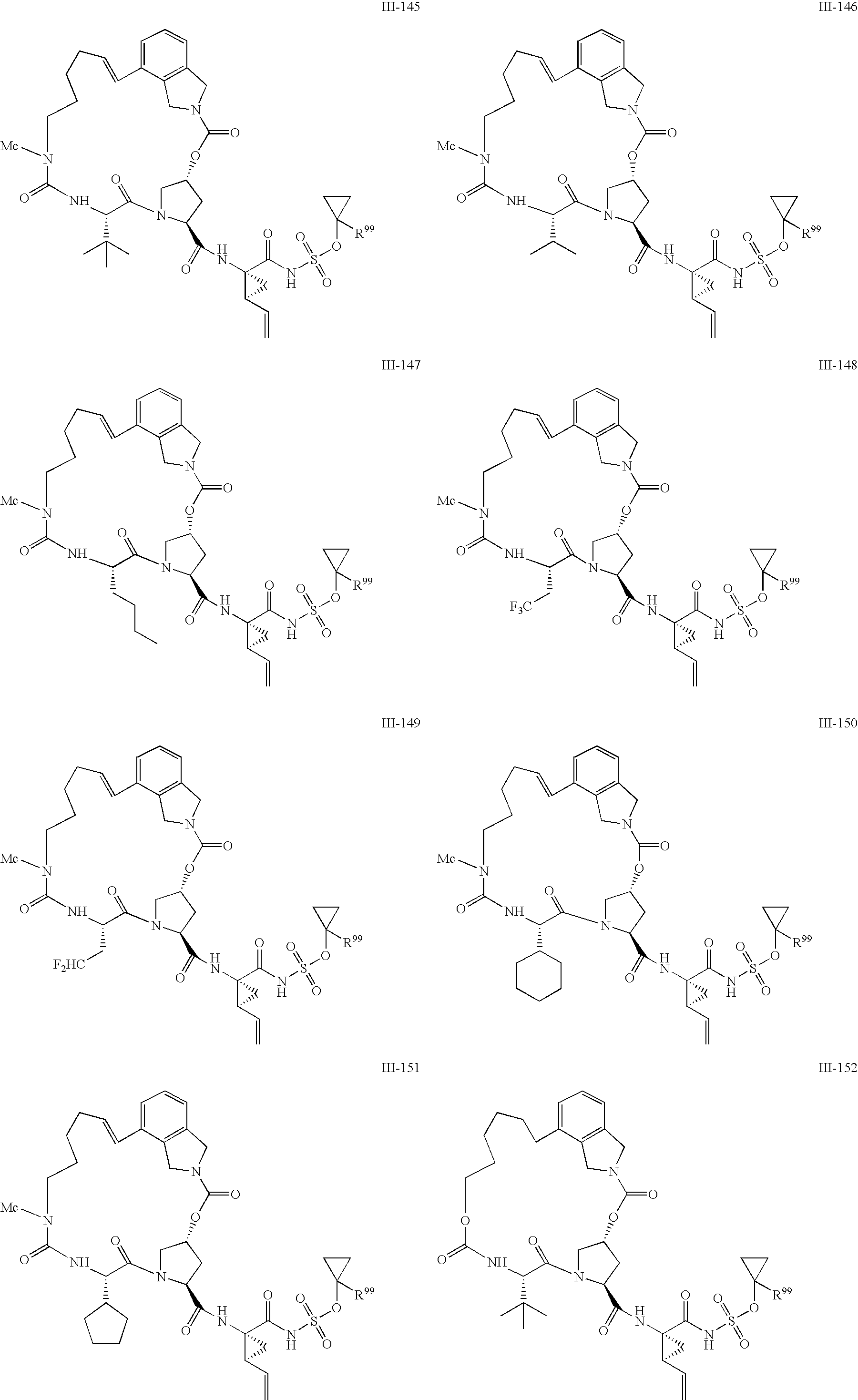

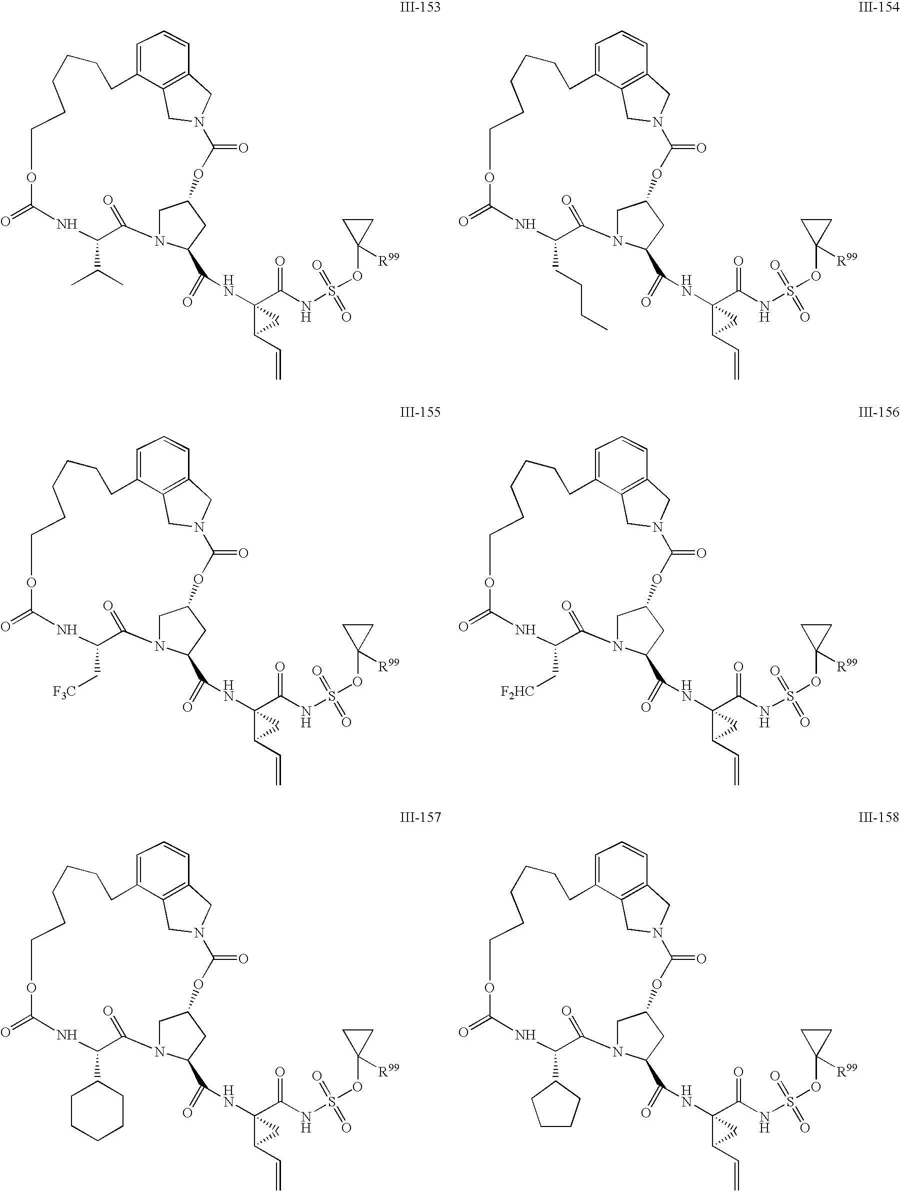

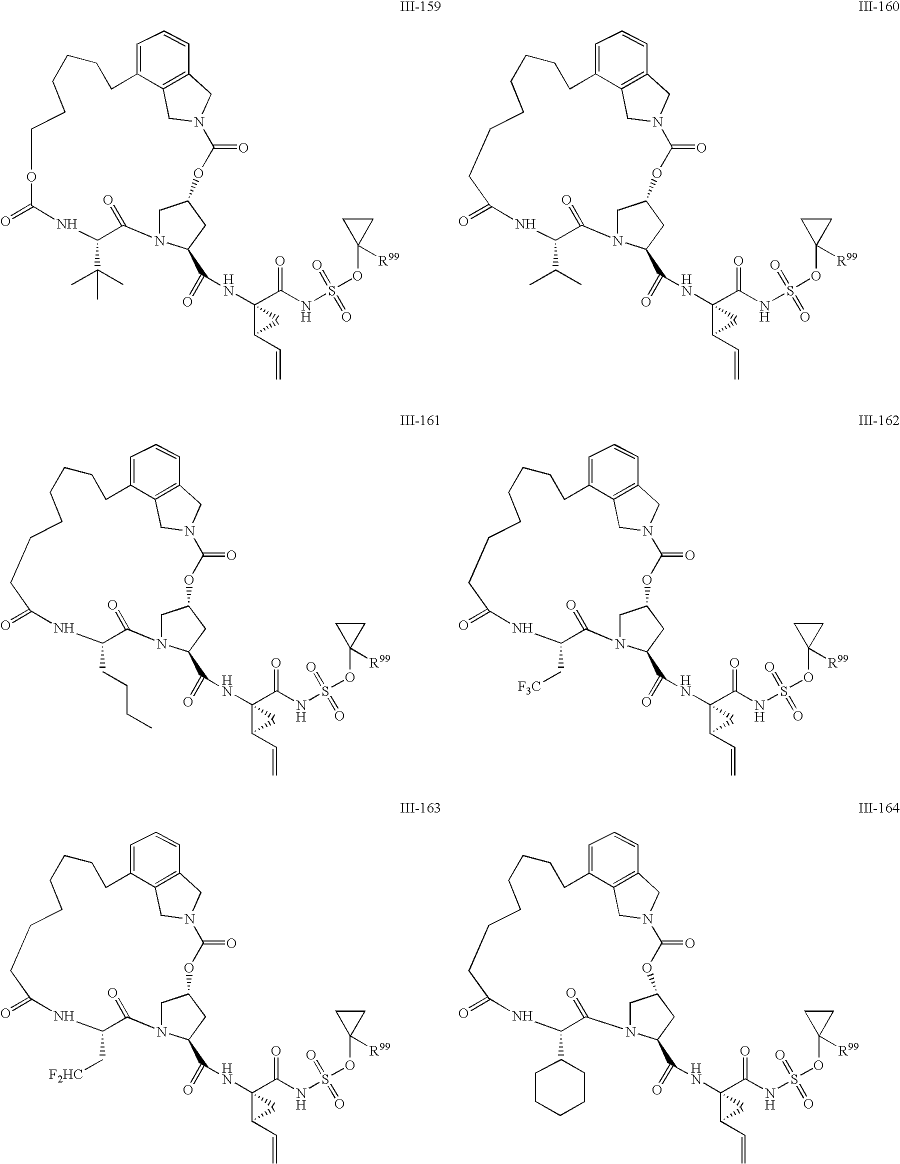

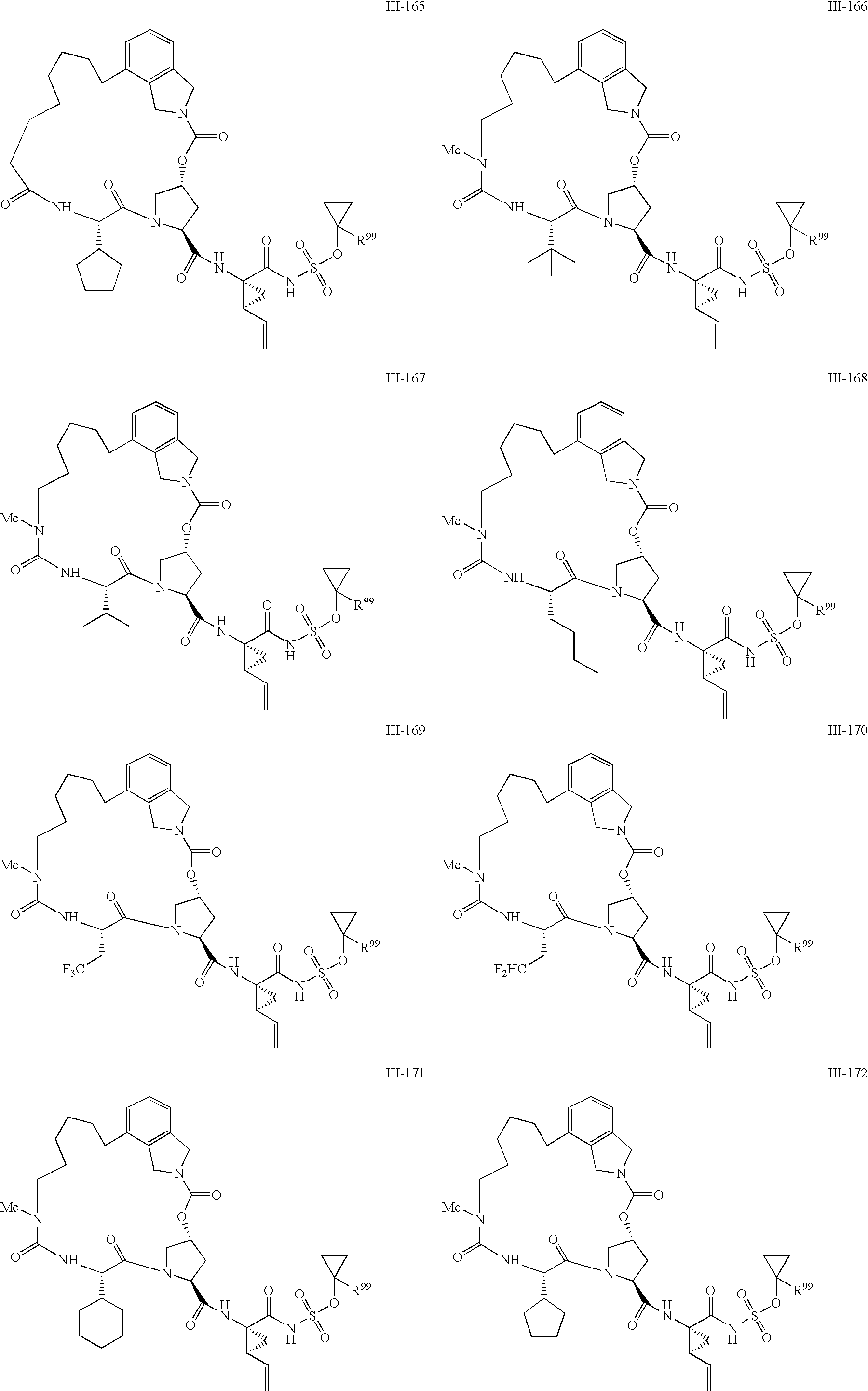

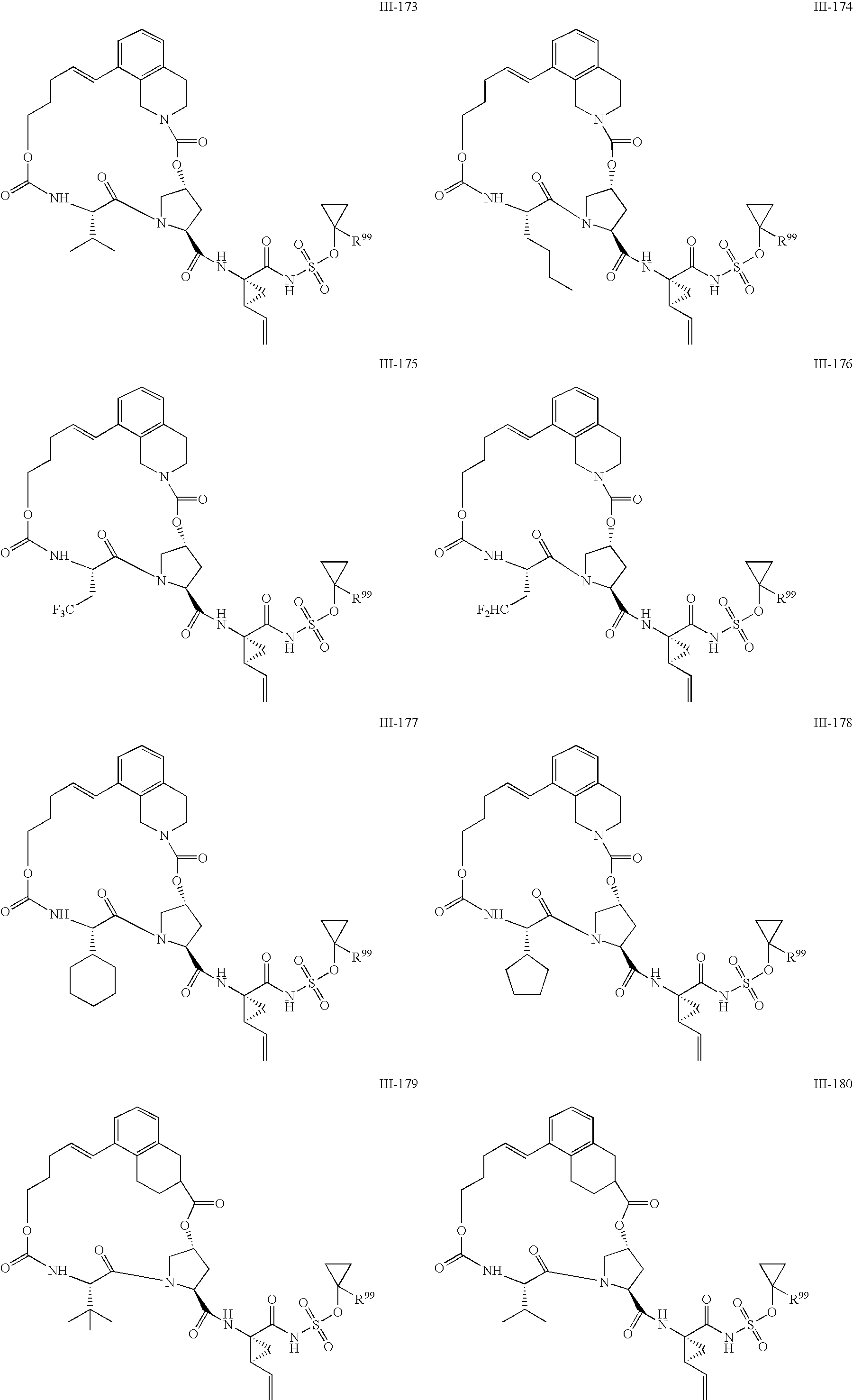

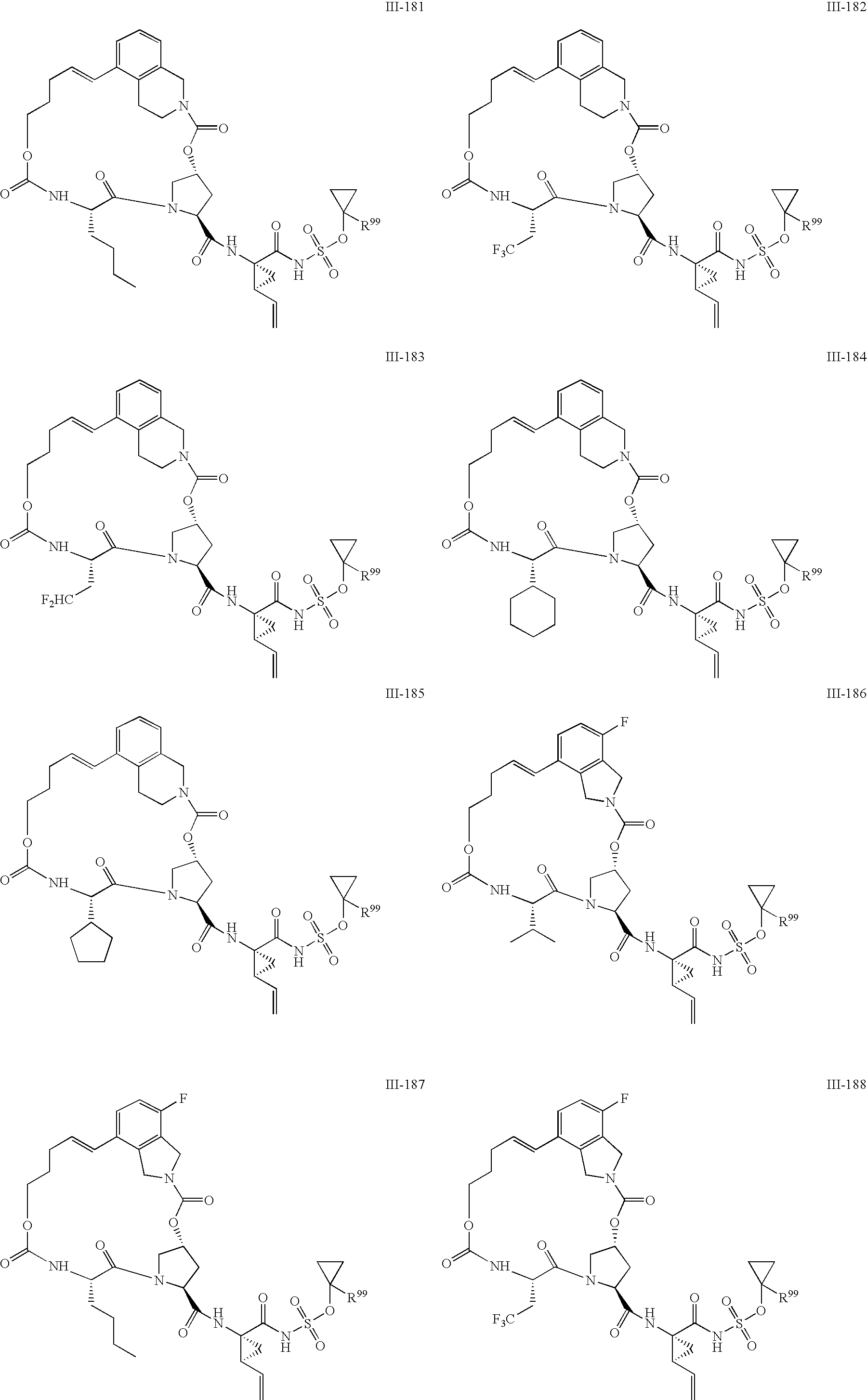

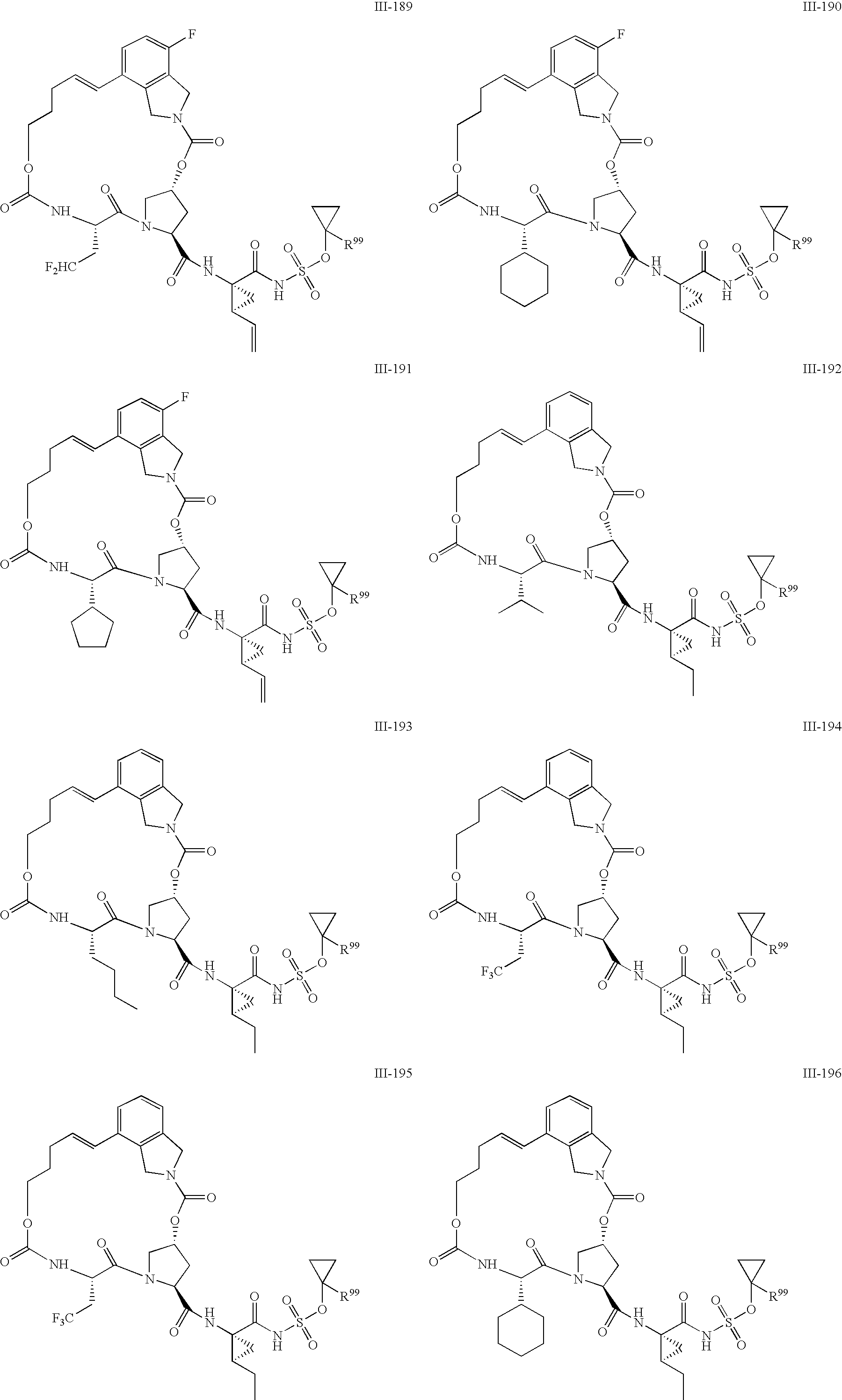

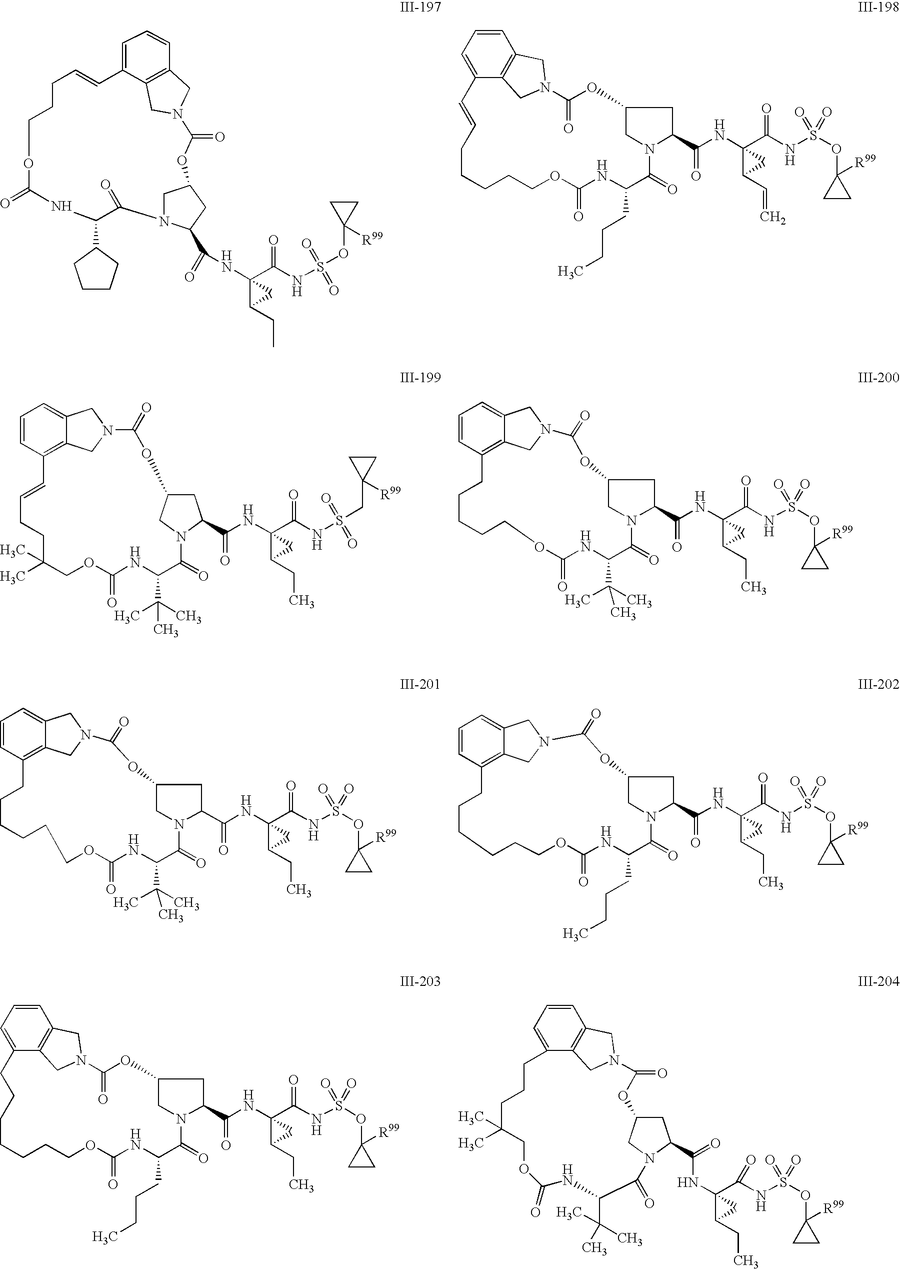

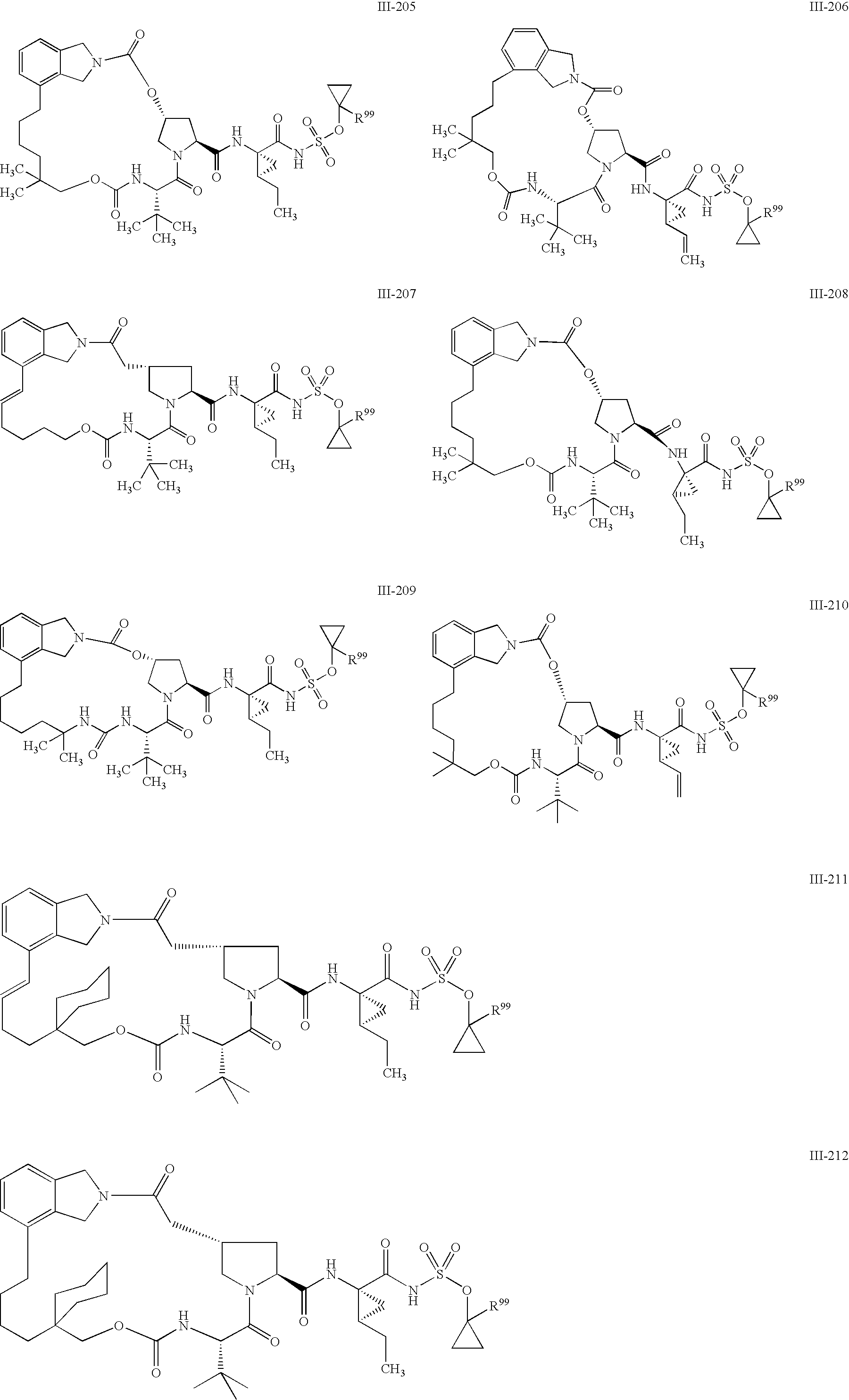

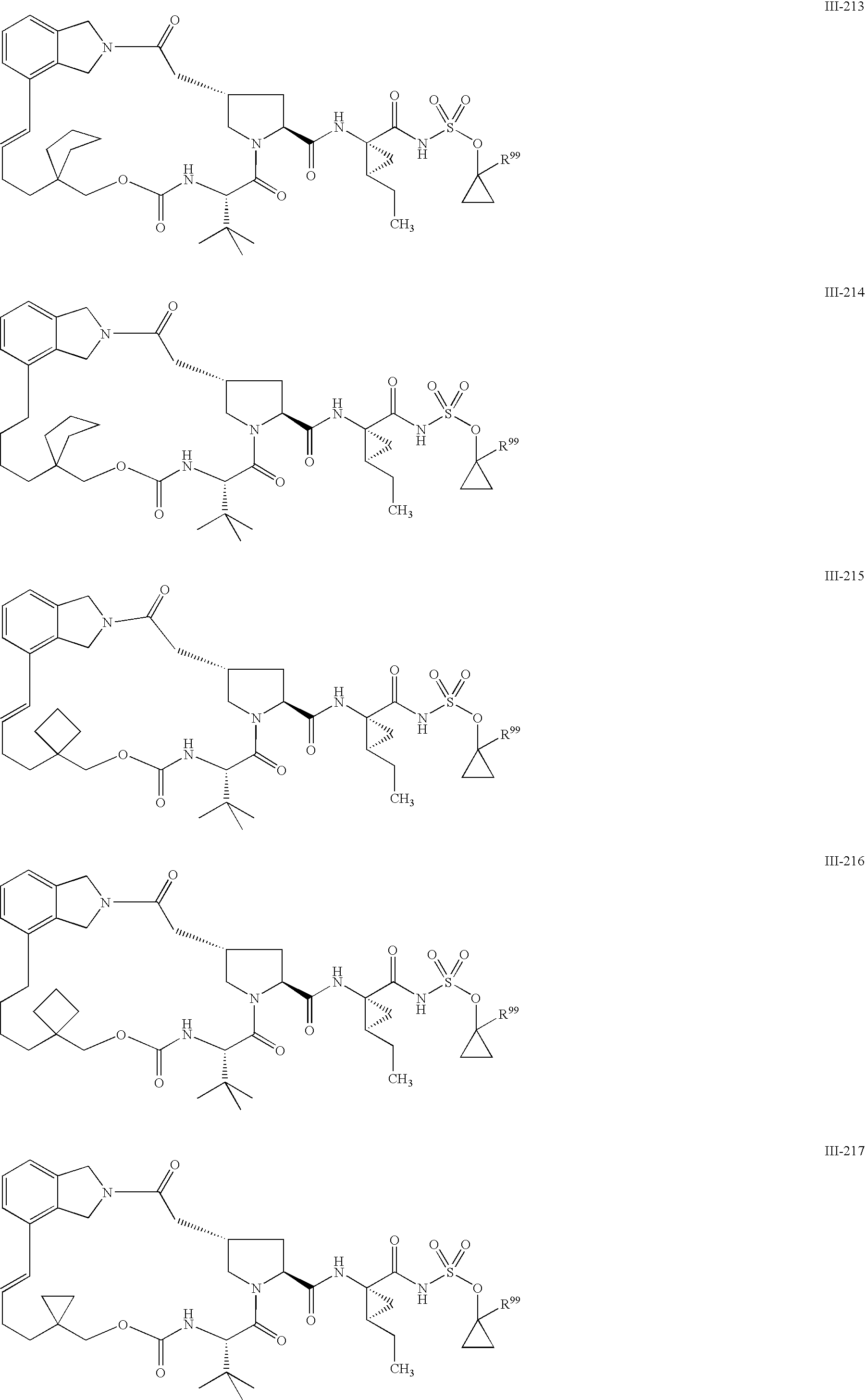

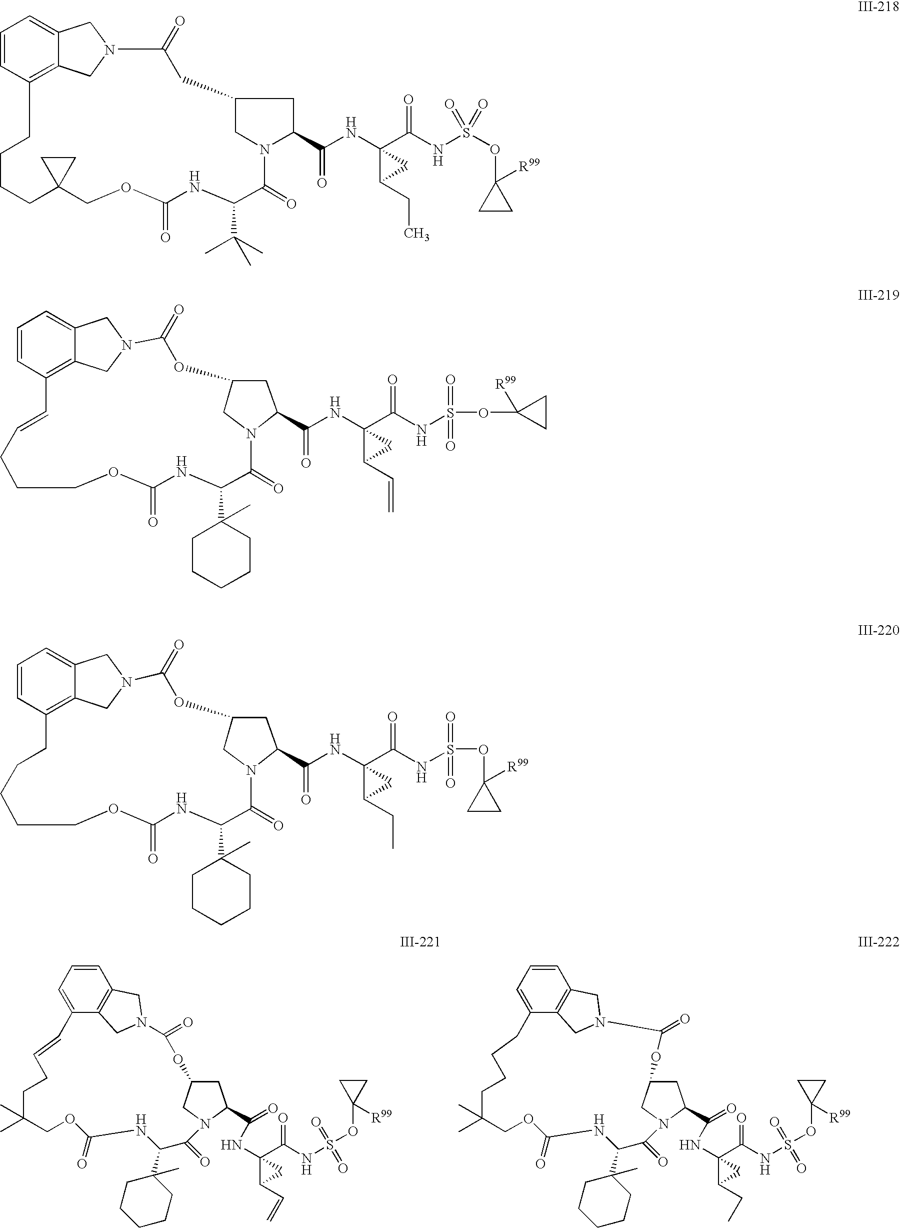

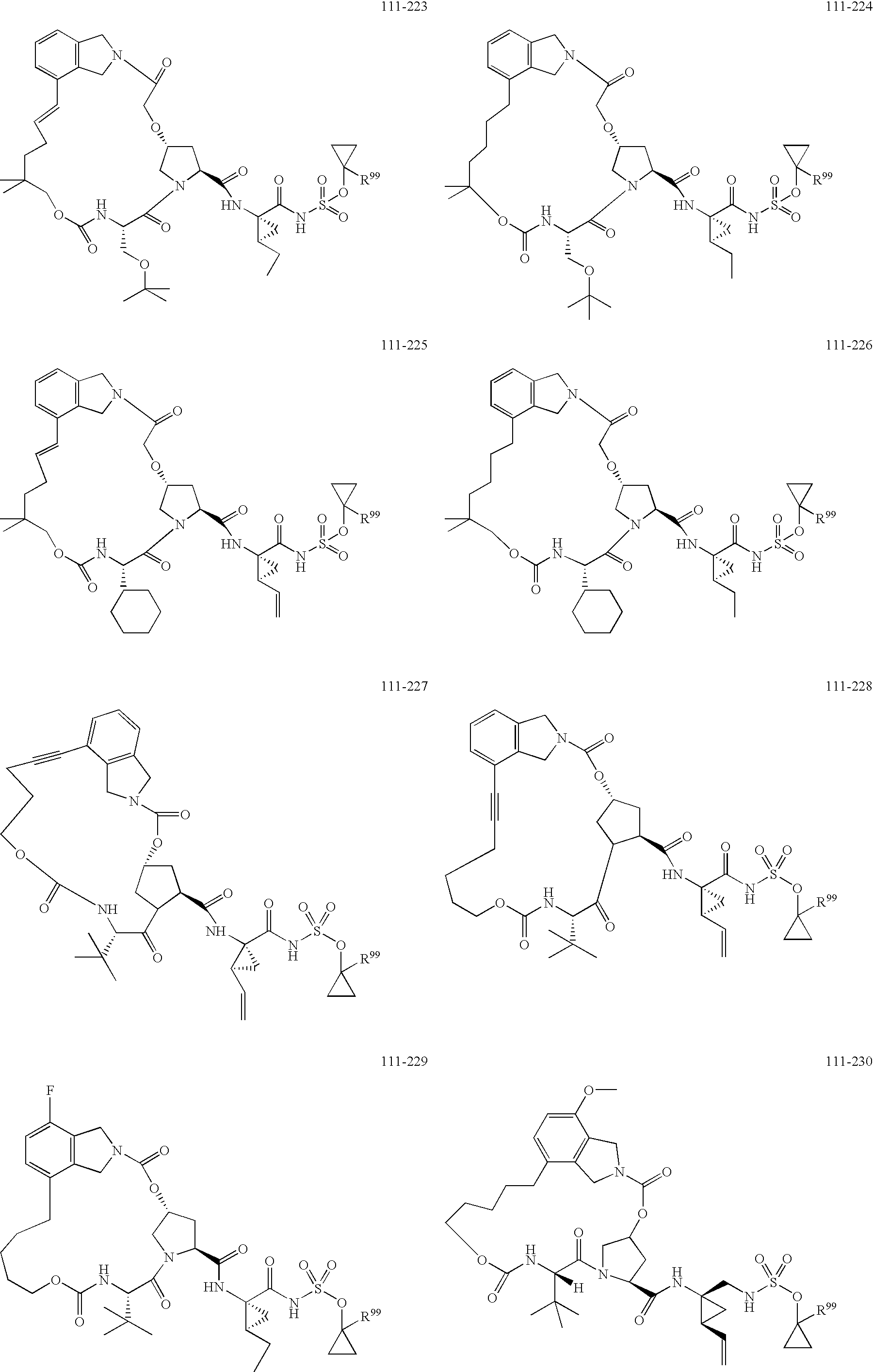

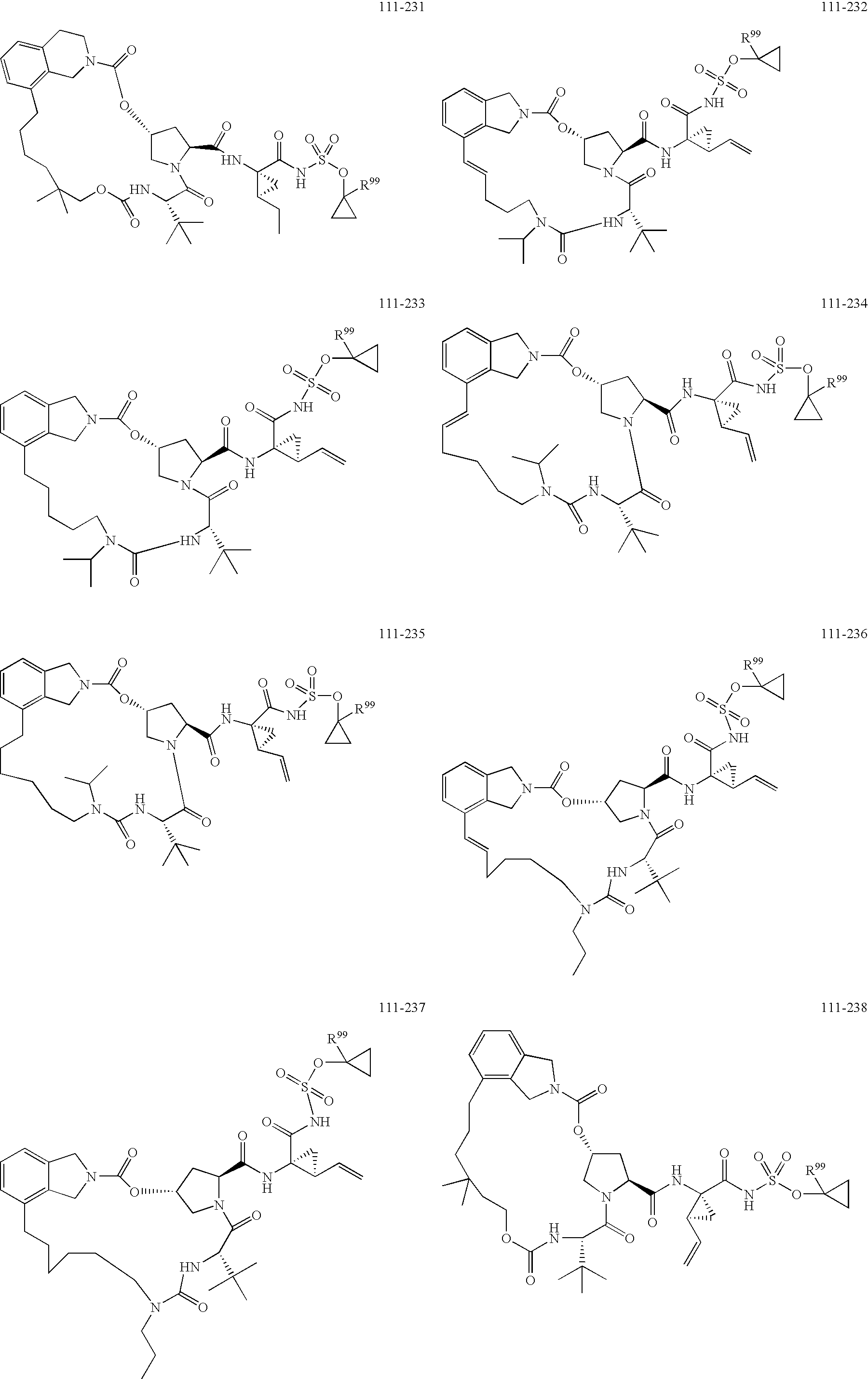

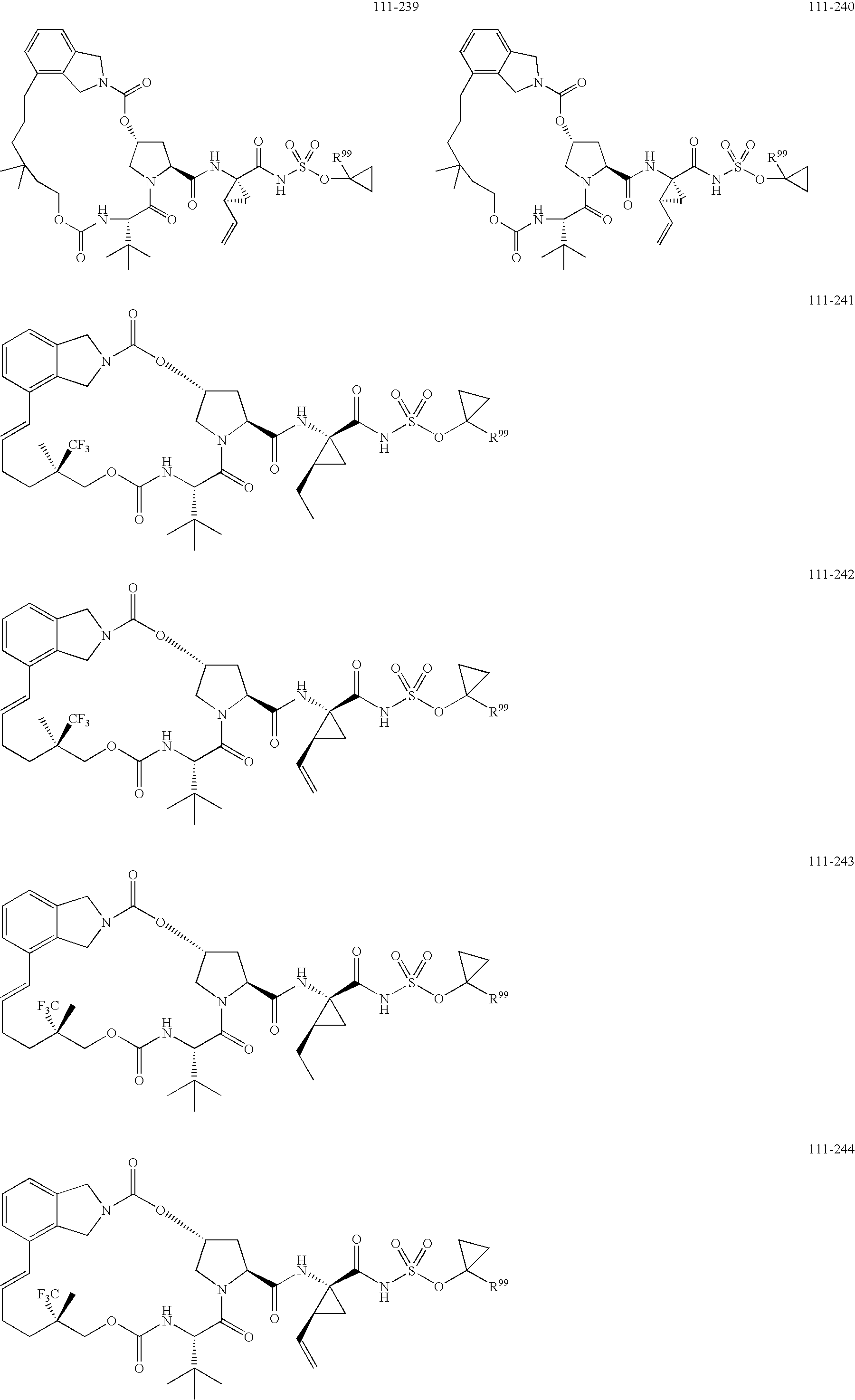

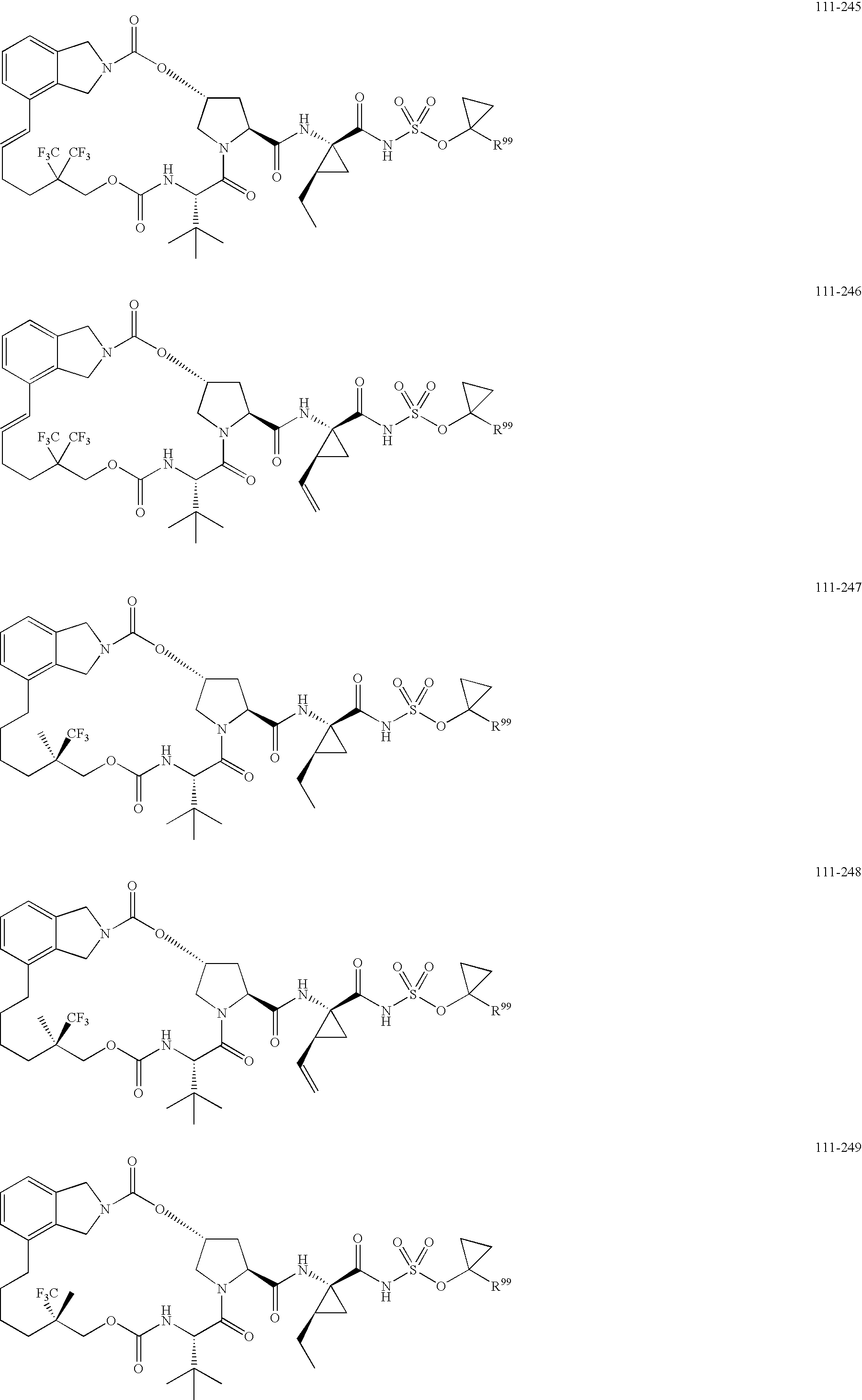

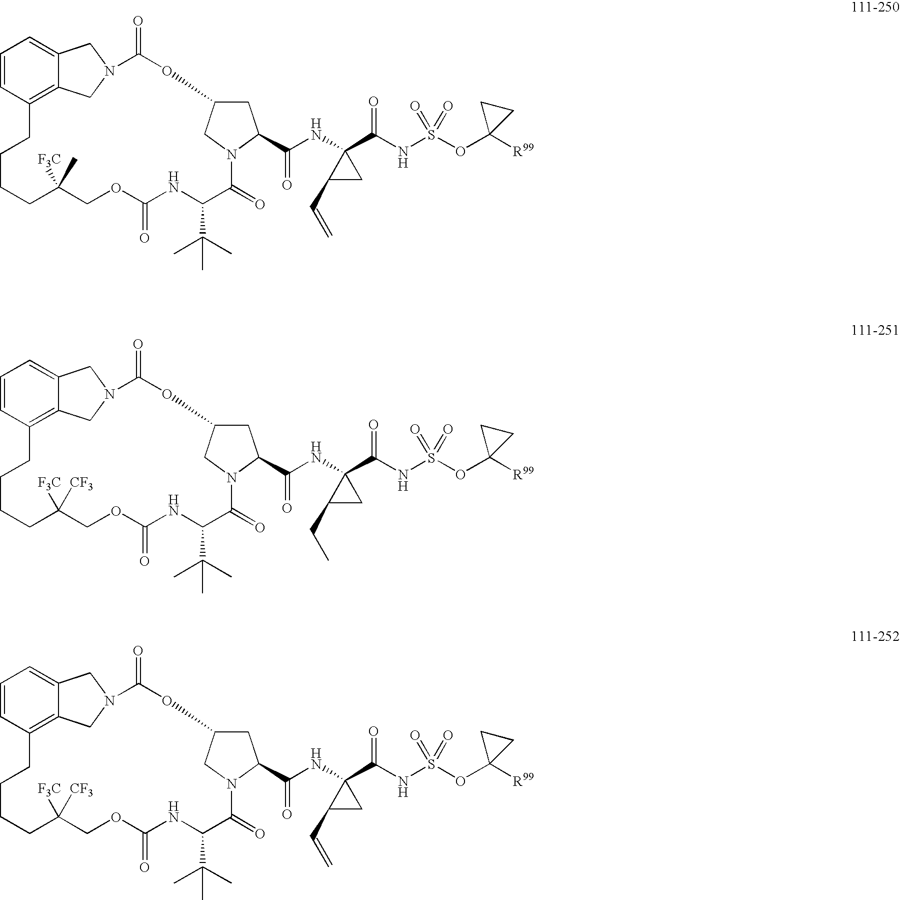

Other embodiments of the present invention include the following:

(a) A pharmaceutical composition comprising an effective amount of a compound of formula I, II, II-a, II-b, II-c, II-d, III, III-a, III-b, III-c or III-d and a pharmaceutically acceptable carrier.

(b) The pharmaceutical composition of (a), further comprising a second therapeutic agent selected from the group consisting of a HCV antiviral agent, immunomodulator, and an anti-infective agent.

(c) The pharmaceutical composition of (b), wherein the HCV antiviral agent is an antiviral selected from the group consisting of a HCV protease inhibitor and a HCV NS5B polymerase inhibitor.

(d) A pharmaceutical combination which is (i) a compound of formula I, II, II-a, II-b, II-c, II-d, III, III-a, III-c or III-d and (ii) a second therapeutic agent selected from the group consisting of a HCV antiviral agent, an immunomodulator, and an anti-infective agent; wherein the compound of formula I, II, II-a, II-b, II-c, II-d, III, III-a, III-c or III-d and the second therapeutic agent are each employed in an amount that renders the combination effective for inhibiting HCV NS3 protease, or for treating or preventing infection by HCV.

(e) The combination of (d), wherein the HCV antiviral agent is an antiviral selected from the group consisting of a HCV protease inhibitor and a HCV NS5B polymerase inhibitor.

(f) A method of inhibiting HCV NS3 protease in a subject in need thereof which comprises administering to the subject an effective amount of a compound of formula I, II, II-a, II-b, II-c, II-d, III, III-a, III-b, III-c or III-d.

(g) A method of preventing or treating infection by HCV in a subject in need thereof which comprises administering to the subject an effective amount of a compound of formula I, II, II-a, II-b, II-c, II-d, III, III-a, III-b, III-c or III-d.

(h) The method of (g), wherein the compound of formula I, II, II-a, II-b, II-c, II-d, III, III-a, III-b, III-c or III-d is administered in combination with an effective amount of at least one second therapeutic agent selected from the group consisting of a HCV antiviral agent, an immunomodulator, and an anti-infective agent.

(i) The method of (h), wherein the HCV antiviral agent is an antiviral selected from the group consisting of a HCV protease inhibitor and a HCV NS5B polymerase inhibitor.

(j) A method of inhibiting HCV NS3 protease in a subject in need thereof which comprises administering to the subject the pharmaceutical composition of (a), (b), or (c) or the combination of (d) or (e).

(k) A method of preventing or treating infection by HCV in a subject in need thereof which comprises administering to the subject the pharmaceutical composition of (a), (b), or (c) or the combination of (d) or (e).

The present invention also includes a compound of the present invention (i) for use in, (ii) for use as a medicament for, or (iii) for use in the preparation of a medicament for: (a) inhibiting HCV NS3 protease, or (b) preventing or treating infection by HCV. In these uses, the compounds of the present invention can optionally be employed in combination with one or more second therapeutic agents selected from HCV antiviral agents, anti-infective agents, and immunomodulators.

Additional embodiments of the invention include the pharmaceutical compositions, combinations and methods set forth in (a)-(k) above and the uses set forth in the preceding paragraph, wherein the compound of the present invention employed therein is a compound of one of the embodiments, aspects, classes, sub-classes, or features of the compounds described above. In all of these embodiments, the compound may optionally be used in the form of a pharmaceutically acceptable salt or hydrate as appropriate.

Whenever a compound described herein is substituted with more than one of the same designated group, e.g., “R¹¹¹” or “A³”, then it will be understood that the groups may be the same or different, i.e., each group is independently selected.

By way of example and not limitation, A³, A² and R¹¹¹ are all recursive substituents in certain embodiments. Typically, each of these may independently occur 20, 19, 18, 17, 16, 15, 14, 13, 12, 11, 10, 9, 8, 7, 6, 5, 4, 3, 2, 1, or 0, times in a given embodiment. More typically, each of these may independently occur 12 or fewer times in a given embodiment. Whenever a compound described herein is substituted with more than one of the same designated group, e.g., “R¹¹¹” or “A³”, then it will be understood that the groups may be the same or different, i.e., each group is independently selected. Wavy lines indicate the site of covalent bond attachments to the adjoining groups, moieties, or atoms.

The compounds of the invention have inhibitory activity toward HCV protease. Unexpectedly, it has been found that compounds possessing the acyl sulfamate group of the following formula:

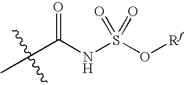

are suitably stable under physiological conditions. Additionally, it has been determined that representative compounds possessing this sulfamate group are unexpectedly potent inhibitors of HCV NS3 protease.

The entire content of International Patent Application Publication Numbers WO 2007/016441, WO 2008/051514, WO 2006/119061 as well as the entire content of United States Patent Application US 2007/0027071 is hereby incorporated herein by reference. In particular, information relating to suitable synthetic routes for preparing the compounds of formulae (Ia), (Ib), (Ic) therein are hereby incorporated herein by reference.

As used herein, the term “alkyl” refers to any linear or branched chain alkyl group having a number of carbon atoms in the specified range. Thus, for example, “C₁₋₆ alkyl” (or “C₁-C₆ alkyl”) refers to all of the hexyl alkyl and pentyl alkyl isomers as well as n-, iso-, sec- and t-butyl, n- and isopropyl, ethyl and methyl. As another example, “C₁₋₄ alkyl” refers to n-, iso-, sec- and t-butyl, n- and isopropyl, ethyl and methyl.

The term “haloalkyl” refers to an alkyl group wherein a hydrogen has been replaced by a halogen. The term “alkoxy” refers to an “alkyl-O—” group.

The term “alkylene” refers to any linear or branched chain alkylene group (or alternatively “alkanediyl”) having a number of carbon atoms in the specified range. Thus, for example, “—C₁₋₆ alkylene-” refers to any of the C₁ to C₆ linear or branched alkylenes. A class of alkylenes of particular interest with respect to the invention is —(CH2)1-6-, and sub-classes of particular interest include —(CH₂)₁₋₄₋, —(CH₂)₁₋₃₋, —(CH₂)₁₋₂₋, and —CH₂—. Also of interest is the alkylene —CH(CH₃₋.

The terms “cycloalkyl” refers to any cyclic ring of an alkane or alkene having a number of carbon atoms in the specified range. Thus, for example, “C₃₋₈ cycloalkyl” (or cycloalkyl”) refers to cyclopropyl, cyclobutyl, cycloheptyl, cyclohexyl, cycloheptyl, and cyclooctyl. The term “cycloalkoxy” refers to a “cycloalkyl-O—” group.

The term “halogen” (or “halo”) refers to fluorine, chlorine, bromine and iodine (alternatively referred to as fluoro, chloro, bromo, and iodo).

“Heterocycle” as used herein includes by way of example and not limitation these heterocycles described in Paquette, Leo A.; Principles of Modern Heterocyclic Chemistry (W.A. Benjamin, New York, 1968), particularly Chapters 1, 3, 4, 6, 7, and 9; The Chemistry of Heterocyclic Compounds, A Series of Monographs” (John Wiley & Sons, New York, 1950 to present), in particular Volumes 13, 14, 16, 19, and 28; and J. Am. Chem. Soc. (1960) 82:5566. In one specific embodiment of the invention ‘heterocycle” includes a “carbocycle” as defined herein, wherein one or more (e.g. 1, 2, 3, or 4) carbon atoms have been replaced with a heteroatom (e.g. 0, N, or 5).

Examples of heterocycles include by way of example and not limitation pyridyl, dihydroypyridyl, tetrahydropyridyl (piperidyl), thiazolyl, tetrahydrothiophenyl, sulfur oxidized tetrahydrothiophenyl, pyrimidinyl, (uranyl, thienyl, pyrrolyl, pyrazolyl, imidazolyl, tetrazolyl, benzofuranyl, thianaphthalenyl, indolyl, indolenyl, quinolinyl, benzimidazolyl, piperidinyl, 4-piperidonyl, pyrrolidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, azocinyl, triazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, pteridinyl, 4H-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, isatinoyl, and bis-tetrahydrofuranyl:

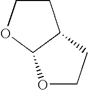

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, itnidazole, imidazolidine, 2-inaida7oline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or carboline. Still more typically, nitrogen bonded heterocycles include 1-aziridyl, 1-azetedyl, 1-pyrrolyl, 1-imidazolyl, 1-pyrazolyl, and 1-piperidinyl.

“Carbocycle” refers to a saturated, unsaturated or aromatic ring having up to about 25 carbon atoms. Typically, a carbocycle has about 3 to 7 carbon atoms as a monocycle, about 7 to 12 carbon atoms as a bicycle, and up to about 25 carbon atoms as a polycycle. Monocyclic carbocycles typically have 3 to 6 ring atoms, still more typically 5 or 6 ring atoms. Bicyclic carbocycles typically have 7 to 12 ring atoms, e.g., arranged as a bicyclo [4,5], [5,5], [5,6] or [6,6] system, or 9 or 10 ring atoms arranged as a bicyclo [5,6] or [6,6] system. The term carbocycle includes “cycloalkyl” which is a saturated or unsaturated carbocycle. Examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, phenyl, spiryl and naphthyl.

The term “PRT” is selected from the terms “prodrug moiety” and “protecting group” as defined herein.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity. The invention includes all stereoisomers of the compounds described herein.

Unless expressly stated to the contrary, all ranges cited herein are inclusive. For example, a heteroaryl ring described as containing from “1 to 3 heteroatoms” means the ring can contain 1, 2, or 3 heteroatoms. It is also to be understood that any range cited herein includes within its scope all of the sub-ranges within that range. The oxidized forms of the heteroatoms N and S are also included within the scope of the present invention.

When any variable (e.g., fe and Fe′) occurs more than one time in any constituent or in formula I, II, II-a, II-b, II-c, II-d, III, III-a, III-b, III-c or III-d or in any other formula depicting and describing compounds of the invention, its definition on each occurrence is independent of its definition at every other occurrence. Also, combinations of substituents and/or variables are permissible only if such combinations result in stable compounds.

Unless expressly stated to the contrary, substitution by a named substituent is permitted on any atom in a ring (e.g., aryl, a heteroaromatic ring, or a saturated heterocyclic ring) provided such ring substitution is chemically allowed and results in a stable compound. A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

As a result of the selection of substituents and substituent patterns, certain of the compounds of the present invention can have asymmetric centers and can occur as mixtures of stereoisomers, or as individual diastereomers, or enantiomers. All isomeric forms of these compounds, whether isolated or in mixtures, are within the scope of the present invention.

As would be recognized by one of ordinary skill in the art, certain of the compounds of the present invention can exist as tautomers. For the purposes of the present invention a reference to a compound of formula I, II, II-a, II-b, II-c, II-d, III, III-a, III-b, III-c or III-d is a reference to the compound per se, or to any one of its tautomers per se, or to mixtures of two or more tautomers.

The compounds of the present inventions are useful in the inhibition of HCV protease (e.g., HCV NS3 protease) and the prevention or treatment of infection by HCV. For example, the compounds of this invention are useful in treating infection by HCV after suspected past exposure to HCV by such means as blood transfusion, exchange of body fluids, bites, accidental needle stick, or exposure to patient blood during surgery.

The compounds of this invention are useful for isolating enzyme mutants, which are excellent screening tools for more powerful antiviral compounds. Furthermore, the compounds of this invention are useful in establishing or determining the binding site of other antivirals to HCV protease, e.g., by competitive inhibition. Thus the compounds of this invention are commercial products to be sold for these purposes.

The compounds of the present invention may be administered in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof). Suitable salts include acid addition salts which may, for example, be formed by mixing a solution of the compound of the present invention with a solution of a pharmaceutically acceptable acid such as hydrochloric acid, sulfuric acid, acetic acid, trifluoroacetic acid, or benzoic acid. Many of the compounds of the invention carry an acidic moiety, in which case suitable pharmaceutically acceptable salts thereof can include alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

The term “administration” and variants thereof (e.g., “administering” a compound) in reference to a compound of the invention mean providing the compound or a prodrug of the compound to the individual in need of treatment When a compound of the invention or a prodrug thereof is provided in combination with one or more other active agents (e.g., antiviral agents useful for treating HCV infection), “administration” and its variants are each understood to include concurrent and sequential provision of the compound or salt (or hydrate) and other agents.

As used herein, the term “composition” is intended to encompass a product comprising the specified ingredients, as well as any product which results, directly or indirectly, from combining the specified ingredients.

By “pharmaceutically acceptable” is meant that the ingredients of the pharmaceutical composition must be compatible with each other and not deleterious to the recipient thereof.

The term “subject” (alternatively referred to herein as “patient”) as used herein refers to an animal, preferably a mammal, most preferably a human, who has been the object of treatment, observation or experiment

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active compound sufficient to inhibit HCV NS3 protease and thereby elicit the response being sought (i.e., an “inhibition effective amount”). When the active compound (i.e., active ingredient) is administered as the salt, references to the amount of active ingredient are to the free acid or free base form of the compound.

For the purpose of inhibiting HCV NS3 protease and preventing or treating HCV infection, the compounds of the present invention, optionally in the form of a salt or a hydrate, can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen mute of administration and standard pharmaceutical practice. The compounds of the invention can, for example, be administered orally, parenterally (including subcutaneous injections, intravenous, intramuscular, intrasternal injection or infusion techniques), by inhalation spray, or rectally, in the form of a unit dosage of a pharmaceutical composition containing an effective amount of the compound and conventional non-toxic pharmaceutically-acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the blce. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose.

Further description of methods suitable for use in preparing pharmaceutical compositions of the present invention and of ingredients suitable for use in said compositions is provided in Remington's Pharmaceutical Sciences, 18th edition, edited by A. R. Gennaro, Mack Publishing Co., 1990.

The compounds of this invention can be administered orally in a dosage range of 0.001 to 1000 mg/kg of mammal (e.g., human) body weight per day in a single dose or in divided doses. One preferred dosage range is 0.01 to 500 mg/kg body weight per day orally in a single dose or in divided doses. Another preferred dosage range is 0.1 to 100 mg/kg body weight per day orally in single or divided doses. For oral administration, the compositions can be provided in the form of tablets or capsules containing 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### Combination Therapy

Combinations of one or more compounds of the present invention and one or more additional pharmaceutically active agent(s) may be used in the practice of the present invention to treat human beings having an HCV infection. Useful active therapeutic agents for treating an HCV infection include interferons, ribavirin or its analogs, HCV NS3 protease inhibitors, alpha-glucosidase 1 inhibitors, hepatoprotectants, nucleoside or nucleotide inhibitors of HCV NS5B polymerase, non-nucleoside inhibitors of HCV N55B polymerase, HCV NS5A inhibitors, TLR-7 agonists, cyclophillin inhibitors, HCV IRES inhibitors, and pharmacokinetic enhancers.

More specifically, other active therapeutic ingredients or agents for treating HCV include:

(1) interferons selected from the group consisting of pegylated rIFN-alpha 2b (PEG-Intron), pegylated rIFN-alpha 2a (Pegasys), rIFN-alpha 2b (Intron A), rIFN-alpha 2a (Roferon-A), interferon alpha (MOR-22, OPC-18, Alfaferone, Alfanative, Multiferon, subalin), interferon alfacon-1 (Infergen), interferon alpha-nl (Wellferon), interferon alpha-n3 (Alferon), interferon-beta (Avonex, DL-8234), interferon-omega (omega DUROS, Biomed 510), albinterferon alpha-2b (Albuferon), IFN alpha-2b XL, BLX-883 (Locteron), DA-3021, glycosylated interferon alpha-2b (AVI-005), PEG-Infergen, PEGylated interferon lambda-1 (PEGylated IL-29), belerofon, and mixtures thereof;

(2) ribavirin and its analogs selected from the group consisting of ribavirin (Rebetol, Copegus), taribavirin (Viramidine), and mixtures thereof;

(3) HCV NS3 protease inhibitors selected from the group consisting of boceprevir (SCH-503034, SCH-7), telaprevir (VX-950), TMC-435350, BI-1335, BI-1230, MK-7009, VBY-376, VX-500, BMS-790052, BMS-605339, PHX-1766, AS-101, YH-5258, YH5530, YH5531, ITMN-191, and mixtures thereof;

(4) alpha-glucosidase 1 inhibitors selected from the group consisting of celgosivir (MX-3253), Miglitol, UT-231B, and mixtures thereof;

(5) hepatoprotectants selected from the group consisting of IDN-6556, ME 3738, LB-84451, silibilin, MitoQ, and mixtures thereof;

(6) nucleoside or nucleotide inhibitors of HCV NS5B polymerase selected from the group consisting of R1626, R7128 (R4048), 1DX184, IDX-102, BCX-4678, valopicitabine (NM-283), MK-0608, and mixtures thereof;

(7) non-nucleoside inhibitors of HCV NS5B polymerase selected from the group consisting of PF-868554, VCH-759, VCH-916, JTK-652, MK-3281, VBY-708, VCH-222, A848837, ANA-598, GL60667, GL59728, A-63890, A-48773, A-48547, BC-2329, VCH-796 (nesbuvir), GSK625433, BILN-1941, XTL-2125, GS-9190, and mixtures thereof;

(8) HCV NS5A inhibitors selected from the group consisting of AZD-2836 (A-831), A-689, and mixtures thereof;

(9) TLR-7 agonists selected from the group consisting of ANA-975, SM-360320, and mixtures thereof;

(10) cyclophillin inhibitors selected from the group consisting of DEBIO-025, SCY-635, NIM811, and mixtures thereof;

(11) HCV IRES inhibitors selected from the group consisting of MCI-067,

(12) pharmacokinetic enhancers selected from the group consisting of BAS-100, SPI-452, PF-419-4477, TMC-41629, roxythromycin, and mixtures thereof; and

(13) other drugs for treating HCV selected from the group consisting of thymosin alpha 1 (Zadaxin), nitazoxanide (Alinea, NTZ), BIVN-401 (virostat), PYN-17 (altirex), KPE02003002, actilon (CPG-10101), KRN-7000, civacir, GI-5005, XTL-6865, BIT225, PTX-111, ITX2865, TT-033i, ANA 971, NOV-205, tarvacin, EHC-18, VGX-410C, EMZ-702, AVI 4065, BMS-650032, BMS-791325, Bavituximab, MDX-1106 (ONO-4538), Oglufanide, VX-497 (merimepodib), and mixtures thereof.

Thus, in a further embodiment, the present invention provides a combination pharmaceutical composition comprising:

a) a compound of the present invention or a pharmaceutically acceptable salt thereof; and

b) a second pharmaceutically active agent (or pharmaceutically acceptable salt thereof) effective to treat HCV.

In yet another embodiment, the present application provides a method for treating an HCV infection, wherein the method comprises the step of co-administering, to a human being in need thereof; a therapeutically effective amount of a compound of the present invention and one or more of the additional active agents described herein that are effective to treat HCV.

In the practice of this aspect of the invention, typically the amounts of a compound of the present invention and the one or more additional therapeutic agent(s) are individually therapeutic, but it is within the scope of the invention for the amounts of the compound of the present invention (referred to as “the compound”) and the one or more additional therapeutic agent(s) to be subtherapeutic by themselves, but the combination of the compound of the present invention and the one or more additional therapeutic agent(s) is therapeutic.

Co-administration of the compound of the present invention with one or more other active agents generally refers to simultaneous or sequential administration of the compounded one or more other active agents, such that the compounded one or more other active agents are both present in the body of the patient. Simultaneous administration of the compounded one or more additional therapeutic agents can be achieved, for example, by miming the compounded one or more additional therapeutic agents in a single dosage form, such as a tablet or injectable solution. Again by way of example, simultaneous administration of the compounded one or more additional therapeutic agents can be achieved by co-packaging, for example in a blister pack, the compounded at least one other therapeutic agent, so that a patient can remove and consume individual doses of the compounded the other therapeutic agent.

Co-administration includes administration of unit dosages of the compound before or after administration of unit dosages of one or more other active agents, for example, administration of the compound within seconds, minutes, or hours of the administration of one or more other active agents. For example, a unit dose of the compound can be administered first, followed within seconds or minutes by administration of a unit dose of one or more other active agents. Alternatively, a unit dose of one or more other active agents can be administered first, followed by administration of a unit dose of the compound within seconds or minutes. In some cases, it may be desirable to administer a unit dose of the compound first, followed, after a period of hours (e.g., 1-12 hours), by administration of a unit dose of one or more other active agents. In other cases, it may be desirable to administer a unit dose of one or more other active agents first, followed, after a period of hours (e.g., 1-12 hours), by administration of a unit dose of the compound.

In still yet another embodiment, the present application provides for the use of a compound of the present invention, or a pharmaceutically acceptable salt thereof, for the preparation of a medicament for treating an HCV infection.

The HCV NS3 protease inhibitory activity of the present compounds may be tested using assays known in the art. One such assay is HCV NS3 protease time-resolved fluorescence (TRF) assay as described in Example 56. Other examples of such assays are described in e.g., International patent publication W02005/046712. Compounds useful as HCV NS3 protease inhibitors would have a Ki less than 50 [tM, more preferably less than 10 [tM, and even more preferably less than 100 nM.

The present invention also includes processes for making compounds of formula I, II, II-a, II-b, II-c, II-d, III, III-a, III-b, III-c, or III-d. The compounds of the present invention can be readily prepared according to the following reaction schemes and examples, or modifications thereof, using readily available starting materials, reagents and conventional synthesis procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples. Unless otherwise indicated, all variables are as defined above. The following reaction schemes and examples serve only to illustrate the invention and its practice. The examples are not to be construed as limitations on the scope or spirit of the invention.

### General Description of Synthesis:

The compounds of the present invention may be synthesized as outlined in the general Schemes 1 through 3.

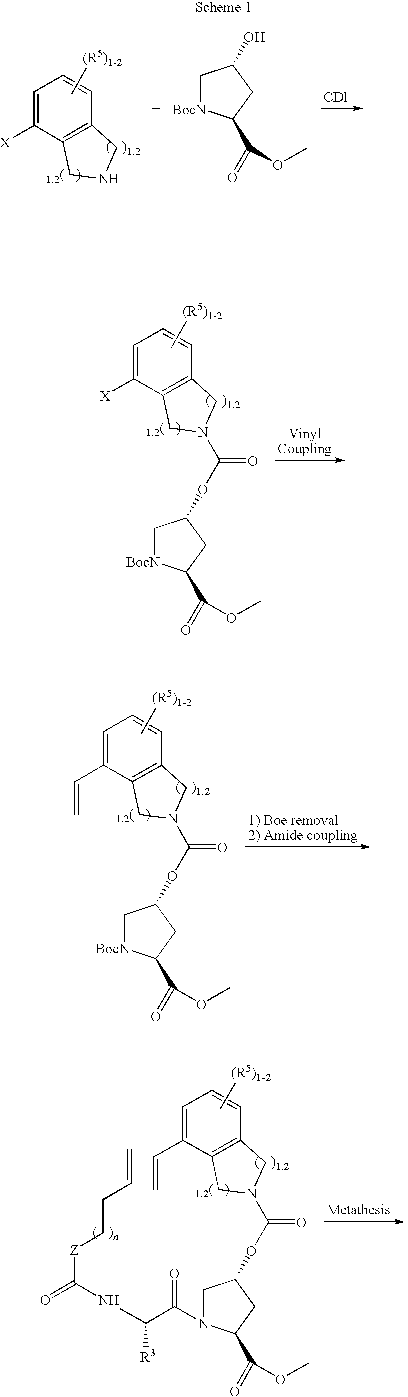

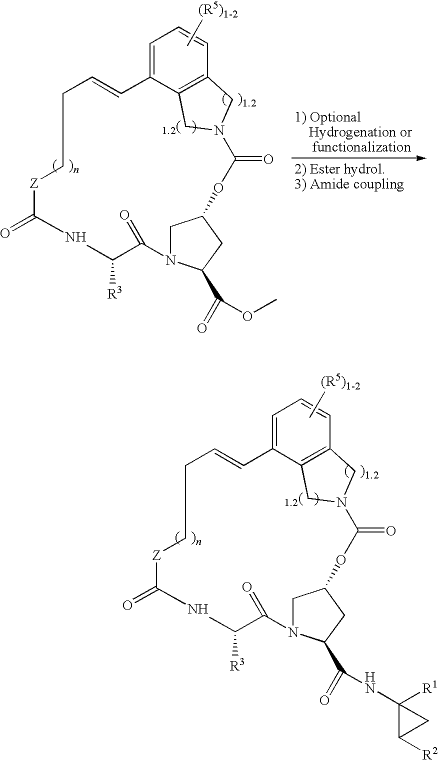

Scheme 1 (n=0-9) outlines the synthesis of a representative molecule. An appropriately protected 4-hydroxyproline derivative (for example, a carbamate protected nitrogen and an ester protected acid can be reacted with carborlyldiimidazole or equivalent reagent and then reacted with an appropriately substituted isoindoline or tetrahydroisoquinoline. The alkenyl functionality may be introduced at this or a later stage by palladium catalyzed reaction of a halide substituent such as chloride, bromide and iodide, or other functionality such as a triflate with an organometallic reagent such as a vinyl or allyltialkyltin. Alternatively, the alkenyl functionality may be introduced prior to the reaction with protected prolinol.

Scheme 2 describes the synthesis of the olefin containing amino acid portion. An amino acid (either commercially available or may be prepared readily using known methods in the art) in which the acid functionality is protected as an ester (for example, R=methyl) can be converted to amides A by coupling an olefinic carboxylic acid utilizing a wide range of peptide coupling agents known to those skilled in the art such as DCC, EDC, BOP, TBTU, etc. Preparation of the sulfonamides B can be accomplished by reaction with the appropriate sulfonyl chloride in an organic solvent (e.g., THF) with an amine base as scavenger. Urea derivatives C may be prepared by reacting the aminoester with a reagent such as carbonyldiimidazole, to form an intermediate isocyanate (Catalano et at, WO 03/062192) followed by addition of a second olefin containing amine. Alternatively, phosgene, diphosgene or triphosgene may be used in place of carbonyldiimidazole. Cyanoguanidine derivatives D can be prepared by reaction of the amino acid ester with diphenyl C-cyanocarbonimidate in an organic solvent, followed by addition of a second olefin containing amine. Carbamate derivatives B may be prepared by reacting an olefin containing alcohol with carbonyldiimidazole (or phosgene, triphosgene or diphosgene) in an organic solvent, followed by addition of the amino ester.

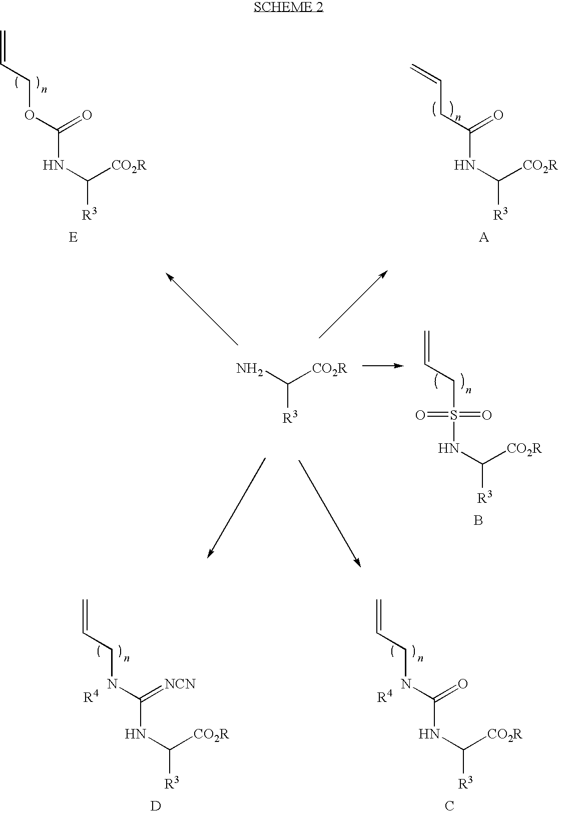

Scheme 3 describes a synthesis route to obtain a halo-substituted olefin alcohol that could be utilized in sequences described in Scheme 2 to generate halo-substituted olefin containing amino acids. Beginning with 2-methyl-2-trifluoromethylcyclohexanone, a Baeyer-Villiger oxidation could be performed using a mixture of TFPA and TFA similar to methods described by Mikami and coworkers in Org. Lett. 2003, 25, 4803. Following an acidic ring opening esterification, activation of the terminal hydroxyl to a suitable leaving group (such as tosylate, mesylate, halide or others known in the art) would enable an elimination using an appropriate sterically hindered kinetic base such as LDA or LiTMP or others known in the art. The necessary alcohol could then be revealed via exhaustive reduction using LAH or a similar reducing agent. Alternate routes to this or similar compounds would be well known to those skilled in the art.

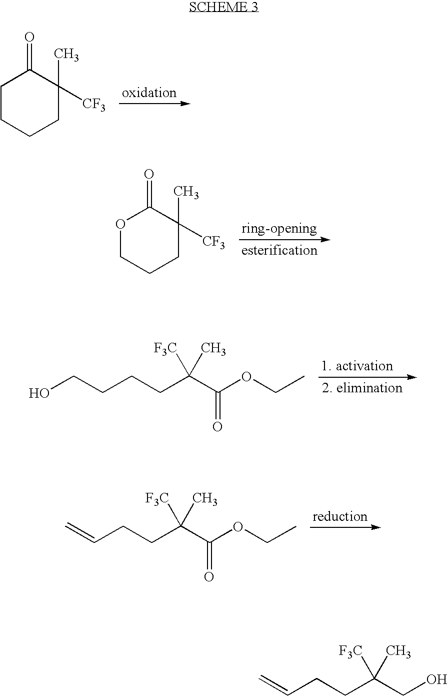

Scheme 4 describes an alternate synthetic route to obtain a hoal-substituted olefin alcohol that could be utilized in sequences described in Scheme 2 to generate halo-substituted olefin containing amino acids. Beginning with methyl, 1,1-bis(trifluoromethyl)acetate, an iridium catalyzed C—H bond activation/alkylation could be performed to obtain methyl 5-oxo-bis(trifluoromethyl)acetate as described by Murahashi and coworkers in Angew, Chem. Int. Ed. 2009, 48, 2047. Following a chemo-selective reduction of the 5-oxo group using sodium borohydride or a similar reagent, activation of the hydroxyl to a suitable leaving group (such as tosylate, mesylate, halide or others known in the art) would enable an elimination using an appropriate sterically hindered kinetic base such as LDA or LiTMP or others known in the art. The necessary alcohol could then be obtained via exhaustive reduction using LAH or a similar reducing agent. Alternate routes to this or similar compounds would be well known to those skilled in the art.

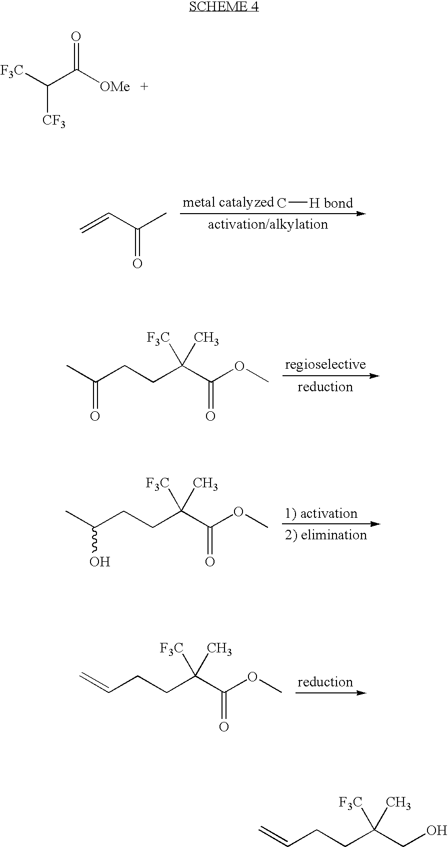

Scheme 5 describes the synthesis of the sulfamate containing portion. A sulfamic acid ester is prepared via a two step process beginning with reduction of chlorosulfonyl isocyanate with formic acid to chlorosulfonyl amide. The chlorosulfonyl amide can then undergo esterification with an alcohol in a suitable organic solvent (such as NMP) to form the corresponding sulfamic acid ester (sulfamate), which can be readily isolated by crystallization or chromatography. The sulfamate can then be coupled diretly to the N-protected cyclopropylamino acid using HATU and a suitable organic base such as DIPEA to form the N-protected cyclopropylaminoacyl sulfamate. The protecting group can then be removed by treatment with an acid such as HCl in dioxane to produce the HCl salt of the amine group suitable for further peptide coupling.

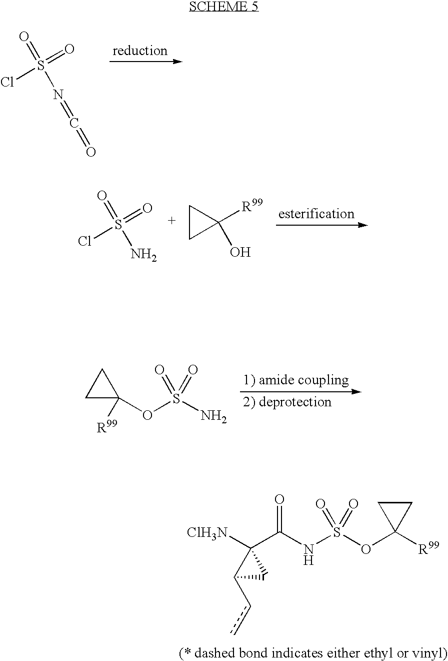

Following functionalization of the amine, the ester can be hydrolyzed under a range of basic conditions known to those skilled in the art (Theodora W. Greene, Protective Groups in Organic Synthesis, Third Edition, John Wiley and Sons, 1999).

Deprotection of the carbamate protecting group on the praline portion may be carried out by a variety of methods known to persons skilled in the art (Theodora W. Greene, Protective Groups in Organic Synthesis, Third Edition, John Wiley and Sons, 1999).

To complete the synthesis of the compounds of this invention, the amino acid derivative can be coupled to the proline derivative via a wide range of peptide coupling reagents such as DCC, EDC, BOP, TBTU etc (see Scheme 1). Macrocyclization is then achieved by an olefin metathesis using a range of catalysts that have been described in the literature for this purpose. At this stage the olefinic bond produced in the ring closing metathesis may be optionally hydrogenated to give a saturated linkage or functionalized in alternative ways such as cyclopropanation. The proline ester is then hydrolyzed under basic conditions and coupled with the cyclopropylamino acid ester (the appropriate alkenyl or alkylcyclopropane portion of the molecule can be prepared as described previously (Llinas-Brunet et al., U.S. Pat. No. 6,323,180) and subjected to an additional basic hydrolysis step. The final compounds are provided via an amide coupling between the product of the second basic hydrolysis step and the desired sulfamate to produce final compounds containing an acyl sulfamate moiety. The proline ester can also be hydrolyzed and directly coupled to an appropriately functionalized cyclopropylamino acid acyl sulfamate to provide the final compounds

Olefin metathesis catalysts include the following Ruthenium based species: F: Miller et al J. Am. Chem. Soc, 1996, 118, 9606; G: Kingsbury et in J. Am. Chem. Soc 1999, 121, 791; H: Scholl, et al. Org. Lett. 1999, 1, 953; Hoveyda, et at 1152002/0107138; K Furstner et al J. Org. Chem. 1999, 64, 8275. The utility of these catalysts in ring closing metathesis is well known in the literature (e.g. Trnka and Grubbs, Acc. Chem. Res. 2001, 34, 18).

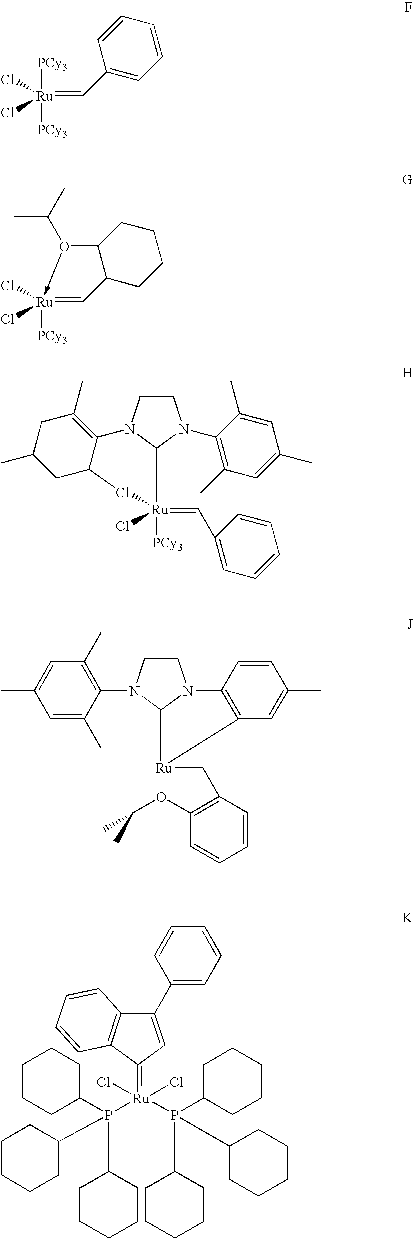

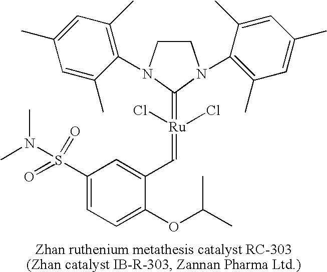

### LIST OF ABBREVIATIONS

BOP Benzotriazole-1-yl-oxy-tris-(dimethylamino)-phosphonium hexafluorophosphate

### CH₃CN Acetonitrile

### CH₂C1₂ Dichloromethane

DBU 1,8-Diazabicyclo[5.4.0]undec-7-ene

### DCC Dicyclohexylcarbodiimide

### DCE Dichloroethane

### DCM Dichloromethane

### DIPEA Diisoproylethylamine

DMAP 4-Dimethylamino pyridine

### DMF Dimethylformamide

DMSO Dimethyl sulfoxide
EDC N-(3-Dimethylaminopropy1)-N′-ethylcarbodiimide

### Et₃N Triethylamine

Et₂0 Diethyl ether
EtOAc Ethyl acetate

### EtOH Ethanol

HATU 0-(7-Azabenzotriazol-1-y1)-N,N,N′,N¹-tetramethyluronium hexafluorophosphate
HBr Hydrobromic acid
HCl Hydrochloric acid

### Hex Hexane

HOAc Acetic acid
HOAt 1-Hydroxy-7-azabenzotriazole
LiOH Lithium hydroxide

### Me0H Methanol

### Mg50₄ Magnesium Sulfate

MTBE methyl t-butyl ether
Na₂SO₄ Sodium sulfate
NaHCO₃ Sodium bicarbonate
NaOH Sodium hydroxide
NH₄C₁ Ammonium chloride
NH₄OH Ammonium hydroxide

### NMP N-methylpyrrolidinone

PDC Pyridinium dichromate
Pd/C Palladium on carbon
Pd(PPh₃)₄ tetrakis(triphenylphosphine)palladium (0)

### PhMe Toluene

### PPh₃ Triphenylphosphine

RT room temperature
TBTU 0-Benzotriazol-1-yl-N,N,N¹,N¹-tetramethyluronium tetrafluoroborate

### THF Tetrahydrofuran

### Example 1

### (5R,7S,10S)-10-tert-butyl-N-((1R,2R)-12-ethyl-1-(1-methyl-cyclopropoxysulfonylaminocarbonyl)-cyclopropyl]-15,15-dimethyl-3,9,12-trioxo-6,7,9,10,11,12,14,15,16,17,18,19-dodecahydro-1H,5H-2,23:5,8-dimethano-4,13,2,8,11-benzodioxatriazacyclohenicosine-7-carboxamide [III-205 (R⁹⁹═CH₃)]

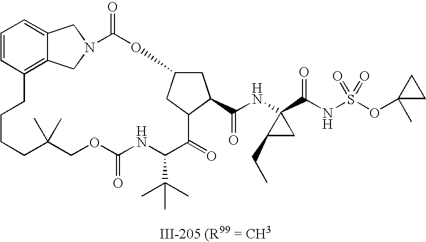

### Step 1: 1-Bromo-2,3-bis(bromomethyl)benzene

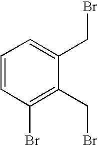

To a suspension of 3-bromo-o-xylene (999 g, 5.40 mol) in chlorobenzene (9 L) at RT was added N-bromosuccinimide (1620 g, 9.1 mol) and benzoyl peroxide (2.6 g, 10.8 mmol). The reaction mixture was heated to 80° C. and stirred under nitrogen for 18 h. The reaction mixture was cooled to 70° C. and an additional portion of NBS (302 g, 1.7 mol) was added. The reaction mixture was heated to 80° C. and stirred under nitrogen for 22 h. The reaction mixture was cooled to RT, diluted with heptane (6 L) and filtered. The filter cake was washed with heptane (4 L) and the combined filtrates were evaporated. The crude product was dissolved in heptane (2 L) and chloroform (200 mL) and filtered through basic alumina (500 g). The alumina pad was washed with heptane (4 L) and the combined filtrates were evaporated to give 1-bromo-2,3-bis(bromomethyl)benzene (1760 g, crude weight) which was used without further purification. ¹H NMR (CDCl₃) δ (ppm) 7.56 (d, J=8.0 Hz, 1H), 7.31 (d, J=8.0 Hz, 1H), 7.26 (s, 1H), 7.16 (t, J=8.0 Hz, 1H), 4.84 (s, 2H).

### Step 2: 2-Benzyl-4-bromoisoindoline hydrochloride

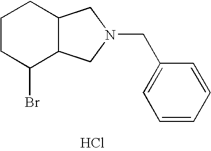

Potassium bicarbonate (657 g, 6.56 mol) was suspended in MeCN (17 L) and the mixture was heated to 80° C. Solutions of crude 1-bromo-2,3-bis(bromomethyl)benzene (900 g, 2.63 mol in 1 L MeCN) and benzylamine (281 g, 2.63 mol in 1 L MeCN) were added concurrently via addition funnels over 2 h. The reaction mixture was stirred at 77° C. for 2 h and then cooled to RT and stirred for 16 h. The contents of the reaction flask were cooled, filtered and the solvent removed by evaporation. The reaction was partitioned between water (6 L) and EtOAc (2 L). The pH was adjusted to >9 by the addition of 1M K₂CO₃, the layers were separated and the aqueous phase extracted with an additional portion of EtOAc (2 L). The combined organics were washed with brine, dried with anhydrous Na₂SO₄, filtered, and evaporated. The crude oil was diluted with EtOH (300 mL) and cooled to 0° C. Methanolic HC1 was added until the mixture was acidic, followed by MTBE (700 mL) and the mixture sonicated, then stirred for 15 h. MTBE (1 L) was added and the mixture was filtered and washed with 20% EtOH in MTBE followed by MTBE. The solid was air dried to give 2-benzyl-4-bromoisoindoline hydrochloride (211 g). An additional portion of product (86 g) was isolated by concentration of the mother liquors. LRMS (ESI) m/z 289 [(M+H)⁺; calcd for C₁₃H₁₃BrN: 289].

### Step 3: 4-Bromoisoindoline

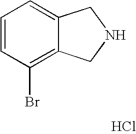

To a solution of 2-benzyl-4-bromoisoindoline hydrochloride (11 g, 30.96 mmol) in 200 mL EtOAc was added 1M NaOH (100 mL) and the mixture stirred for 30 min. The organic layer was separated, washed with brine, dried over anhydrous Na₂SO₄ and solvent evaporated to an oil which was azeotroped once with toluene (50 mL). The oil was dissolved in chlorobenzene (50 mL) and 4 A molecular sieves (5 g) added to the stirred solution. After 10 min, 1-chloroethylchloroformate (5.6 mL, 51 mmol) was added dropwise over 5 min. The reaction mixture was then heated to 90° C. for 2 h, cooled to room temperature and filtered. The solids were washed with chlorobenzene (5 mL) and methanol (40 mL). The filtrate was heated to 70° C. for 1 h, allowed to cool and stirred at room temperature overnight The solids were filtered, washed with chlorobenzene (2 mL) and hexane and dried to give 6.84 g of title compound. LRMS (ESI) m/z 198.1 [(M+H)⁺; calcd for C₈H₉BrN: 198.0).

### Step 4: 1-t-Butyl 2-methyl (2S,4R)-4-(11(4-bromo-1,3-dthydro-2H-isoindol-2-yl)carbonyl]oxy}pyrrolidine-1,2-dicarboicylate

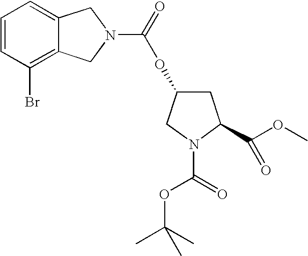

To a solution of (2S,4R)-B0C-4-hydroxyproline methyl ester (126.3 g, 515 mmol) in DMF (960 mL) at 0° C. was added N,N′-carbonyldiimidazole (83.51 g, 515 mmol). The reaction mixture was stirred at room temperature for 3 h. 4-Bromoisoindoline hydrochloride (120 g, 515 mmol) and diisopropylethylamine (96.3 mL, 540 mmol) were added and the reaction mixture heated to 50° C. for 6 h then allowed to cool to room temperature and stirred overnight The reaction mixture was partitioned between EtOAc (3 L) and 10% aqueous KHSO4 (6 L), the aqueous re-extracted with EtOAc (2 L) and the combined organic phases washed with 10% aqueous NaHCO₃, brine, dried over Na₂SO₄ and solvent evaporated to a foam (239 g). LRMS (ESI) m/z 471.0 [(M+14)* ; calcd for C₂₀H₂₀BrN₂O₆: 471.1].

### Step 5: 1-t-Butyl 2-methyl (2S,4R)-4-{[(4-viny1-1,3-dihydro-2H-isoindo1-2-yl)carbonyl]oxy}pyrrolidine-1,2-dicarboxylate

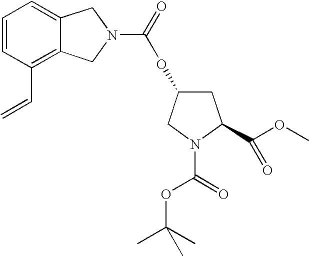

To a solution of 1-t-butyl 2-methyl (25,4R)-4-{[(4-bromo-1,3-dihydro-2H-isoindol-2-yl)carbonyl]oxylpyrrolidine-4,2-dicarboxylate (10.0 g, 21.3 mmol) in ethanol (200 mL) was added potassium vinyltrifluoroborate (4.28 g, 32 mmol) and triethylamine (4 5 mL, 32 mmol) followed by dichloro[1,1-bis(diphenylphosphino)ferrocene] palladium (II) chloride dichloromethane adduct (175 mg, 0.21 mmol). The reaction mixture was heated to reflux for 6 h, cooled to room temperature, diluted with 10% aqueous KFISO₄, and the ethanol removed by evaporation in maw. The aqueous residue was extracted with EtOAc and the organic phase washed with brine, dried over Na₂SO₄, solvent evaporated and crude product purified by chromatography on silica eluting with 40-60% EtOAc/hexane to give, after evaporation, the title compound (8.18 g). LRMS (ESI) m/z 417.2 [(M+H)⁺; calcd for C₂₂H₂₉N₂0₆: 417.2].

### Step 6: (3R,5S)-5-(Methoxycarbonyl)pyrrolidin-3-yl-4-viny1-1,3-dihydro-2H-isoindole-2-carboxylate hydrochloride

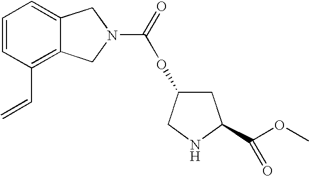

A mixture of 1-t-butyl 2-methyl (2S,4R)-4-{[(4-vinyl-1,3-dihydro-2H-isoindo1-2-yl)carbonyl]oxy)pyrrolidine-1,2-dicarboxylate (18.0 g, 43.2 mmol) and HCl/dioxane (4 M) (43.2 mL, 173 mmol) was stirred at RT for 211. The reaction mixture was concentrated to remove the dioxane followed by concentration from Et₂O to give (3R,5S)-5-(methoxycarbonyl)pyrroliclin-3-yl-4-viny1-1,3-dihydro-2H-isoindole-2-carboxylate hydrochloride as an off-white solid (15 g) which was used without further purification. LRMS (EST) m/z 317 [(M+H)⁺; calcd. for C₁₇H₂₁N₂0₄: 317].

### Step 7: Methyl N-{[(2,2-dimethylhex-5-en-1-yl)oxylcarbony1}-3-methyl-L-valy1-(4R)-4-{[(4-vinyl-1,3-dihydro-2H-isoindol-2-yl)carbonyl]oxy}-L-prolinate

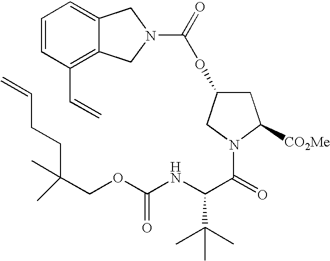

To a solution of (3R,5S)-5-(methoxycarbonyl)pyrrolidin-3-y1-4-viny1-1,3-dihydro-2H-isoindole-2-carboxylate hydrochloride (5.0 g, 14.2 mmol) and N-{[(2,2-dimethylhex-5-enyl)oxy]carbonyl)-3-methyl-L-valine (4.0 g, 14.2 mmol) in DMF (20 mL) at RT was added DIPEA (2.5 mL, 14.2 mmol), EDC (5.5 g, 28.4 mmol), and HOAt (1.9 g, 14.2 mmol). After 18 h the reaction mixture was poured into Et₂O, and extracted with 1 N HC¹. The aqueous layer was extracted with EtOAc and the combined organic layers were washed with 1 N HO, water, NaHCO₃, and brine. The organic layer was dried over MgSO₄ and the solvent was removed in vacuo. The crude product was purified on silica (30% EtOAc in hexanes) to yield 4.2 g of the title compound as a thick oil. LRMS (PSI) m/z 584.4 [(M+H)⁺; calcd for C₃₂H₄₆N₃0₇: 584.3].

### Step 8: Methyl (5R,7S,10S,18E)-10-tert-butyl-15,15-dimethy1-3,9,12-triaxo-6,7,9,10,11,12,14,16,17-decahydro-1H,5H-2, 23:5,8-dimethano-4,13,2,8,11-benzodioxatriazacyclohenicosine-7-carboxylate

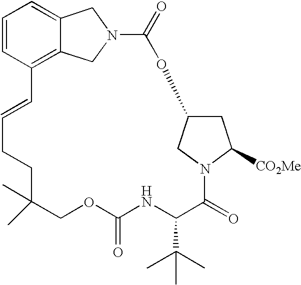

To a solution of methyl N-{[(2,2-dimethylhex-5-en-1-yl)oxy]carbonyl}-3-methyl-L-valy1-(4R)-4-{[(4-vinyl-1,3-dihydro-2H-isoindo1-2-yl)carbonyl]oxy}-L-prolinate (4.7 g, 8.05 mmol) in degassed (nitrogen bubbling for 30 min) DCM (1410 mL) was added Zhan 1B catalyst (Zhan catalyst 1B, RC-303, Zannan Pharma Ltd.) (0.591 g, 0.805 mmol). The mixture was then stirred at RT under an N atmosphere. After 19 h, the reaction was complete and DMSO (57 mL, 0.805 mmol) was added. The mixture was stirred for 2 h and the mixture was concentrated in vacuo to ˜70 mL. The crude product was then directly purified on silica (gradient elution, 0-50% EtOAc in hexanes) to yield 4.4 g of the title compound as an oil. LRMS (ESI) m/z 556.3 [(M+H)⁺; calcd for C₃₀H₄₂N₃0₇: 556.3].

### Step 9: Methyl (5R,7S,10S)-10-tert-butyl-15,15-dirnethy 1-3,9,12-trioxo-6,7,9,10,11,12,14,15,16,17,18,19-dodecahydro 1H,5H-2,23:5,8-dimethano-4,13,2,18,11-benzodioxatriazacyclohenicosine-7-carboxylate

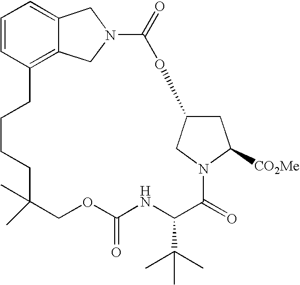

To a solution of methyl (5R,7S,10S,18E)-10-tertbutyl1-15,15-dimethyl-3,9,12-trioxo-6,7,9,10,11,12,14,15,16,17-decahydro-1H,5H-2, 23:5,8-dimethano-4,13,2,8,11-benzodioxatriazacyclohenicosine-7-carboxylate (4.4 g, 7.92 mmol) in EtOAc (79 mL) was added Pd/C (0.421 g, 0.396 mmol). A H₂ balloon was then placed on the reaction flask. The flask was evacuated quickly and filled with H₂. After 17 h, the reaction was complete as determined by LC-MS. The Pd/C was filtered through glass wool, and the crude product was purified on silica (gradient elution, 0-60% EtOAc in hexanes) to yield 4.01 g of the title compound as a white powder. LRMS (ESI) m/z 558.4 [(M+H)⁺; calcd for C₃₀H₄₄N₃0₇: 558.3].

### Step 10: (5R,7S,10S)-10-tert-Butyl-15,15-dimethyl-3,9,12-trioxo-6,7,9,10,11,12,14,15,16,17,18,19-dodecahydro-1H,5H 2, 23:5,8-dimethano-4,13,2,8,11-benzodioxatriazacyclohenicosine-7-carboxylic acid

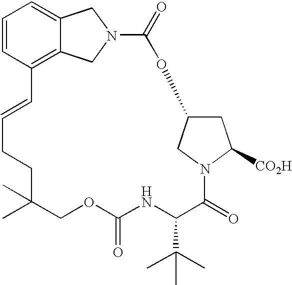

To a solution of methyl (5R,7S,10S)-10-tert-buty1-15,15-dimethyl-3,9,12-trioxo-6,7,9,10,11,12,14,15,16,17,18,19-dodecahydro-1H,5H-2,23:5,8-dimethano-4,13,2,8,1′-benzodioxatriazacyclohenicosine-7-carboxylate (5.76 g, 10.33 mmol) in THF (41.3 mL), MeOH (41.3 mL), and water (20.7 mL) at RT was added LiOH (4.33 g, 103 mmol). After full conversion (45 min), as judged by LC-MS, the reaction was worked up by partitioning between Et₂O and 1N HCl. The aqueous layer was then extracted with EtOAc. The combined organic layers were dried over MgSO₄ and the solvent was removed in in vacuo to yield 5.53 g of the title compound, which was used without further purification. LRMS (ESI) m/z 544.4 [(M+H)⁺ calcd for C₂₉H₄₂N₃0₂: 544.3].

### Step 11: (5R,7S,10S)-10-tent-butyl-N-((1R,2R)-1-[methyl carboxylate]-2-ethylcyclopropyl)-15,15-dimethyl-3,9,12-trioxo-6,7,9,10,11,12,14,15,16,17,18,19-dodecahydro-1H,5H-2, 23:5,8-dimethano-4,13,2,8,11-benzodioxatriazacyclohenicosine-7-carboxamide

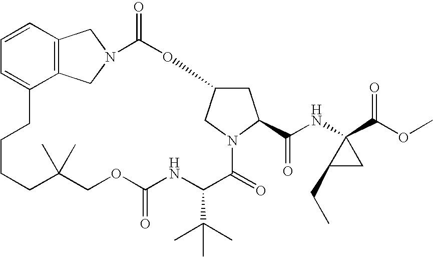

To a solution of (5R,7S,10S)-10-tert-butyl-15,15-dimethyl-3,9,12-trioxo-6,7,9,10,11,12,14,15,16,17,18,19-dodecahydro-1H,5H-2, 23:5,8-dimethano-4,13,2,8,11-benzodioxatriazacyclohenicosine-7-carboxylic acid (3 g, 5.5 mmol) in DMF (30 mL) is added (1R,2R)-1-amino-2-ethylcyclopropanecarboxylic acid methyl ester hydrochloride (1.19 g, 6.62 mmol), DIPEA (4.8 mL, 27.6 mmol), and HATU (3.15 g, 8.28 mmol) at rt. After 3 h, the reaction solution is partitioned between EtOAc and 1 M HCl solution (50 mL each). The aqueous layer is extracted with EtOAc (3×30 ml.) and the combined organics are washed with brine, dried over anhydrous MgSO₄, and concentrated. The resulting oil is purified via column chromatography on SiO₂ using 10-100% EtOAc/Hex to produce 2.37 g (64%) of (5R,7S,10S)-10-tert-butyl-N-((1R,2R)-1[methyl carboxylate]-2-ethylcyclopropy1)-15,15-dimethy1-3,9,12-trioxo-6,7,9,10,11,12,14,15,16,17,18,19-dodecahydro-1H,5H-2, 23:5,8-dimethano-4,13,2,8,11-benzodioxatriazacyclohenicosine-7-carboxamide as a brown foam: (LC/MS: m/z 668.9 (M+)).

### Step 12: (5R,7S,10S)-10-tert-butyl-N-((1R,2R)-1-[carboxy]-2-ethylcyclopropy1)-15,15-dimethyl-3,9,12-trioxo 6,7,9,10,11,12,14,15,16,17,18,19-dodecahydro-1H,5H-2,23:5,8-dimethano-4,13,2,8,11-benzodioxatriazacyclohenicosine-7-carboxamide

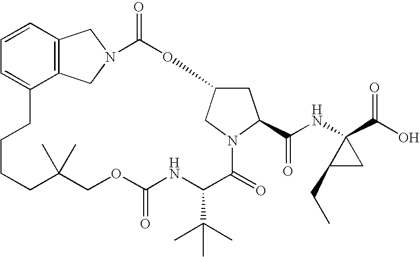

To a solution of (5R,7S,10S)-10-tert-butyl-N-((1R,2R)-1-[methylcarboxylate]-2-ethylcyclopropyl]-15,15-dimethyl-3,9,12-trioxo-6,7,9,10,11,12,14,15,16,17,18,19-dodecahydro-1H,5H-2, 23:5,8-dimethano-4,13,2,8,11-benzodioxatriazacyclohenicosine-7-carboxamide (2.37 g, 3.54 mmol) in THF/MeOH (142 mL each) is added a solution of LiOH (1.49 g, 35.4 mmol) in H₂O (7.1 mL). The resulting solution is warmed to 40° C. for 3 h. The solution is allowed to cool, diluted with Et₂O (50 mL) and acidified to pH 3 by dropwise addition of concentrated HC1. Following separation and extraction with EtOAc, the combined organics are washed with brine, dried over anhydrous Na₂SO₄, and concentrated to produce a quantitative recovery of (5R,7S,10S)-10-tent-butyl-N-((1R,2R)-1-[carboxy]-2-ethylcyclopropy1)-15,15-dimethy1-3,9,12-trioxo-6,7,9,10,11,12,14,15,16,17,18,19-dodecahydro-1H,5H-2, 23:5,8-dimethano-4,13,2,8,11-benzodioxatriazacyclohenicosine-7-carboxamide as an off-white foam that is utilized without further purification: (LC/MS: m/z 655.18 (M+H)⁺).

### Step 13: (5R,7S,10S)-10-tert-butyl-N4((1R,2R)-[2-ethyl-1-(1-methyl-cyclopropoxysulfonylaminocarbonyl)-cyclopropyl]-15,15-dimethy1-3,9,12-trioxo-6,7,9,10,11,12,14,15,16,17,18,19-dodecahydro-1H,5H-2,23:5,8-dimethano-4,13,2,8,11-benzodioxatriazacyclohenicosine-7-carboxamide (III-205; R⁹⁹═CH₃)

To a solution of (5R,7S,10S)-10-tert-butyl-N-((1R,2R)-1-[carboxy]-2-ethylcyclopropyl)-15,15-dimethyl-3,9,12-trioxo-6,7,9,10,11,12,14,15,16,17,18,19-dodecahydro-1H,5H-2, 23:5,8-dimethano-4,13,2,8,11-benzodioxatriazacyclohe-nicosine-7-carboxamide (0.85 g, 1.3 mmol) in DMF (10 mL) is added DIPEA (0.34 mL, 1.95 mmol) and HATU (0.64 g, 1.68 mmol). After 30 min, DSU (0.39 mL, 2.6 mmol) and sulfamic acid 1-methyl-cyclopropyl ester (0.295 g, 1.95 mmol) are added and the solution is aged overnight at rt. The reaction mixture is then purified by reverse phase preparatory HPLC to afford 415 mg (40%) of (5R,7S,10S)-10-tent-butyl-N-((1R,2R)-[2-ethy1-1-(1-methyl-cyclopropoxysulfonylaminocarbonyl)-cyclopropyl]-15,15-dimethy1-3,9,12-trioxo-6,7,9,10,11,12,14,15,16,17,18,19-dodecahydro-1H,5H-2, 23:5,8-dimethano-4,13,2,8,11-benzodioxatriazacyclohenicosine-7-carboxamide as an amorphous yellow solid: (LC/MS: m/z 655.18 (M+H)⁺); ¹H-NMR (500 MHz, CD₃OD): δ 7.21 (t, 1H); 7.13 (d, 1H); 7.08 (d, 1H); 5.34 (m, 1H); 4.68 (q, 2H); 4.59 (q, 2H); 4.41 (m, 1H); 4.40 (m, 1H); 4.37 (d, 1H); 4.19 (m, 1H); 3.91 (d, 1H); 3.26 (d, 1H); 2.58 (m, 1H); 2.51 (m, 1H); 2.45 (m, 1H); 2.12 (m, 1H); 1.68 (s, 3H); 1.62 (m, 1H); 1.57 (m, 1H); 1.53 (m, 1H); 1.52 (m, 2H); 1.51 (m, 1H); 1.33 (m, 1H); 1.32 (m, 2H); 1.29 (m, 2H); 1.20 (m, 1H); 1.18 (m, 1H); 1.04 (s, 9H); 1.00 (s, 3H); 0.96 (t, 3H); 0.80 (s, 3H); 0.68 (m, 2H).

### Preparation of sulfamic acid 1-methylcyclopropyl ester

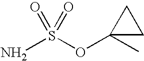

1-Methyl-cyclopropanol is synthesized according to a previously published procedure (Synthesis 1991, 3, 234). Alterations to the workup procedure are employed to improve yield and minimize unwanted byproducts. After acidic quench of the reaction, the separated organic layer is stirred vigorously over basic alumina and PDC on silica (20% loading) for 10 mins. MgSO₄ is then added to further dry the organics and the mixture is filtered through a silica gel plug. After removal of solvents, the residual slightly yellow liquid is used directly in the following esterification without further purification.

A three necked round bottom equipped with a reflux condenser is charged with chlorosulfonyl isocyanate (5.25 ml, 0.06 mol) and cooled to 0° C. Formic acid (2.25 mL, 0.06 mol) is added dropwise with rapid stirring and rapid gas evolution observed. Upon complete addition of formic acid, the reaction is let warm to room temperature. After 2 h, the resultant reaction vessel containing the solid sulfamoyl chloride is cooled to 0° C. and 1-methylcyclopropanol (2 g, ˜0.02 mol) dissolved in NMP (25 mL) is added dropwise via an addition funnel. The reaction is allowed to warm to room temperature. After 3 h stirring, the reaction mixture is poured into cold saturated aqueous NaC1 (120 mL) and extracted with EtOAc. After removal of the separated organic solvent, the crude product is purified by column chromatography on silica (35% EtOAc/hexane) to provide sulfamic acid 1-methylcyclopropyl ester (1.6 g, 53%): ¹H-NMR (CDC1₃, 300 MHz) δ 4.83 (bs, 2H), 1.70 (s, 3H), 1.32 (m, 2H), 0.68 (m, 2H).

### Preparation of (1R,2R)-1-amino-2-ethyl-cyclopropanecarboxylic acid methyl ester hydrochloride

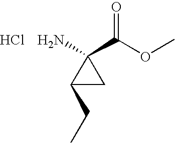

### Step 1: (1R,2R)-1-tert-butoxycarbonylamino-2-ethyl-cyclopropanecarboxylic acid methyl ester

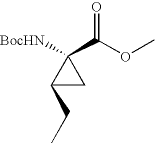

To a solution of (1R,2S)-1-tert-butoxycarbonylamino-2-vinyl-cyclopropanecarboxylic acid methyl ester (Wang, et al. WO2003/099274; 11.44 g, 47.4 mmol) in EtOAc (250 mL) at rt is added 5% Rh on alumina (6.86 g, 2.4 mmol). The atmosphere is replaced with H₂ using a balloon and the reaction is allowed to stir vigorously for 2.5 h. The reaction mixture is filtered through a pad of celite, concentrated and purified on SiO₂ eluting with 0-20% EtOAc/Hex to produce 7.04 g (61%) of (1R,2R)-1-tert-butoxycarbonylamino-2-ethyl-cyclopropanecarboxylic acid methyl ester as a colorless oil. (LC/MS: m/z 266.1 (M+Na)⁺).

### Step 2: (1R,2R)-1-amino-2-ethyl-cyclopropanecarboxylic acid methyl ester hydrochloride

To a solution of (1R,2R)-1-tert-butoxycarbonylamino-2-ethyl-cyclopropanecarboxylic acid methyl ester (4.44 g, 18.25 mmol) in THF (20 mL) is added 4M HCl in dioxane (45.5 mL, 182.5 mmol). After 2 h, the solution was concentrated to dryness to produce a quantitative yield of (1R,2R)-1-amino-2-ethyl-cyclopropanecarboxylic acid methyl ester hydrochloride as a white amorphous solid. ¹H NMR (CD₃OD, 400 MHz) δ 3.85 (s, 3H); 1.68 (m, 2H); 1.56 (m, 1H); 1.50 (q, 2H); 0.99 (s, 3H).

### Preparation of (1R,2R)-1-amino-2-ethylcyclopropanecarbonyl)-sulfamic acid 1-methylcyclopropyl ester hydrochloride

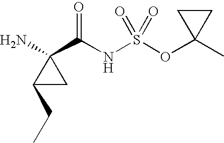

### Step 1: (1R,2R)-1-tert-butoxycarbonylamino-2-ethylcyclopropanecarboxylic acid

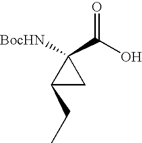

To a solution of (1R,2R)-1-tert-butoxycarbonylamino-2-ethyl-cyclopropanecarboxylic acid methyl ester (4.95 g, 20.3 mmol) in a mixture of THF (40 mL) and MeOH (40 mL) is added aqueous LiOH (2.5M, 40 mL, 100 mmol, 5 equiv.). The solution is heated to 45° C. (external temperature) for 5 h before cooling to room temperature. Aqueous HC1 (6M, 20 mL) is added and the volatiles were removed in vacuo. The residue is diluted with EtOAc and the aqueous layer separated. The organic layer is washed with brine, dried over Na₂SO₄ and concentrated to give (1R,2R)-1-tert-butoxycarbonylamino-2-ethyl-cyclopropane-carboxylic acid which is used without further purification. ¹H NMR (CDC1₃, 300 MHz) δ 5.21 (br s, 1H); 1.61 (m, 2H); 1.54-1.41 (m, 2H); 1.45 (s, 9H); 1.38-1.22 (m, 1H); 0.99 (t, 3H).

### Step 2: (1R,2R)-[2-Ethyl-1-(1-methylcyclopropoxysulfonylaminocarbonyl)cyclopropyl]-carbamic acid tert-butyl ester

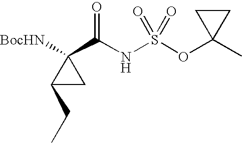

To a solution of (1R,2R)-1-tert-butoxycarbonylamino-2-ethyl-cyclopropane-carboxylic acid (2.02 g, 8.8 mmol) in CH₂Cl₂ (45 mL) was added sulfamic acid 1-methylcyclopropyl ester (2.0 g, 13.26 mmol), HATU (3.68 g, 9.7 mmol) and DIPEA (8.0 mL, 45.9 mmol). The reaction mixture was stirred at room temperature for 3 days before dilution with CH₂C1₂. The solution was washed twice with aqueous HCl (1M) and once with brine. The aqueous layers were backextracted with CH₂C1₂. The organic layers were combined, dried over Na₂SO₄, and concentrated in vacuo. The crude sulfamate was purified by column chromatography (20→100% EtOAc/hexanes) to provide (1R,2R)-[2-ethy1-1-(1-methyl-cyclopropoxysulfonylamino-carbonyl)-cyclopropyl]-carbamic acid tert-butyl ester (2.8 g, 89%): ¹H NMR (d₃-MeOD, 300 MHz) δ10.05 (s, 1H), 1.69 (s, 3H), 1.47-1.52 (m, 2H), 1.45 (s, 9H), 1.29-1.41 (m, 4H), 1.06 (m, 1H), 0.975 (t, 3H), 0.65 (m, 2H).

### Step 3: (1R,2R)-1-Amino-2-ethylcyclopropanecarbonyl)-sulfamic acid 1-methyl-cyclopropyl ester hydrochloride

To a solution of (1R,2R)-[2-ethyl-1-(1-methyl-cyclopropoxysulfonylamino-carbonyl)-cyclopropyl]-carbamic acid tert-butyl ester (2.51 g, 6.91 mmol) in CH₂Cl₂ (15 mL) is slowly added 4M HCl in dioxane (17.3 mL, 69.11 mmol). After 3 h, the volatiles are removed in vacuo to afford a quantitive yield of (1R,2R)-1-amino-2-ethylcyclopropanecarbonyl)-sulfamic acid 1-methyl-cyclopropyl ester hydrochloride as a colorless syrup. (LC/MS: m/z 262.65 (M⁺)); ¹H NMR (d₃-MeOD, 400 MHz) δ 1.84 (t, 1H); 1.68 (s, 3H); 1.62 (m, 2H); 1.50 (m, 2H); 1.28 (m, 2H); 1.02 (t, 3H); 0.71 (m, 2H).

### BIOLOGICAL ASSAYS

NS3 Enzymatic Potency: Purified NS3 protease is complexed with NS4A peptide and then incubated with serial dilutions of compound (DMSO used as solvent). Reactions are started by addition of dual-labeled peptide substrate and the resulting kinetic increase in fluorescence is measured. Non-linear regression of velocity data is performed to calculate IC₅₀s. Activity are initially tested against genotype 1b protease. Depending on the potency obtained against genotype 1b, additional genotypes (1a, 2a, 3) and or protease inhibitor resistant enzymes (D168Y, D168V, or A156T mutants) may be tested. BILN-2061 is used as a control during all assays. Representative compounds of the invention were evaluated in this assay and were typically found to have IC₅₀ values of less than about 1 μm.

Replicon Potency and Cytotoxicity: Huh-luc cells (stably replicating Bartenschlager's 13891uc-ubi-neo/NS3-3′/ET genotype 1b replicon) is treated with serial dilutions of compound (DMSO is used as solvent) for 72 hours.

Replicon copy number is measured by bioluminescence and non-linear regression is performed to calculate EC₅₀s. Parallel plates treated with the same drug dilutions are assayed for cytotoxicity using the Promega CellTiter-Glo cell viability assay. Depending on the potency achieved against the 1b replicon, compounds may be tested against a genotype 1a replicon and/or inhibitor resistant replicons encoding D168Y or A156T mutations. BILN-2061 is used as a control during all assays. Representative compounds of the invention were evaluated in this assay and were typically found to have EC₅₀ values of less than about 5 μm.

### Effect of Serum Proteins on Replicon Potency

Replicon assays are conducted in normal cell culture medium (DMEM 10% FBS) supplemented with physiologic concentrations of human serum albumin (40 mg/mL) or α-acid glycoprotein (1 mg/mL). EC₅₀s in the presence of human serum proteins are compared to the EC₅₀ in normal medium to determine the fold shift in potency.

Enyzmatic Selectivity: The inhibition of mammalian proteases including Porcine Pancreatic Elastase, Human Leukocyte Elastase, Protease 3, and Cathepsin D are measured at Kₘ for the respective substrates for each enzyme. IC₅₀ for each enzyme is compared to the IC₅₀ obtained with NS31b protease to calculate selectivity. Representative compounds of the invention have shown activity.

MT-4 Cell Cytotoxicity: MT4 cells are treated with serial dilutions of compounds for a five day period. Cell viability is measured at the end of the treatment period using the Promega CellTiter-Glo assay and non-linear regression is performed to calculate CC₅₀.

Compound Concentration Associated with Cells at EC₅₀: Huh-luc cultures are incubated with compound at concentrations equal to EC₅₀. At multiple time points (0-72 hours), cells are washed 2× with cold medium and extracted with 85% acetonitrile; a sample of the media at each time-point will also be extracted. Cell and media extracts are analyzed by LC/MS/MS to determine the Molar concentration of compounds in each fraction. Representative compounds of the invention have shown activity.

Solubility and Stability: Solubility is determined by taking an aliquot of 10 mM DMSO stock solution and preparing the compound at a final concentration of 100 μM in the test media solutions (PBS, pH 7.4 and 0.1 N HC1, pH 1.5) with a total DMSO concentration of 1%. The test media solutions are incubated at room temperature with shaking for 1 hr. The solutions will then be centrifuged and the recovered supernatants are assayed on the HPLC/UV. Solubility will be calculated by comparing the amount of compound detected in the defined test solution compared to the amount detected in DMSO at the same concentration. Stability of compounds after an 1 hour incubation with PBS at 37° C. will also be determined.

Stability in Cryopreserved Human, Dog, and Rat Hepatocytes: Each compound is incubated for up to 1 hour in hepatocyte suspensions (100 μL, 80,000 cells per well) at 37° C. Cryopreserved hepatocytes are reconstituted in the serum-free incubation medium. The suspension is transferred into 96-well plates (50 μL/well). The compounds are diluted to 2M in incubation medium and then are added to hepatocyte suspensions to start the incubation. Samples are taken at 0, 10, 30 and 60 minutes after the start of incubation and reaction will be quenched with a mixture consisting of 0.3% formic acid in 90% acetonitrile/10% water. The concentration of the compound in each sample is analyzed using LC/MS/MS. The disappearance half-life of the compound in hepatocyte suspension is determined by fitting the concentration-time data with, a monophasic exponential equation. The data will also be scaled up to represent intrinsic hepatic clearance and/or total hepatic clearance.

Stability in Hepatic 59 Fraction from Human, Dog, and Rat: Each compound is incubated for up to 1 hour in S9 suspension (500 μL, 3 mg protein/mL) at 37° C. (n=3). The compounds are added to the 59 suspension to start the incubation. Samples are taken at 0, 10, 30, and 60 minutes after the start of incubation. The concentration of the compound in each sample is analyzed using LC/MS/MS. The disappearance half-life of the compound in 59 suspension is determined by fitting the concentration-time data with a monophasic exponential equation.

Caco-2 Permeability: Compounds are assayed via a contract service (Absorption Systems, Exton, Pa.). Compounds are provided to the contractor in a blinded manner. Both forward (A-to-B) and reverse (B-to-A) permeability will be measured. Caco-2 monolayers are grown to confluence on collagen-coated, macroporous, polycarbonate membranes in 12-well Costar Transwell® plates. The compounds are dosed on the apical side for forward permeability (A-to-B), and are dosed on the basolateral side for reverse permeability (B-to-A). The cells are incubated at 37° C. with 5% CO₂ in a humidified incubator. At the beginning of incubation and at 1 hr and 2 hr after incubation, a 200-μL aliquot is taken from the receiver chamber and replaced with fresh assay buffer. The concentration of the compound in each sample is determined with LC/MS/MS. The apparent permeability, Papp, is calculated.

### Plasma Protein Binding:

Plasma protein binding is measured by equilibrium dialysis. Each compound is spiked into blank plasma at a final concentration of 2 μM. The spiked plasma and phosphate buffer is placed into opposite sides of the assembled dialysis cells, which will then be rotated slowly in a 37° C. water bath. At the end of the incubation, the concentration of the compound in plasma and phosphate buffer is determined. The percent unbound is calculated using the following equation:

$$\% Unbound = 100 \cdot ( \frac{{C}_{f}}{{C}_{h} + {C}_{f}} )$$

Where C_{f} and C_{b} are free and bound concentrations determined as the post-dialysis buffer and plasma concentrations, respectively.

### CYP450 Profiling:

Each compound is incubated with each of 5 recombinant human CYP450 enzymes, including CYP1A2, CYP2C9, CYP3A4, CYP2D6 and CYP2C19 in the presence and absence of NADPH. Serial samples will be taken from the incubation mixture at the beginning of the incubation and at 3, 13, 30, 45 and 60 min after the start of the incubation. The concentration of the compound in the incubation mixture is determined by LC/MS/MS. The percentage of the compound remaining after incubation at each time point is calculated by comparing with the sampling at the start of incubation.

### Stability in Rat, Dog, Monkey and Human Plasma.

Compounds will be incubated for up to 2 hours in plasma (rat, dog, monkey, or human) at 37° C. Compounds are added to the plasma at final concentrations of 1 and 10 μg/mL. Aliquots are taken at 0, 5, 15, 30, 60, and 120 mm after adding the compound. Concentration of compounds and major metabolites at each timepoint are measured by LC/MS/MS.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A compound of formula (Ia): 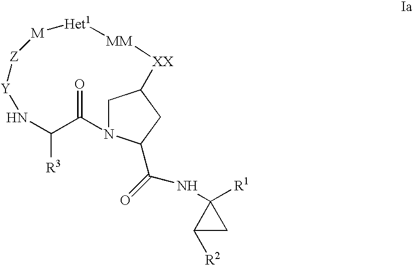
R¹ is: 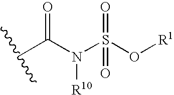
MM is CO or a bond;
XX is O, NH, N(C₁-C₄ alkyl), a bond or CH₂;
Het¹ is a heterocycle and can be substituted with up to ten groups selected independently from WW or R⁵; R^{f} is A³;
each WW is independently H, halo, OR⁷⁷, C₁-C₆ alkyl, CN, CF₃, NO₂, SR⁷⁷, CO₂R⁷⁷, CON(R⁷⁷)₂, C(O)R⁷⁷, N(R¹⁰⁰)C(O)R⁷⁷, SO₂(C₁-C₆ alkyl), S(O)(C₁-C₆ alkyl), C₃-C₈ cycloalkyl, C₃-C₆ cycloalkoxy, C₁-C₆ haloalkyl, N(R⁷⁷)₂, NH(C₁-C₆ alkyl)O(C₁-C₆ alkyl), halo(C₁-C₆ alkoxy), NR¹⁰⁰SO₂R⁷⁷, SO₂N(R⁷⁷)₂, NHCOOR⁷⁷, NHCONHR⁷⁷, aryl, heteroaryl or heterocyclyl; wherein aryl is phenyl or naphthyl, heteroaryl is a 5- or 6-membered aromatic ring having 1, 2 or 3 heteroatoms selected from N, 0 and S, attached through a ring carbon or nitrogen, and heterocyclyl is a 5- to 7-membered saturated or unsaturated nonaromatic ring having 1, 2, 3 or 4 heteroatoms selected from N, 0 and S, attached through a ring carbon or nitrogen; and wherein 2 adjacent WW moieties are optionally taken together with the atoms to which they are attached to form a 5- to 6-membered saturated, unsaturated non-aromatic, or aromatic cyclic ring having 0-2 heteroatoms selected from N, 0 and S;
A³ is independently selected from PRT, H, —OH, —C(O)OH, cyano, alkyl, alkenyl, alkynyl, amino, amido, imido, imino, halogen, CF₃, CH₂CF₃, cycloalkyl, nitro, aryl, aralkyl, alkoxy, aryloxy, heterocycle, —C(A²)₃, —C(A²)₂—C(O)A², —C(O)A², —C(O)OA², —O(A²), —N(A²)₂, —S(A²), —CH₂P(Y¹)(A²)(OA²), —CH₂P(Y¹)(A²)(N(A²)₂), —CH₂P(Y¹)(OA²)(OA²), —OCH₂P(Y¹)(OA²)(0A²), —OCH₂P(Y¹)(A²)(OA²), —OCH₂P(Y¹)(A²)(N(A²)₂), —C(O)OCH₂P(Y¹)(OA²)(OA²), —C(O)OCH₂P(Y¹)(A²)(OA²), —C(O)OCH₂P(Y¹)(A²)(N(A²)₂), —CH₂P(Y¹)(OA²)(N(A²)₂), —OCH₂P(Y¹)(OA²)(N(A²)₂), —C(O)OCH₂P(Y¹)(OA²)(N(A²)₂), —CH₂P(Y¹)(N(A²)₂)(N(A²)₂), —C(O)OCH₂P(Y¹)(N(A²)₂)(N(A²)₂), —OCH₂P(Y¹)(N(A²)₂)(N(A²)₂), —(CH₂)ₘ-heterocycle, —(CH₂)ₘC(O)Oalkyl, —O—(CH₂)ₘ—O—C(O)—Oalkyl, —O—(CH₂)ᵣ—O—C(O)—(CH₂)ₘ-alkyl, —(CH₂)ₘO—C(O)—O-alkyl, —(CH₂)ₘO—C(O)—O-cycloalkyl, —N(H)C(Me)C(O)O-alkyl, SRᵣ, S(O)Rᵣ, S(O)₂Rᵣ, or alkoxy arylsulfonamide, wherein each A³ may be optionally substituted with
1 to 4
—R¹¹¹, —P(Y¹)(OA²)(OA²), —P(Y¹)(OA²)(N(A²)₂), —P(Y¹)(A²)(OA²), —P(Y¹)(A²)(N(A²)₂), or P(Y¹)(N(A²)₂)(N(A²)₂), —C(═O)N(A²)₂), halogen, alkyl, alkenyl, alkynyl, aryl, carbocycle, heterocycle, aralkyl, aryl sulfonamide, aryl alkylsulfonamide, aryloxy sulfonamide, aryloxy alkylsulfonamide, aryloxy arylsulfonamide, alkyl sulfonamide, alkyloxy sulfonamide, alkyloxy alkylsulfonamide, arylthio, —(CH₂)ₘheterocycle, —(CH₂)ₘ—C(O)O-alkyl, —O(CH₂)ₘOC(O)Oalkyl, —O—(CH₂)ₘ—O—C(O)—(CH₂)ₘ-alkyl, —(CH₂)ₘ—O—C(O)—O-alkyl, —(CH₂)ₘ—O—C(O)-β-cycloalkyl, —N(H)C(CH₃)C(O)O-alkyl, or alkoxy arylsulfonamide, optionally substituted with R¹¹¹;
A² is independently selected from PRT, H, alkyl, alkenyl, alkynyl, amino, amino acid, alkoxy, aryloxy, cyano, haloalkyl, cycloalkyl, aryl, heteroaryl, heterocycle, alkylsulfonamide, or arylsulfonamide, wherein each A² is optionally substituted with A³;
R¹¹¹ is independently selected from H, alkyl, alkenyl, alkynyl, aryl, cycloalkyl, heterocycle, halogen, haloalkyl, alkylsulfonamido, arylsulfonamido, —C(O)NHS(O)₂—, or —S(O)₂—, optionally substituted with one or more A³;
R² is C₂-C₆ alkyl, C₂-C₆ alkenyl or C₃-C₆ cycloalkyl, wherein said alkyl, alkenyl or cycloalkyl is optionally substituted with 1 to 3 halo;
R³ is C₁-C₈ alkyl, C₃-C₈ cycloalkyl, C₃-C₈ cycloalkyl(C₁-C₆)alkyl, aryl(C₁-C₈)alkyl, or Het, wherein aryl is phenyl or naphthyl and said alkyl, cycloalkyl, or aryl is optionally substituted with 1 to 3 substituents selected from the group consisting of halo, OR¹⁰, SR¹⁰, N(R¹⁰)₂, NH(C₁-C₆ alkyl)O(C₁-C₆ alkyl), C₁-C₆ alkyl, C₁-C₆haloalkyl, halo(C₁-C₆ alkoxy), NO₂, CN, CF₃, SO₂(C₁-C₆ alkyl), S(O)(C₁-C₆ alkyl), NR¹⁰SO₂R⁶, SO₂N(R⁶)₂, NHCOOR⁶, NHCOR⁶, NHCONHR⁶, CO₂R¹⁰, C(O)R¹⁰, and CON(R¹⁰)₂;
Het is a 5-6 membered saturated cyclic ring having 1 or 2 heteroatoms selected from N, 0 and S, wherein said ring is optionally substituted with 1 to 3 substituents selected from halo, OR¹⁰, SR¹⁰, N(R¹⁰)₂, NH(C₁-C₆ alkyl)O(C₁-C₆ alkyl), C₁-C₆ alkyl, C₁-C₆ haloalkyl, halo(C₁-C₆ alkoxy), NO₂, CN, CF₃, SO₂(C₁-C₆ alkyl), S(O)(C₁-C₆ alkyl), NR¹⁰SO₂R⁶, SO₂N(R⁶)₂, NHCOOR⁶, NHCOR⁶, NHCONHR⁶, CO₂R¹⁰, C(O)R¹⁰, and CON(R¹⁰)₂;
R⁴ is H, C₁-C₆ alkyl, C₃-C₈ cycloalkyl(C₁-C₈)alkyl, or aryl(C₁-C₈)alkyl; wherein aryl is phenyl or naphthyl and said alkyl, cycloalkyl, or aryl is optionally substituted with 1 to 3 substituents selected from the group consisting of halo, OR¹⁰, SR¹⁰, N(R¹⁰)₂, NH(C₁-C₆ alkyl)O(C₁-C₆ alkyl), C₁-C₆ alkyl, C₁-C₆ haloalkyl, halo(C₁-C₆ alkoxy), NO₂, CN, CF₃, SO₂(C₁-C₆ alkyl), S(O)(C₁-C₆ alkyl), NR¹⁰SO₂R⁶, SO₂N(R⁶)₂, NHCOOR⁶, NHCOR⁶, NHCONHR⁶, CO₂R¹⁰, C(O)R¹⁰, and CON(R¹⁰)₂;
R⁵ is H, halo, OR¹⁰, C₁-C₆ alkyl, CN, CF₃, SR¹⁰, SO₂(C₁-C₆ alkyl), C₃-C₈ cycloalkyl, C₃-C₈ cycloalkoxy, C₁-C₆ haloalkyl, N(R⁷)₂, aryl, heteroaryl or heterocyclyl; wherein aryl is phenyl or naphthyl, heteroaryl is a 5- or 6-membered aromatic ring having 1, 2 or 3 heteroatoms selected from N, 0 and S, attached through a ring carbon or nitrogen, and heterocyclyl is a 5- to 7-membered saturated or unsaturated non-aromatic ring having 1, 2, 3 or 4 heteroatoms selected from N, 0 and S, attached through a ring carbon or nitrogen; and wherein said aryl, heteroaryl, heterocyclyl, cycloalkyl, cycloalkoxy, alkyl or alkoxy is optionally substituted with 1 to 4 substituents selected from the group consisting of halo, OR¹⁰, SR¹⁰, N(R⁷)₂, NH(C₁-C₆ alkyl)O(C₁-C₆ alkyl), C₁-C₆ alkyl, C₁-C₆ haloalkyl, halo(C₁-C₆ alkoxy), C₃-C₆ cycloalkyl, C₃-C₆ cycloalkoxy, NO₂, CN, CF₃, SO₂(C₁-C₆ alkyl), NR¹⁰SO₂R⁶, SO₂N(R⁶)₂, S(O)(C₁-C₆ alkyl), NHCOOR⁶, NHCOR⁶, NHCONHR⁶, CO₂R¹⁰, C(O)R¹⁰, and CON(R¹⁰)₂; wherein the 2 adjacent substituents of said cycloalkyl, cycloalkoxy, aryl, heteroaryl or heterocyclyl are optionally taken together to form a 3-6 membered cyclic ring containing 0-3 heteroatoms selected from N, 0 and S;
R⁶ is C₁-C₆ alkyl, C₃-C₆ cycloalkyl, C₃-C₆ cycloalkyl(C₁-C₅)alkyl, aryl, aryl(C₁-C₄)alkyl, heteroaryl, heteroaryl(C₁-C₄ alkyl), heterocyclyl, or heterocyclyl(C₁-C₈ alkyl), wherein said alkyl, cycloalkyl, aryl, heteroaryl, or heterocyclyl is optionally substituted or heteroaryl, or heterocyclyl is optionally substituted with 1 to 2 W substituents; and wherein each aryl is independently phenyl or naphthyl, each heteroaryl is independently a 5- or 6-membered aromatic ring having 1, 2 or 3 heteroatoms selected from N, 0 and S, attached through a ring carbon or nitrogen, and each heterocyclyl is independently a 5- to 7-membered saturated or unsaturated non-aromatic ring having 1, 2, 3 or 4 heteroatoms selected from N, 0 and S, attached through a ring carbon or nitrogen;
each R⁷⁷ is independently H, C₁-C₆ alkyl, C₃-C₆ cycloalkyl, C₃-C₆ cycloalkyl(C₁-C₈)alkyl, aryl, aryl(C₁-C₄)alkyl, heteroaryl, heteroaryl(C₁-C₄ alkyl), heterocyclyl, or heterocyclyl(C₁-C₆ alkyl), wherein said alkyl, cycloalkyl, aryl, heteroaryl, or heterocyclyl is optionally substituted with 1 to 2 W′ substituents; and wherein each aryl is independently phenyl or naphthyl, each heteroaryl is independently a 5- or 6-membered aromatic ring having 1, 2 or 3 heteroatoms selected from N, 0 and S, attached through a ring carbon or nitrogen, and each heterocyclyl is independently a 5- to 7-membered saturated or unsaturated non-aromatic ring having 1,2, 3 or 4 heteroatoms selected from N, 0 and S, attached through a ring carbon or nitrogen;
each W′ is independently halo, OR¹⁰⁰, C₁-C₆ alkyl, CN, CF₃, NO₂, SR¹⁰⁰, CO₂R¹⁰⁰, CON(R¹⁰⁰)₂, C(O)R¹⁰⁰, N(R¹⁰⁰)C(O)R¹⁰⁰, SO₂(C₁-C₆ alkyl), S(O)(C₁-C₆ alkyl), C₃-C₆ cycloalkyl, C₃-C₆ cycloalkoxy, C₁-C₆ haloalkyl, N(R¹⁰⁰)₂, NH(C₁-C₆ alky1)O(C₁-C₆ alkyl), halo(C₁-C₆ alkoxy), NR¹⁰⁰SO₂R¹⁰⁰, SO₂N(R¹⁰⁰)₂, NHCOOR¹⁰⁰, NHCONHR¹⁰⁰, aryl, heteroaryl or heterocyclyl; wherein aryl is phenyl or naphthyl, heteroaryl is a 5- or 6-membered aromatic ring having 1, 2 or 3 heteroatoms selected from N, 0 and S, attached through a ring carbon or nitrogen, and heterocyclyl is a 5- to 7-membered saturated or unsaturated non-aromatic ring having 1,2, 3 or 4 heteroatoms selected from N, 0 and S, attached through a ring carbon or nitrogen; and wherein 2 adjacent W′ moieties are optionally taken together with the atoms to which they are attached to form a 5- to 6-membered saturated, unsaturated non-aromatic, or aromatic cyclic ring having 0-2 heteroatoms selected from N, 0 and S;
each Rᵣ is independently H, (C₂-C₁₀)alkenyl, (C2-C₁₀) alkynyl, (C₁-C₁₀)alkanoyl, or (C₁-C₁₀)alkoxycarbonyl;
Y is C(═O), 502, or C(═N—CN);
Y¹ is independently O, S, N(A³), N(O)(A³), N(OA³), N(O)(OA³) or N(N(A³)(A³));
Z is C(R¹⁰)₂, or N(R⁴):
M is C₁-C₁₂ alkylene or C₂-C₁₂ alkenylene, wherein said alkylene or alkenylene is optionally substituted with 1 or 2 substituents selected from the group consisting of C₁-C₈ alkyl, C₃-C₈ cycloalkyl(C₁-C₈ alkyl), and aryl(C₁-C₈ alkyl) and further which M can be substituted by up to nine halo; and 2 substituents of M are optionally taken together to form a 3-6 membered cyclic ring containing 0-3 heteroatoms selected from N, 0 and S; and optionally one substituent of M can be taken together with a ring atom within M to form a 3-6 membered ring system containing 0-3 heteroatoms selected, from N, 0, and S where the 3-6 membered ring system is fused to the macrocyclic ring system;
each R⁷ is independently H, C₁-C₆ alkyl, C₃-C₆ cycloalkyl, C₃-C₆ cycloalkyl(C₁-C₅)allyl, aryl, aryl(C₁-C₄)alkyl, heteroaryl, heteroaryl(C₁-C₄ alkyl), heterocyclyl, or heterocyclyl(C₁-C₈ alkyl), wherein said alkyl, cycloalkyl, aryl, heteroaryl, or heterocyclyl is optionally substituted with 1 to 2 W substituents; and wherein each aryl is independently phenyl or naphthyl, each heteroaryl is independently a 5- or 6-membered aromatic ring having 1, 2 or 3 heteroatoms selected from N, 0 and S, attached through a ring carbon or nitrogen, and each heterocyclyl is independently a 5- to 7-membered saturated or unsaturated non-aromatic ring having 1, 2, 3 or 4 heteroatoms selected from N, 0 and S, attached through a ring carbon or nitrogen;
each W is independently halo, OR¹⁰, C₁-C₆ alkyl, CN, CF₃, NO₂, SR¹⁰, CO₂R¹⁰, CON(R¹⁰)₂, C(O)R¹⁰, N(R¹⁰)C(O)R¹⁰, SO₂(C₁-C₆ alkyl), S(O)(C₁-C₆ alkyl), C₃-C₈ cycloalkyl, C₃-C₈ cycloalkoxy, C₁-C₆ haloalkyl, N(R¹⁰)₂, NH(C₁-C₆ alky1)O(C₁-C₆ alkyl), halo(C₁-C₆ alkoxy), NR¹⁰SO₂R¹⁰, SO₂N(R¹⁰), NHCOOR¹⁰, NHCONHR¹⁰, aryl, heteroaryl or heterocyclyl; wherein aryl is phenyl or naphthyl, heteroaryl is a 5- or 6-membered aromatic ring having 1, 2 or 3 heteroatoms selected from N, 0 and S, attached through a ring carbon or nitrogen, and heterocyclyl is a 5- to 7-membered saturated or unsaturated non-aromatic ring having 1, 2, 3 or 4 heteroatoms selected from N, 0 and S, attached through a ring carbon or nitrogen;
each R¹⁰ is independently H or C₁-0₆ alkyl;
each R¹⁰⁰ is independently H or C₁-C₆ alkyl;
r is 0 to 6;
m is 0 to 6.

2. The compound of claim 1 wherein the compound is of formula (Ib) 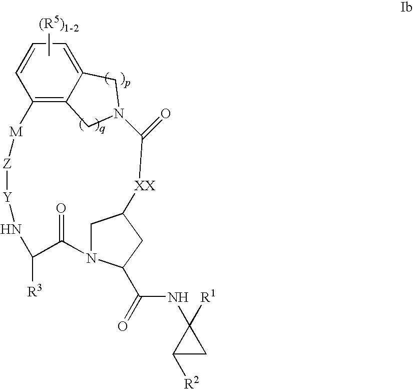
wherein:
p and q are independently 1 or 2.

3. The compound of claim 1 wherein the compound is of formula (Ic) 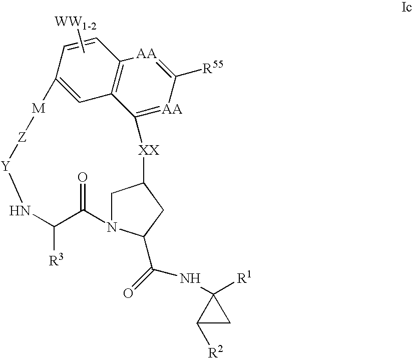
R⁵⁵ is H, halo, OH, C₁-C₆ alkoxy, C₁-C₆ alkyl, CN, CF3, SR¹⁰, SO₂(C₁-C₆ alkyl), C₃-C₆ cycloalkyl, C₃-C₆ cycloalkoxy, C₃-C₆ haloalkyl, N(R⁷⁷)₂, aryl, heteroaryl or heterocyclyl; wherein aryl is phenyl or naphthyl, heteroaryl is a 5- or 6-membered aromatic ring having 1, 2 or 3 heteroatoms selected from N, 0 and S, attached through a ring carbon or nitrogen, and heterocyclyl is a 5- to 7-membered saturated or unsaturated non-aromatic ring having 1, 2, 3 or 4 heteroatoms selected from N, 0 and S, attached through a ring carbon or nitrogen; and wherein said aryl, heteroaryl, heterocyclyl, cycloalkyl, cycloalkoxy, alkyl or alkoxy is optionally substituted with 1 to 4 substituents selected from the group consisting of halo, OR¹⁰, SR¹⁰, N(R⁷⁷)₂, NH(C₁-C₆ alky1)O(C₁-C₆ alkyl), C₁-C₆ alkyl, C₁-C₆ haloalkyl, halo(C₁-C₆ alkoxy), C₃-C₆ cycloalkyl, C₃-C₆ cycloalkoxy, NO₂, CN, CF₃, SO₂(C₁-C₆ alkyl), NR¹⁰⁰OSO₂R⁶, SO₂N(R⁶)₂, S(O)(C₁-C₆ alkyl), NHCOOR⁶, NHCOR⁶, NHCONHR⁶, CO₂R¹⁰, C(O)R¹⁰₂, and)CON(R¹⁰)₂; wherein the 2 adjacent substituents of said cycloalkyl, cycloalkoxy, aryl, heteroatyl or heterocyclyl are optionally taken together to form a 3-6 membered cyclic ring containing 0-3 heteroatoms selected from N, 0 and S;
R⁶⁶ is C₁-C₆ alkyl, C₃-C₆ cycloalkyl, C₃-C₆ cycloalkyl(C₁-C₅)alkyl, aryl, aryl(C₁-C₄)alkyl, heteroaryl, heteroaryl(C₁-C₄ alkyl), heterocyclyl, or heterocyclyl(C₁-C₆ alkyl), wherein said alkyl, cycloalkyl, aryl, heteroaryl, or heterocyclyl is optionally substituted with 1 to 2 W′ substituents; and wherein each aryl is independently phenyl or naphthyl, each heteroaryl is independently a 5- or 6-membered aromatic ring having 1, 2 or 3 heteroatoms selected from N, 0 and S, attached through a ring carbon or nitrogen, and each heterocyclyl is independently a 5- to 7-membered saturated or unsaturated non-aromatic ring having 1, 2, 3 or 4 heteroatoms selected from N, 0 and S, attached through a ring.carbon or nitrogen;
AA is C(R¹¹⁰) or N;
When R⁵⁵ is other than H, R¹¹⁰ is H, C₁-C₆ alkyl, halo, OR¹⁰⁰, SR¹⁰⁰, or N(R¹⁰⁰)₂;
when R⁵⁵ is H, C₁-C₆ alkyl, halo, OH, C₁-C₆ alkoxy, CN, CF₃, SR¹⁰⁰, SO₂(C₁-C₆ alkyl), C₃-C₈ cycloalkyl, C₃-C₈ cycloalkoxy, C₁-C₆ halo alkyl, N(R⁷⁷)₂, aryl, heteroaryl or heterocyclyl; wherein aryl is phenyl or naphthyl, heteroaryl is a 5- or 6-membered aromatic ring having 1, 2 or 3 heteroatoms selected from N, 0 and S, attached through a ring carbon or nitrogen, and heterocyclyl is a 5- to 7-membered saturated or unsaturated non-aromatic ring having 1, 2, 3 or 4 heteroatoms selected from N, 0 and S, attached through a ring carbon or nitrogen; and wherein said aryl, heteroaryl, heterocyclyl, cycloalkyl, cycloalkoxy, alkyl or alkoxy is optionally substituted with 1 to 4 substituents selected from the group consisting of halo, OR¹⁰, SR¹⁰, N(R⁷⁷)₂, NH(C₁-C₆ alkyl)O(C₁-C₆ alkyl), C₁-C₆ alkyl, C₁-C₆ haloalkyl, halo(C₁-C₆ alkoxy), C₃-C₆ cycloalkyl, C₃-C₆ cycloalkoxy, NO₂, CN, CF₃, SO₂(C₁-C₆ alkyl), NR¹⁰⁰SO₂R⁶⁶, SO₂N(R⁶⁶)₂, S(O)(C₁-C₆ alkyl), NHCOOR⁶⁶, NHCOR⁶⁶NHCONHR⁶⁶, CO₂R¹⁰⁰, C(O)R¹⁰⁰, and CON(R¹⁰⁰)₂; wherein the 2 adjacent substituents of said cycloalkyl, cycloalkoxy, aryl, heteroaryl or heterocyclyl are optionally taken together to form a 3-6 membered cyclic ring containing 0-3 heteroatoms selected from N, 0 and S;
or R⁵⁵ and R¹⁰⁰ are optionally taken together to form a 5- to 6-membered saturated, unsaturated non-aromatic, or aromatic cyclic ring having 0-2 heteroatoms selected from N, 0 and S;

4. The compound of claim 1 wherein R^{f} is H, alkyl, alkenyl, alkynyl, aryl, heteroaryl, or cycloalkyl, which W is optionally substituted with one or more R_{g};
each R_{g} is independently H, alkyl, alkenyl, alkynyl, halo, hydroxy, cyano, arylthio, cycloalkyl, aryl, heteroaryl, alkoxy, NRₕRᵢ, —C(═O)NRₕRᵢ, or —C(═O)OR_{d}, wherein each aryl and heteroaryl is optionally substituted with one or more alkyl, halo, hydroxy, cyano, nitro, amino, alkoxy, alkoxycarbonyl, alkanoyloxy, haloalkyl, or haloalkoxy; wherein each alkyl of R_{g} is optionally substituted with one or more halo, alkoxy, or cyano;
each Rₕ and Rᵢ is independently H, alkyl, or haloalkyl; and
R_{d} and Rₑ are each independently H, (C₁-C₁₀)alkyl, or aryl, which is optionally substituted with one or more halo

5. The compound of claim 2 wherein R^{f} is H, alkyl, alkenyl, alkynyl, aryl, heteroaryl, or cycloalkyl, which R^{f} is optionally substituted with one or more R_{g};
each R_{g} is independently H, alkyl, alkenyl, alkynyl, halo, hydroxy, cyano, arylthio, cycloalkyl, aryl, heteroaryl, alkoxy, NRₕRᵢ —C(═O)NRₕRᵢ, or —C(═O)OR_{d}, wherein each aryl and heteroaryl is optionally substituted with one or more alkyl, halo, hydroxy, cyano, nitro, amino, alkoxy, alkoxycarbonyl, alkanoyloxy, haloalkyl, or haloalkoxy;
wherein each alkyl of R_{g} is optionally substituted with one or more halo, alkoxy, or cyano;
each Rₕand Rᵢ is independently H, alkyl, or haloalkyl; and
R_{d} and Rₑ are each independently H, (C₁-C₁₀)alkyl, or aryl, which is optionally substituted with one or more halo.

6. The compound of claim 3 wherein R^{f} is H, alkyl, alkenyl, alkynyl, aryl, heteroaryl, or cycloalkyl, which R^{f} is optionally substituted with one or more R_{g};
each R_{g} is independently H, alkyl, alkenyl, alkynyl, halo, hydroxy, cyano, arylthio, cycloalkyl, aryl, heteroaryl, alkoxy, NRₕRᵢ, —C(═O)NRₕRᵢ or —C(═O)OR_{d}, wherein each aryl and heteroaryl is optionally substituted with one or more alkyl, halo, hydroxy, cyano, nitro, amino, alkoxy, alkoxycarbonyl, alkanoyloxy, haloalkyl, or haloalkoxy; wherein each alkyl of R_{g} is optionally substituted with one or more halo, alkoxy, or cyano;
each Rₕ and R is independently H, alkyl, or haloalkyl; and
R_{d} and Rₑ are each independently H, (C₁-C₁₀)alkyl, or aryl, which is optionally substituted with one or more halo.

7. The compound of claim 1 wherein R^{f} is H, alkyl, alkenyl, alkynyl, aryl, heteroaryl, or cycloalkyl, which R^{f} is optionally substituted with one or more R_{g};
each R_{g} is independently H, alkyl, alkenyl, alkynyl, halo, hydroxy, cyano, arylthio, cycloalkyl, aryl, heteroaryl, alkoxy, NRₕRᵢ, —C(═O)NRₕRᵢ, wherein each aryl and heteroaryl is optionally substituted with one or more alkyl, halo, hydroxy, cyano, nitro, amino, alkoxy, alkoxycarbonyl, alkanoyloxy, haloalkyl, or haloalkoxy;
each Rₕ and Rᵢ is independently H, alkyl, or haloalkyl.

8. The compound of claim 2 wherein R^{f} is H, alkyl, alkenyl, alkynyl, aryl, heteroaryl, or cycloalkyl, which R^{f} is optionally substituted with one or more R_{g};
each R_{g} is independently H, alkyl, alkenyl, alkynyl, halo, hydroxy, cyano, arylthio, cycloalkyl, aryl, heteroaryl, alkoxy, NRₕRᵢ, —C(═O)NRₕRᵢ, wherein each aryl and heteroaryl is optionally substituted with one or more alkyl, halo, hydroxy, cyano, nitro, amino, alkoxy, alkoxycarbonyl, alkanoyloxy, haloalkyl, or haloalkoxy;
each Rₕ and Rᵢ is independently H, alkyl, or haloalkyl.

9. The compound of claim 3 wherein R^{f} is H, alkyl, alkenyl, alkynyl, aryl, heteroaryl, or cycloalkyl, which R^{f} is optionally substituted with one or more R_{g};
each R_{g}, is independently H, alkyl, alkenyl, alkynyl, halo, hydroxy, cyano, arylthio, cycloalkyl, aryl, heteroaryl, alkoxy, NRₕRᵢ, —C(═O)NRₕRᵢ, wherein each aryl and heteroaryl is optionally substituted with one or more alkyl, halo, hydroxy, cyano, nitro, amino, alkoxy, alkoxycarbonyl, alkanoyloxy, haloalkyl, or haloalkoxy;
each Rₕ and R is independently H, alkyl, or haloalkyl.

10. The compound of claim 1 wherein R^{f} is alkyl, aryl, cycloalkyl, which R^{f} is optionally substituted with one or more R_{g} independently selected from alkyl, halo, —C(═O)OR_{d}, or trifluoromethyl, wherein each alkyl of R_{g} is optionally substituted with one or more halo, alkoxy, or cyano.

11. The compound of claim 2 wherein R^{f} is alkyl, aryl, cycloalkyl, which R^{f} is optionally substituted with one or more R_{g} independently selected from alkyl, halo, —C(═O)OR^{d}, or trifluoromethyl, wherein each alkyl of R_{g} is optionally substituted with one or more halo, alkoxy, or cyano.

12. The compound of claim 3 wherein R^{f} is alkyl, aryl, cycloalkyl, which R^{f} is optionally substituted with one or more R_{g} independently selected from alkyl, halo, —C(═O)OR_{d}, or trifluoromethyl, wherein each alkyl of R_{g} is optionally substituted with one or more halo, alkoxy, or cyano.

13. The compound of claim 1 wherein R^{f} is aryl, heteroaryl, or cycloalkyl, which R^{f} is optionally substituted with one to three A³.

14. The compound of claim 2 wherein R^{f} is aryl, heteroaryl, or cycloalkyl, which R^{f} is optionally substituted with one to three A³.

15. The compound of claim 3 wherein R^{f} is aryl, heteroaryl, or cycloalkyl, which R^{f} is optionally substituted with one to three A³.

16. The compound of claim 1 wherein R^{f} is cyclopropyl which R^{f} is optionally substituted by up to four A³.

17. The compound of claim 2 wherein R^{f} is cyclopropyl which R^{f} is optionally substituted by up to four A³.

18. The compound of claim 3 wherein R^{f} is cyclopropyl which R^{f} is optionally substituted by up to four A³.

19. The compound of claim 1 wherein R^{f} is cyclopropyl which R^{f} is optionally substituted by up to three C₁-C₆ alkyl.

20. The compound of claim 2 wherein R^{f} is cyclopropyl which R^{f} is optionally substituted by up to three C₁-C₆ alkyl

21. The compound of claim 3 wherein R^{f} is cyclopropyl which R^{f} is optionally substituted by up to three C₁-C₆ alkyl.

22. The compound of claim 1 wherein R^{f} is phenyl, cyclopropyl, 2-fluorophenyl, 4-chlorophenyl, 2-chlorophenyl, 2,6-dimethylphenyl, 2-methylphenyl, 2,2-dimethylpropyl, 2,2-difluoroethyl, 2,2,2-trifluoroethyl, or 1-methylcyclopropyl.

23. The compound of claim 2 wherein R^{f} is phenyl, cyclopropyl, 2-fluorophenyl, 4-chlorophenyl, 2-chlorophenyl, 2,6-dimethylphenyl, 2-methylphenyl, 2,2-dimethylpropyl, 2,2-difluoroethyl, 2,2,2-trifluoroethyl, or 1-methylcyclopropyl.

24. The compound of claim 3 wherein R^{f} is phenyl, cyclopropyl, 2-fluorophenyl, 4-chlorophenyl, 2-chlorophenyl, 2,6-dimethylphenyl, 2-methylphenyl, 2,2-dimethylpropyl, 2,2-difluoroethyl, 2,2,2-trifluoroethyl, or 1-methylcyclopropyl.

25. The compound of claim 1 wherein R^{f} is cyclopropyl.

26. The compound of claim 2 wherein R^{f} is cyclopropyl.

27. The compound of claim 3 wherein R^{f} is cyclopropyl.

28. The compound of claim 1 wherein R^{f} is 1-methylcyclopropyl.

29. The compound of claim 2 wherein R^{f} is 1-methylcyclopropyl.

30. The compound of claim 3 wherein R^{f} is 1-methylcyclopropyl.

31. The compound of claim 1, wherein the compound is of formula 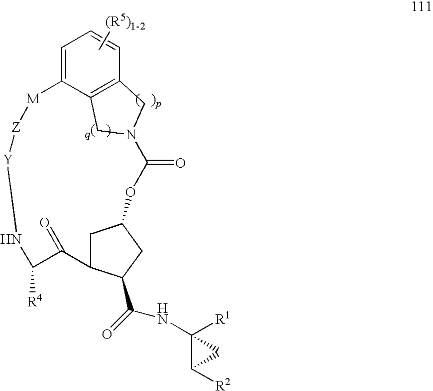
wherein the sum of p and q is 3.

32. The compound of claim 31, wherein R² C₂-C₄ alkenyl or C₂-C₄ alkyl.

33. The compound of claim 32, wherein R³ is C₅-C₆ cycloalkyl or C₃-C₆ alkyl optionally substituted with C₁-C₆ alkyl or C₁-C₆ alkyl optionally substituted with 1 to 3 substituents selected from halo and OR¹⁰.

34. The compound of claim 33, wherein R⁵ is H, halo or C₁-C₆alkoxy.

35. The compound of claim 34, wherein Y is C═O.

36. The compound of claim 35, wherein Z is O, C(R¹⁰)₂, NH or N(C₁-C₈ alkyl).

37. The compound of claim 36, wherein M is unsubstituted C₄-C₈ alkylene or unsubstituted C₄-C₈ alkenylene.

38. The compound of claim 1, wherein the compound is selected from the group consisting of compounds III-I to III-252 wherein R⁹⁹ is H, methyl, C₂-C₈ alkyl or C₂-C₈ haloalkyl:

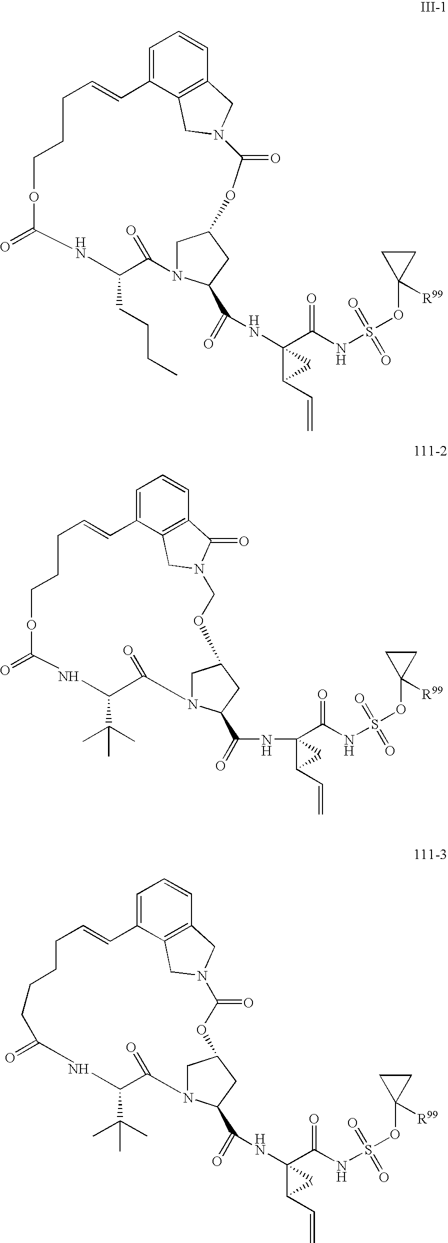

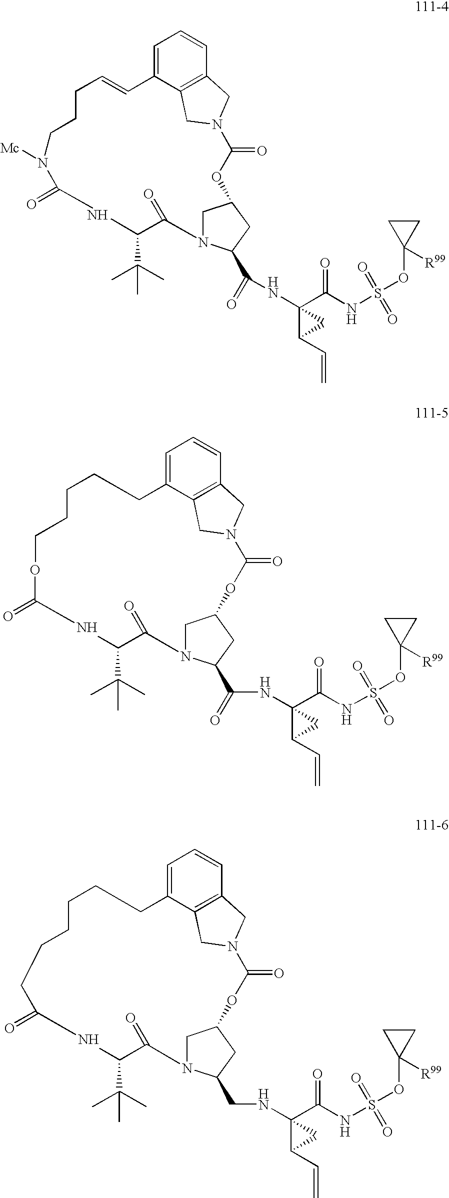

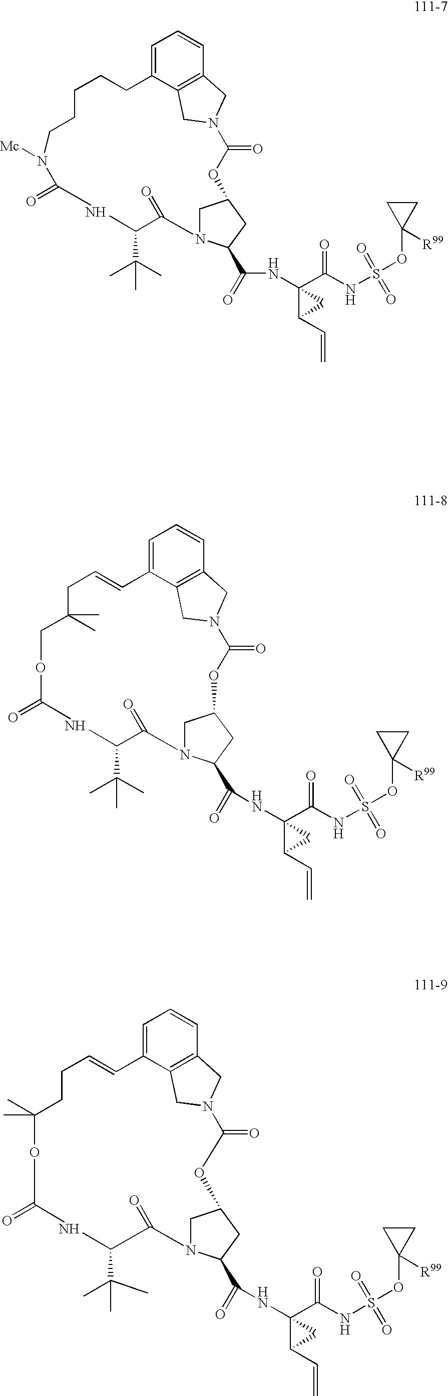

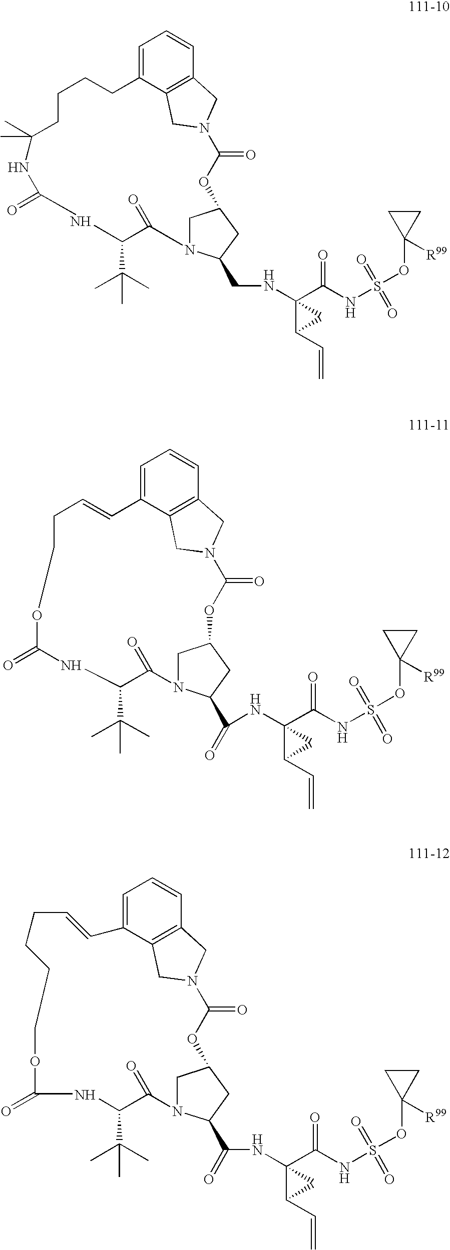

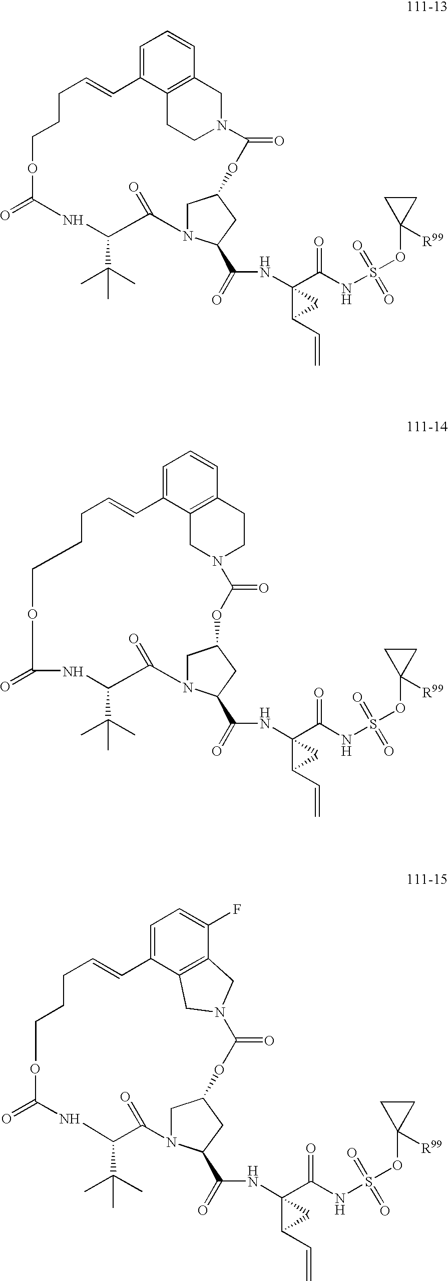

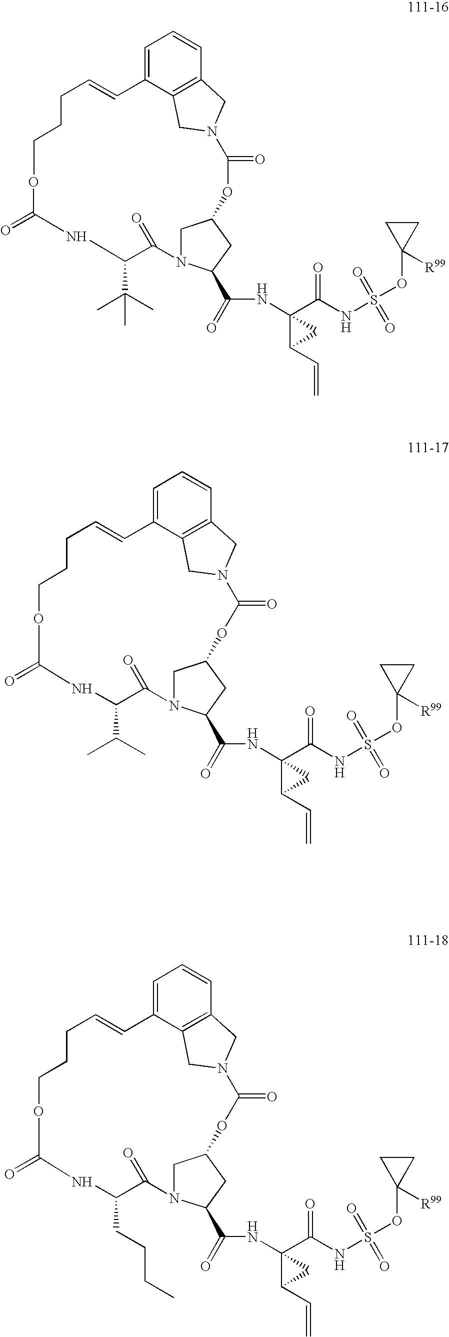

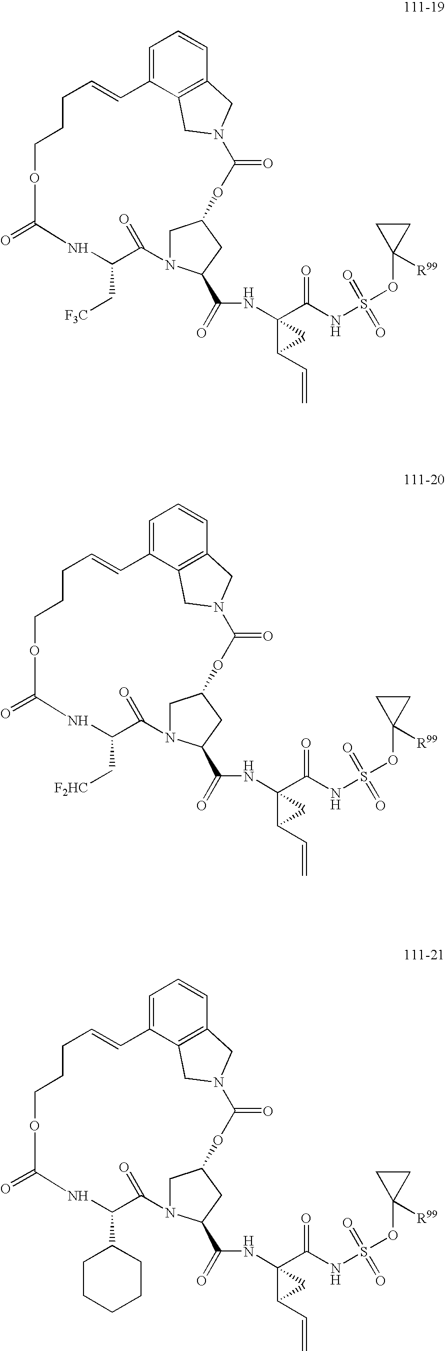

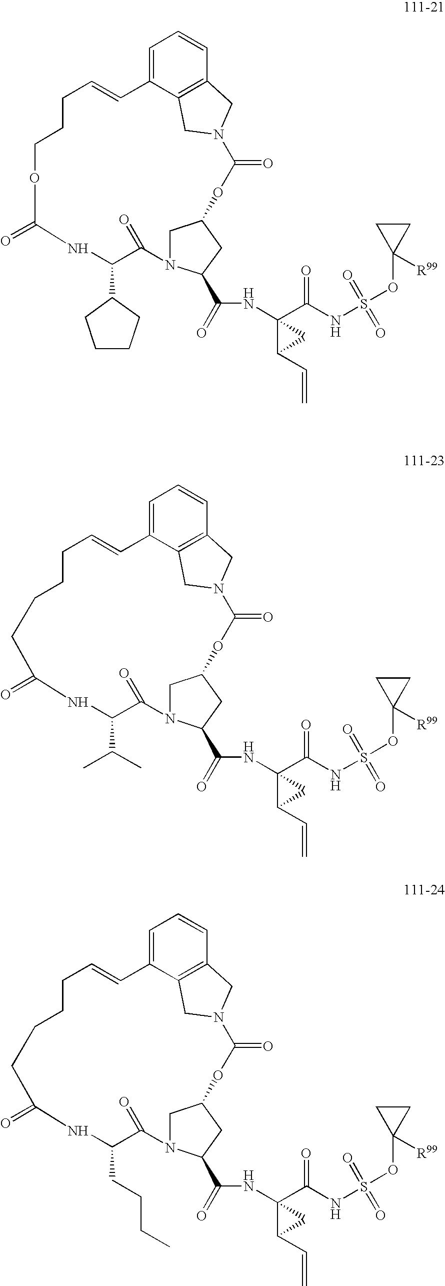

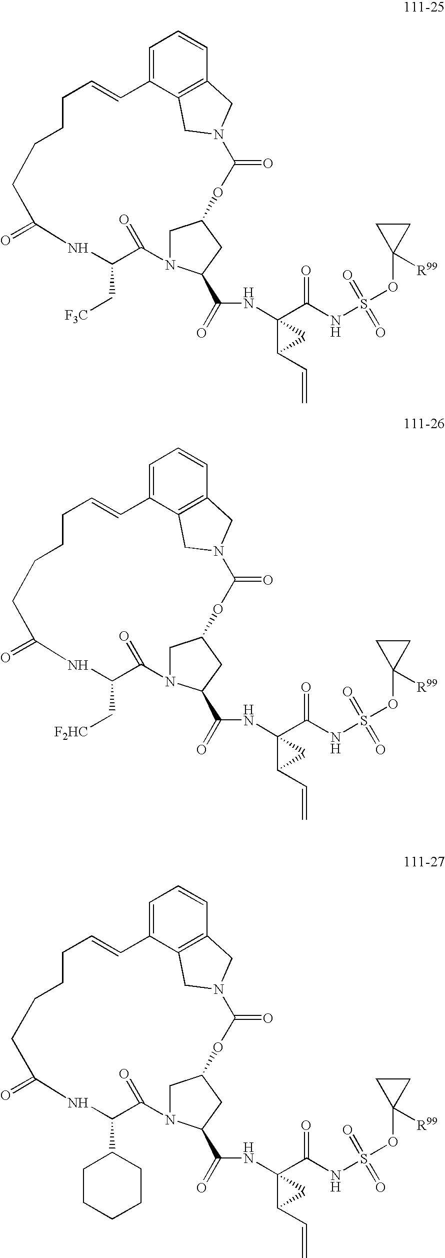

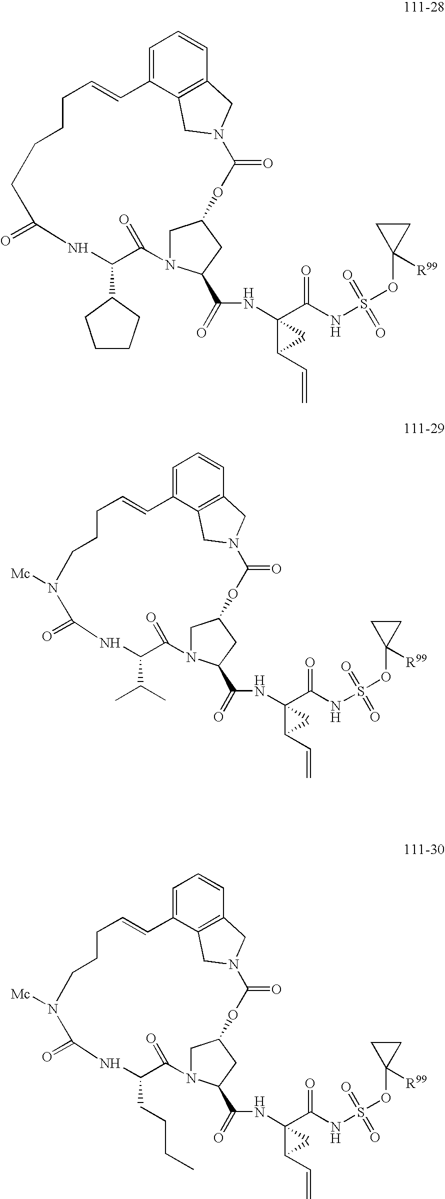

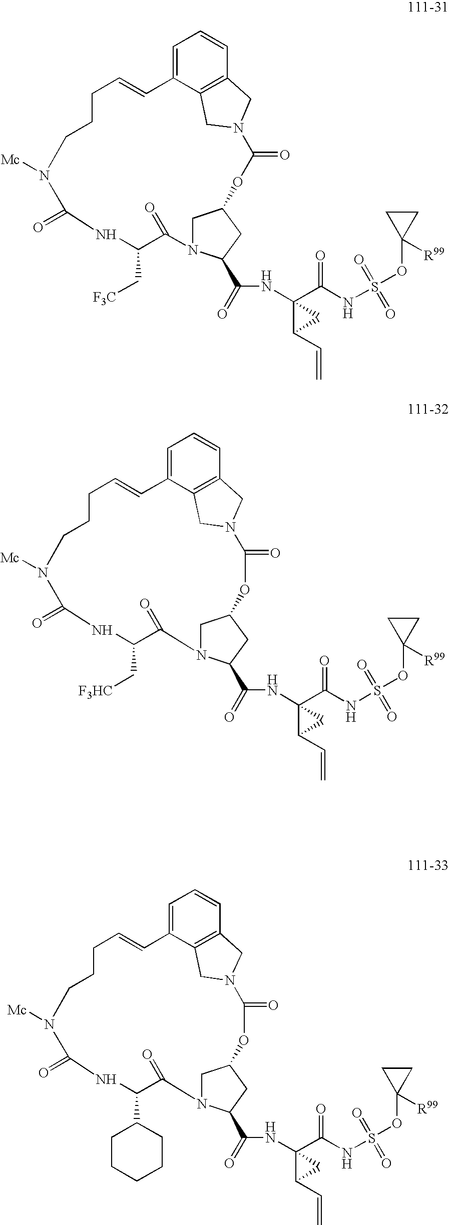

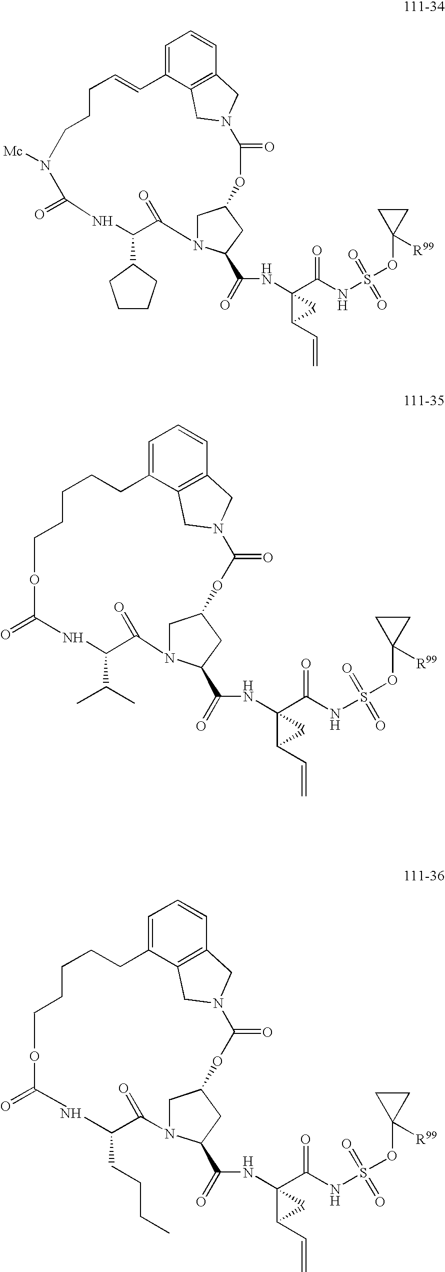

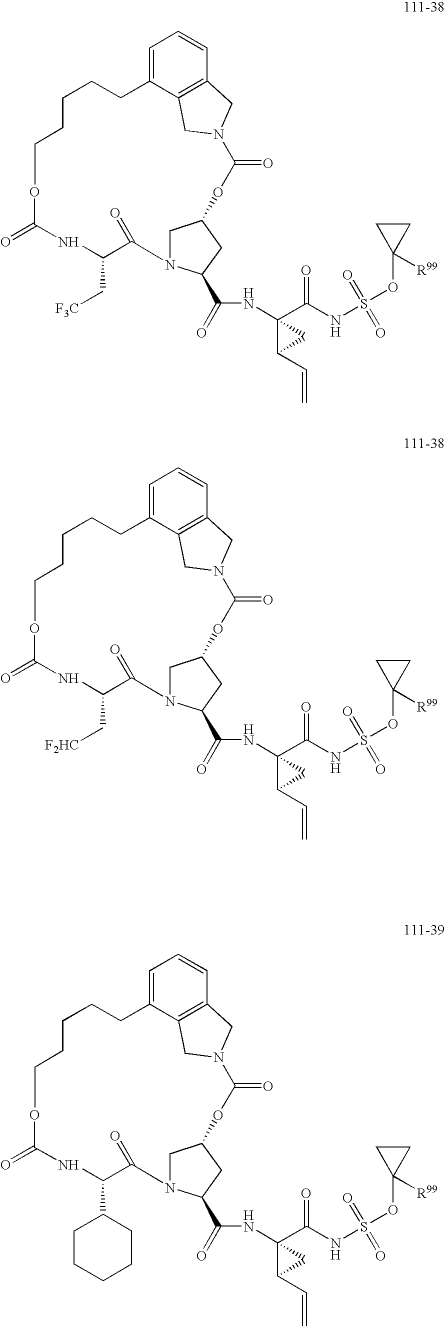

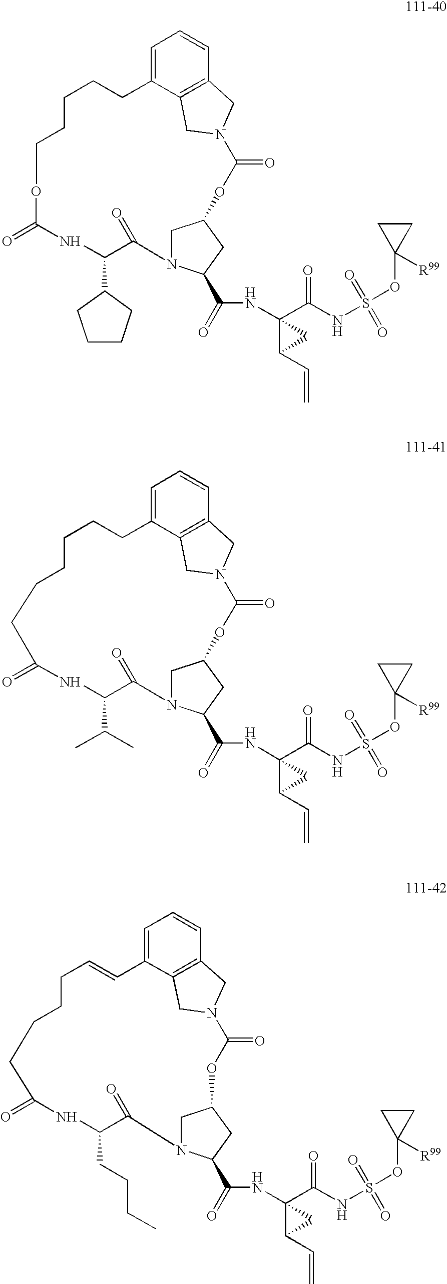

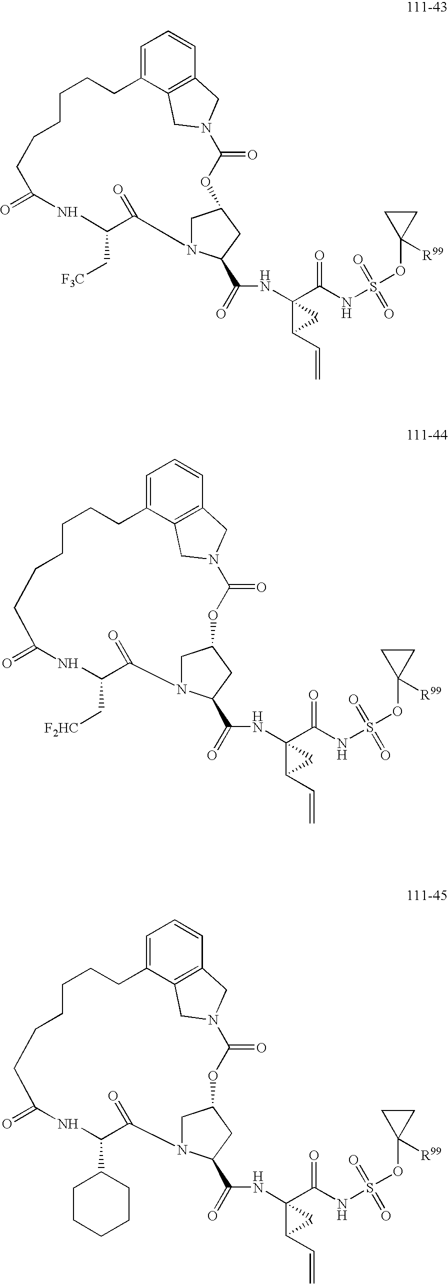

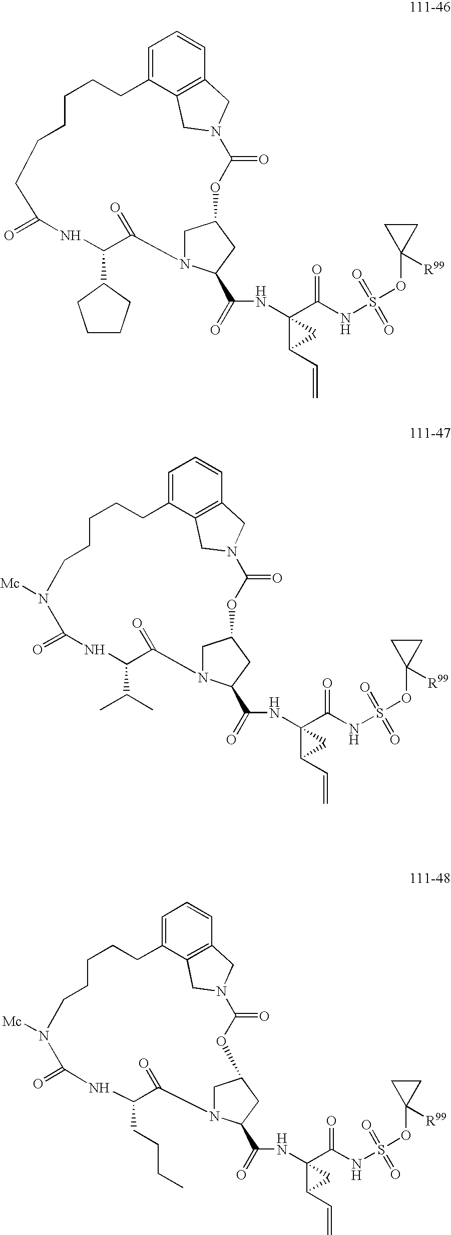

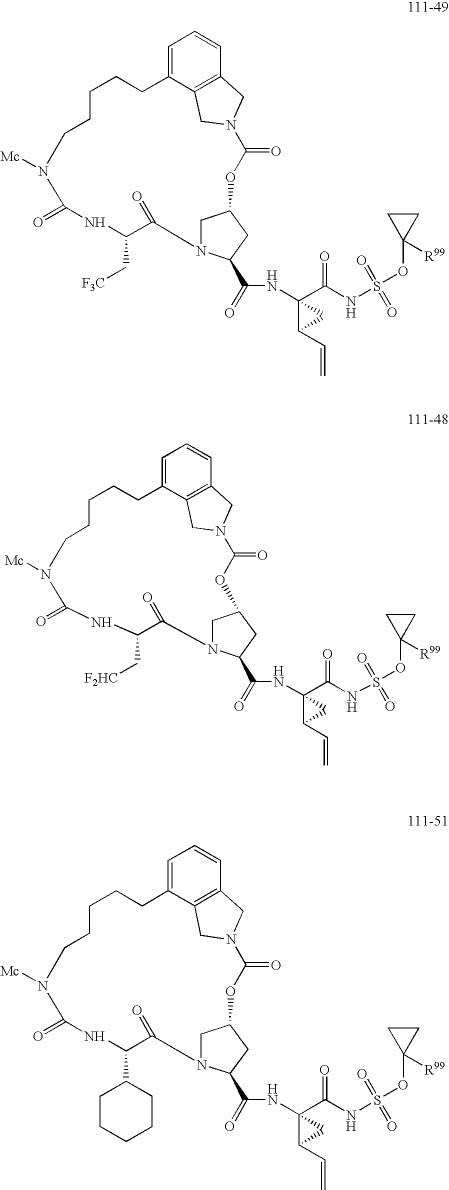

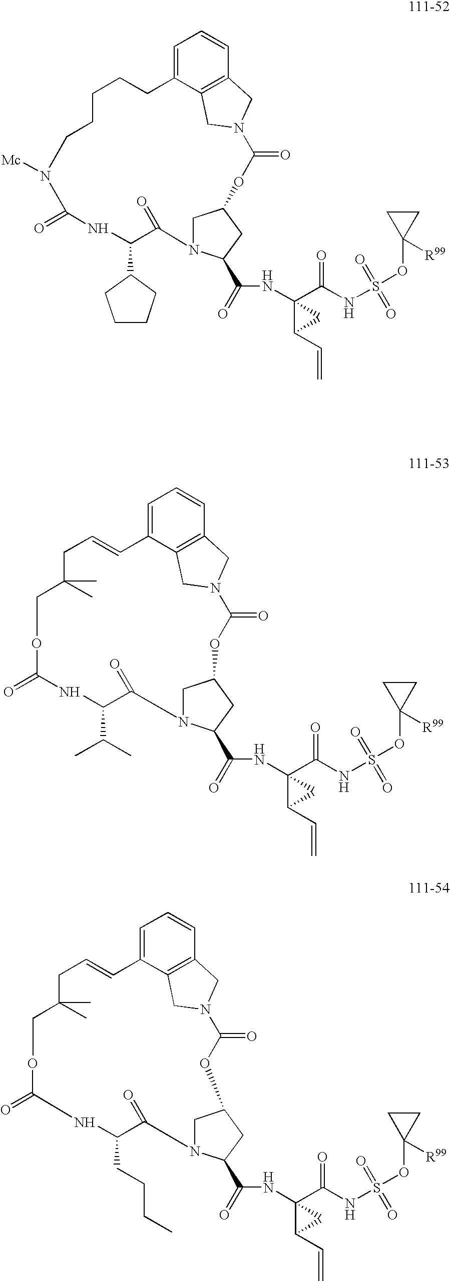

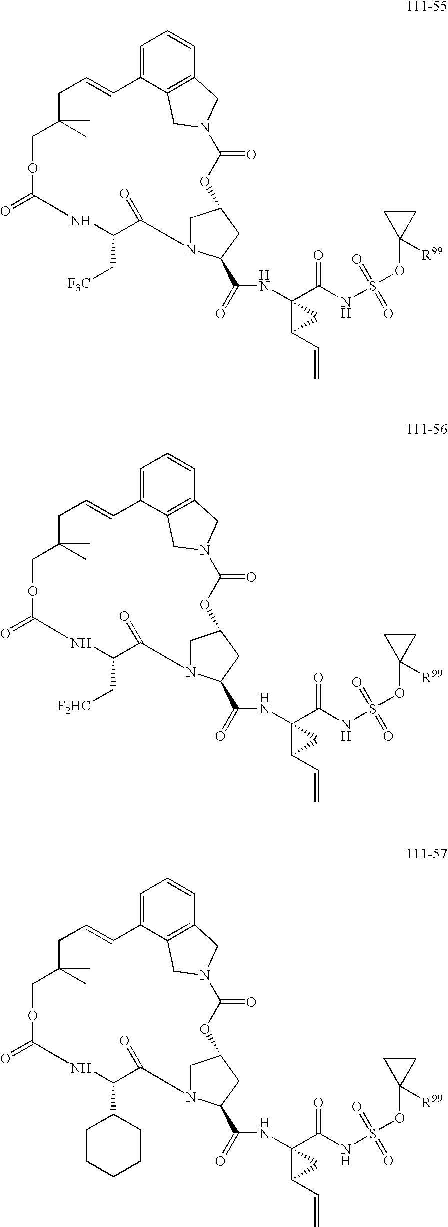

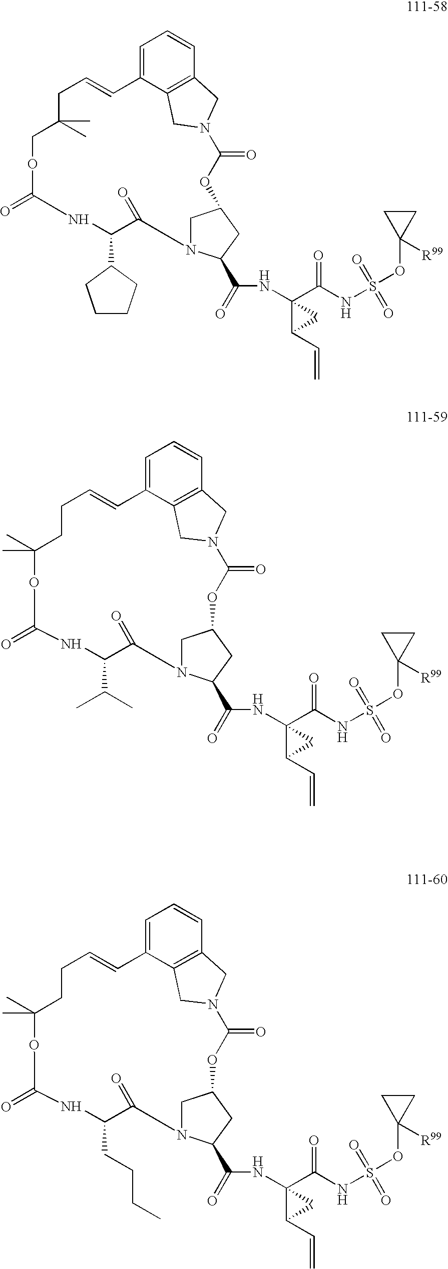

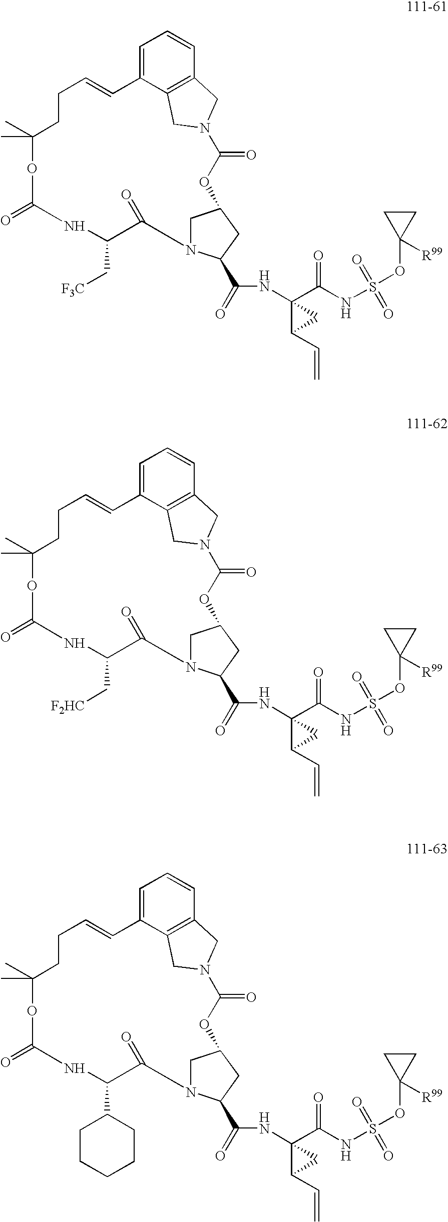

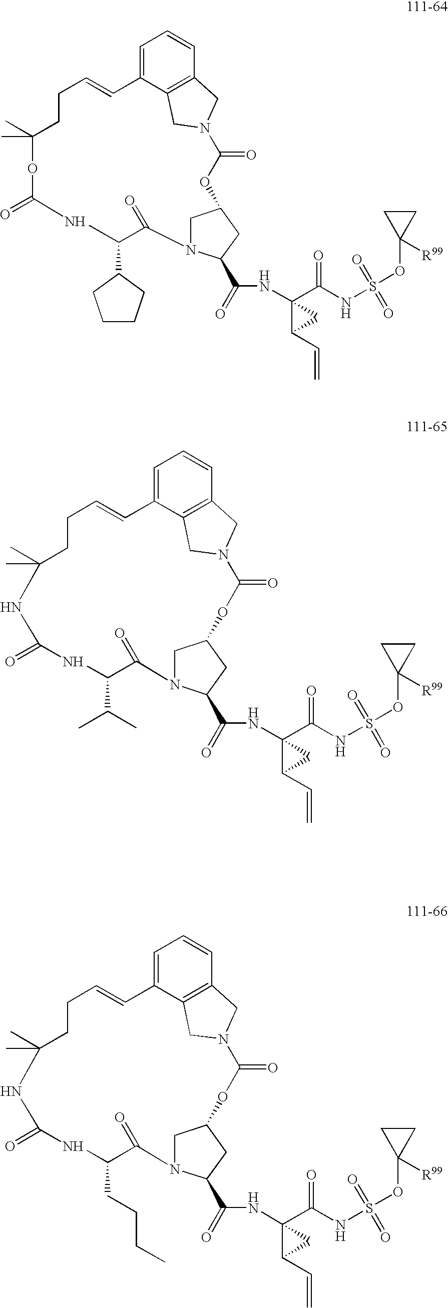

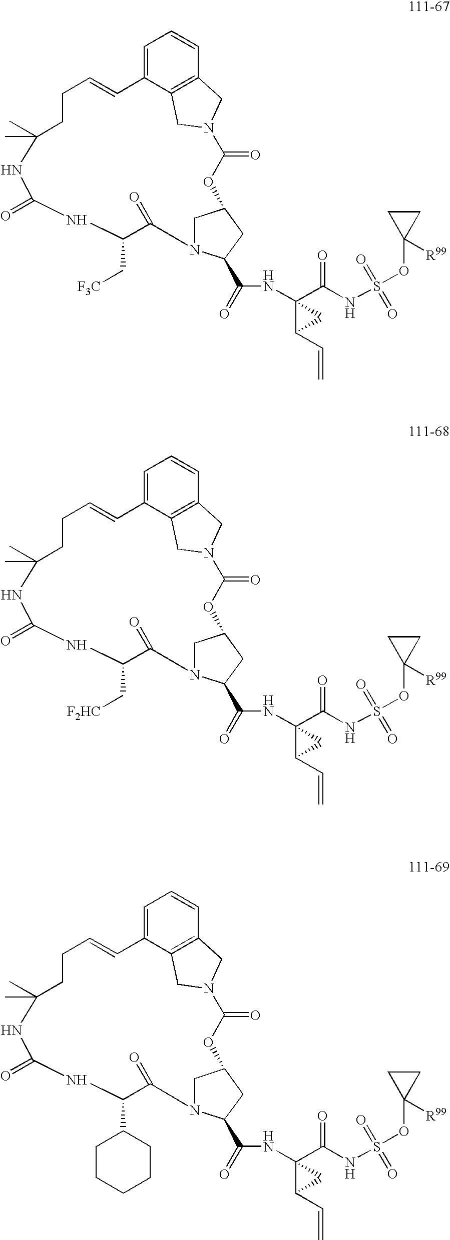

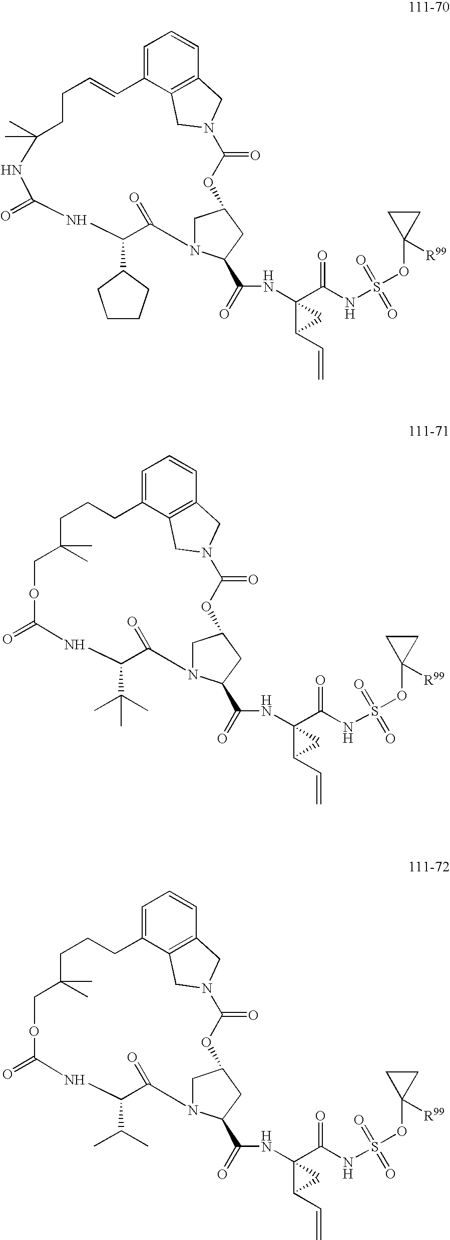

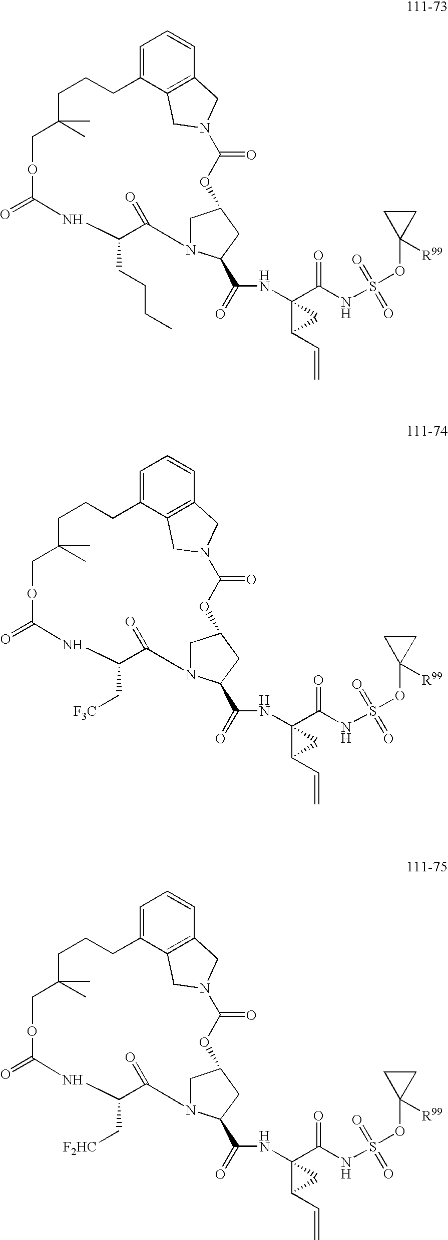

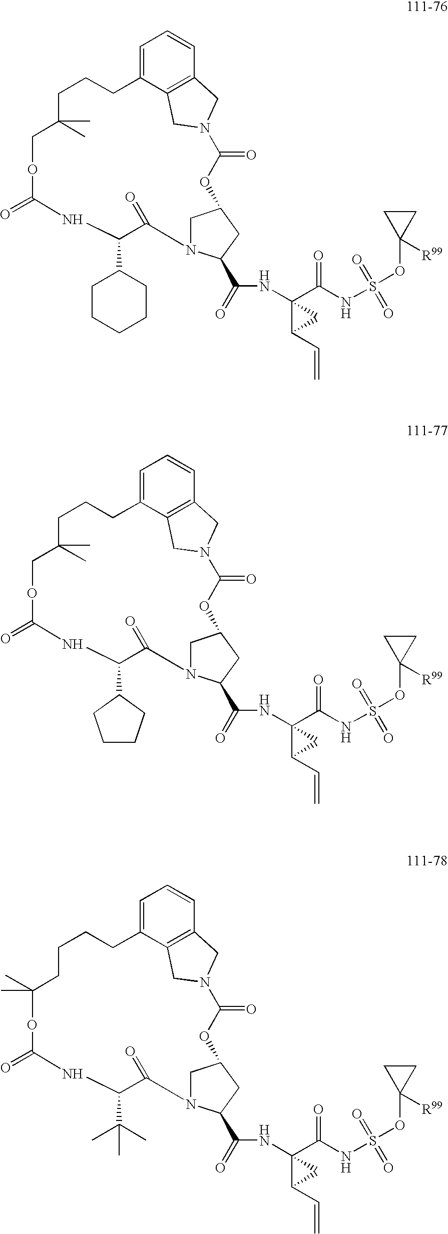

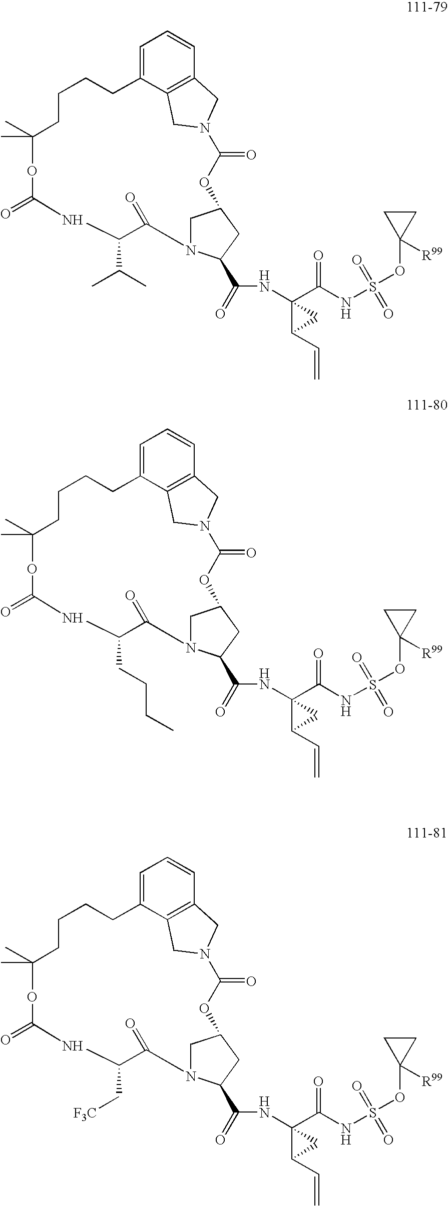

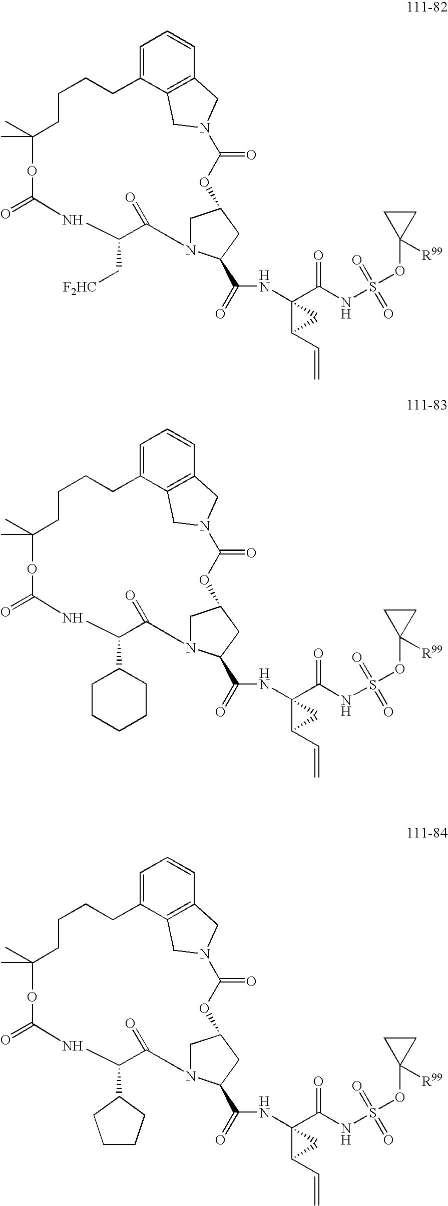

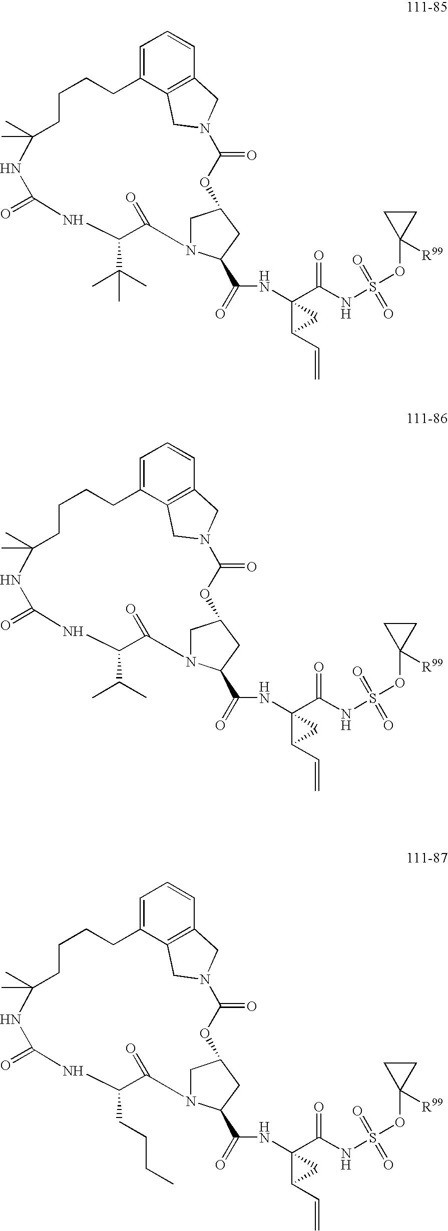

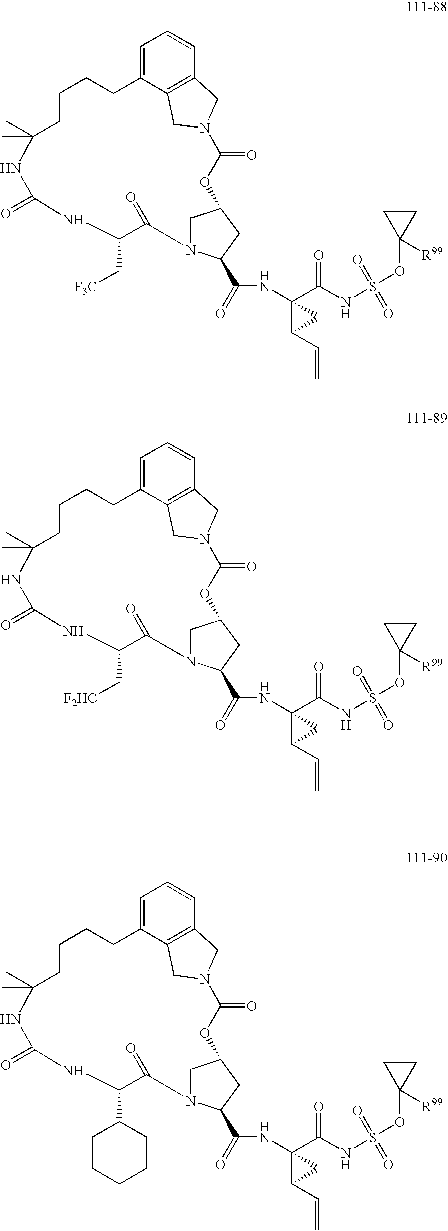

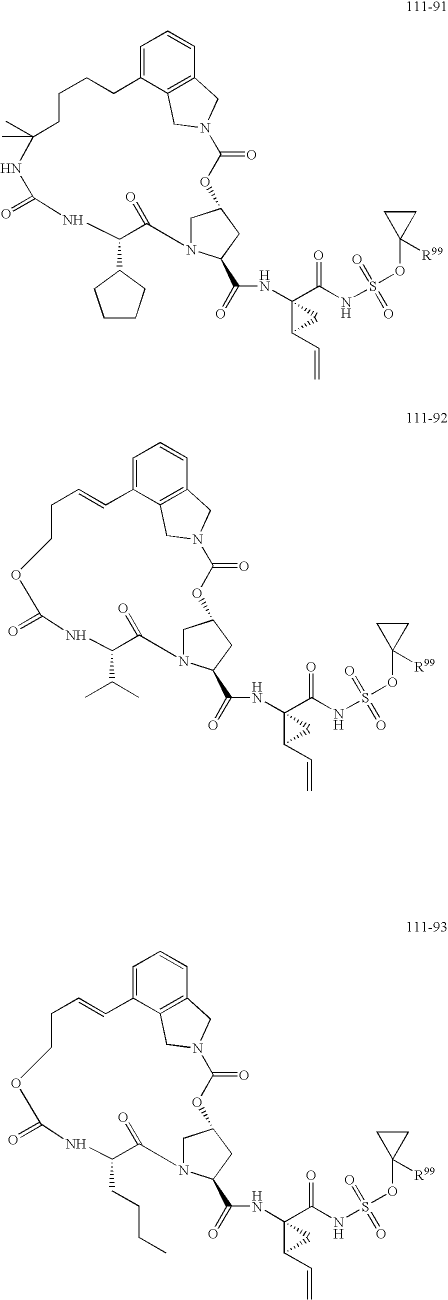

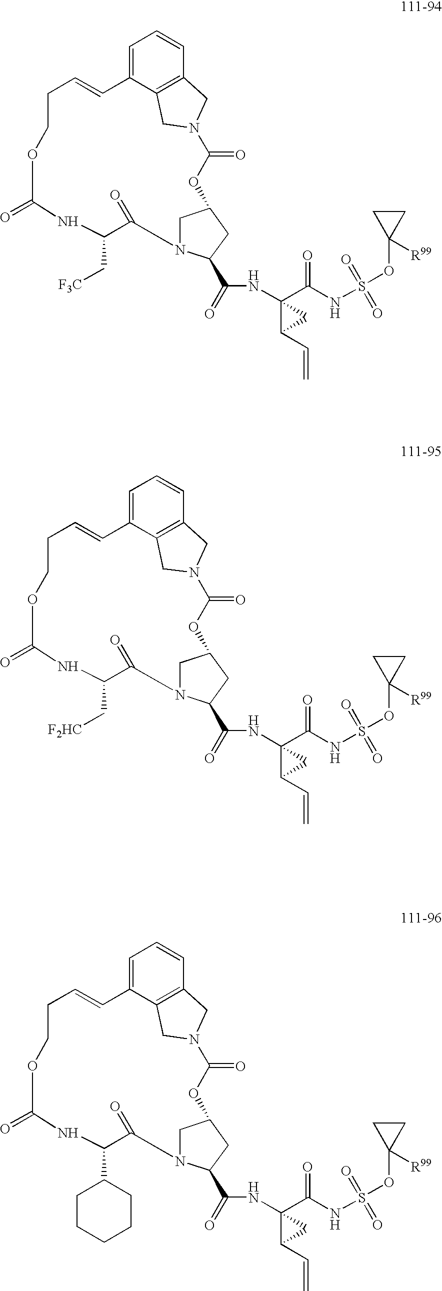

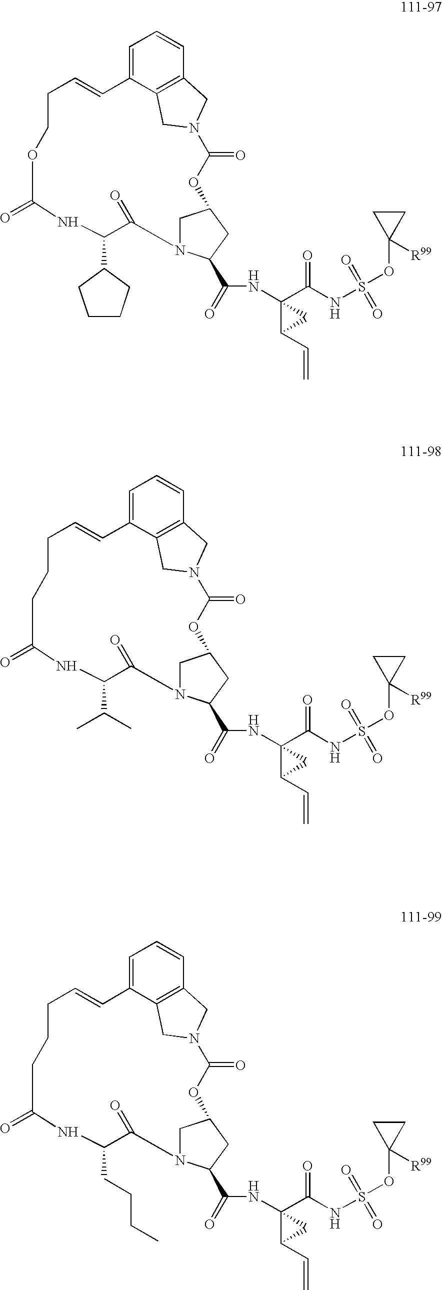

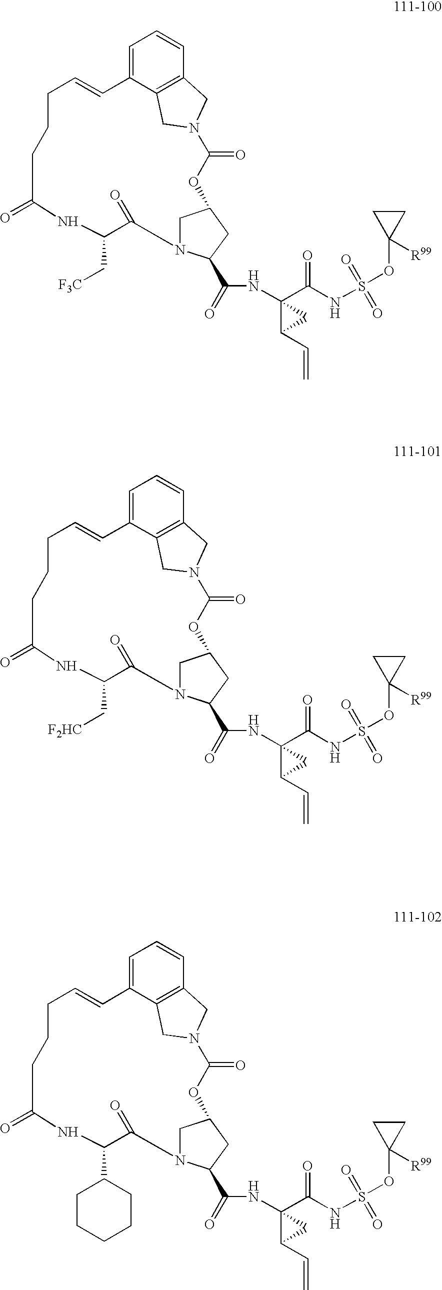

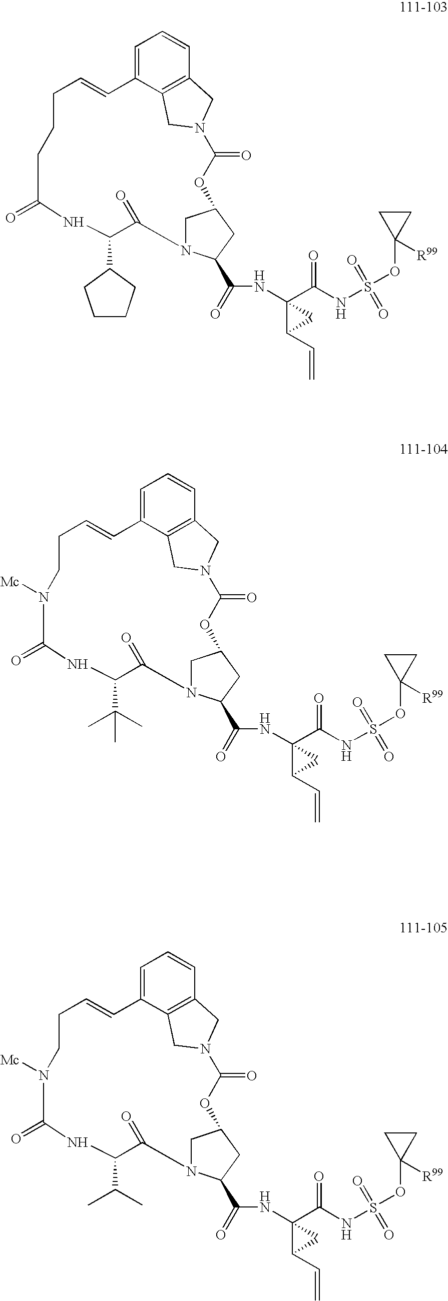

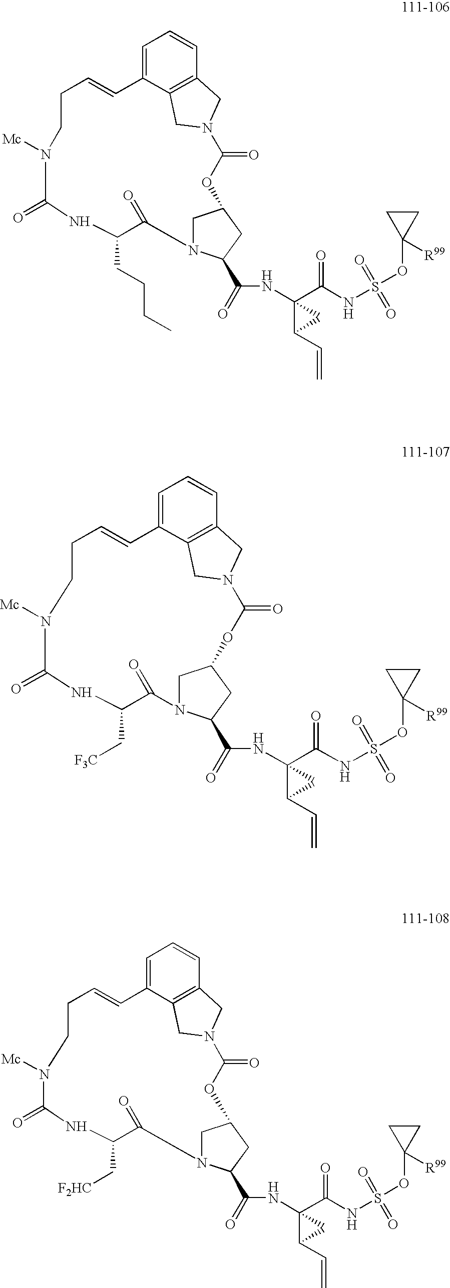

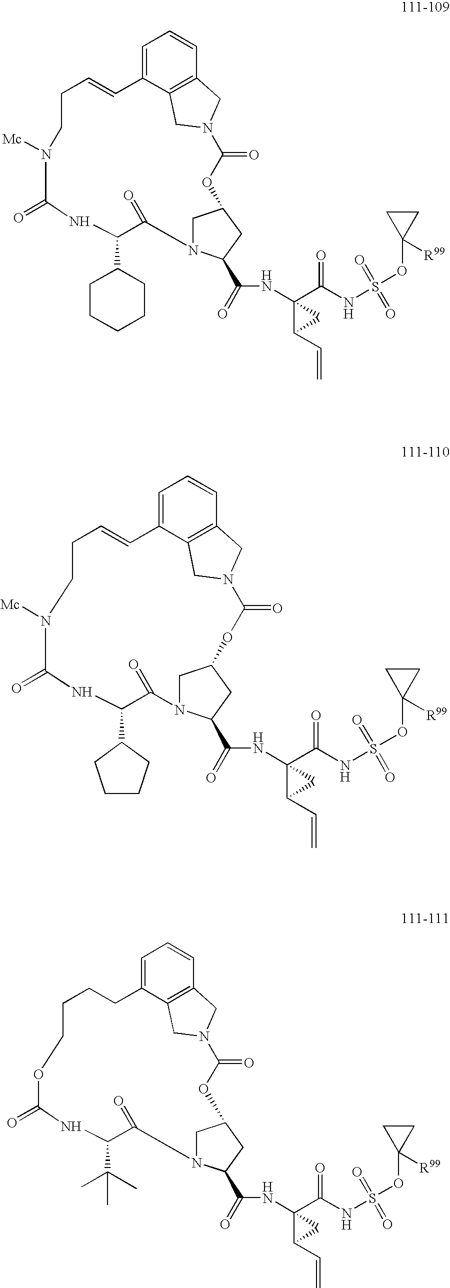

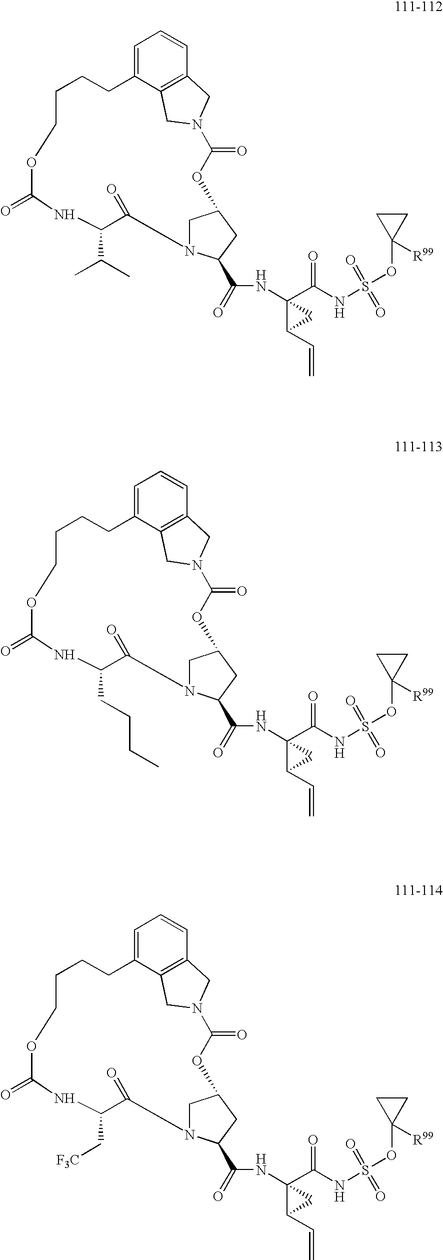

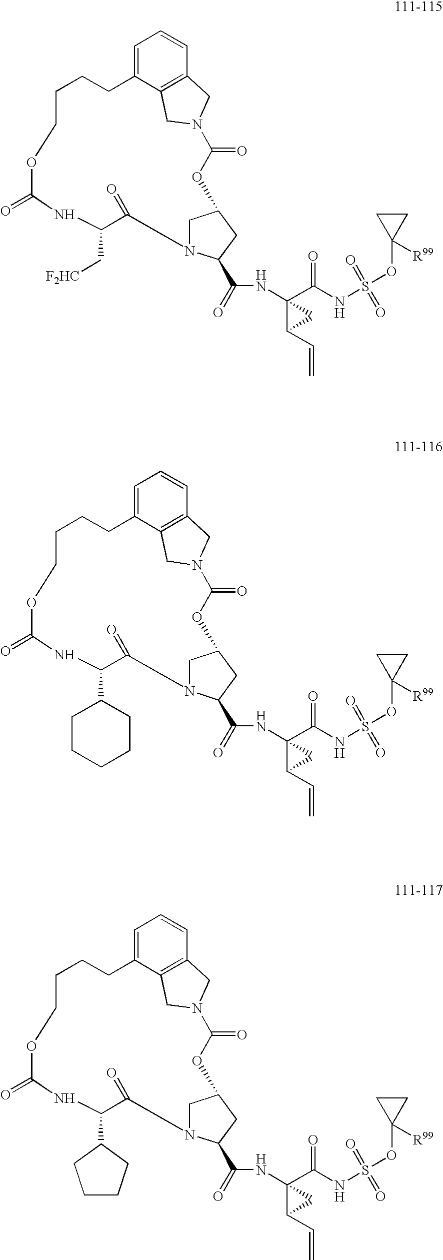

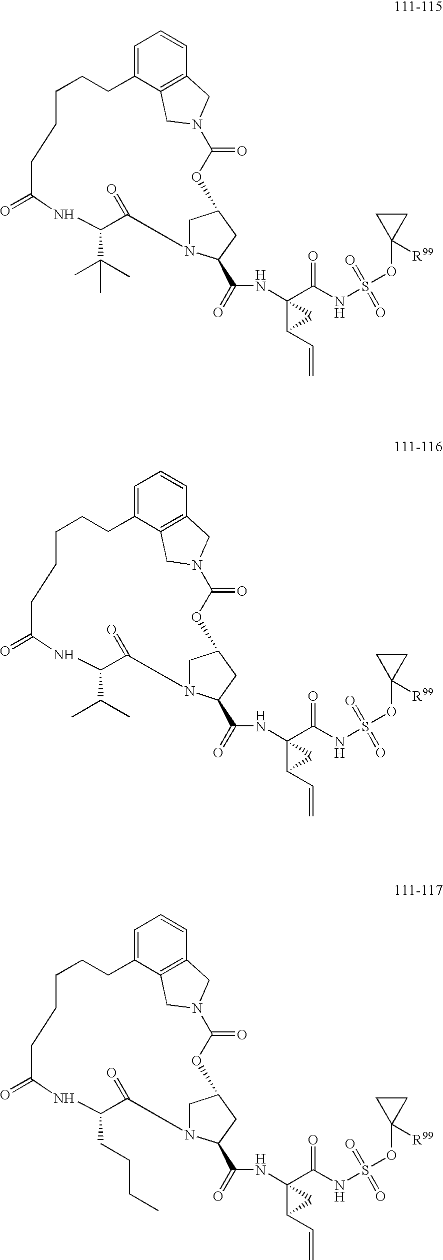

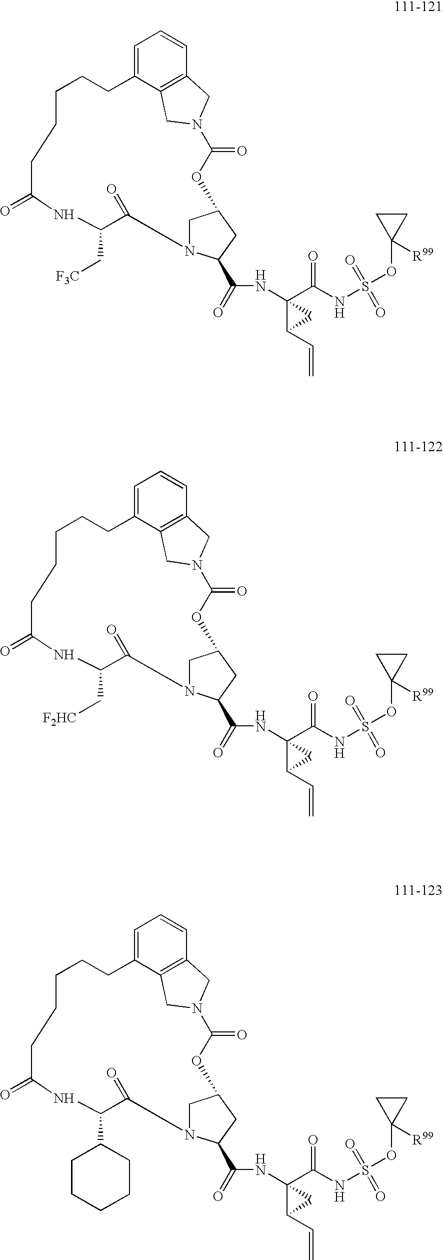

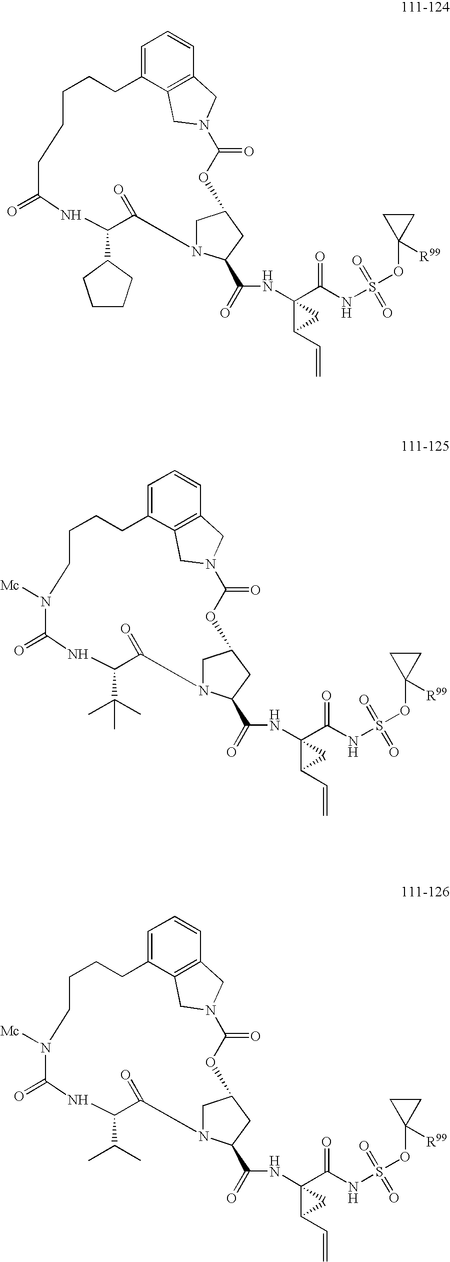

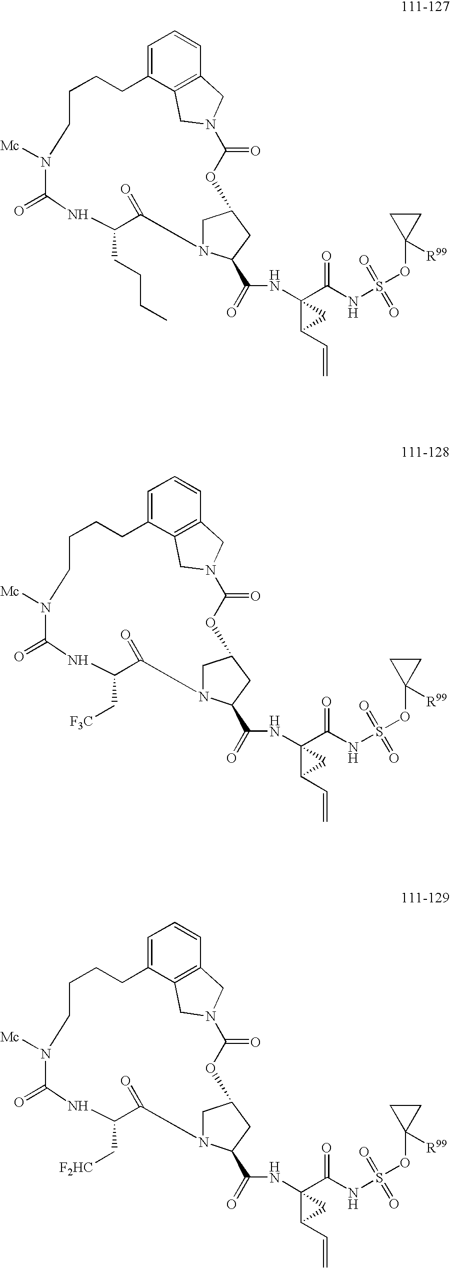

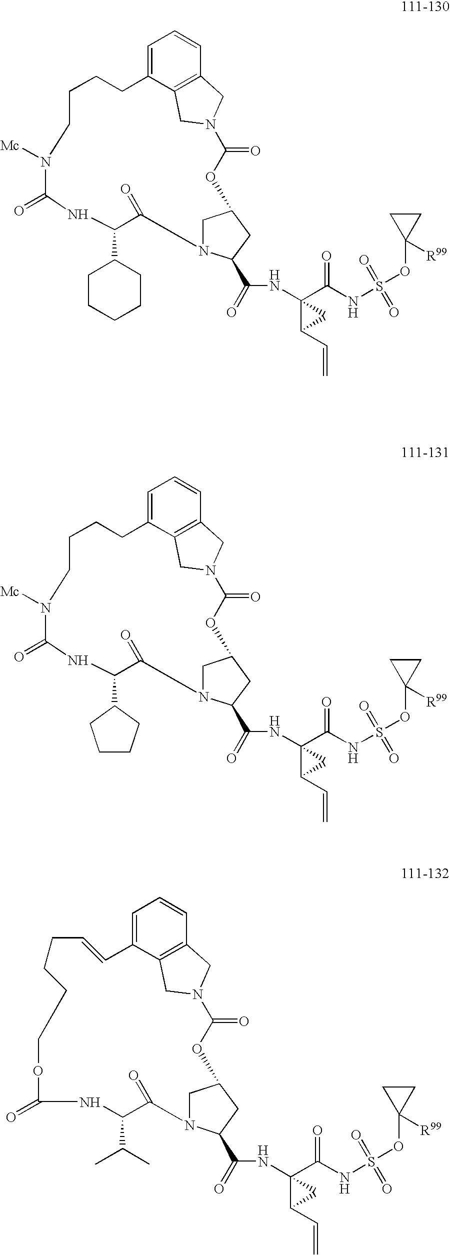

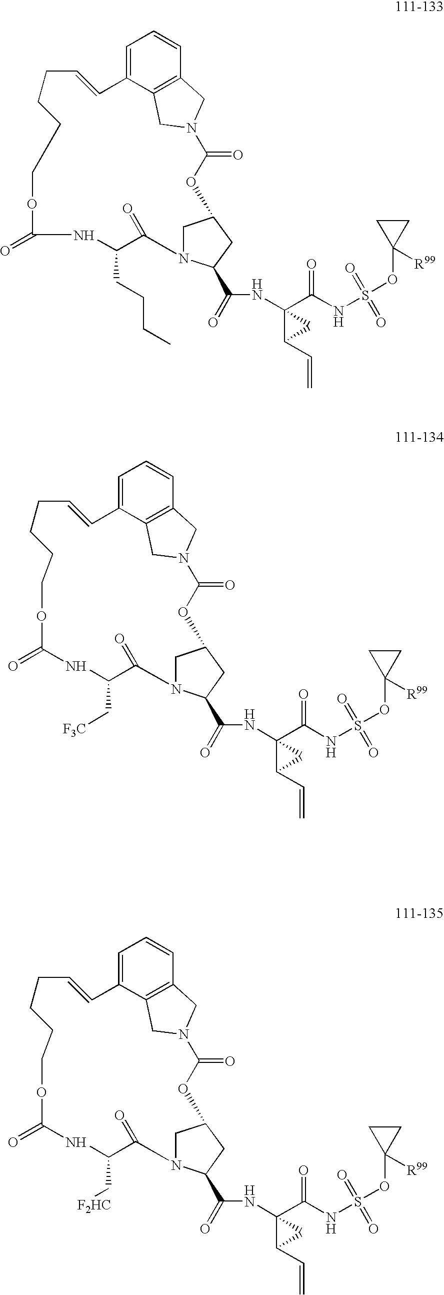

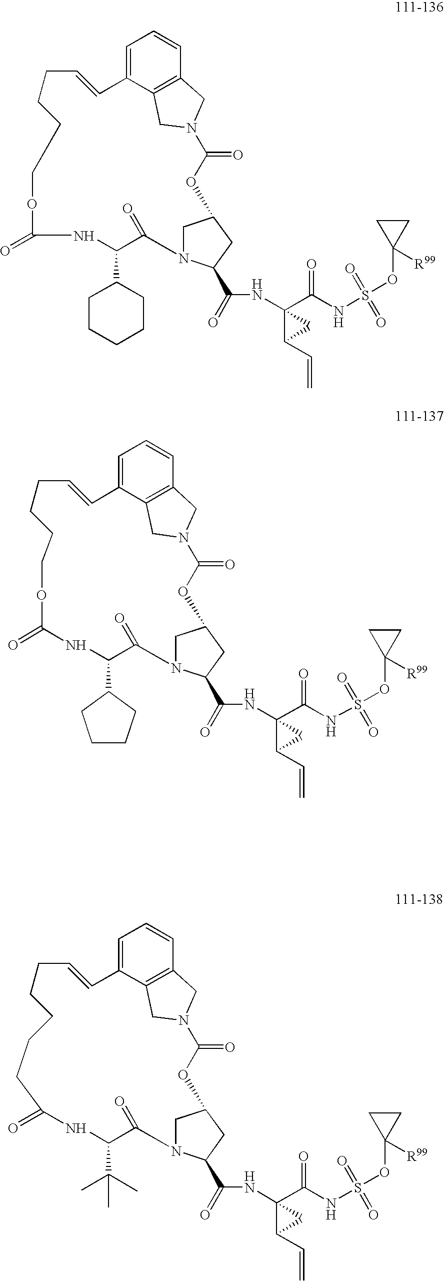

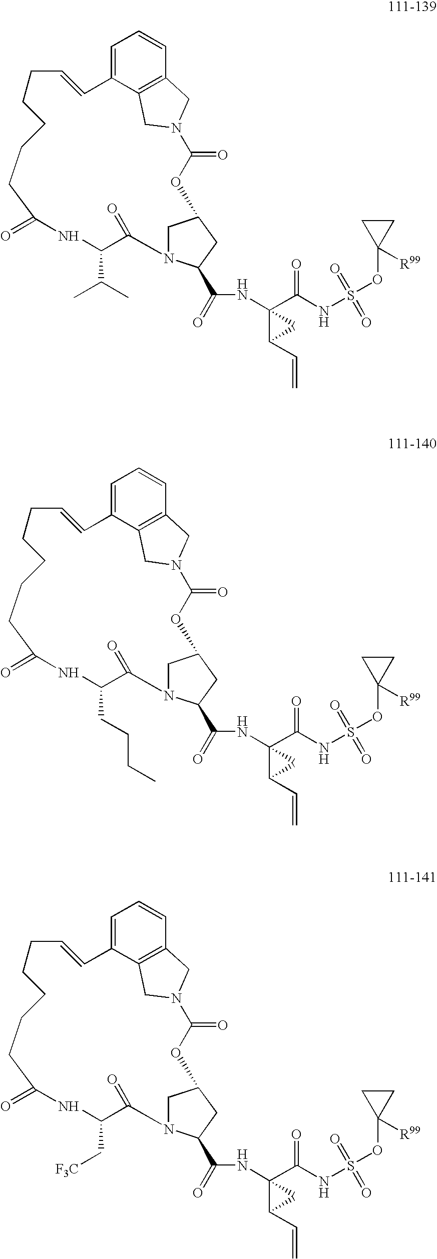

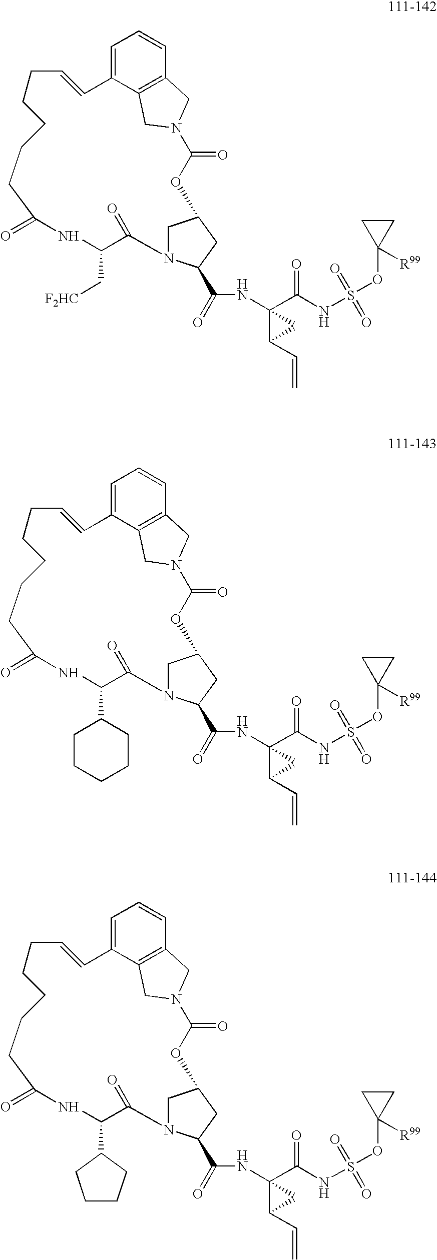

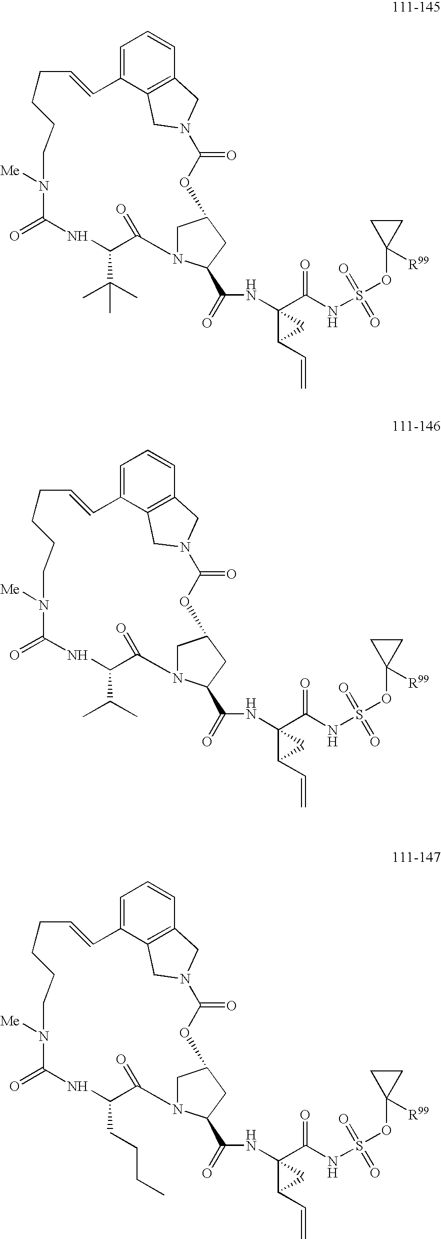

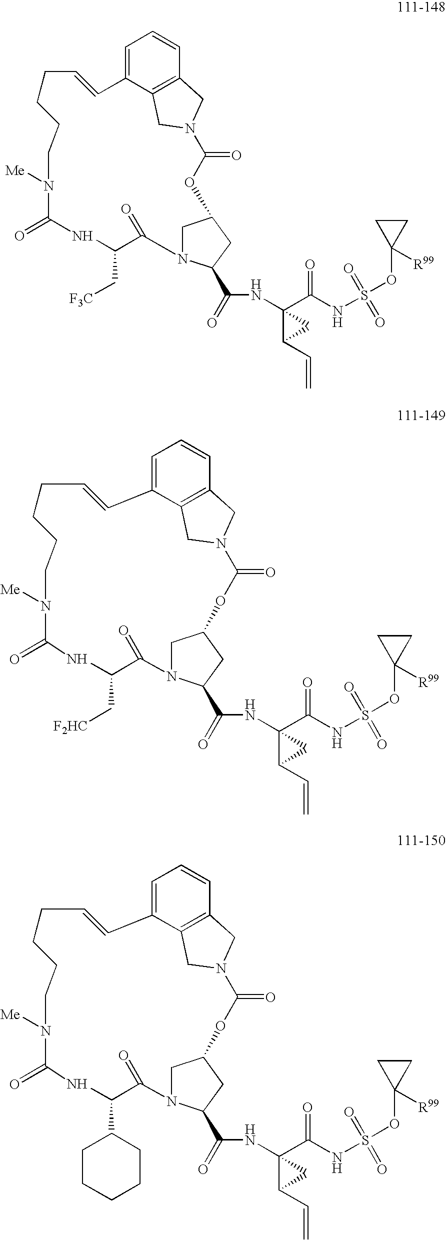

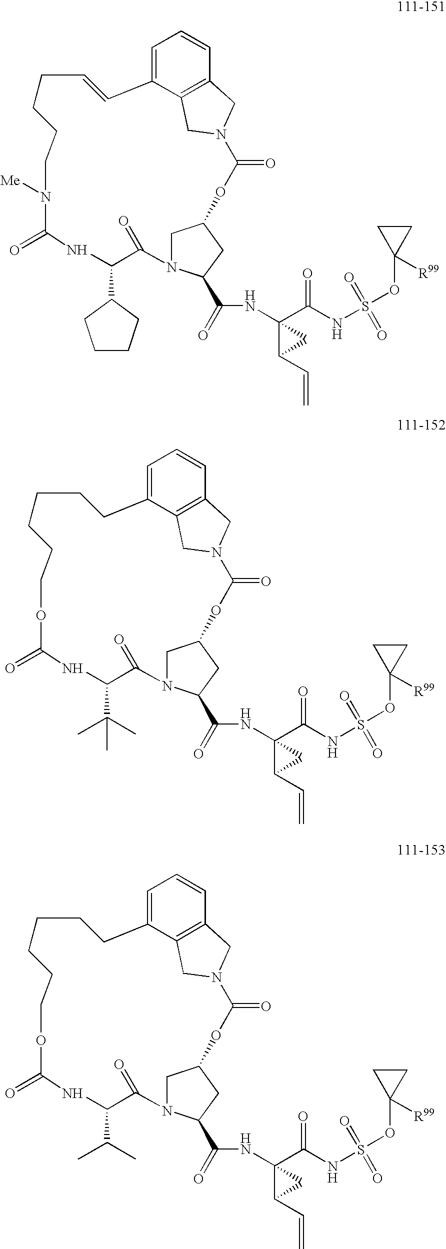

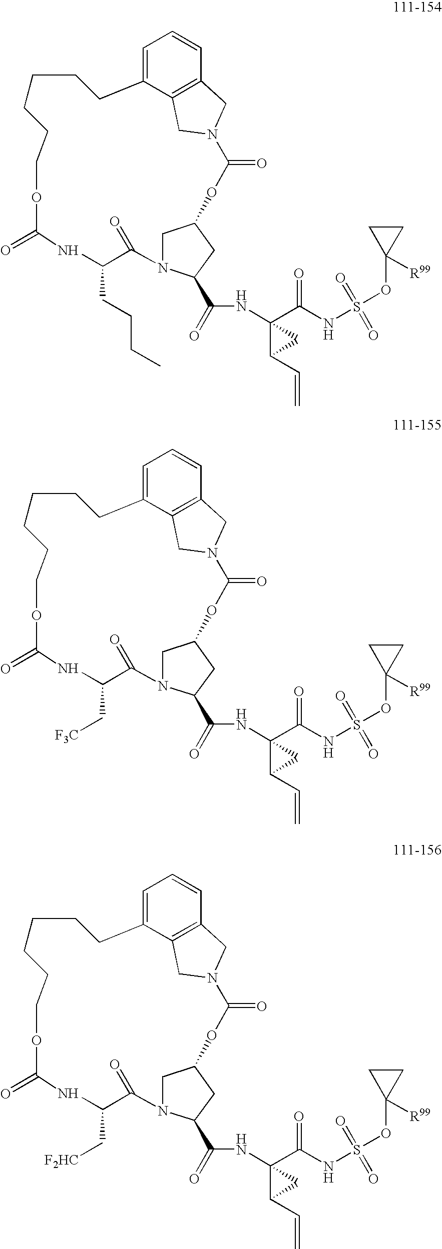

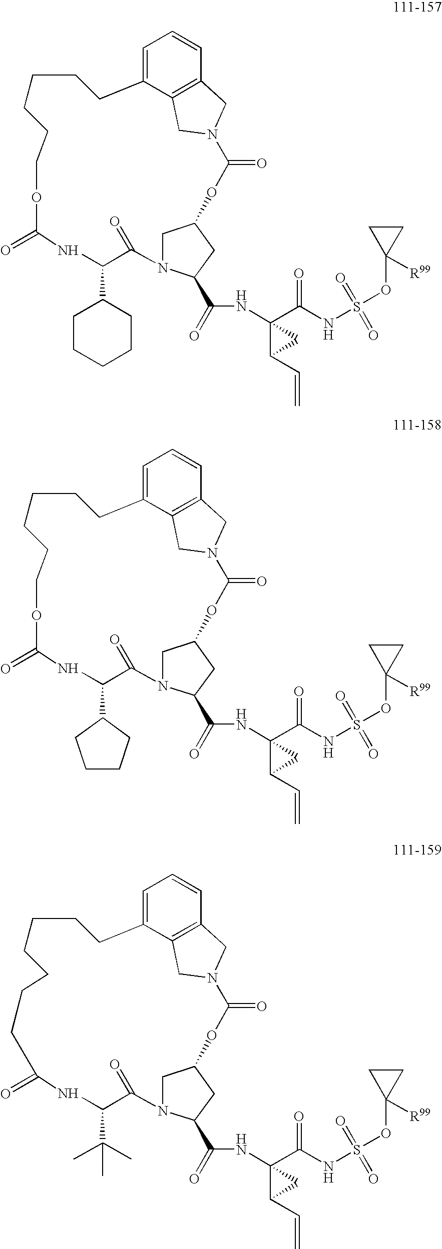

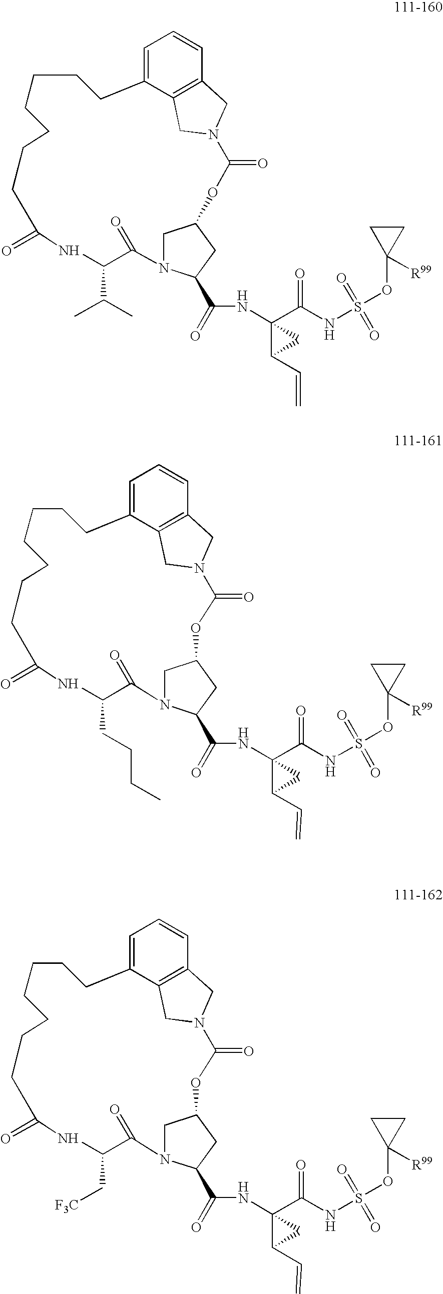

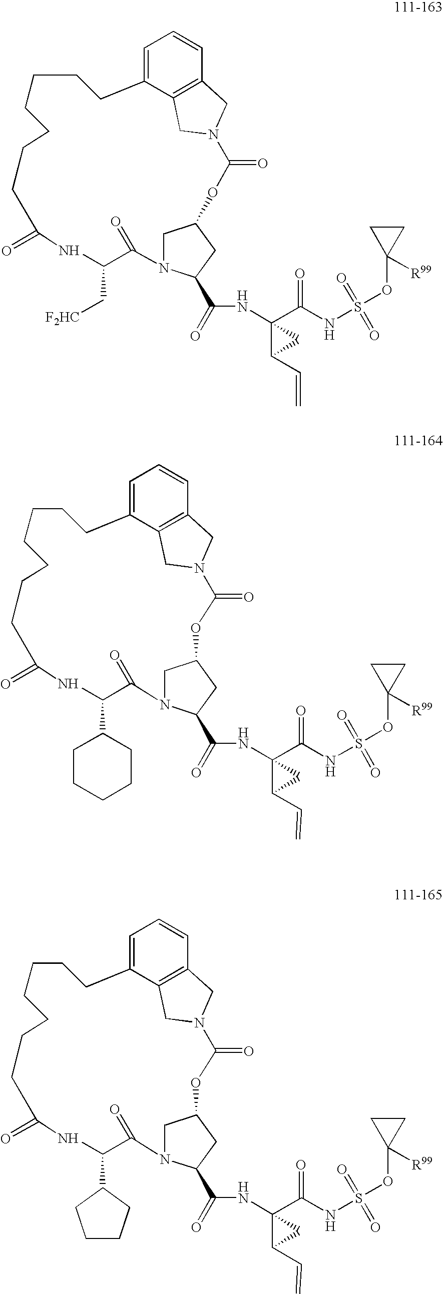

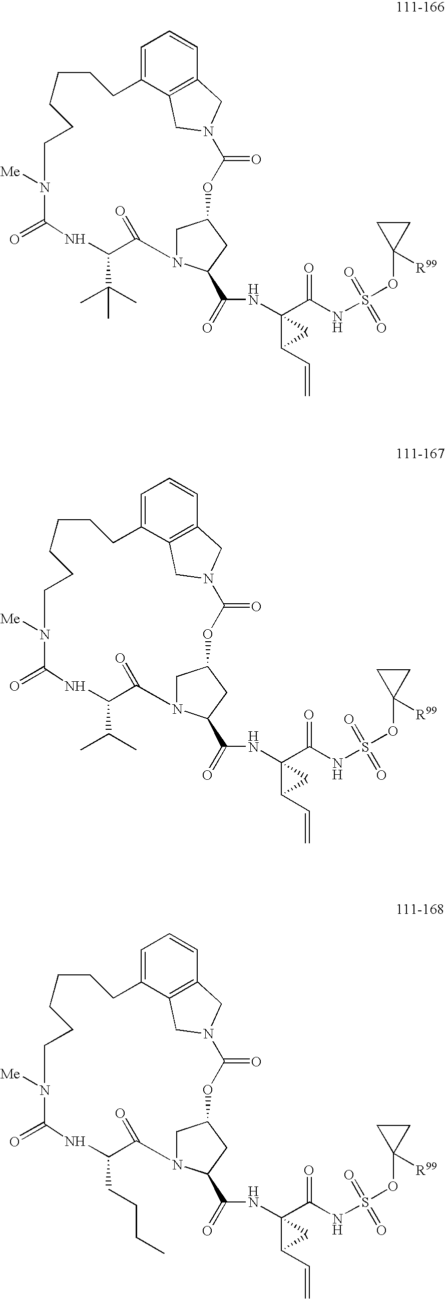

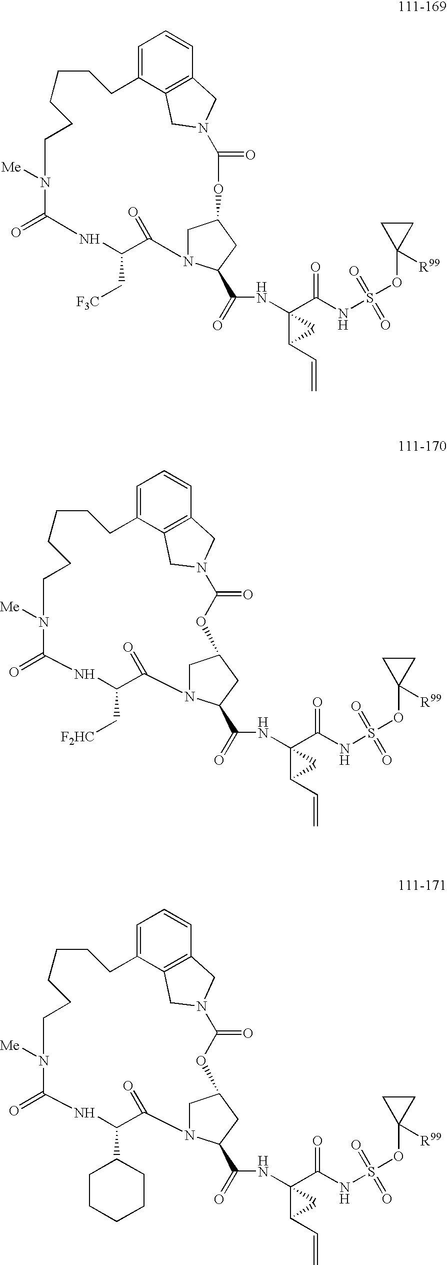

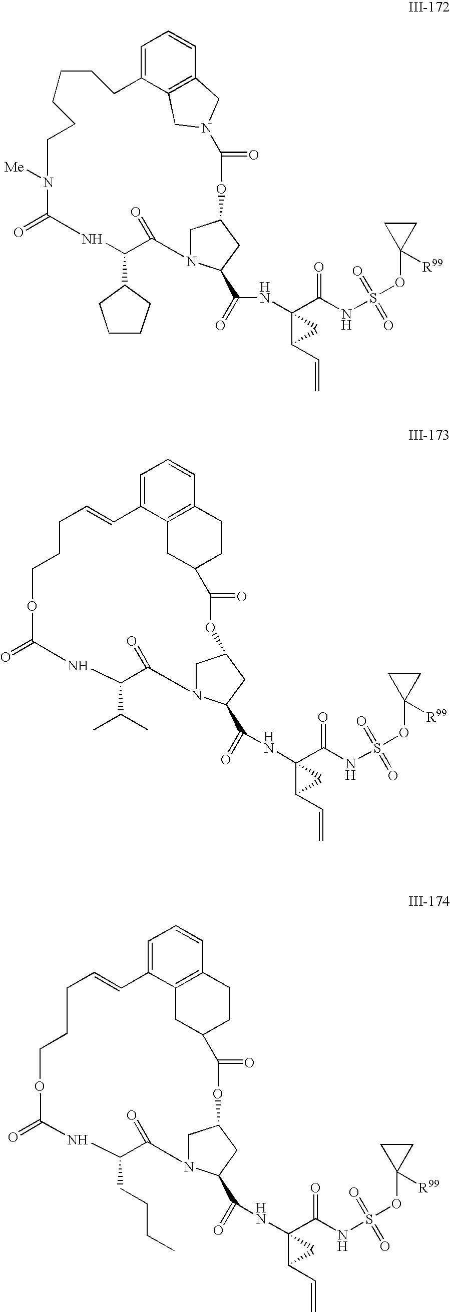

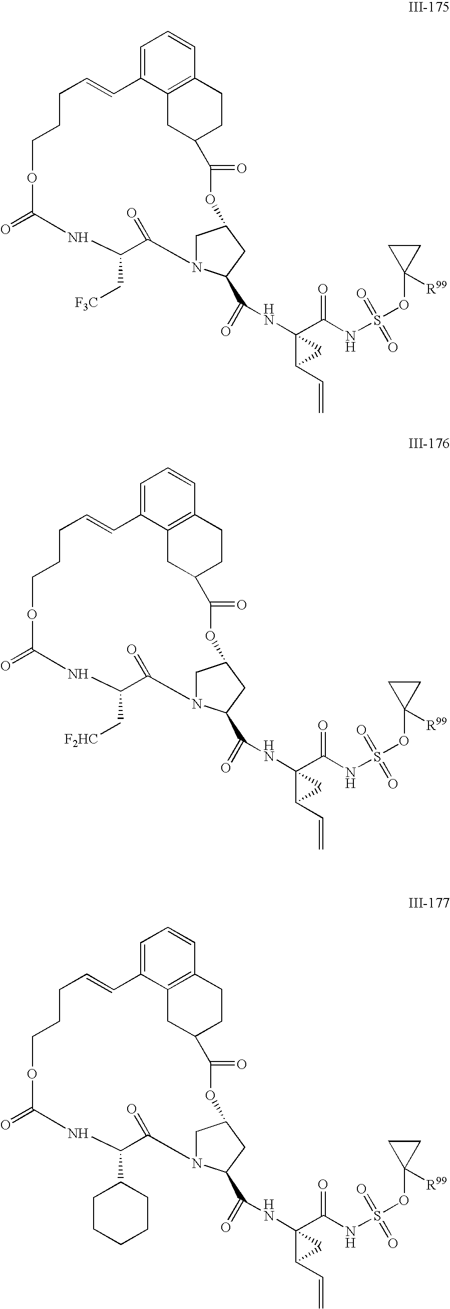

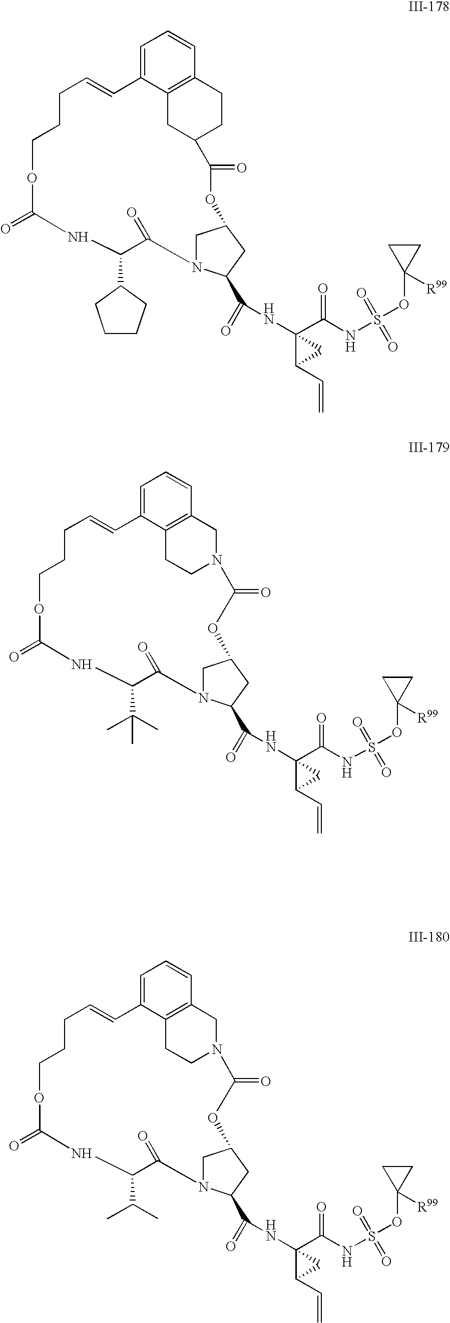

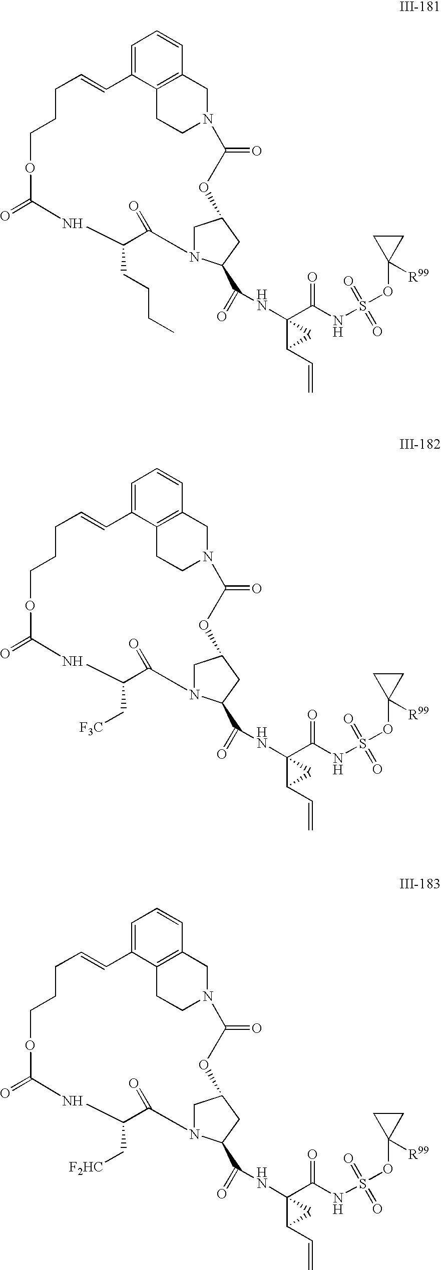

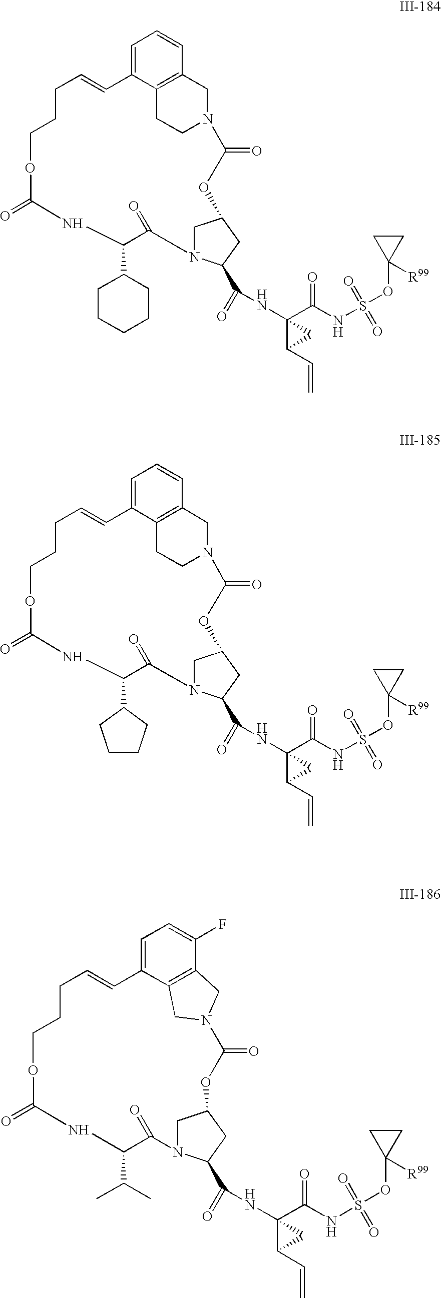

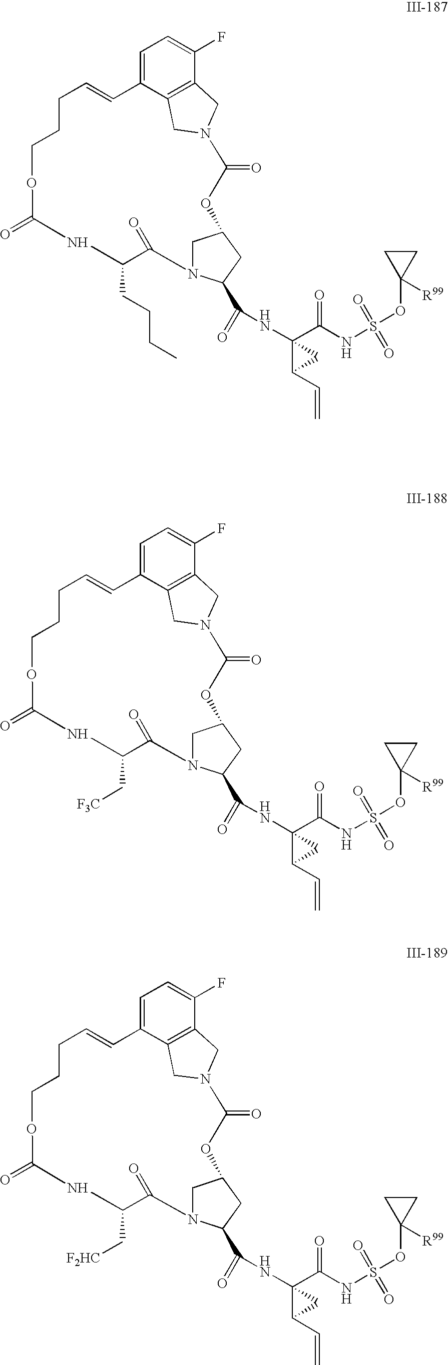

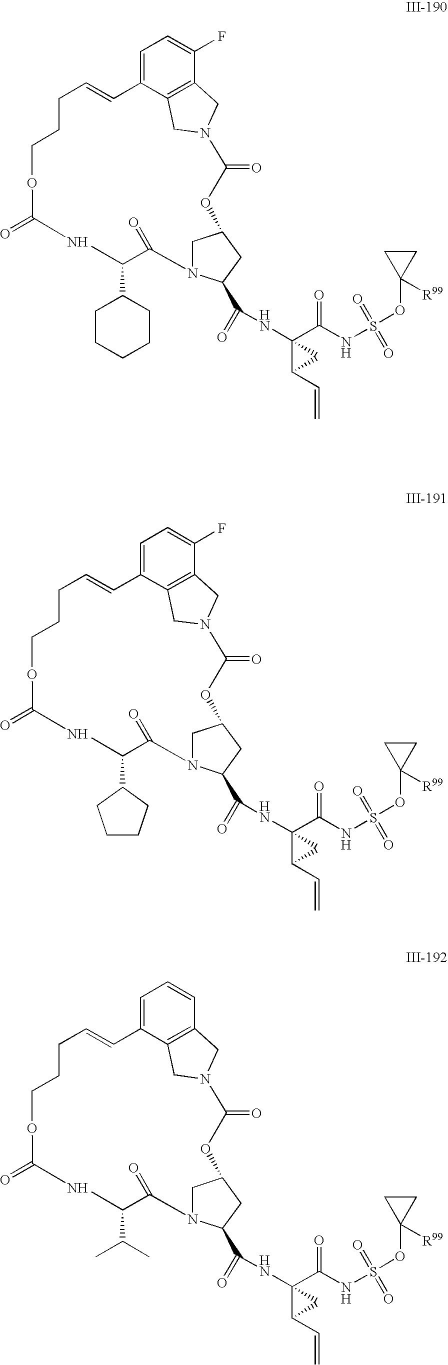

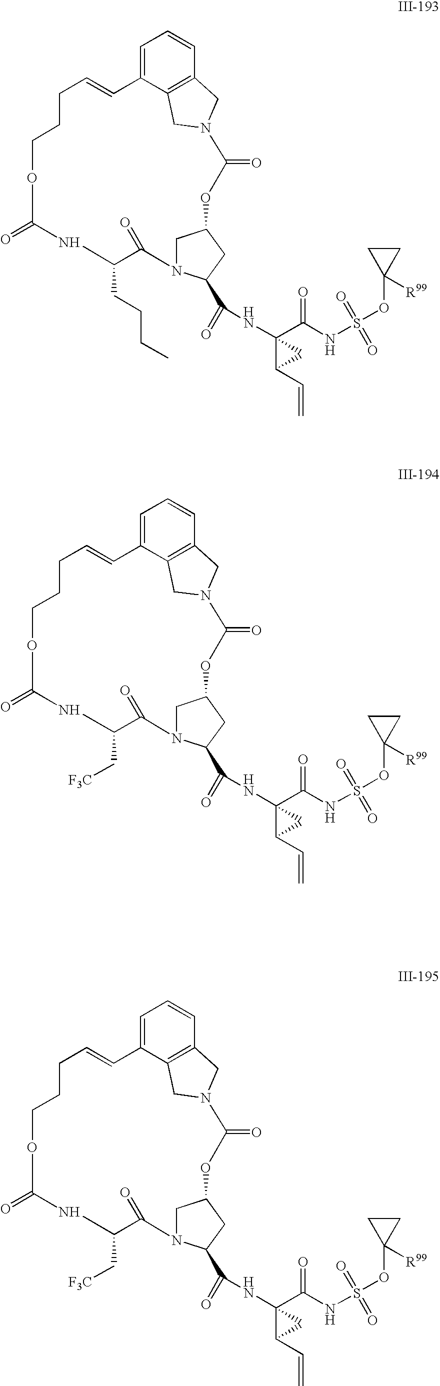

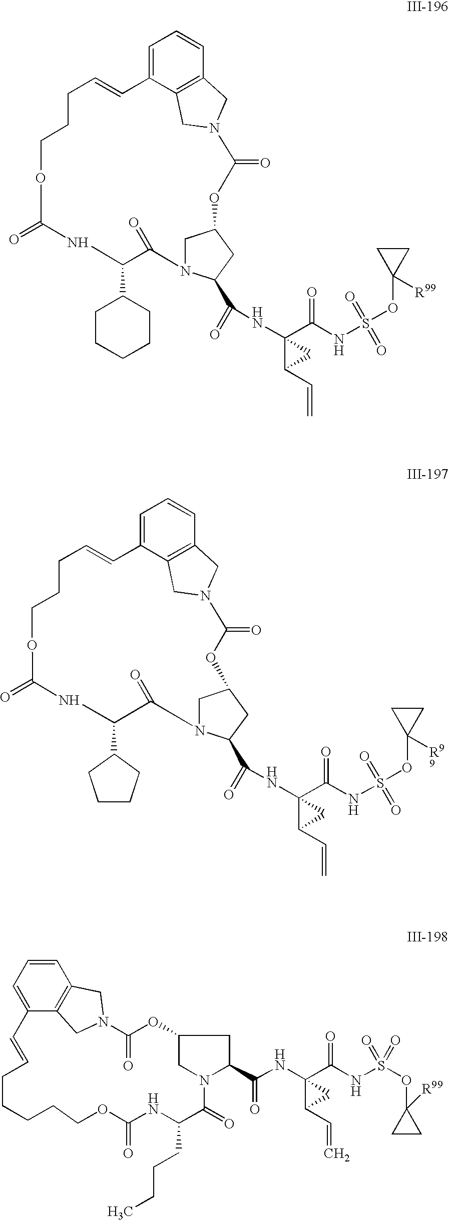

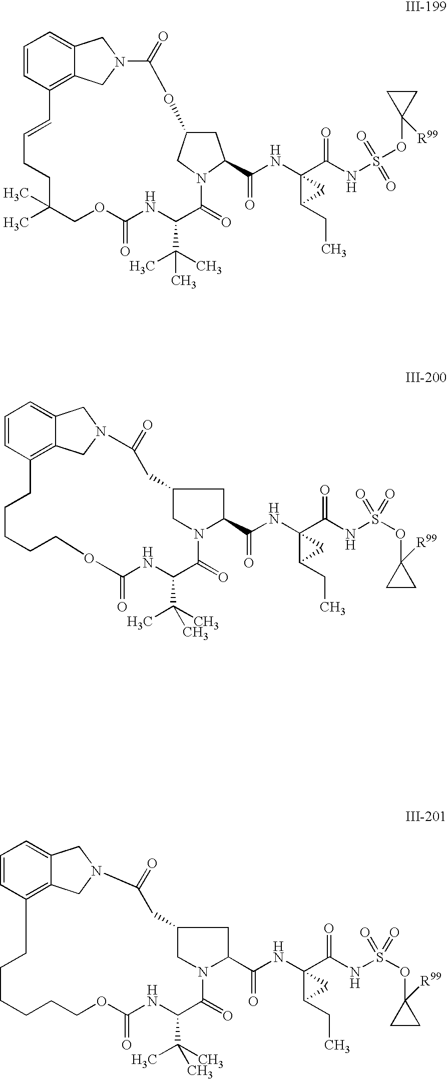

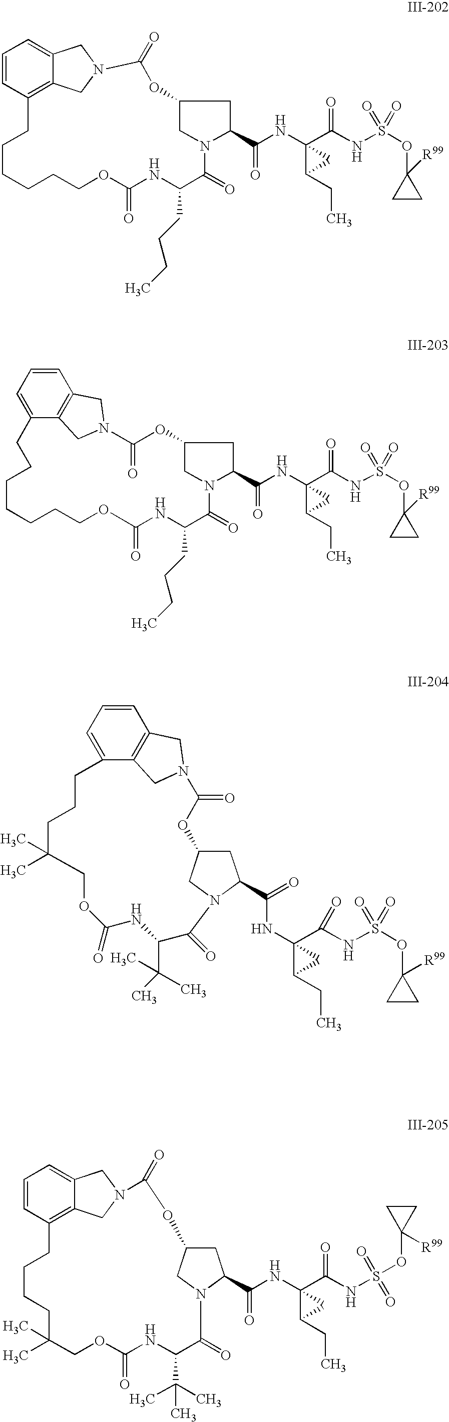

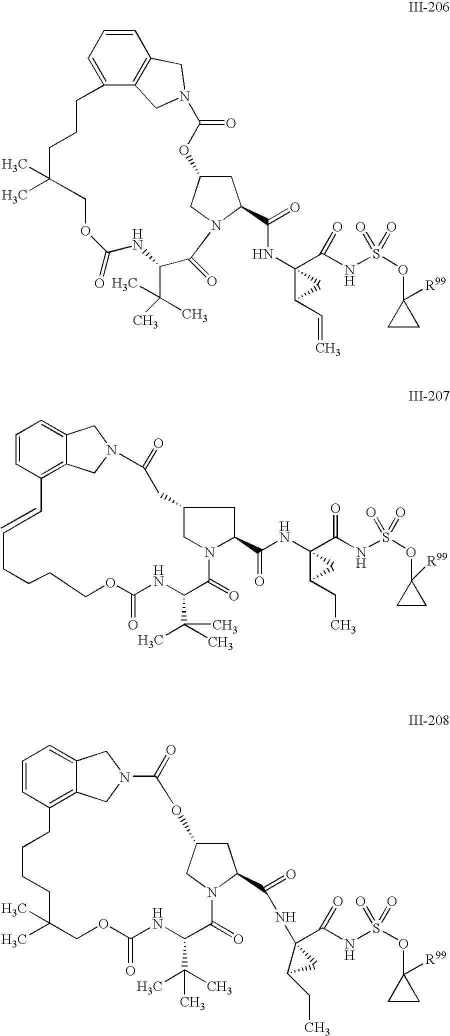

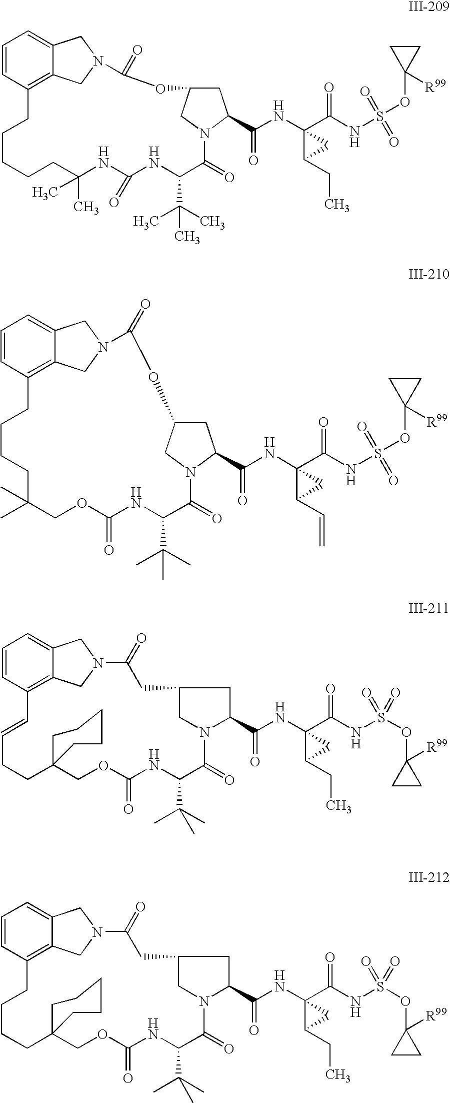

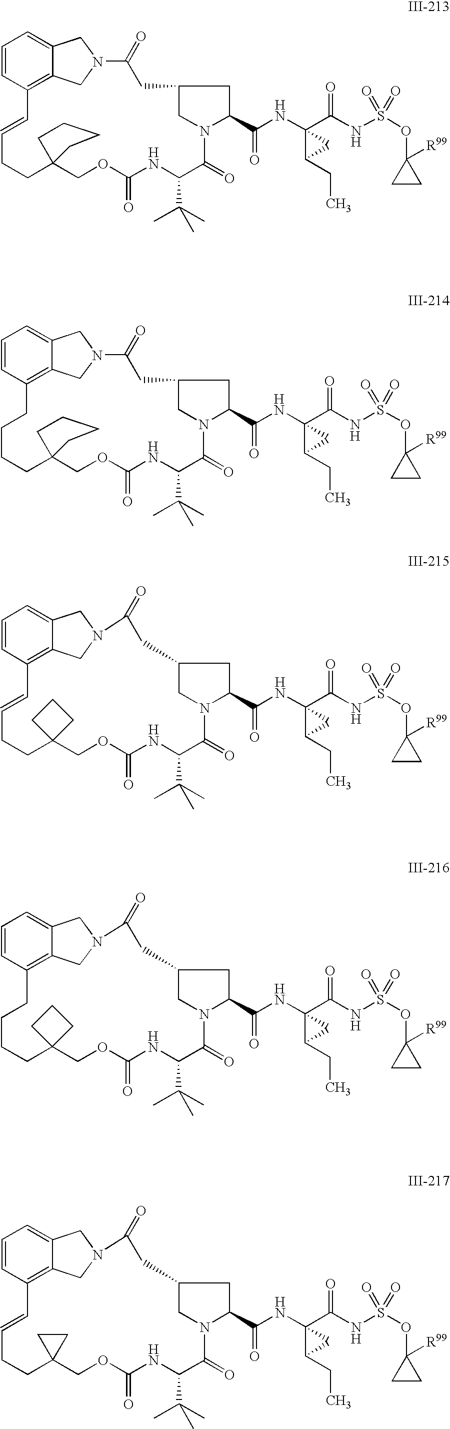

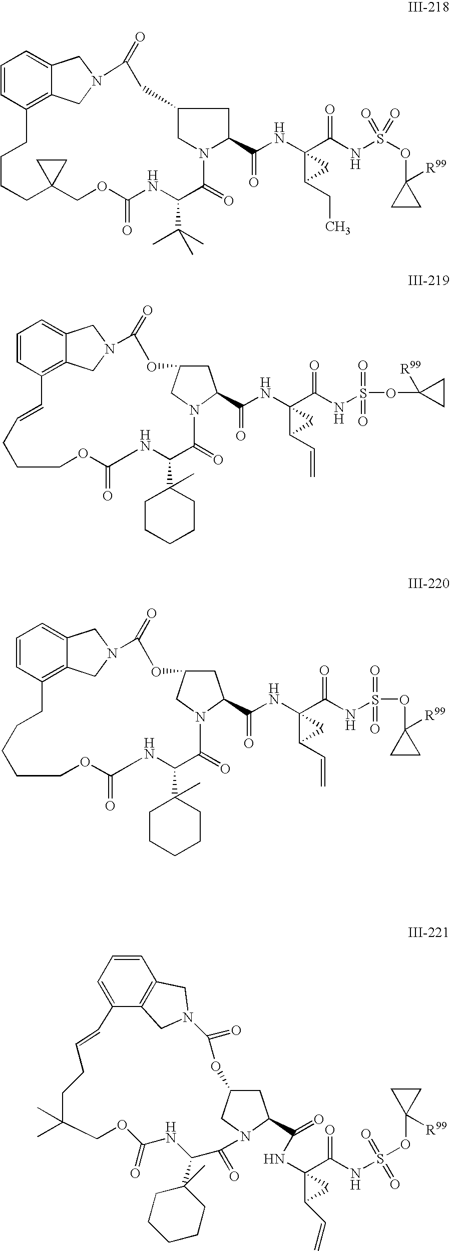

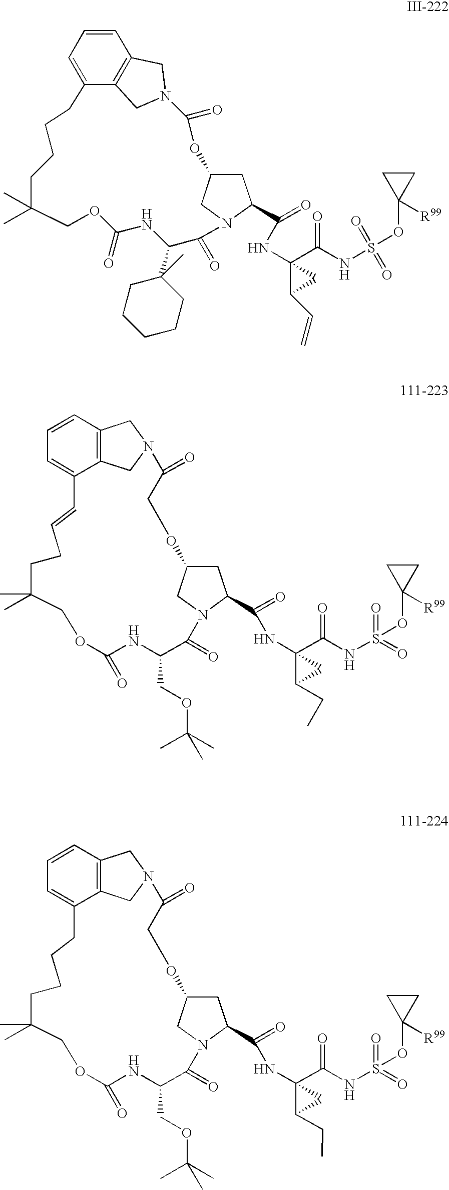

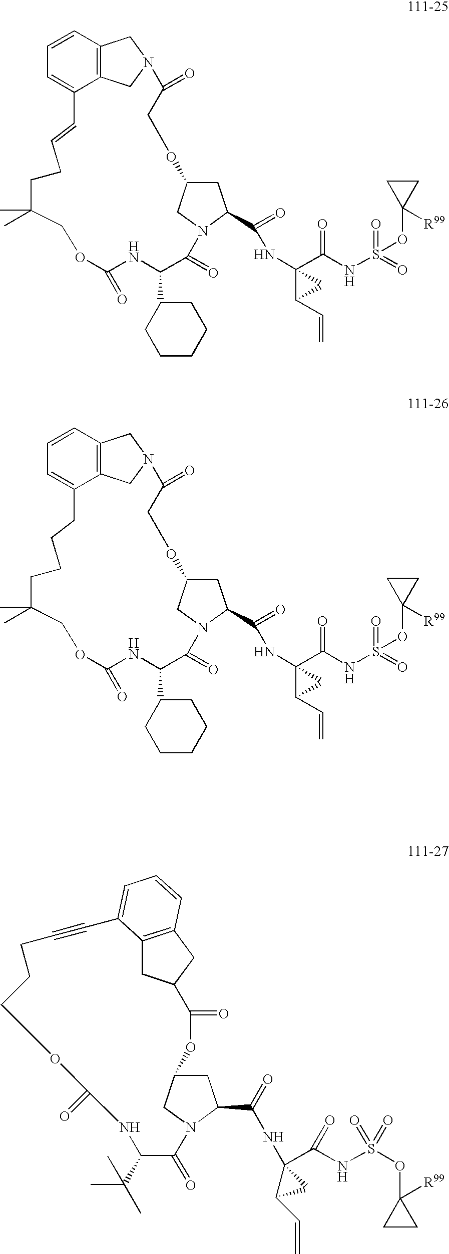

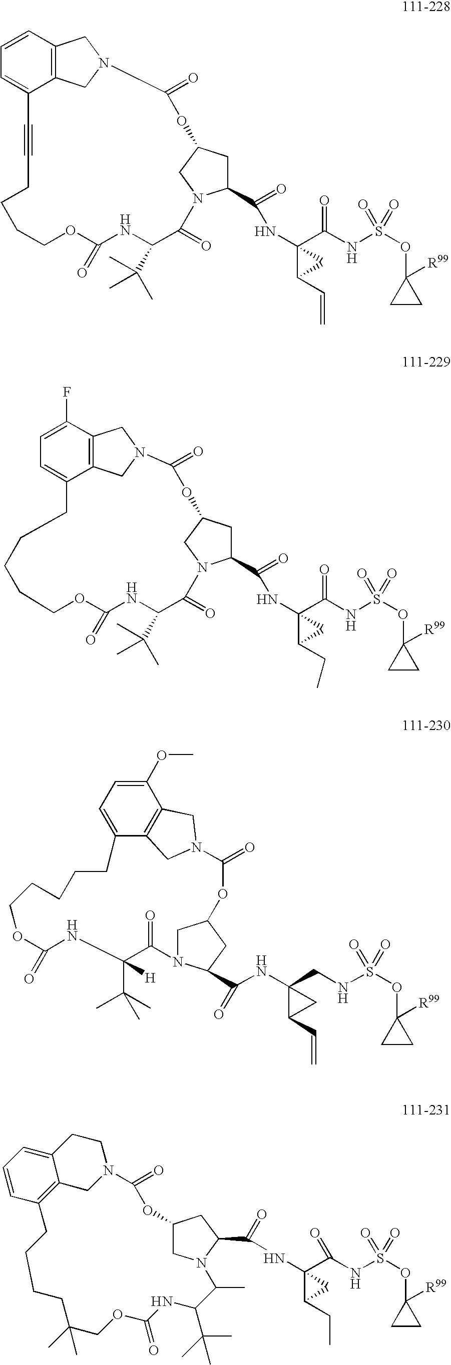

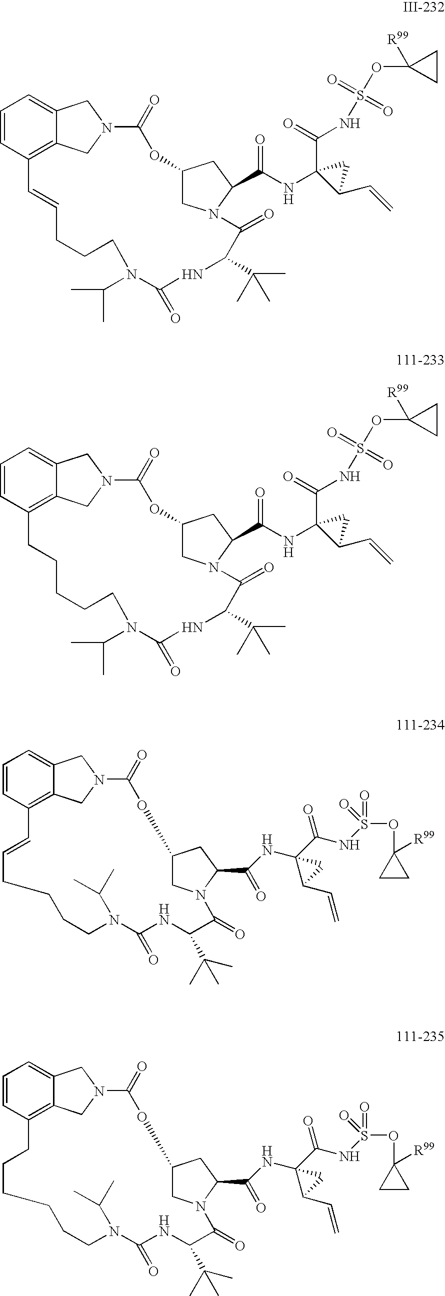

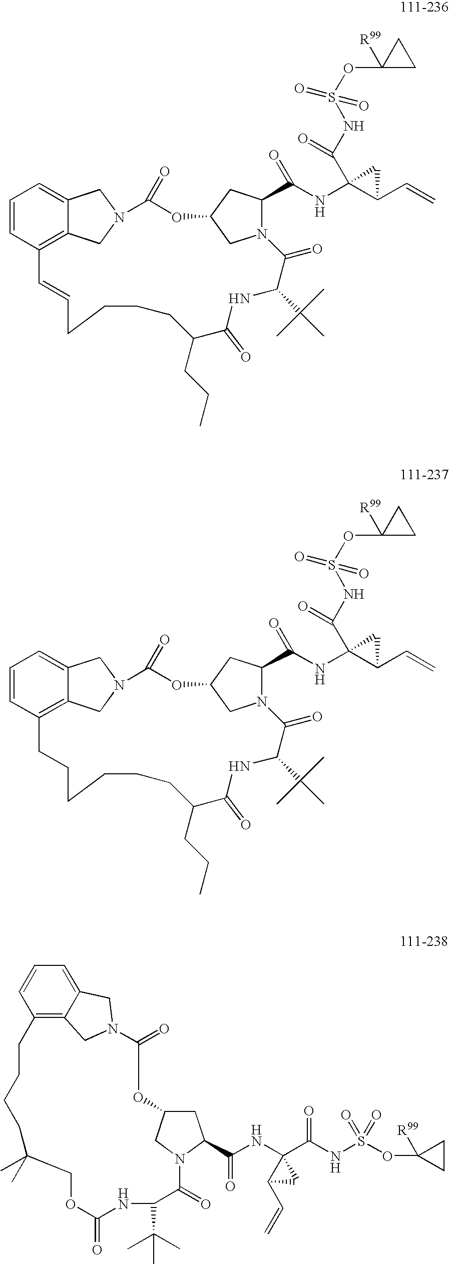

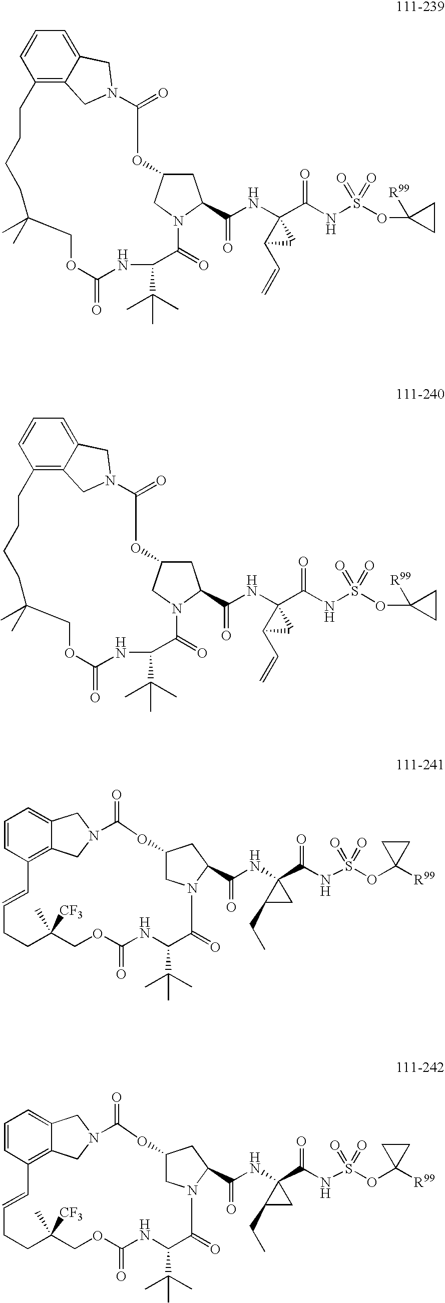

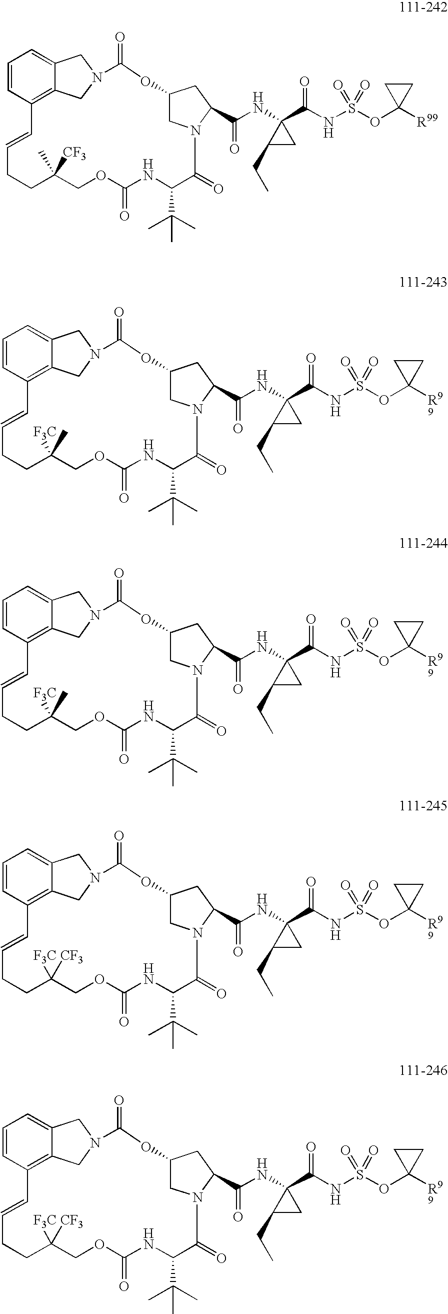

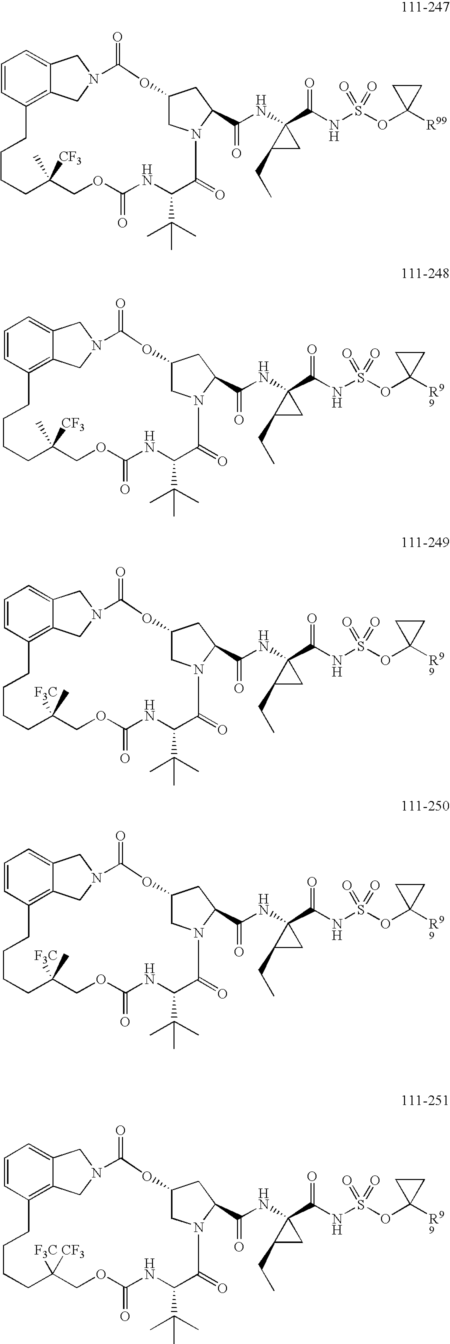

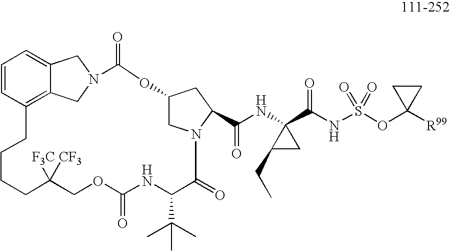

39. A pharmaceutical composition comprising an effective amount of a compound of claim 1, and a pharmaceutically acceptable carrier.

40. The pharmaceutical composition of claim 39, further comprising a second therapeutic agent selected from the group consisting of a HCV antiviral agent, an immunomodulator, and an anti-infective agent.

41. The pharmaceutical composition of claim 40, wherein the HCV antiviral agent is an antiviral selected from the group consisting of a HCV protease inhibitor and a HCV NS5B polymerase inhibitor.

42. A use of a compound of claim 1 in the preparation of a medicament for inhibiting HCV N53 protease activity in a subject in need thereof.

43. A use of a compound of claim 1 in the preparation of a medicament for preventing or treating infection by HCV in a subject in need thereof.

44. The use of claim 43, wherein said medicament further comprises at least one second therapeutic agent selected from the group consisting of a HCV antiviral agent, an immunomodulator, and an anti-infective agent.]

45. The use of claim 44, wherein the HCV antiviral agent is an antiviral selected from the group consisting of a HCV protease inhibitor and a HCV NS5B polymerase inhibitor.
